(19) United States
(12) Patent Application Publication
(10) Pub. No.: US 2025/0075216 A1
(43) Pub. Date: Mar. 6, 2025

(54) Treatment Of Muscle Disorder With Folliculin Interacting Protein 1 (FNIP1) Inhibitors And/Or Folliculin (FLCN) Inhibitors

(71) Applicant: Regeneron Pharmaceuticals, Inc., Tarrytown, NY (US)

(72) Inventors: Luca Andrea Lotta, Tarrytown, NY (US); George Hindy, Tarrytown, NY (US); Viktoria Gusarova, Tarrytown, NY (US); Mark Sleeman, Tarrytown, NY (US); Rene Christian Adam, Tarrytown, NY (US)

(21) Appl. No.: 18/817,818

(22) Filed: Aug. 28, 2024

Related U.S. Application Data
(60) Provisional application No. 63/535,082, filed on Aug. 29, 2023.
(60) Provisional application No. 63/547,633, filed on Nov. 7, 2023.

(51) Int. Cl. C12N 15/113; A61K 45/06; A61P 21/00; C12Q 1/6883

(52) U.S. Cl. CPC C12N 15/1135; A61K 45/06; A61P 21/00; C12Q 1/6883; C12N 2310/11; C12N 2310/14; C12Q 2600/156

(57) ABSTRACT

The present disclosure generally relates to the treatment of subjects having muscle disorder or at risk of developing muscle disorder by administering a Folliculin Interacting Protein 1 (FNIP1) inhibitor and/or a Folliculin (FLCN) inhibitor to the subject.

### REFERENCE TO SEQUENCE LISTING

This application includes a Sequence Listing filed electronically as an XML file named 381204284SEQ, created on Aug. 21, 2024, with a size of 106,382 bytes. The Sequence Listing is incorporated herein by reference.

### FIELD

The present disclosure generally relates to the treatment of subjects having muscle disorder or at risk of developing muscle disorder by administering a Folliculin Interacting Protein 1 (FNIP1) inhibitor and/or a Folliculin (FLCN) inhibitor to the subject, and to methods of identifying subjects having an increased risk of developing muscle disorder.

### BACKGROUND

Causes of muscle disorders include: injury or overuse, such as sprains or strains, cramps or tendinitis; a genetic disorder, such as muscular dystrophy; some cancers; inflammation, such as myositis, diseases of nerves that affect muscles; infections; and certain medicines. Muscle disorders include sarcopenia, Duchenne Muscular Dystrophy, and Pompe disease.

Folliculin (FLCN) is encoded by a 25 kb gene located at 17p11.2. FLCN is 579 amino acids long and is a 64 kDa multi-functional protein involved in both the cellular response to amino acid availability and in the regulation of glycolysis. Specifically, FLCN regulates the mTORC1 signaling cascade controlling the MiT/TFE factors TFEB and TFE3 in the cellular response to amino acid availability and regulates glycolysis by binding to lactate dehydrogenase LDHA, acting as an uncompetitive inhibitor. In addition, FLCN activates mTORC1 by acting as a GTPase-activating protein by stimulating GTP hydrolysis by RRAGC/RagC or RRAGD/RagD, promoting the conversion to the GDP-bound state of RRAGC/RagC or RRAGD/RagD, and thereby activating the kinase activity of mTORC1.

Folliculin Interacting Protein 1 (FNIP1) is encoded by a 155 kb gene located at 5q31.1. FNIP1 is 1166 amino acids long and is a 130 kDa protein that participates in the regulation of cellular metabolism and nutrient sensing by modulating the AMP-activated protein Kinase (AMPK) and target of rapamycin signaling pathways. In addition, FNIP1 binds to the tumor suppressor protein folliculin as well as associates with the molecular chaperone heat shock protein-90 (Hsp90) and negatively regulates its ATPase activity by facilitating its association with folliculin.

### SUMMARY

The present disclosure provides methods of treating a subject having muscle disorder or at risk of developing muscle disorder, the methods comprising administering an FNIP1 inhibitor and/or an FLCN inhibitor to the subject.

The present disclosure also provides methods of treating a subject having muscle disorder or at risk of developing muscle disorder by administering a muscle disorder therapeutic agent, the methods comprising: determining or having determined whether the subject has an FNIP1 variant nucleic acid molecule and/or an FLCN variant nucleic acid molecule, by: obtaining or having obtained a biological sample from the subject; and performing or having performed a sequence analysis on the biological sample to determine if the subject has a genotype comprising an FNIP1 variant nucleic acid molecule and/or an FLCN variant nucleic acid molecule; and administering or continuing to administer the muscle disorder therapeutic agent in a standard dosage amount, and/or administering an FNIP1 inhibitor and/or an FLCN inhibitor to a subject that is FNIP1 reference and/or FLCN reference; administering or continuing to administer the muscle disorder therapeutic agent in an amount that is the same as or less than a standard dosage amount, and/or administering an FNIP1 inhibitor and/or an FLCN inhibitor to a subject that is heterozygous for the FNIP1 variant nucleic acid molecule and/or the FLCN variant nucleic acid molecule; or administering or continuing to administer the muscle disorder therapeutic agent in an amount that is the same as or less than a standard dosage amount to a subject that is homozygous for the FNIP1 variant nucleic acid molecule and/or an FLCN variant nucleic acid molecule; wherein the presence the FNIP1 variant nucleic acid molecule and/or the FLCN variant nucleic acid molecule indicates the subject has a decreased risk of developing muscle disorder.

The present disclosure also provides methods of identifying a subject having an increased risk of developing muscle disorder, the methods comprising: determining or having determined the presence or absence of an FNIP1 variant nucleic acid molecule and/or an FLCN variant nucleic acid molecule in a biological sample obtained from the subject; wherein: when the subject is FNIP1 reference and/or FLCN reference, then the subject has an increased risk of developing muscle disorder; and when the subject is heterozygous or homozygous for the FNIP1 variant nucleic acid molecule and/or an FLCN variant nucleic acid molecule, then the subject has a decreased risk of developing muscle disorder.

The present disclosure also provides muscle disorder therapeutic agents for use in the treatment or prevention of muscle disorder in a subject having an FNIP1 variant nucleic acid molecule and/or an FLCN variant nucleic acid molecule.

The present disclosure also provides FNIP1 inhibitors for use in the treatment or prevention of muscle disorder in a subject that is FNIP1 reference or is heterozygous for an FNIP1 variant nucleic acid molecule.

The present disclosure also provides FLCN inhibitors for use in the treatment or prevention of muscle disorder in a subject that is FLCN reference or is heterozygous for an FLCN variant nucleic acid molecule.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows identification of FNIP1 rare coding variation associated with blood lipids and liver enzymes in ExWAS; the largest effect was observed with lower triglycerides and ALT.

FIG. 2 shows identification of genome-wide association analysis with a common missense (5:131672501:T:C—Gln648Arg) variant in FNIP1 to be associated with TGL:HDL ratio.

FIG. 3 shows association of FNIP1 rare coding variants with higher HDLC and lower triglycerides and LDLC levels; FLCN shows similar pattern of associations.

FIG. 4 shows association of FNIP1 and FLCN rare coding variants with lower ALT levels; FLCN pLoFs are associated with lower MRI derived liver fat and inflammation.

FIG. 5 shows the impact of FNIP1 and FLCN rare coding variants on ALT and PDFF.

FIG. 6 shows association of FNIP1 rare coding variants with lower odds for NALD.

FIG. 7 shows association of common FNIP1 missense variant with liver enzymes.

FIG. 8 shows association of FNIP1 and FLCN rare coding variants with lower waist to hip ratio and body fat percentage.

FIG. 9 shows the impact of rare variation in FNIP1 on body fat distribution.

FIG. 10 shows association of FNIP1 and FLCN rare coding variants with lower HbA1c levels and lower odds of type 2 diabetes.

FIG. 11 shows association of FNIP1 and FLCN rare coding variants with lower eGFR.

FIG. 12 shows FNIP1 pLoF had general protective cardiometabolic associations.

FIG. 13 shows FNIP1 pLoF had general protective cardiometabolic associations.

FIG. 14 shows FNIP1 common missense variant phenome-wide associations are similar to FNIP1 rare coding associations.

FIG. 15 shows FNIP1 common missense variant shows strongest association with lower odds for T2D.

FIG. 16 shows FNIP1 or FLCN inhibition in skeletal muscle induced an increase in oxidative muscle fibers and mitochondrial gene expression; AAVmyo2A was obtained from SignaGen Laboratories (Catalog #SL100862).

FIG. 17 shows FNIP1 or FLCN inhibition in skeletal muscle enhanced vascularization and muscle glycogen content.

FIG. 18 shows FLCN inhibition in skeletal muscle induced an increase in oxidative muscle fibers, enhanced vascularity, and increased glycogen content.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horses, cows, and pigs), companion animals (such as, for example, dogs and cats), laboratory animals (such as, for example, mice, rats, and rabbits), and non-human primates. In some embodiments, the subject is a human. In some embodiments, the human is a patient under the care of a physician.

It has been observed in accordance with the present disclosure that rare FNIP1 variant nucleic acid molecule and rare FLCN variant nucleic acid molecules (whether these variants are homozygous or heterozygous in a particular subject) associate with a decreased risk of developing muscle disorder. It is believed that FNIP1 variant nucleic acid molecule and/or an FLCN variant nucleic acid molecules have not been associated with muscle disorder in humans. Therefore, subjects that are FNIP1 reference and/or FLCN reference or heterozygous for an FNIP1 variant nucleic acid molecule and/or an FLCN variant nucleic acid molecule may be treated with an FNIP1 inhibitor and/or an FLCN inhibitor such that muscle disorder is inhibited or prevented, the symptoms thereof are reduced or prevented, and/or development of symptoms is repressed or prevented. It is also believed that such subjects having muscle disorder may further be treated with one or more muscle disorder therapeutic agents. In addition, the present disclosure provides methods of leveraging the presence or absence of FNIP1 variant nucleic acid molecule and/or an FLCN variant nucleic acid molecules in subjects to identify or stratify risk is such subjects of developing muscle disorder, or to diagnose subjects as having an increased risk of developing muscle disorder.

For purposes of the present disclosure, any particular subject, such as a human, can be categorized as having one of three FNIP1 genotypes: i) FNIP1 reference; ii) heterozygous for an FNIP1 variant nucleic acid molecule; or iii) homozygous for an FNIP1 variant nucleic acid molecule. A subject is FNIP1 reference when the subject does not have a copy of an FNIP1 variant nucleic acid molecule. A subject is heterozygous for an FNIP1 variant nucleic acid molecule when the subject has a single copy of an FNIP1 variant nucleic acid molecule. A subject is homozygous for an FNIP1 variant nucleic acid molecule when the subject has two copies of an FNIP1 variant nucleic acid molecule.

For purposes of the present disclosure, any particular subject, such as a human, can be categorized as having one of three FLCN genotypes: i) FLCN reference; ii) heterozygous for an FLCN variant nucleic acid molecule; or iii) homozygous for an FLCN variant nucleic acid molecule. A subject is FLCN reference when the subject does not have a copy of an FLCN variant nucleic acid molecule. A subject is heterozygous for an FLCN variant nucleic acid molecule when the subject has a single copy of an FLCN variant nucleic acid molecule. A subject is homozygous for an FLCN variant nucleic acid molecule when the subject has two copies of an FLCN variant nucleic acid molecule.

In any of the embodiments described herein, the FNIP1 variant nucleic acid molecule can be any nucleic acid molecule (such as, a genomic nucleic acid molecule, an mRNA molecule, or a cDNA molecule produced from an mRNA molecule) encoding an FNIP1 variant polypeptide having a partial loss-of-function, a complete loss-of-function, a predicted partial loss-of-function, or a predicted complete loss-of-function. A subject who has an FNIP1 polypeptide having a partial loss-of-function (or predicted partial loss-of-function) is hypomorphic for FNIP1. In some embodiments, the FNIP1 variant nucleic acid molecule results in decreased or aberrant expression or activity of FNIP1 mRNA or polypeptide. In some embodiments, the FNIP1 variant nucleic acid molecule is associated with a reduced in vitro response to FNIP1 ligands compared with reference FNIP1. In some embodiments, the FNIP1 variant nucleic acid molecule is a splice-site variant, a stop-gain variant, a start-loss variant, a stop-loss variant, a frameshift variant, an in-frame indel variant, or a variant that encodes a truncated FNIP1 variant polypeptide. In some embodiments, the FNIP1 variant nucleic acid molecule is a missense variant nucleic acid molecule. In some embodiments, the FNIP1 variant nucleic acid molecule comprises a single nucleotide polymorphism (SNP). In some embodiments, the FNIP1 variant nucleic acid molecule comprises a variation in a coding region. In some embodiments, the FNIP1 variant nucleic acid molecule does not comprise a variation in a non-coding region, except for a splice acceptor region (two bases before the start of any exon except the first). In some embodiments, the FNIP1 variant nucleic acid molecule results or is predicted to result in a premature truncation of an FNIP1 polypeptide compared to the reference FNIP1. In some embodiments, the FNIP1 variant nucleic acid molecule is a variant that is predicted to be damaging to the protein function (and hence, in this case, protective to the human) by in vitro prediction algorithms such as Polyphen, SIFT, or similar algorithms. In some embodiments, the FNIP1 variant nucleic acid molecule is a variant that causes or is predicted to cause a nonsynonymous amino acid substitution in an FNIP1 nucleic acid molecule and whose allele frequency is less than 1/100 alleles in the population from which the subject is selected. In some embodiments, the FNIP1 variant nucleic acid molecule is any rare missense variant (allele frequency <0.1%; or 1 in 1,000 alleles), or any splice-site, stop-gain, start-loss, stop-loss, frameshift, or in-frame indel, or other frameshift FNIP1 variant.

In any of the embodiments described herein, the FLCN variant nucleic acid molecule can be any nucleic acid molecule (such as, a genomic nucleic acid molecule, an mRNA molecule, or a cDNA molecule produced from an mRNA molecule) encoding an FLCN variant polypeptide having a partial loss-of-function, a complete loss-of-function, a predicted partial loss-of-function, or a predicted complete loss-of-function. A subject who has an FLCN polypeptide having a partial loss-of-function (or predicted partial loss-of-function) is hypomorphic for FLCN. In some embodiments, the FLCN variant nucleic acid molecule results in decreased or aberrant expression or activity of FLCN mRNA or polypeptide. In some embodiments, the FLCN variant nucleic acid molecule is associated with a reduced in vitro response to FLCN ligands compared with reference FLCN. In some embodiments, the FLCN variant nucleic acid molecule is a splice-site variant, a stop-gain variant, a start-loss variant, a stop-loss variant, a frameshift variant, an in-frame indel variant, or a variant that encodes a truncated FLCN variant polypeptide. In some embodiments, the FLCN variant nucleic acid molecule is a missense variant nucleic acid molecule. In some embodiments, the FLCN variant nucleic acid molecule comprises a single nucleotide polymorphism (SNP). In some embodiments, the FLCN variant nucleic acid molecule comprises a variation in a coding region. In some embodiments, the FLCN variant nucleic acid molecule does not comprise a variation in a non-coding region, except for a splice acceptor region (two bases before the start of any exon except the first). In some embodiments, the FLCN variant nucleic acid molecule results or is predicted to result in a premature truncation of an FLCN polypeptide compared to the reference FLCN. In some embodiments, the FLCN variant nucleic acid molecule is a variant that is predicted to be damaging to the protein function (and hence, in this case, protective to the human) by in vitro prediction algorithms such as Polyphen, SIFT, or similar algorithms. In some embodiments, the FLCN variant nucleic acid molecule is a variant that causes or is predicted to cause a nonsynonymous amino acid substitution in an FLCN nucleic acid molecule and whose allele frequency is less than 1/100 alleles in the population from which the subject is selected. In some embodiments, the FLCN variant nucleic acid molecule is any rare missense variant (allele frequency <0.1%; or 1 in 1,000 alleles), or any splice-site, stop-gain, start-loss, stop-loss, frameshift, or in-frame indel, or other frameshift FLCN variant.

In any of the embodiments described herein, the FNIP1 variant genomic nucleic acid molecule may include one or more variations at any of the positions of chromosome 5 (i.e., positions 131,641,714-131,797,017) using the nucleotide sequence of the FNIP1 reference genomic nucleic acid molecule in the GRCh38/hg38 human genome assembly (see, ENSG00000217128.13, ENST00000510461.6 annotated in the in the Ensembl database (URL: world wide web at “http://useast.ensembl.org/Homo_sapiens/Gene/Summary?g=ENSG00000217128;r=5:131641714-131797017;transcript=ENST00000510461.6”)) as a reference sequence. The sequences provided in these transcripts for the FNIP1 genomic nucleic acid molecule are only exemplary sequences. Other sequences for the FNIP1 genomic nucleic acid molecule are also possible.

In any of the embodiments described herein, the FNIP1 variant nucleic acid molecule may comprise any one or more of the following genetic variations in the genomic nucleic acid molecule (referring to the chromosome:positions set forth in the GRCh38/hg38 human genome assembly): 5:131644687:A:G, 5:131644689:A:T, 5:131644701:G:A, 5:131644702:C:T, 5:131644707:T:C, 5:131644707:T:G, 5:131644708:A:C, 5:131644717:G:A, 5:131644717:G:T, 5:131644720:T:C, 5:131644721:G:C, 5:131644725:G:A, 5:131644726:C:T, 5:131644728:A:G, 5:131644734:G:A, 5:131644735:C:A, 5:131644735:C:T, 5:131644738:G:C, 5:131644744:G:A, 5:131644759:C:T, 5:131644761:A:G, 5:131647089:C:T, 5:131647090:C:A, 5:131647090:C:T, 5:131647091:C:A, 5:131647093:A:G, 5:131647093:A:T, 5:131647100:C:A, 5:131647106:G:C, 5:131647107:C:A, 5:131647111:T:C, 5:131647112:T:C, 5:131647117:T:G, 5:131647118:G:A, 5:131647123:C:CG, 5:131647123:C:G, 5:131647123:C:T, 5:131647124:G:A, 5:131647124:G:C, 5:131647125:C:T, 5:131647129:T:C, 5:131647132:C:G, 5:131647135:C:G, 5:131647145:C:T, 5:131647151:G:C, 5:131647154:T:G, 5:131647162:T:C, 5:131647165:A:T, 5:131647166:A:G, 5:131647174:T:C, 5:131647175:C:T, 5:131647183:C:A, 5:131647183:C:T, 5:131647184:G:A, 5:131647187:C:G, 5:131647188:T:G, 5:131647190:C:T, 5:131647195:T:C, 5:131647195:T:G, 5:131647196:G:GCATT, 5:131647199:T:A, 5:131647199:T:C, 5:131647202:C:T, 5:131647206:C:CTG, 5:131651807:T:C, 5:131651810:G:A, 5:131651810:G:T, 5:131651814:C:G, 5:131651814:CA:C, 5:131651817:GT:G, 5:131651818:T:A, 5:131651819:T:G, 5:131651821:T:C, 5:131651824:T:A, 5:131651824:T:C, 5:131651825:T:C, 5:131651828:A:G, 5:131651831:G:C, 5:131651834:G:T, 5:131651834:GAA:G, 5:131651837:G:C, 5:131651839:G:A, 5:131651840:T:C, 5:131651842:G:A, 5:131651842:G:T, 5:131651844:A:T, 5:131651849:G:A, 5:131651854:T:C, 5:131651863:A:G, 5:131651866:C:A, 5:131651875:A:G, 5:131651878:A:G, 5:131651879:C:T, 5:131651880:T:A, 5:131651885:T:C, 5:131651887:C:G, 5:131651896:T:C, 5:131651908:C:T, 5:131651909:G:A, 5:131651909:G:C, 5:131651911:C:G, 5:131651914:T:C, 5:131651921:T:C, 5:131651927:C:T, 5:131651930:G:A, 5:131651930:G:C, 5:131651930:GA:G, 5:131651933:C:T, 5:131651935:G:C, 5:131651944:T:C, 5:131651946:C:T, 5:131651947:A:C, 5:131651947:A:G, 5:131651948:T:C, 5:131651956:A:G, 5:131651963:A:G, 5:131651963:A:T, 5:131651966:C:G, 5:131651966:C:T, 5:131651968:G:C, 5:131651969:C:T, 5:131651972:C:T, 5:131651978:T:C, 5:131651993:C:G, 5:131651995:G:A, 5:131651999:G:T, 5:131670467:A:G, 5:131670468:C:T, 5:131670471:C:T, 5:131670472:A:T, 5:131670473:T:C, 5:131670473:T:G, 5:131670474:G:T, 5:131670476:G:A, 5:131670476:G:C, 5:131670477:A:C, 5:131670482:T:A, 5:131670483:C:G, 5:131670483:C:T, 5:131670487:C:T, 5:131670493:A:T, 5:131670500:C:A, 5:131670500:C:T, 5:131670501:G:A, 5:131670502:G:C, 5:131670503:A:T, 5:131670505:C:G, 5:131670506:C:T, 5:131670510:C:T, 5:131670511:A:C, 5:131670514:A:C, 5:131670519:C:A, 5:131670521:A:G, 5:131670540:C:G, 5:131670543:G:T, 5:131670546:C:T, 5:131670550:A:AT, 5:131670551:G:A, 5:131670552:A:G, 5:131670554:G:A, 5:131670554:G:T, 5:131670560:T:C, 5:131670561:A:G, 5:131670579:T:A, 5:131670582:C:T, 5:131670585:A:G, 5:131670593:A:G, 5:131670594:T:C, 5:131670596:T:A, 5:131670605:A:T, 5:131670606:C:G, 5:131670606:C:T, 5:131670610:A:C, 5:131670611:C:A, 5:131670611:C:T, 5:131670612:T:C, 5:131670614:A:G, 5:131670618:C:T, 5:131670621:T:C, 5:131670626:T:C, 5:131670632:C:T, 5:131671506:C:A, 5:131671509:G:A, 5:131671509:G:C, 5:131671514:G:C, 5:131671516:T:C, 5:131671522:A:C, 5:131671522:A:T, 5:131671524:C:T, 5:131671527:C:T, 5:131671533:C:A, 5:131671533:C:G, 5:131671533:C:T, 5:131671534:C:A, 5:131671538:T:A, 5:131671539:C:G, 5:131671543:T:G, 5:131671545:G:C, 5:131671548:C:G, 5:131671551:C:T, 5:131671554:C:T, 5:131671557:A:C, 5:131671559:T:C, 5:131671562:C:T, 5:131671569:C:T, 5:131671572:G:C, 5:131671572:G:T, 5:131671574:C:T, 5:131671575:T:C, 5:131671584:C:T, 5:131671586:T:C, 5:131671589:C:A, 5:131671592:G:A, 5:131671595:T:C, 5:131671595:T:G, 5:131671601:C:T, 5:131671604:T:C, 5:131671604:T:G, 5:131671610:T:C, 5:131671610:T:G, 5:131671613:C:T, 5:131671614:C:T, 5:131671617:G:A, 5:131671628:G:A, 5:131671632:T:C, 5:131671634:T:C, 5:131671634:T:G, 5:131671639:T:G, 5:131671646:A:T, 5:131671647:T:A, 5:131671647:T:C, 5:131671647:T:G, 5:131671658:G:A, 5:131671658:G:C, 5:131671659:T:C, 5:131671661:C:T, 5:131671664:A:C, 5:131671665:C:T, 5:131671668:C:A, 5:131671669:A:C, 5:131671670:A:AT, 5:131671670:A:T, 5:131671673:T:C, 5:131671677:T:A, 5:131671680:C:T, 5:131671683:A:C, 5:131671685:C:A, 5:131671686:T:C, 5:131671691:T:C, 5:131671692:T:C, 5:131671694:T:A, 5:131671697:C:A, 5:131671700:T:C, 5:131671700:TG:T, 5:131671701:G:A, 5:131671703:G:A, 5:131671703:G:C, 5:131671704:G:A, 5:131671705:GA:G, 5:131671706:A:T, 5:131671710:G:T, 5:131671712:A:C, 5:131671713:T:C, 5:131671713:TG:T, 5:131671718:A:G, 5:131671718:A:T, 5:131671726:T:A, 5:131671732:G:C, 5:131671734:C:T, 5:131671740:G:C, 5:131671742:G:A, 5:131671742:G:T, 5:131671745:A:G, 5:131671751:G:A, 5:131671751:G:T, 5:131671754:T:C, 5:131671755:T:A, 5:131671758:A:G, 5:131671760:G:A, 5:131671761:A:T, 5:131671762:A:C, 5:131671763:T:C, 5:131671764:C:G, 5:131671764:C:T, 5:131671767:G:C, 5:131671769:G:A, 5:131671770:G:A, 5:131671770:G:T, 5:131671772:A:C, 5:131671772:A:G, 5:131671773:C:A, 5:131671775:G:A, 5:131671776:T:C, 5:131671778:T:C, 5:131671779:C:G, 5:131671779:C:T, 5:131671780:T:C, 5:131671782:T:C, 5:131671784:C:T, 5:131671788:T:G, 5:131671788:TA:T, 5:131671794:A:G, 5:131671795:T:A, 5:131671796:T:C, 5:131671797:C:G, 5:131671797:C:T, 5:131671798:A:T, 5:131671802:T:A, 5:131671802:T:C, 5:131671812:T:C, 5:131671814:T:A, 5:131671814:T:C, 5:131671816:T:G, 5:131671821:T:C, 5:131671823:C:T, 5:131671825:C:A, 5:131671825:C:G, 5:131671832:T:G, 5:131671842:C:G, 5:131671844:A:G, 5:131671846:C:T, 5:131671847:A:G, 5:131671847:A:T, 5:131671848:T:C, 5:131671850:C:T, 5:131671851:A:T, 5:131671853:C:T, 5:131671854:A:T, 5:131671860:C:G, 5:131671866:T:C, 5:131671867:A:T, 5:131671868:T:C, 5:131671869:C:T, 5:131671874:C:T, 5:131671878:T:C, 5:131671882:A:T, 5:131671887:G:C, 5:131671889:A:G, 5:131671889:A:T, 5:131671890:C:A, 5:131671895:T:A, 5:131671896:C:T, 5:131671899:T:C, 5:131671899:T:TA, 5:131671905:T:C, 5:131671907:G:A, 5:131671908:T:C, 5:131671908:T:G, 5:131671911:C:T, 5:131671912:G:C, 5:131671914:T:C, 5:131671918:A:C, 5:131671922:T:A, 5:131671922:T:C, 5:131671924:A:T, 5:131671925:T:C, 5:131671926:T:G, 5:131671928:A:G, 5:131671933:T:A, 5:131671935:C:T, 5:131671938:C:T, 5:131671939:G:C, 5:131671941:A:G, 5:131671946:C:T, 5:131671950:T:C, 5:131671954:T:G, 5:131671956:C:T, 5:131671959:G:C, 5:131671963:TG:T, 5:131671964:G:A, 5:131671965:A:C, 5:131671967:T:C, 5:131671969:ATTCC:A, 5:131671970:T:C, 5:131671979:G:A, 5:131671980:G:C, 5:131671988:C:T, 5:131671991:G:A, 5:131671992:G:T, 5:131671997:T:G, 5:131671998:C:T, 5:131672000:G:A, 5:131672003:A:G, 5:131672004:C:T, 5:131672024:T:C, 5:131672025:C:CTCCAGATTGG, 5:131672027:C:T, 5:131672028:C:G, 5:131672030:G:C, 5:131672031:A:G, 5:131672032:T:G, 5:131672033:TGGTCC:T, 5:131672036:T:C, 5:131672037:C:T, 5:131672052:T:C, 5:131672054:G:A, 5:131672055:T:C, 5:131672055:T:G, 5:131672060:T:G, 5:131672062:A:T, 5:131672063:T:G, 5:131672064:G:A, 5:131672064:G:T, 5:131672066:T:C, 5:131672067:G:A, 5:131672067:G:T, 5:131672070:C:T, 5:131672078:CCT:C, 5:131672086:T:G, 5:131672087:T:C, 5:131672088:C:T, 5:131672096:G:A, 5:131672097:G:A, 5:131672100:T:C, 5:131672103:T:A, 5:131672106:G:A, 5:131672111:C:G, 5:131672111:C:T, 5:131672112:T:C, 5:131672116:A:C, 5:131672118:T:A, 5:131672120:T:C, 5:131672121:G:C, 5:131672126:A:G, 5:131672127:C:T, 5:131672130:C:T, 5:131672132:G:A, 5:131672132:G:T, 5:131672133:C:T, 5:131672135:T:A, 5:131672136:G:C, 5:131672141:C:G, 5:131672141:C:T, 5:131672142:G:A, 5:131672145:G:C, 5:131672146:C:G, 5:131672153:T:A, 5:131672156:G:C, 5:131672157:A:C, 5:131672158:A:C, 5:131672160:C:T, 5:131672162:G:A, 5:131672163:G:A, 5:131672163:G:C, 5:131672169:T:C, 5:131672171:G:A, 5:131672171:G:C, 5:131672172:A:C, 5:131672172:A:G, 5:131672172:A:T, 5:131672174:T:A, 5:131672179:A:C, 5:131672180:A:G, 5:131672181:T:A, 5:131672189:G:C, 5:131672192:A:G, 5:131672193:C:T, 5:131672195:T:C, 5:131672201:T:C, 5:131672212:A:C, 5:131672213:C:T, 5:131672233:A:C, 5:131672234:A:G, 5:131672238:T:C, 5:131672240:T:C, 5:131672240:T:G, 5:131672244:G:A, 5:131672246:G:A, 5:131672247:G:GT, 5:131672247:G:T, 5:131672248:T:G, 5:131672249:T:A, 5:131672252:T:C, 5:131672253:T:C, 5:131672254:T:G, 5:131672255:T:C, 5:131672256:C:G, 5:131672256:C:T, 5:131672259:C:G, 5:131672261:A:G, 5:131672262:C:T, 5:131672264:A:G, 5:131672265:T:C, 5:131672267:C:A, 5:131672268:C:T, 5:131672270:G:A, 5:131672271:T:C, 5:131672271:T:G, 5:131672273:G:A, 5:131672274:A:C, 5:131672274:A:T, 5:131672278:C:T, 5:131672279:A:C, 5:131672280:T:C, 5:131672283:C:T, 5:131672285:T:C, 5:131672291:G:A, 5:131672292:T:G, 5:131672296:ACTGT:A, 5:131672303:G:A, 5:131672311:C:A, 5:131672312:A:G, 5:131672320:A:C, 5:131672321:C:A, 5:131672321:C:T, 5:131672324:T:C, 5:131672328:A:G, 5:131672328:A:T, 5:131672342:G:A, 5:131672343:A:C, 5:131672345:T:C, 5:131672346:C:G, 5:131672346:C:T, 5:131672351:C:T, 5:131672352:C:T, 5:131672357:T:G, 5:131672363:A:C, 5:131672363:A:G, 5:131672367:C:T, 5:131672369:C:T, 5:131672370:A:G, 5:131672372:T:A, 5:131672375:T:C, 5:131672376:C:T, 5:131672381:G:C, 5:131672382:G:C, 5:131672382:G:T, 5:131672385:C:T, 5:131672387:G:C, 5:131672389:TC:T, 5:131672390:C:T, 5:131672391:C:T, 5:131672393:G:A, 5:131672394:T:G, 5:131672396:CA:C, 5:131672400:C:A, 5:131672405:G:A, 5:131672406:T:C, 5:131672408:T:C, 5:131672409:C:G, 5:131672417:G:T, 5:131672418:C:A, 5:131672418:C:T, 5:131672420:T:C, 5:131672423:A:G, 5:131672424:A:G, 5:131672424:AG:A, 5:131672427:A:C, 5:131672430:T:A, 5:131672430:T:C, 5:131672436:A:T, 5:131672438:T:G, 5:131672439:T:C, 5:131672440:A:T, 5:131672441:T:C, 5:131672442:C:T, 5:131672444:C:G, 5:131672447:T:C, 5:131672448:A:G, 5:131672449:C:G, 5:131672450:T:C, 5:131672454:T:C, 5:131672457:C:T, 5:131672457:CA:C, 5:131672460:C:T, 5:131672463:C:T, 5:131672465:G:T, 5:131672466:C:T, 5:131672468:T:C, 5:131672471:T:C, 5:131672474:T:C, 5:131672475:C:T, 5:131672477:T:A, 5:131672478:G:A, 5:131672482:G:C, 5:131672486:G:A, 5:131672490:G:A, 5:131672502:G:A, 5:131672504:C:T, 5:131672505:A:G, 5:131672507:T:G, 5:131672508:C:G, 5:131672509:A:C, 5:131672511:C:G, 5:131672517:T:C, 5:131672519:C:T, 5:131672522:A:G, 5:131672527:C:G, 5:131672529:C:T, 5:131672534:G:A, 5:131672543:T:G, 5:131672552:T:C, 5:131672553:C:A, 5:131672553:C:G, 5:131672557:C:G, 5:131672559:C:G, 5:131672559:CTT:C, 5:131672562:G:C, 5:131672562:G:T, 5:131672567:G:A, 5:131672568:A:T, 5:131672575:C:A, 5:131672582:T:G, 5:131672583:T:G, 5:131672588:C:G, 5:131672594:A:G, 5:131672595:G:A, 5:131672595:G:C, 5:131672597:G:A, 5:131672598:G:A, 5:131672602:A:T, 5:131672603:C:T, 5:131672606:C:T, 5:131672610:A:G, 5:131672613:TAC:T, 5:131672621:C:A, 5:131672621:C:G, 5:131672621:C:T, 5:131672622:A:T, 5:131672627:G:A, 5:131672632:TC:T, 5:131672633:C:G, 5:131672648:T:C, 5:131672649:C:G, 5:131672651:T:C, 5:131672651:T:G, 5:131672652:T:C, 5:131672654:A:G, 5:131672655:A:G, 5:131672658:G:A, 5:131672662:A:C, 5:131672664:T:A, 5:131672664:T:C, 5:131672665:G:C, 5:131672669:T:C, 5:131672678:T:C, 5:131672682:T:C, 5:131672684:G:T, 5:131672688:C:G, 5:131672688:C:T, 5:131672695:A:T, 5:131672696:TACTCTGATTC:T, 5:131672699:T:A, 5:131672699:T:G, 5:131672700:C:G, 5:131672710:T:C, 5:131672711:A:T, 5:131672712:T:C, 5:131672714:T:C, 5:131672717:C:T, 5:131672720:TTC:T, 5:131672725:T:G, 5:131672729:G:A, 5:131672730:T:C, 5:131672730:T:G, 5:131672732:G:A, 5:131672732:G:T, 5:131672733:T:C, 5:131672738:A:G, 5:131672742:C:A, 5:131672744:G:C, 5:131672747:C:T, 5:131672748:C:G, 5:131672751:G:A, 5:131672753:A:G, 5:131672757:C:T, 5:131672758:G:C, 5:131672762:G:A, 5:131672767:A:C, 5:131672767:A:T, 5:131672768:T:C, 5:131672769:C:T, 5:131672770:T:G, 5:131672771:T:A, 5:131672783:A:G, 5:131672784:A:C, 5:131672787:G:C, 5:131672792:G:A, 5:131672793:T:C, 5:131672798:T:G, 5:131672799:G:T, 5:131672801:A:G, 5:131672802:G:A, 5:131672816:A:G, 5:131672817:T:A, 5:131672825:G:C, 5:131672826:T:A, 5:131672833:ATAAAG:A, 5:131672834:T:C, 5:131672837:A:T, 5:131672841:G:C, 5:131672843:C:A, 5:131672843:C:T, 5:131672847:G:C, 5:131672850:C:T, 5:131672853:T:C, 5:131672856:C:A, 5:131672856:C:T, 5:131672857:T:G, 5:131672859:G:C, 5:131672859:G:T, 5:131672861:C:A, 5:131672861:C:T, 5:131672862:G:A, 5:131672864:T:C, 5:131672865:T:C, 5:131672870:A:G, 5:131672873:A:G, 5:131672876:A:G, 5:131672877:C:G, 5:131672879:G:C, 5:131672880:T:C, 5:131672886:C:T, 5:131672889:A:T, 5:131672891:C:T, 5:131672892:G:A, 5:131672895:C:A, 5:131672895:C:T, 5:131672900:G:A, 5:131672901:A:G, 5:131672903:C:A, 5:131672903:C:G, 5:131672903:C:T, 5:131672906:A:C, 5:131672906:A:G, 5:131672910:C:A, 5:131672910:C:T, 5:131672912:C:G, 5:131672912:C:T, 5:131672915:T:C, 5:131672916:A:G, 5:131672918:A:G, 5:131672920:G:C, 5:131677702:C:G, 5:131677703:C:T, 5:131677713:C:T, 5:131677721:G:A, 5:131677723:T:C, 5:131677736:T:C, 5:131677738:T:C, 5:131677738:T:G, 5:131677742:C:A, 5:131677748:T:C, 5:131677751:C:T, 5:131677755:A:C, 5:131677756:C:T, 5:131677759:T:C, 5:131677768:T:A, 5:131677780:A:C, 5:131677780:A:G, 5:131677781:A:C, 5:131677782:T:C, 5:131677783:A:G, 5:131677790:T:C, 5:131677790:T:G, 5:131677792:G:C, 5:131677793:G:A, 5:131677796:G:C, 5:131677804:T:C, 5:131677807:G:A, 5:131677811:T:C, 5:131677811:T:G, 5:131677819:G:A, 5:131677823:C:T, 5:131677829:C:T, 5:131677835:G:A, 5:131677837:T:C, 5:131677838:T:A, 5:131677840:G:A, 5:131677840:G:C, 5:131677847:C:G, 5:131677849:G:A, 5:131677856:T:C, 5:131677856:T:G, 5:131677859:G:A, 5:131677859:G:C, 5:131677865:G:A, 5:131677869:G:C, 5:131679030:G:T, 5:131679032:T:C, 5:131679032:T:G, 5:131679033:T:A, 5:131679041:G:A, 5:131679044:T:C, 5:131679048:C:G, 5:131679050:A:T, 5:131679060:T:C, 5:131679061:G:C, 5:131679062:A:T, 5:131679064:C:A, 5:131679066:C:G, 5:131679066:C:T, 5:131679069:T:C, 5:131679070:C:A, 5:131679072:T:A, 5:131679072:T:C, 5:131679073:G:C, 5:131679074:A:C, 5:131679077:C:T, 5:131679078:G:A, 5:131679078:G:T, 5:131679079:A:T, 5:131679080:T:C, 5:131679083:C:A, 5:131679084:A:C, 5:131679087:G:A, 5:131679087:G:C, 5:131679088:G:T, 5:131679089:T:C, 5:131679092:T:C, 5:131679094:C:A, 5:131679102:G:A, 5:131679102:G:T, 5:131679104:G:A, 5:131679107:C:G, 5:131679107:C:T, 5:131679110:G:A, 5:131679112:C:T, 5:131679113:A:G, 5:131679114:T:A, 5:131679117:T:A, 5:131679117:T:C, 5:131679119:G:T, 5:131679120:T:C, 5:131679124:C:T, 5:131679126:A:G, 5:131679129:C:G, 5:131679129:C:T, 5:131679131:G:T, 5:131679132:G:A, 5:131679132:G:T, 5:131679133:T:G, 5:131679135:C:T, 5:131679138:C:A, 5:131679138:C:T, 5:131679143:C:T, 5:131679144:G:A, 5:131679147:G:C, 5:131679147:G:T, 5:131679149:A:G, 5:131679150:T:A, 5:131679152:G:A, 5:131679152:G:T, 5:131679153:T:C, 5:131679154:G:T, 5:131679156:A:G, 5:131679161:T:G, 5:131679164:C:A, 5:131679164:C:G, 5:131679164:C:T, 5:131679165:A:G, 5:131679168:T:C, 5:131679170:G:A, 5:131679170:G:T, 5:131679171:T:A, 5:131679171:T:C, 5:131679173:G:C, 5:131679173:G:T, 5:131679173:GTTC:G, 5:131679175:T:G, 5:131679176:C:T, 5:131679177:T:A, 5:131698916:C:T, 5:131698919:G:C, 5:131698921:A:G, 5:131698930:G:C, 5:131698932:G:A, 5:131698932:G:C, 5:131698932:G:T, 5:131698933:C:A, 5:131698936:C:A, 5:131698936:C:T, 5:131698942:T:C, 5:131698944:C:A, 5:131698944:C:G, 5:131698944:C:T, 5:131698945:G:A, 5:131698947:T:A, 5:131698950:T:A, 5:131698950:T:C, 5:131698951:A:G, 5:131698953:G:A, 5:131698959:C:G, 5:131698963:T:C, 5:131698967:A:T, 5:131698968:C:A, 5:131698968:C:G, 5:131698969:T:C, 5:131698972:C:T, 5:131698974:T:C, 5:131698978:C:G, 5:131698979:TG:T, 5:131698981:A:C, 5:131698981:A:G, 5:131698983:C:T, 5:131698984:T:C, 5:131698986:C:T, 5:131698987:G:A, 5:131698991:C:T, 5:131698993:T:C, 5:131698997:C:G, 5:131698998:A:G, 5:131698999:T:C, 5:131699001:G:A, 5:131699001:G:T, 5:131704062:TA:T, 5:131704072:A:G, 5:131704073:T:C, 5:131704075:G:A, 5:131704079:T:A, 5:131704081:T:C, 5:131704090:T:C, 5:131704094:T:C, 5:131704096:T:A, 5:131704099:C:T, 5:131704108:A:T, 5:131704109:G:C, 5:131704111:G:A, 5:131704112:G:A, 5:131704114:A:C, 5:131704120:G:GA, 5:131704124:A:G, 5:131704127:A:G, 5:131704128:G:T, 5:131704130:A:C, 5:131704131:T:A, 5:131704132:T:A, 5:131704135:T:C, 5:131704137:A:C, 5:131704138:A:G, 5:131704138:A:T, 5:131704140:T:G, 5:131704141:T:G, 5:131704142:T:C, 5:131704144:T:C, 5:131704147:T:C, 5:131704153:T:A, 5:131704157:C:T, 5:131704159:T:A, 5:131704160:CT:C, 5:131704162:T:C, 5:131704165:G:C, 5:131704166:A:G, 5:131704167:C:G, 5:131704171:G:A, 5:131704178:T:A, 5:131704178:T:C, 5:131704181:C:A, 5:131704186:A:G, 5:131704187:T:C, 5:131704193:T:C, 5:131704202:T:G, 5:131704204:C:T, 5:131704205:G:A, 5:131704207:A:C, 5:131704207:A:G, 5:131704208:C:T, 5:131704211:T:C, 5:131704214:C:T, 5:131704216:G:A, 5:131704220:T:G, 5:131704222:G:A, 5:131704222:G:T, 5:131704223:G:A, 5:131704228:C:G, 5:131704228:C:T, 5:131704229:A:G, 5:131704230:G:C, 5:131704231:C:T, 5:131704232:T:G, 5:131704236:A:T, 5:131704237:T:A, 5:131704237:T:C, 5:131704240:G:A, 5:131704250:A:G, 5:131704251:G:C, 5:131704252:C:T, 5:131704259:C:T, 5:131704261:A:G, 5:131704262:T:C, 5:131706409:A:G, 5:131706412:A:G, 5:131706414:C:T, 5:131706418:G:A, 5:131706424:C:T, 5:131706426:C:G, 5:131706426:C:T, 5:131706427:C:T, 5:131706432:T:C, 5:131706438:C:G, 5:131706441:G:C, 5:131706442:T:C, 5:131706445:T:C, 5:131706448:G:C, 5:131706453:C:T, 5:131706454:G:A, 5:131706456:C:T, 5:131706457:G:A, 5:131706462:C:T, 5:131706463:G:A, 5:131706463:G:T, 5:131706465:C:T, 5:131706466:G:A, 5:131706471:T:C, 5:131706480:G:A, 5:131706486:C:T, 5:131706487:T:A, 5:131706489:C:T, 5:131706490:G:A, 5:131706490:G:C, 5:131706491:GGTAA:G, 5:131706501:G:A, 5:131706502:A:C, 5:131706504:T:G, 5:131706507:G:C, 5:131706508:G:C, 5:131706516:A:C, 5:131706519:G:A, 5:131706519:G:C, 5:131706520:G:C, 5:131706523:T:A, 5:131706523:T:C, 5:131706524:G:C, 5:131706525:A:G, 5:131706526:T:C, 5:131706541:G:A, 5:131706548:T:C, 5:131709206:C:T, 5:131709207:G:A, 5:131709210:C:A, 5:131709210:C:G, 5:131709210:C:T, 5:131709213:T:C, 5:131709215:C:T, 5:131709216:C:T, 5:131709218:C:G, 5:131709219:T:A, 5:131709219:T:C, 5:131709226:G:C, 5:131709227:T:C, 5:131709228:C:A, 5:131709233:T:C, 5:131709234:T:C, 5:131709234:T:G, 5:131709237:G:A, 5:131709239:T:C, 5:131709240:C:G, 5:131709243:T:C, 5:131709245:C:T, 5:131709248:G:A, 5:131709248:G:C, 5:131709252:T:C, 5:131709258:T:C, 5:131709258:T:G, 5:131709260:G:C, 5:131709269:T:C, 5:131709270:G:A, 5:131710581:C:A, 5:131710581:C:T, 5:131710582:A:C, 5:131710584:A:C, 5:131710593:C:T, 5:131710594:G:T, 5:131710595:C:T, 5:131710600:G:C, 5:131710604:A:G, 5:131710605:G:C, 5:131710607:C:T, 5:131710608:G:A, 5:131710610:A:C, 5:131710613:G:A, 5:131710613:G:T, 5:131710614:G:C, 5:131710617:C:T, 5:131710618:C:G, 5:131710628:A:G, 5:131710632:C:G, 5:131710632:C:T, 5:131710634:C:T, 5:131710635:G:A, 5:131710637:C:A, 5:131710637:C:G, 5:131710638:T:C, 5:131710640:G:A, 5:131710640:G:C, 5:131710643:C:T, 5:131710644:T:C, 5:131710647:A:C, 5:131710649:A:T, 5:131710653:G:A, 5:131710655:G:A, 5:131710656:A:G, 5:131716564:C:G, 5:131716566:T:C, 5:131716567:A:G, 5:131716570:T:C, 5:131716573:C:A, 5:131716582:C:A, 5:131716582:C:T, 5:131716589:T:C, 5:131716592:C:A, 5:131716592:C:T, 5:131716592:CTG:C, 5:131716600:T:A, 5:131716600:T:C, 5:131716601:T:C, 5:131716606:G:C, 5:131716607:C:A, 5:131716607:C:T, 5:131716613:A:C, 5:131716615:G:T, 5:131716615:GT:G, 5:131716616:T:C, 5:131716617:A:C, 5:131716618:T:C, 5:131716621:T:C, 5:131716622:T:A, 5:131716627:T:C, 5:131716630:T:A, 5:131716630:T:C, 5:131716634:T:C, 5:131716634:T:G, 5:131716638:T:A, 5:131716648:T:C, 5:131716651:T:G, 5:131716652:G:A, 5:131716655:G:T, 5:131718984:A:T, 5:131718985:C:T, 5:131718986:G:A, 5:131718986:G:C, 5:131718986:G:T, 5:131718989:T:A, 5:131718989:T:C, 5:131718989:T:G, 5:131718993:G:A, 5:131718995:C:T, 5:131718998:C:T, 5:131719001:C:A, 5:131719013:C:T, 5:131719014:C:T, 5:131719016:G:A, 5:131719016:G:C, 5:131719019:C:T, 5:131719020:G:A, 5:131719020:G:C, 5:131719025:G:T, 5:131719026:T:C, 5:131719027:A:AAACACTTTGC, 5:131719027:A:C, 5:131719031:A:G, 5:131719032:C:T, 5:131719035:T:G, 5:131719041:G:A, 5:131719042:C:T, 5:131719043:A:C, 5:131719044:T:G, 5:131719046:A:G, 5:131719047:G:C, 5:131719048:C:G, 5:131719049:T:C, 5:131719050:G:A, 5:131719052:G:A, 5:131719056:G:A, 5:131719058:G:A, 5:131719058:G:T, 5:131719317:C:T, 5:131719318:G:A, 5:131719318:G:C, 5:131719320:A:C, 5:131719332:T:C, 5:131719339:T:A, 5:131719339:T:C, 5:131719342:A:G, 5:131719348:T:C, 5:131719350:T:G, 5:131719353:C:A, 5:131719356:A:C, 5:131719357:T:C, 5:131719359:G:A, 5:131719363:C:T, 5:131719366:A:G, 5:131719368:C:A, 5:131719368:C:G, 5:131719374:A:G, 5:131719375:T:C, 5:131719388:C:T, 5:131719390:T:C, 5:131719392:T:C, 5:131719393:T:C, 5:131719396:C:T, 5:131719402:A:G, 5:131719404:G:A, 5:131719405:A:G, 5:131719407:C:A, 5:131719410:C:T, 5:131719411:G:A, 5:131719415:AC:A, 5:131719416:C:T, 5:131719417:C:G, 5:131730903:C:G, 5:131730906:G:T, 5:131730908:T:C, 5:131730909:A:G, 5:131730915:G:C, 5:131730917:C:T, 5:131730921:G:C, 5:131730921:G:T, 5:131730922:G:C, 5:131730922:G:T, 5:131730924:C:G, 5:131730926:T:A, 5:131730927:T:C, 5:131730929:A:G, 5:131730932:T:C, 5:131730933:C:A, 5:131730933:C:G, 5:131730935:G:C, 5:131730936:A:C, 5:131730938:G:T, 5:131730939:A:G, 5:131730945:T:C, 5:131730948:C:A, 5:131730948:C:G, 5:131730948:C:T, 5:131730950:G:C, 5:131730950:G:T, 5:131730950:GAACT:G, 5:131730952:A:C, 5:131730952:A:T, 5:131730953:C:A, 5:131730956:T:C, 5:131730957:C:A, 5:131730959:A:G, 5:131730960:A:C, 5:131730965:G:C, 5:131730968:G:T, 5:131730969:A:T, 5:131730971:C:G, 5:131730971:C:T, 5:131730972:T:C, 5:131730972:T:G, 5:131730974:T:C, 5:131730975:C:T, 5:131730978:C:A, 5:131730978:C:T, 5:131730980:C:T, 5:131730981:C:T, 5:131730983:G:A, 5:131730984:G:C, 5:131730986:T:G, 5:131730987:T:G, 5:131730989:A:G, 5:131730991:T:G, 5:131730992:T:C, 5:131730993:G:A, 5:131730995:C:T, 5:131730999:A:G, 5:131731001:T:C, 5:131731004:C:A, 5:131731004:C:G, 5:131731007:A:C, 5:131731010:A:C, 5:131731011:C:A, 5:131731011:C:T, 5:131731014:T:C, 5:131731016:A:T, 5:131731019:T:G, 5:131731020:G:A, 5:131731022:G:C, 5:131731023:C:T, 5:131731026:C:T, 5:131731028:C:T, 5:131731029:T:C, 5:131731031:C:A, 5:131731035:G:C, 5:131731039:C:G, 5:131731040:T:C, 5:131744563:C:A, 5:131744565:G:A, 5:131744565:G:T, 5:131744568:A:T, 5:131744569:C:T, 5:131744571:G:A, 5:131744572:A:AT, 5:131744572:A:G, 5:131744577:T:A, 5:131744577:T:C, 5:131744578:C:A, 5:131744578:C:T, 5:131744590:T:C, 5:131744596:C:T, 5:131744598:C:A, 5:131744599:T:C, 5:131744601:G:C, 5:131744605:C:G, 5:131744613:A:G, 5:131744616:T:C, 5:131744617:T:G, 5:131744623:C:A, 5:131744623:C:G, 5:131744623:C:T, 5:131744623:CT:C, 5:131744625:C:T, 5:131744626:G:A, 5:131744626:G:C, 5:131744629:T:C, 5:131744632:C:T, 5:131744634:C:G, 5:131744634:C:T, 5:131744635:A:G, 5:131744642:A:C, 5:131744644:A:AT, 5:131744646:A:G, 5:131744647:C:T, 5:131744648:A:C, 5:131744650:T:C, 5:131744655:C:T, 5:131744656:G:A, 5:131744658:A:G, 5:131744659:T:C, 5:131744660:C:G, 5:131744662:G:A, 5:131744662:G:C, 5:131744668:G:T, 5:131744670:T:G, 5:131744671:C:G, 5:131744671:C:T, 5:131744673:A:G, 5:131744675:C:A, 5:131744676:T:C, 5:131744676:T:G, 5:131744677:C:G, 5:131744679:G:A, 5:131744680:G:A, 5:131796828:A:G, 5:131796829:C:T, 5:131796830:C:G, 5:131796834:A:G, 5:131796838:G:C, 5:131796843:C:A, 5:131796843:C:G, 5:131796845:G:A, 5:131796849:C:A, 5:131796851:C:G, 5:131796851:C:T, 5:131796852:G:A, 5:131796852:G:C, 5:131796858:C:A, 5:131796860:C:T, 5:131796863:C:A, 5:131796863:C:T, 5:131796866:G:C, 5:131796867:G:A, 5:131796872:C:A, 5:131796872:C:G, 5:131796875:A:G, 5:131796878:C:T, 5:131796879:C:T, 5:131796883:C:G, 5:131796884:C:G, 5:131796887:T:C, 5:131796889:G:C, 5:131796891:T:A, 5:131796897:G:C, 5:131796899:T:C, 5:131796900:T:C, 5:131796901:C:G, 5:131796904:G:C, 5:131796906:A:G, 5:131796911:G:A, 5:131796911:G:T, 5:131796912:T:A, 5:131796914:G:C, 5:131796915:G:A, or 5:131796917:G:A, or an mRNA molecule produced therefrom, or a cDNA molecule produced from the mRNA molecule.

For subjects that are genotyped or determined to be FNIP1 reference, such subjects have an increased risk of developing muscle disorder. For subjects that are genotyped or determined to be either FNIP1 reference or heterozygous for an FNIP1 variant nucleic acid molecule, such subjects can be treated with an FNIP1 inhibitor.

In any of the embodiments described herein, the FLCN variant genomic nucleic acid molecule may include one or more variations at any of the positions of chromosome 17 (i.e., positions 17,212,212-17,237,188) using the nucleotide sequence of the FLCN reference genomic nucleic acid molecule in the GRCh38/hg38 human genome assembly (see, ENSG00000154803.13, ENST00000285071.9 annotated in the in the Ensembl database (URL: world wide web at “http://useast.ensembl.org/Homo_sapiens/Gene/Summary?g=ENSG00000154803;r=17:17212212-17237188;transcript=ENST00000285071.9”)) as a reference sequence. The sequences provided in these transcripts for the FLCN genomic nucleic acid molecule are only exemplary sequences. Other sequences for the FLCN genomic nucleic acid molecule are also possible.

In any of the embodiments described herein, the FLCN variant nucleic acid molecule may comprise any one or more of the following genetic variations in the genomic nucleic acid molecule (referring to the chromosome:positions set forth in the GRCh38/hg38 human genome assembly): 17:17213659:TTCCGAGAC:T, 17:17213680:G:T, 17:17213686:C:G, 17:17213686:C:T, 17:17213686:CG:C, 17:17213687:G:A, 17:17213695:G:A, 17:17213703:G:C, 17:17213705:G:A, 17:17213705:GT:G, 17:17213711:T:C, 17:17213712:G:C, 17:17213712:G:T, 17:17213737:C:T, 17:17213738:A:G, 17:17213747:G:C, 17:17213749:A:G, 17:17213758:T:C, 17:17213789:G:T, 17:17213796:C:G, 17:17213796:CTG:C, 17:17213798:G:A, 17:17213805:C:G, 17:17213815:C:CT, 17:17213815:C:T, 17:17213816:G:A, 17:17213823:C:CA, 17:17213834:A:C, 17:17213836:T:C, 17:17213848:T:C, 17:17213849:T:C, 17:17213852:C:T, 17:17213855:T:C, 17:17214984:C:G, 17:17214984:C:T, 17:17214993:C:G, 17:17214995:C:T, 17:17214996:C:A, 17:17215001:T:A, 17:17215001:T:G, 17:17215004:G:A, 17:17215006:C:T, 17:17215013:G:A, 17:17215015:C:T, 17:17215016:A:G, 17:17215022:C:A, 17:17215024:A:C, 17:17215036:G:A, 17:17215036:G:C, 17:17215037:A:C, 17:17215048:T:C, 17:17215057:G:C, 17:17215070:T:C, 17:17215073:T:G, 17:17215082:TG:T, 17:17215087:C:T, 17:17215184:C:G, 17:17215184:C:T, 17:17215188:G:A, 17:17215190:T:TC, 17:17215224:A:AC, 17:17215228:G:C, 17:17215236:TGA:T, 17:17215256:CA:C, 17:17215257:AC:A, 17:17215263:C:CAGGGTGG, 17:17215264:AGGGTGGAGGGTGGAACGTGC:A, 17:17215282:T:TGCGGCTGCGTGGACCTC, 17:17215302:C:T, 17:17215311:CA:C, 17:17215313:A:C, 17:17215317:CTG:C, 17:17216382:G:A, 17:17216394:T:TG, 17:17216394:TG:T, 17:17216395:G:T, 17:17216400:G:A, 17:17216400:G:C, 17:17216400:G:T, 17:17216401:G:A, 17:17216401:G:T, 17:17216418:C:A, 17:17216418:C:CT, 17:17216424:C:A, 17:17216424:C:T, 17:17216426:C:CA, 17:17216427:AG:A, 17:17216433:T:C, 17:17216435:G:T, 17:17216440:G:A, 17:17216443:A:T, 17:17216453:G:C, 17:17216454:T:C, 17:17216458:G:A, 17:17216464:T:C, 17:17216466:T:C, 17:17216466:T:TA, 17:17216476:T:TG, 17:17216478:C:A, 17:17216479:G:C, 17:17216482:C:A, 17:17216484:C:A, 17:17216484:C:T, 17:17216485:A:G, 17:17216487:C:G, 17:17216491:C:CG, 17:17217068:C:G, 17:17217068:CCCGAAGTACTTCAAAAGCTGACT:C, 17:17217074:G:T, 17:17217086:C:G, 17:17217092:G:A, 17:17217112:C:CT, 17:17217116:TC:T, 17:17217124:A:T, 17:17217127:TG:T, 17:17217128:G:A, 17:17217133:C:T, 17:17217147:C:T, 17:17217161:G:A, 17:17217163:A:G, 17:17217168:AG:A, 17:17217174:AC:A, 17:17217175:C:A, 17:17217183:C:A, 17:17217184:T:A, 17:17217184:T:C, 17:17219017:A:C, 17:17219018:C:T, 17:17219030:G:A, 17:17219032:C:T, 17:17219033:G:A, 17:17219038:G:A, 17:17219038:G:T, 17:17219051:G:GCAGATTCCGGGGCTGC, 17:17219082:TGAGA:T, 17:17219126:C:CTTCTGTACTCTCTGGCAACACAGGGGCT, 17:17219133:ACT:A, 17:17219147:CAG:C, 17:17219148:A:AG, 17:17219174:C:CA, 17:17219176:G:C, 17:17219177:A:G, 17:17219183:C:G, 17:17219187:GCTTT:G, 17:17219190:TTC:T, 17:17219194:G:C, 17:17219194:G:T, 17:17219201:C:G, 17:17219206:A:C, 17:17221535:A:G, 17:17221549:C:T, 17:17221555:G:A, 17:17221567:C:A, 17:17221570:C:T, 17:17221575:G:A, 17:17221576:G:C, 17:17221597:C:T, 17:17221599:G:A, 17:17221605:C:T, 17:17221612:C:T, 17:17221614:C:T, 17:17221617:G:A, 17:17221617:G:C, 17:17221618:C:A, 17:17221618:C:G, 17:17221627:G:A, 17:17221628:C:T, 17:17221630:T:C, 17:17222500:C:A, 17:17222501:C:G, 17:17222516:T:G, 17:17222517:G:A, 17:17222519:A:G, 17:17222525:G:GC, 17:17222532:G:T, 17:17222540:T:C, 17:17222541:CACTT:C, 17:17222546:G:T, 17:17222558:A:C, 17:17222558:A:G, 17:17222559:G:C, 17:17222561:G:A, 17:17222564:C:T, 17:17222565:G:A, 17:17222565:G:C, 17:17222582:C:T, 17:17222590:T:TA, 17:17222615:A:C, 17:17222618:C:A, 17:17222628:G:A, 17:17222632:TG:T, 17:17222636:C:T, 17:17222649:C:T, 17:17222657:A:C, 17:17222662:C:T, 17:17222663:T:G, 17:17223929:G:A, 17:17223930:C:T, 17:17223937:C:A, 17:17223937:C:G, 17:17223942:G:A, 17:17223951:T:A, 17:17223955:TC:T, 17:17223959:C:A, 17:17223960:G:A, 17:17223966:T:A, 17:17223975:G:C, 17:17223978:A:T, 17:17223980:G:A, 17:17223981:G:T, 17:17223983:C:A, 17:17223986:G:A, 17:17223989:T:G, 17:17223995:A:G, 17:17223996:G:A, 17:17223998:T:C, 17:17224001:A:G, 17:17224001:A:T, 17:17224002:T:A, 17:17224004:C:T, 17:17224005:G:A, 17:17224008:C:A, 17:17224012:CATGATGG:C, 17:17224017:T:A, 17:17224021:G:C, 17:17224030:G:T, 17:17224032:A:G, 17:17224032:A:T, 17:17224035:A:G, 17:17224037:C:T, 17:17224038:G:A, 17:17224039:C:G, 17:17224041:G:A, 17:17224053:C:A, 17:17224062:C:A, 17:17224073:A:G, 17:17224076:G:T, 17:17224077:T:A, 17:17224083:T:C, 17:17224088:A:G, 17:17224089:C:T, 17:17224091:A:G, 17:17224092:A:C, 17:17224095:C:T, 17:17224099:C:CTG, 17:17224099:CT:C, 17:17224104:C:T, 17:17224107:C:T, 17:17224110:C:T, 17:17224111:G:C, 17:17224111:G:T, 17:17224115:A:C, 17:17224121:G:A, 17:17224121:G:C, 17:17224124:C:A, 17:17224125:C:T, 17:17224128:C:T, 17:17224130:C:A, 17:17224130:C:T, 17:17224131:G:A, 17:17224136:G:A, 17:17224136:G:C, 17:17224137:G:T, 17:17224143:C:A, 17:17224143:C:T, 17:17226174:A:C, 17:17226186:A:C, 17:17226187:G:C, 17:17226192:C:T, 17:17226193:G:A, 17:17226193:G:C, 17:17226202:C:T, 17:17226207:C:G, 17:17226208:G:A, 17:17226210:A:G, 17:17226220:A:C, 17:17226224:C:CT, 17:17226224:C:G, 17:17226243:T:A, 17:17226245:G:GT, 17:17226247:G:A, 17:17226250:T:TG, 17:17226252:A:T, 17:17226262:T:C, 17:17226270:T:C, 17:17226274:T:A, 17:17226276:T:C, 17:17226277:C:G, 17:17226282:C:T, 17:17226291:C:G, 17:17226294:G:A, 17:17226294:G:C, 17:17226295:G:C, 17:17226297:T:G, 17:17226298:G:A, 17:17226301:C:T, 17:17226315:C:T, 17:17226316:G:A, 17:17226318:C:T, 17:17226321:C:A, 17:17226324:T:C, 17:17227887:G:A, 17:17227887:G:T, 17:17227893:C:T, 17:17227899:TCCGA:T, 17:17227904:CT:C, 17:17227923:CT:C, 17:17227930:C:A, 17:17227948:CG:C, 17:17227950:G:T, 17:17227979:CT:C, 17:17228025:C:CT, 17:17228040:TC:T, 17:17228073:G:A, 17:17228077:A:G, 17:17228079:A:T, 17:17228082:A:G, 17:17228085:G:T, 17:17228088:C:A, 17:17228088:C:T, 17:17228089:G:A, 17:17228091:G:A, 17:17228095:C:T, 17:17228104:C:G, 17:17228104:C:T, 17:17228105:G:C, 17:17228105:G:T, 17:17228109:A:T, 17:17228112:T:C, 17:17228114:G:T, 17:17228115:C:T, 17:17228119:G:C, 17:17228124:A:C, 17:17228133:T:C, 17:17228136:A:G, or 17:17228137:T:C, or an mRNA molecule produced therefrom, or a cDNA molecule produced from the mRNA molecule.

For subjects that are genotyped or determined to be FLCN reference, such subjects have an increased risk of developing muscle disorder. For subjects that are genotyped or determined to be either FLCN reference or heterozygous for an FLCN variant nucleic acid molecule, such subjects can be treated with an FLCN inhibitor.

In any of the embodiments described herein, the subject in whom muscle disorder is prevented by administering an FNIP1 inhibitor and/or an FLCN inhibitor may be anyone at risk for developing muscle disorder including, but not limited to, subjects with a genetic predisposition for developing muscle disorder. In some embodiments, administering an FNIP1 inhibitor and/or an FLCN inhibitor to a subject having muscle disorder may be carried out to prevent development of another occurrence of muscle disorder in a subject who has already had muscle disorder. In any of the embodiments described herein, the methods can be used to improve muscle disorder.

In any of the embodiments described herein, the FNIP1 predicted loss-of-function polypeptide can be any FNIP1 polypeptide having a partial loss-of-function, a complete loss-of-function, a predicted partial loss-of-function, or a predicted complete loss-of-function.

In any of the embodiments described herein, the FLCN predicted loss-of-function polypeptide can be any FLCN polypeptide having a partial loss-of-function, a complete loss-of-function, a predicted partial loss-of-function, or a predicted complete loss-of-function.

Any one or more (i.e., any combination) of the FNIP1 variant nucleic acid molecules and/or FLCN variant nucleic acid molecules described herein can be used within any of the methods described herein to determine whether a subject has an increased or decreased risk of developing muscle disorder. The combinations of particular variants can form a mask used for statistical analysis of the particular correlation of FNIP1 and/or FLCN and an increased or decreased risk of developing muscle disorder. In some embodiments, the mask used for statistical analysis of the particular correlation of FNIP1 and/or FLCN and an increased or decreased risk of developing muscle disorder can exclude any one or more of these FNIP1 variant nucleic acid molecule and/or an FLCN variant nucleic acid molecules described herein.

In any of the embodiments described herein, the subject can have muscle disorder. In any of the embodiments described herein, the subject can be at risk of developing muscle disorder. In some embodiments, the muscle disorder comprises sarcopenia, Duchenne Muscular Dystrophy, and Pompe disease. In some embodiments, the muscle disorder comprises sarcopenia. In some embodiments, the muscle disorder comprises Duchenne Muscular Dystrophy. In some embodiments, the muscle disorder comprises Pompe disease.

The present disclosure provides methods of treating a subject having muscle disorder or at risk of developing muscle disorder, the methods comprising administering an FNIP1 inhibitor and/or an FLCN inhibitor to the subject.

The present disclosure provides methods of treating a subject having a complication of muscle disorder or at risk of developing a complication of muscle disorder, the methods comprising administering an FNIP1 inhibitor and/or an FLCN inhibitor to the subject.

In some embodiments, the FNIP1 inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of an FNIP1 nucleic acid molecule. In some embodiments, the antisense RNA, siRNA, or shRNA hybridizes to a sequence within an FNIP1 genomic nucleic acid molecule or mRNA molecule and decreases expression of the FNIP1 polypeptide in a cell in the subject. In some embodiments, the FNIP1 inhibitor comprises an antisense molecule that hybridizes to an FNIP1 genomic nucleic acid molecule or mRNA molecule and decreases expression of the FNIP1 polypeptide in a cell in the subject. In some embodiments, the FNIP1 inhibitor comprises an siRNA that hybridizes to an FNIP1 genomic nucleic acid molecule or mRNA molecule and decreases expression of the FNIP1 polypeptide in a cell in the subject. In some embodiments, the FNIP1 inhibitor comprises an shRNA that hybridizes to an FNIP1 genomic nucleic acid molecule or mRNA molecule and decreases expression of the FNIP1 polypeptide in a cell in the subject.

In some embodiments, the FLCN inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of an FLCN nucleic acid molecule. In some embodiments, the antisense RNA, siRNA, or shRNA hybridizes to a sequence within an FLCN genomic nucleic acid molecule or mRNA molecule and decreases expression of the FLCN polypeptide in a cell in the subject. In some embodiments, the FLCN inhibitor comprises an antisense molecule that hybridizes to an FLCN genomic nucleic acid molecule or mRNA molecule and decreases expression of the FLCN polypeptide in a cell in the subject. In some embodiments, the FLCN inhibitor comprises an siRNA that hybridizes to an FLCN genomic nucleic acid molecule or mRNA molecule and decreases expression of the FLCN polypeptide in a cell in the subject. In some embodiments, the FLCN inhibitor comprises an shRNA that hybridizes to an FLCN genomic nucleic acid molecule or mRNA molecule and decreases expression of the FLCN polypeptide in a cell in the subject. Representative FLCN siRNA molecules are disclosed in, for example, WO 2022/178411.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophore-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthine-9-yl (I), and 2-aminoadenine-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense:
mN*mN*/i2FN/mN/i2FN/mN/i2FN/mN/i2FN/mN/i2FN/mN/
i2FN/mN/i2FN/mN/i2FN/*mN*/32FN/
Antisense:
/52FN/*/i2FN/*mN/i2FN/mN/i2FN/mN/i2FN/mN/i2FN/mN/
i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

In any of the embodiments described herein, the inhibitory nucleic acid molecules may be administered, for example, as one to two hour i.v. infusions or s.c. injections. In any of the embodiments described herein, the inhibitory nucleic acid molecules may be administered at dose levels that range from about 50 mg to about 900 mg, from about 100 mg to about 800 mg, from about 150 mg to about 700 mg, or from about 175 mg to about 640 mg (2.5 to 9.14 mg/kg; 92.5 to 338 mg/m²—based on an assumption of a body weight of 70 kg and a conversion of mg/kg to mg/m² dose levels based on a mg/kg dose multiplier value of 37 for humans).

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the FNIP1 inhibitor or FLCN inhibitor comprises a nuclease agent that induces one or more nicks or double-strand breaks at a recognition sequence(s) or a DNA-binding protein that binds to a recognition sequence within an FNIP1 or FLCN genomic nucleic acid molecule. The recognition sequence can be located within a coding region of the FNIP1 or FLCN gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the FNIP1 or FLCN gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify FNIP1 or FLCN genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of FNIP1 or FLCN nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an FNIP1 or FLCN genomic nucleic acid molecule or it can be a nickase that creates a single-strand break in an FNIP1 or FLCN genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. In some embodiments, a Cas system, such as Cas12a, can have multiple gRNAs encoded into a single crRNA. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of FNIP1 or FLCN genomic nucleic acid molecules can be generated by contacting a cell with a Cas protein and one or more gRNAs that hybridize to one or more gRNA recognition sequences within a target genomic locus in the FNIP1 or FLCN genomic nucleic acid molecule. The gRNA recognition sequence can include or be proximate to the start codon of an FNIP1 or FLCN genomic nucleic acid molecule or the stop codon of an FNIP1 or FLCN genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an FNIP1 or FLCN genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an FNIP1 or FLCN genomic nucleic acid molecule. An exemplary gRNA is a gRNA effective to direct a Cas enzyme to bind to or cleave an FNIP1 or FLCN genomic nucleic acid molecule, wherein the gRNA comprises a DNA-targeting segment that hybridizes to a gRNA recognition sequence within the FNIP1 or FLCN genomic nucleic acid molecule. Exemplary gRNAs comprise a DNA-targeting segment that hybridizes to a gRNA recognition sequence present within an FNIP1 or FLCN genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

The Cas protein and the gRNA form a complex, and the Cas protein cleaves the FNIP1 or FLCN genomic nucleic acid molecule. The Cas protein can cleave the nucleic acid molecule at a site within or outside of the nucleic acid sequence present in the FNIP1 or FLCN genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the FNIP1 or FLCN genomic nucleic acid molecule to which a DNA-targeting segment of a gRNA will bind.

Such methods can result, for example, in an FNIP1 or FLCN genomic nucleic acid molecule in which a region of the FNIP1 or FLCN genomic nucleic acid molecule is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the FNIP1 or FLCN genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the FNIP1 and/or FLCN gRNA molecules can be pooled. For example, the FNIP1 gRNA molecules can be pooled in 15 gRNAs per gene, and can be packaged as multiple (4, 5, or 6, for example) gRNAs per vector (for example, AAV). Likewise, the FLCN gRNA molecules can be pooled in 15 gRNAs per gene, and can be packaged as multiple (4, 5, or 6, for example) gRNAs per vector (for example, AAV).

In any of the methods of treatment or prevention described herein, the subject being treated may comprise an FNIP1 variant nucleic acid molecule and/or an FLCN variant nucleic acid molecule. In some embodiments, the subject being treated is heterozygous for the FNIP1 variant nucleic acid molecule and/or an FLCN variant nucleic acid molecule. In some embodiments, the subject being treated is homozygous for the FNIP1 variant nucleic acid molecule and/or an FLCN variant nucleic acid molecule. In some embodiments, the subject being treated is FNIP1 and/or FLCN reference. The FNIP1 variant nucleic acid molecule and/or an FLCN variant nucleic acid molecule can be any of the FNIP1 variant nucleic acid molecule and/or an FLCN variant nucleic acid molecules disclosed herein. In some embodiments, the FNIP1 variant nucleic acid molecule and/or an FLCN variant nucleic acid molecule is a FNIP1 or FLCN variant genomic nucleic acid molecule that comprises any one or more of the genetic variations described herein, or is an mRNA molecule produced therefrom, or is a cDNA molecule produced from the mRNA molecule.

In some embodiments, the methods of treatment or prevention further comprise detecting the presence or absence of an FNIP1 variant nucleic acid molecule and/or an FLCN variant nucleic acid molecule in a biological sample from the subject. In some embodiments, the FNIP1 variant nucleic acid molecule and/or an FLCN variant nucleic acid molecule can be any of the FNIP1 variant nucleic acid molecule and/or an FLCN variant nucleic acid molecules disclosed herein. In some embodiments, the FNIP1 variant nucleic acid molecule and/or an FLCN variant nucleic acid molecule is an FNIP1 or FLCN variant genomic nucleic acid molecule that comprises any one or more of the genetic variations described herein, or is an mRNA molecule produced therefrom, or is a cDNA molecule produced from the mRNA molecule.

The present disclosure also provides methods of treating a subject with a muscle disorder therapeutic agent, wherein the subject has muscle disorder or is at risk of developing muscle disorder. The methods comprise determining whether the subject has an FNIP1 variant nucleic acid molecule and/or an FLCN variant nucleic acid molecule by obtaining or having obtained a biological sample from the subject, and performing or having performed a sequence analysis on the biological sample to determine if the subject has a genotype comprising the FNIP1 variant nucleic acid molecule and/or an FLCN variant nucleic acid molecule. In embodiments where the subject is FNIP1 and/or FLCN reference, the methods further comprise administering or continuing to administer the muscle disorder therapeutic agent in a standard dosage amount to the subject, and/or administering an FNIP1 inhibitor and/or an FLCN inhibitor to the subject. In embodiments where the subject is heterozygous for the FNIP1 variant nucleic acid molecule and/or an FLCN variant nucleic acid molecule, the methods further comprise administering or continuing to administer the muscle disorder therapeutic agent in an amount that is the same as or less than a standard dosage amount to the subject, and/or administering an FNIP1 inhibitor and/or an FLCN inhibitor to the subject. In embodiments where the subject is homozygous for the FNIP1 variant nucleic acid molecule and/or an FLCN variant nucleic acid molecule, the methods further comprise administering or continuing to administer the muscle disorder therapeutic agent in an amount that is the same as or less than a standard dosage amount. The presence of an FNIP1 variant nucleic acid molecule and/or an FLCN variant nucleic acid molecule indicates the subject has a decreased risk of developing muscle disorder. In some embodiments, the subject is FNIP1 and/or FLCN reference. In some embodiments, the subject is heterozygous for an FNIP1 variant nucleic acid molecule and/or an FLCN variant nucleic acid molecule. In some embodiments, the subject is homozygous for an FNIP1 variant nucleic acid molecule and/or an FLCN variant nucleic acid molecule. In any of the embodiments described herein, the FNIP1 inhibitor or FLCN inhibitor is an example of a muscle disorder therapeutic agent. In some embodiments, the FNIP1 variant nucleic acid molecule and/or an FLCN variant nucleic acid molecule is a FNIP1 or FLCN variant genomic nucleic acid molecule that comprises any one or more of the genetic variations described herein, or is an mRNA molecule produced therefrom, or is a cDNA molecule produced from the mRNA molecule.

For subjects that are genotyped or determined to be either FNIP1 and/or FLCN reference or heterozygous for an FNIP1 variant nucleic acid molecule and/or an FLCN variant nucleic acid molecule, such subjects can be administered an FNIP1 inhibitor and/or an FLCN inhibitor, as described herein.

Detecting the presence or absence of an FNIP1 variant nucleic acid molecule and/or an FLCN variant nucleic acid molecule in a biological sample from a subject and/or determining whether a subject has an FNIP1 variant nucleic acid molecule and/or an FLCN variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is FNIP1 reference, the subject is administered a muscle disorder therapeutic agent in an amount that is the same as or less than a standard dosage amount, and/or an FNIP1 inhibitor. In some embodiments, when the subject is heterozygous for an FNIP1 variant nucleic acid molecule, the subject is administered a muscle disorder therapeutic agent in an amount that is the same as or less than a standard dosage amount, and/or an FNIP1 inhibitor.

In some embodiments, when the subject is FLCN reference, the subject is administered a muscle disorder therapeutic agent in an amount that is the same as or less than a standard dosage amount, and/or an FLCN inhibitor. In some embodiments, when the subject is heterozygous for an FLCN variant nucleic acid molecule, the subject is administered a muscle disorder therapeutic agent in an amount that is the same as or less than a standard dosage amount, and/or an FLCN inhibitor.

In some embodiments, the treatment or prevention methods comprise detecting the presence or absence of a decrease in the expression of an FNIP1 variant mRNA or polypeptide and/or FLCN variant mRNA or polypeptide in a biological sample from the subject. In some embodiments, when the subject does not have a decrease in the expression of an FNIP1 variant mRNA or polypeptide and/or FLCN variant mRNA or polypeptide, the subject is administered a muscle disorder therapeutic agent in a standard dosage amount, and/or an FNIP1 inhibitor and/or an FLCN inhibitor. In some embodiments, when the subject has a decrease in the expression of an FNIP1 variant mRNA or polypeptide and/or FLCN variant mRNA or polypeptide, the subject is administered a muscle disorder therapeutic agent in an amount that is the same as or less than a standard dosage amount.

The present disclosure also provides methods of treating a subject with a muscle disorder therapeutic agent, wherein the subject has muscle disorder or is at risk of developing muscle disorder. The methods comprise determining whether the subject has a decrease in the expression of an FNIP1 variant mRNA or polypeptide and/or an FLCN variant mRNA or polypeptide by obtaining or having obtained a biological sample from the subject, and performing or having performed an assay on the biological sample to determine if the subject a decrease in the expression of an FNIP1 variant mRNA or polypeptide and/or an FLCN variant mRNA or polypeptide. In embodiments where the subject does not have a decrease in the expression of an FNIP1 variant mRNA or polypeptide and/or an FLCN variant mRNA or polypeptide, the methods further comprise administering or continuing to administer the muscle disorder therapeutic agent in a standard dosage amount to the subject, and/or administering an FNIP1 inhibitor and/or an FLCN inhibitor to the subject. In embodiments where the subject has a decrease in the expression of an FNIP1 variant mRNA or polypeptide and/or an FLCN variant mRNA or polypeptide, the methods further comprise administering or continuing to administer the muscle disorder therapeutic agent in an amount that is the same as or less than a standard dosage amount to the subject. The presence of a decrease in the expression of an FNIP1 variant mRNA or polypeptide and/or an FLCN variant mRNA or polypeptide indicates the subject has a decreased risk of developing muscle disorder. In some embodiments, the subject has a decrease in the expression of an FNIP1 variant mRNA or polypeptide and/or an FLCN variant mRNA or polypeptide. In some embodiments, the subject does not have a decrease in the expression of an FNIP1 variant mRNA or polypeptide and/or an FLCN variant mRNA or polypeptide. In any of the embodiments described herein, the FNIP1 inhibitor or FLCN inhibitor is an example of a muscle disorder therapeutic agent. In some embodiments, the FNIP1 variant nucleic acid molecule and/or an FLCN variant nucleic acid molecule is an FNIP1 variant genomic nucleic acid molecule or FLCN variant genomic nucleic acid molecule that comprises any one or more of the genetic variations described herein, or is an mRNA molecule produced therefrom, or is a cDNA molecule produced from the mRNA molecule.

Detecting a decrease in the expression of an FNIP1 variant mRNA or polypeptide and/or an FLCN variant mRNA or polypeptide can be carried out by a variety of known methods. In some embodiments, these methods can be carried out in vitro. In some embodiments, these methods can be carried out in situ. In some embodiments, these methods can be carried out in vivo. In any of these embodiments, the mRNA or polypeptide can be present within a cell obtained from the subject.

In some embodiments, the treatment or prevention methods comprise detecting the presence or absence of an FNIP1 variant polypeptide and/or an FLCN variant polypeptide in a biological sample from the subject. In some embodiments, when the subject does not have an FNIP1 variant polypeptide and/or an FLCN variant polypeptide, the subject is administered a muscle disorder therapeutic agent in an amount that is the same as or less than a standard dosage amount, and/or an FNIP1 inhibitor and/or an FLCN inhibitor. In some embodiments, when the subject has an FNIP1 variant polypeptide and/or an FLCN variant polypeptide, the subject is administered a muscle disorder therapeutic agent in standard dosage amount.

The present disclosure also provides methods of treating a subject with a muscle disorder therapeutic agent, wherein the subject has muscle disorder or is at risk of developing muscle disorder. The methods comprise determining whether the subject has an FNIP1 variant polypeptide and/or an FLCN variant polypeptide by obtaining or having obtained a biological sample from the subject and performing or having performed an assay on the biological sample to determine if the subject has an FNIP1 variant polypeptide and/or an FLCN variant polypeptide. When the subject does not have an FNIP1 variant polypeptide and/or an FLCN variant polypeptide, the subject is administered the muscle disorder therapeutic agent in an amount that is the same as or less than a standard dosage amount, and/or an FNIP1 inhibitor and/or an FLCN inhibitor. When the subject has an FNIP1 variant polypeptide and/or an FLCN variant polypeptide, the subject is administered the muscle disorder therapeutic agent in a standard dosage amount. The presence of an FNIP1 variant polypeptide and/or an FLCN variant polypeptide indicates the subject has a decreased risk of developing muscle disorder. In some embodiments, the subject has an FNIP1 variant polypeptide and/or an FLCN variant polypeptide. In some embodiments, the subject does not have an FNIP1 variant polypeptide and/or an FLCN variant polypeptide.

The present disclosure also provides methods of preventing a subject from developing muscle disorder by administering a muscle disorder therapeutic agent. In some embodiments, the method comprises determining whether the subject has an FNIP1 variant polypeptide and/or an FLCN variant polypeptide by obtaining or having obtained a biological sample from the subject and performing or having performed an assay on the biological sample to determine if the subject has an FNIP1 variant polypeptide and/or an FLCN variant polypeptide. When the subject does not have an FNIP1 variant polypeptide and/or an FLCN variant polypeptide, the subject is administered the muscle disorder therapeutic agent in an amount that is the same as or less than a standard dosage amount, and/or an FNIP1 inhibitor and/or an FLCN inhibitor. When the subject has an FNIP1 variant polypeptide and/or an FLCN variant polypeptide, the subject is administered the muscle disorder therapeutic agent in an amount that is the same as or less than a standard dosage amount. The presence of an FNIP1 variant polypeptide and/or an FLCN variant polypeptide indicates the subject has a decreased risk of developing muscle disorder. In some embodiments, the subject has an FNIP1 variant polypeptide and/or an FLCN variant polypeptide. In some embodiments, the subject does not have an FNIP1 variant polypeptide and/or an FLCN variant polypeptide.

Detecting the presence or absence of an FNIP1 variant polypeptide and/or an FLCN variant polypeptide in a biological sample from a subject and/or determining whether a subject has an FNIP1 variant polypeptide and/or an FLCN variant polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the FNIP1 inhibitor and/or FLCN inhibitor is a small molecule. In some embodiments, the small molecule is low molecular weight (<900 daltons) organic compound.

In some embodiments, the FNIP1 inhibitor and/or FLCN inhibitor comprises an antibody, or antigen-binding fragment thereof. In some embodiments, the antibody, or antigen-binding fragment thereof, binds specifically to human FNIP1 or FLCN. In some embodiments, the antibody is a fully human monoclonal antibody (mAb), or antigen-binding fragment thereof, that specifically binds and neutralizes, inhibits, blocks, abrogates, reduces, or interferes with, at least one activity of FNIP1 or FLCN, in particular, human FNIP1 or FLCN. In some embodiments, an antibody or fragment thereof can neutralize, inhibit, block, abrogate, reduce, or interfere with, an activity of FNIP1 or FLCN by binding to an epitope of FNIP1 or FLCN that is directly involved in the targeted activity of FNIP1 or FLCN. In some embodiments, an antibody or fragment thereof can neutralize, inhibit, block, abrogate, reduce, or interfere with, an activity of FNIP1 or FLCN by binding to an epitope of FNIP1 or FLCN that is not directly involved in the targeted activity of FNIP1 or FLCN, but the antibody or fragment binding thereto sterically or conformationally inhibits, blocks, abrogates, reduces, or interferes with, the targeted activity of FNIP1 or FLCN. In some embodiments, an antibody or fragment thereof binds to an epitope of FNIP1 or FLCN that is not directly involved in the targeted activity of FNIP1 or FLCN (i.e., a non-blocking antibody), but the antibody or fragment binding thereto results in the enhancement of the clearance of FNIP1 or FLCN from the circulation, compared to the clearance of FNIP1 or FLCN in the absence of the antibody or fragment thereof, thereby indirectly inhibiting, blocking, abrogating, reducing, or interfering with, an activity of FNIP1 or FLCN. Clearance of FNIP1 or FLCN from the circulation can be particularly enhanced by combining two or more different non-blocking antibodies that do not compete with one another for specific binding to FNIP1 or FLCN. The antibodies can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al., J. Immunol., 2000, 164, 1925-1933).

In some embodiments, the antibody or antigen-binding fragment thereof specifically binds to FNIP1 or FLCN with an equilibrium dissociation constant (K_{D}) of about 7 nM or less, about 6 nM or less, about 5 nM or less, about 4 nM or less, about 3 nM or less, about 2 nM or less, or about 1 nM or less, as measured by surface plasmon resonance assay (for example, BIACORE™). In some embodiments, the antibody exhibits a K_{D} of about 800 pM or less, about 700 pM or less; about 600 pM or less; about 500 pM or less; about 400 pM or less; about 300 pM or less; about 200 pM or less; about 100 pM or less; or about 50 pM or less.

In some embodiments, the anti-FNIP1 antibodies and/or anti-FLCN antibodies have a modified glycosylation pattern. In some applications, modification to remove undesirable glycosylation sites may be useful, or e.g., removal of a fucose moiety to increase antibody dependent cellular cytotoxicity (ADCC) function (see, Shield et al., J. Biol. Chem., 2002, 277, 26733). In other applications, removal of N-glycosylation site may reduce undesirable immune reactions against the therapeutic antibodies or increase affinities of the antibodies. In yet other applications, modification of galactosylation can be made in order to modify complement dependent cytotoxicity (CDC).

In some embodiments, the muscle disorder therapeutic agent includes, but is not limited to, a glucocorticosteroid (such as, for example, prednisone, prednisolone, deflazacort, and others), testosterone and other androgenic hormones or analogues, or an androgen receptor modulator (such as, for example, enobosarm), ataluren, an oligonucleotide (such as, for example, eteplirsen, golodirsen, viltolarsen, and casimersen), losmapimod, zetomipzomib, venoglobulin-IH, an activin II receptor antagonist (such as, for example, bimagrumab), a myostatin inhibitor (such as, for example, trevogrumab and domagrozumab), an activin A antagonist (such as, for example, garetosmab), and reldesemtiv.

The present disclosure also provides compositions comprising a combination of an antibody or antigen-binding fragment thereof and a muscle disorder therapeutic agent.

In some embodiments, the muscle disorder therapeutic agent can be combined with an FNIP1 inhibitor and/or an FLCN inhibitor.

In some embodiments, the dose of the muscle disorder therapeutic agents can be decreased by about 10%, by about 20%, by about 30%, by about 40%, by about 50%, by about 60%, by about 70%, by about 80%, or by about 90% for subjects that are heterozygous for an FNIP1 variant nucleic acid molecule and/or an FLCN variant nucleic acid molecule or FNIP1 reference and/or FLCN reference (i.e., a less than the standard dosage amount) compared to subjects that are homozygous for an FNIP1 variant nucleic acid molecule and/or an FLCN variant nucleic acid molecule (who may receive a standard dosage amount). In some embodiments, the dose of the muscle disorder therapeutic agents can be decreased by about 10%, by about 20%, by about 30%, by about 40%, or by about 50%. In some embodiments, the dose of the muscle disorder therapeutic agents can be decreased by about 10%, by about 20%, by about 30%, by about 40%, by about 50%, by about 60%, by about 70%, by about 80%, or by about 90% for subjects that are heterozygous for an FNIP1 variant nucleic acid molecule and/or an FLCN variant nucleic acid molecule compared to subjects that are or FNIP1 reference and/or FLCN reference. In addition, subjects that are heterozygous for an FNIP1 variant nucleic acid molecule and/or an FLCN variant nucleic acid molecule or FNIP1 reference and/or FLCN reference can be administered the muscle disorder therapeutic agents less frequently compared to subjects that are heterozygous for the FNIP1 variant nucleic acid molecule and/or an FLCN variant nucleic acid molecule.

Administration of the muscle disorder therapeutic agents and/or FNIP1 inhibitors and/or FLCN inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the muscle disorder therapeutic agents and/or FNIP1 inhibitors and/or FLCN inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients, or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in muscle disorder, a decrease/reduction in the severity of muscle disorder (such as, for example, a reduction or inhibition of development of muscle disorder), a decrease/reduction in symptoms and disease-related effects, delaying the onset of symptoms and disease-related effects, reducing the severity of symptoms of disease-related effects, reducing the number of symptoms and disease-related effects, reducing the latency of symptoms and disease-related effects, an amelioration of symptoms and disease-related effects, reducing secondary symptoms, reducing secondary infections, preventing relapse to muscle disorder, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of muscle disorder development/progression (such as, for example, a complete or partial avoidance/inhibition or a delay), and an increased survival time of the affected host animal, following administration of a therapeutic protocol. Treatment of muscle disorder encompasses the treatment of a subject already diagnosed as having any form of muscle disorder at any clinical stage or manifestation, the delay of the onset or evolution or aggravation or deterioration of the symptoms or signs of muscle disorder, and/or preventing and/or reducing the severity of muscle disorder.

In some embodiments, the FNIP1 inhibitor and/or FLCN inhibitor and the muscle disorder therapeutic agent are disposed within a pharmaceutical composition. In some embodiments, the FNIP1 inhibitor and/or FLCN inhibitor is disposed within a first pharmaceutical composition and the muscle disorder therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

In any of the embodiments described herein, administration of the FNIP1 inhibitor and/or FLCN inhibitor can be targeted to a specific tissue. For example, in some embodiments, the FNIP1 inhibitor and/or FLCN inhibitor can be targeted to muscle tissue, muscle cells, skeletal muscle tissue, or skeletal muscle cells. In some embodiments described herein, administration of the FNIP1 inhibitor and/or FLCN inhibitor can be targeted to muscle tissue, muscle cells, skeletal muscle tissue, or skeletal muscle cells. For example, the FNIP1 inhibitor and/or FLCN inhibitor can be linked to CACNG1 antibody, or an antigen-binding fragment thereof. In some embodiments, a muscle-specific FNIP1 siRNA (CACNG1 mAB-siRNA) is used to promote oxidative metabolism and lysosomal biogenesis in muscle fibers.

In some embodiments, the CACNG1 antibody, or antigen-binding fragment thereof, comprises a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity. In some embodiments, the CACNG1 antibody, or antigen-binding fragment thereof, comprises a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity. In some embodiments, the CACNG1 antibody, or antigen-binding fragment thereof, comprises a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity. In some embodiments, the CACNG1 antibody, or antigen-binding fragment thereof, comprises a light chain CDR1 (LCDR1) comprising an amino acid sequence selected from any of the LCDR1 amino acid sequences listed in Table 1 or a substantially similar sequence thereof having at least 90%, at least 95%, at least 98% or at least 99% sequence identity. In some embodiments, the CACNG1 antibody, or antigen-binding fragment thereof, comprises a light chain CDR2 (LCDR2) comprising an amino acid sequence selected from any of the LCDR2 amino acid sequences listed in Table 1 or a substantially similar sequence thereof having at least 90%, at least 95%, at least 98% or at least 99% sequence identity. In some embodiments, the CACNG1 antibody, or antigen-binding fragment thereof, comprises a light chain CDR3 (LCDR3) comprising an amino acid sequence selected from any of the LCDR3 amino acid sequences listed in Table 1 or a substantially similar sequence thereof having at least 90%, at least 95%, at least 98% or at least 99% sequence identity.

In some embodiments, the CACNG1 antibody, or antigen-binding fragment thereof, comprises an HCVR amino acid sequence listed in Table 1, or a substantially similar sequence thereof having at least 90%, at least 95%, at least 98% or at least 99% sequence identity thereto. In some embodiments, the CACNG1 antibody, or antigen-binding fragment thereof, comprises an LCVR amino acid sequence listed in Table 1, or a substantially similar sequence thereof having at least 90%, at least 95%, at least 98% or at least 99% sequence identity thereto.

In some embodiments, the CACNG1 antibody, or antigen-binding fragment thereof, comprises an HC amino acid sequence selected from any one of SEQ ID NO: 97 to SEQ ID NO: 108, or a substantially similar sequence thereof having at least 90%, at least 95%, at least 98% or at least 99% sequence identity thereto. In some embodiments, the CACNG1 antibody, or antigen-binding fragment thereof, comprises an LC amino acid sequence selected from any one of SEQ ID NO: 109 to SEQ ID NO: 120, or a substantially similar sequence thereof having at least 90%, at least 95%, at least 98% or at least 99% sequence identity thereto.

TABLE 1
anti-CACNG1 Antibodies

| | SEQ ID NOs: | | | | | | | |
|---|---|---|---|---|---|---|---|---|
| Antibody | Heavy Chain | | | | Light Chain | | | |
| Designation | VR | CDR1 | CDR2 | CDR3 | VR | CDR1 | CDR2 | CDR3 |
| 1 | 1 | 13 | 25 | 37 | 49 | 61 | 73 | 85 |
| 2 | 2 | 14 | 26 | 38 | 50 | 62 | 74 | 86 |
| 3 | 3 | 15 | 27 | 39 | 51 | 63 | 75 | 87 |
| 4 | 4 | 16 | 28 | 40 | 52 | 64 | 76 | 88 |
| 5 | 5 | 17 | 29 | 41 | 53 | 65 | 77 | 89 |
| 6 | 6 | 18 | 30 | 42 | 54 | 66 | 78 | 90 |
| 7 | 7 | 19 | 31 | 43 | 55 | 67 | 79 | 91 |
| 8 | 8 | 20 | 32 | 44 | 56 | 68 | 80 | 92 |
| 9 | 9 | 21 | 33 | 45 | 57 | 69 | 81 | 93 |
| 10 | 10 | 22 | 34 | 46 | 58 | 70 | 82 | 94 |
| 11 | 11 | 23 | 35 | 47 | 59 | 71 | 83 | 95 |
| 12 | 12 | 24 | 36 | 48 | 60 | 72 | 84 | 96 |

HCVR Amino Acid Sequences:
(SEQ ID NO: 1)
QVQLVESGGGVVQPGRSLRLSCTASGITFRNYGMHWVRQAPGKGLEWVAVMWYDGSNKYYADSVK
GRFTISGDNSKVYLQMNSLRAEDTAVYYCARRGTIRTAAPFDYWGQGTLVTVSS;
(SEQ ID NO: 2)
QVQLQQWGAGLLKPSATLSRTCAVYGGSFSGYYWNWIRQSPGKGLEWIGEILHSGRTNYNPSLKSRVTI
SVDTSKNQFSLKLTSVTAADTAVYYCAGRIAARHGWFDPWGQGTLVTVSS;
(SEQ ID NO: 3)
QVQLVESGGGVVQPGRSLRLSCAASGFTFSTYGMHWVRQAPGKGLEWVAVIWHDGSDKYYVDSVKG
RFSIARDNSKNTLYLQMNSLRVEDTGIYYCARRGIRGTVFDHWGLGTLVTVSS;
(SEQ ID NO: 4)
QVQLQESGPGLVKPSETLSLTCTVSGDSINNYYWTWLRQPPGKGLEWIGYIYYSGSANYNPSLKSRVTISV
DTSKNQFSLKLNSVTAADTAVYYCARGAVKYFRHWGQGTLVTVSS;
(SEQ ID NO: 5)
QVQLVESGGGVVQPGTSLRLSCAASGFTFSSYGMHWVRQAPGKGLEWVAVIWIDGSNKYYADSVKGR
FTISRDNSKNTLYLQMNSLRAEDTAVYYCARRGGIVVAAPFDYWGQGTLVTVSS;
(SEQ ID NO: 6)
QVQLVESGGGVVQPGRSLRLSCEASGITFRNYGMHWVRQAPGKGLEWVAVMWYDGSNKYYADSVK
GRFTISRDNSKNTVYLQMNSLRAEDTAVYYCARRGHIATAAPFDYWGQGTLVTVSS;
(SEQ ID NO: 7)
QVQLVESGGGVVQPGRSLRLSCTASGFTFRSYGMHWVRQAPGKGLEWVSVIWIDGNNIYYADSVKGR
FTISRDNSKNTLYLQMDSLRAEDTAVYYCARRLAITSAAPFDYWGQGTLVTVSS;
(SEQ ID NO: 8)
QVQLVQSGAEVKKPGASVKVSCKASGYAFTTYGITWVRQAPGQGLEWMGWISAYNGNTNYAEKVQG
RFTMTTDTSTNTAYMELRSLRSDDTAVYFCARKGHYGSGTYYNPFGFDFWGQGTMVTVSS;
(SEQ ID NO: 9)
EVQLVESGGGLVQPGGSLKLSCTASGLTLSDSAMHWVRQASGKGLEWVGRIRNKANRYATEYAASVKG
RFTISRDDSKNTAYLQMNSLKTEDTAVYYCTRNWKIFLFDYWGQGTLVTVSS;
(SEQ ID NO: 10)
EVQLLESGGGLVQPGGSLRLSCAASGFTFNNYGMSWVRQGPGKGLEWVSSISGSGGTTFYADSVKGRF
TISRDNSKNTLYLQMNSLRAEDTAVYYCGKGGYCSSSGCRHYGMDVWGQGTTVTVSS;
(SEQ ID NO: 11)
EVQLVESGGNLVQPGGSLRLSCAASGFTFTSHAMNWVRQAPGKGLEWVSVITGRGFDTHYADSVKGR
FTISRDISKNTLYLQMNSLRAEDTAVYYCAKGLYDSGNYYIDYWGQGTLVTVSS;
(SEQ ID NO: 12)
QVQLVQSGAEVKKPGASVKVSCKASGYAFTTYGITWVRQAPGQGLEWMGWISAYNGNTNYAEKVQG
RFTMTTDTSTNTAYMELRSLRSDDTAVYFCARKGHYGSGTYYNPFGFDFWGQGTMVTVSS.
HCDR1 Amino Acid Sequences:
(SEQ ID NO: 13)
GITFRNYG;
(SEQ ID NO: 14)
GGSFSGYY;
(SEQ ID NO: 15)
GFTFSTYG;
(SEQ ID NO: 16)
GDSINNYY;
(SEQ ID NO: 17)
GFTFSSYG;
(SEQ ID NO: 18)
GITFRNYG;
(SEQ ID NO: 19)
GFTFRSYG;
(SEQ ID NO: 20)
GYAFTTYG;
(SEQ ID NO: 21)
GLTLSDSA;
(SEQ ID NO: 22)
GFTFNNYG
(SEQ ID NO: 23)
GFTFTSHA;
(SEQ ID NO: 24)
GYAFTTYG.
HCDR2 Amino Acid Sequences:
(SEQ ID NO: 25)
MWYDGSNK;
(SEQ ID NO: 26)
ILHSGRT;
(SEQ ID NO: 27)
IWHDGSDK;
(SEQ ID NO: 28)
IYYSGSA;
(SEQ ID NO: 29)
IWIDGSNK;
(SEQ ID NO: 30)
MWYDGSN;
(SEQ ID NO: 31)
IWIDGNNI;
(SEQ ID NO: 32)
ISAYNGN;
(SEQ ID NO: 33)
IRNKANRYAT;
(SEQ ID NO: 34)
SGSGGT;
(SEQ ID NO: 35)
ITGRGFDT;
(SEQ ID NO: 36)
ISAYNGNT.
HCDR3 Amino Acid Sequences:
(SEQ ID NO: 37)
ARRGTIRTAAPFDY;
(SEQ ID NO: 38)
AGRIAARHGWFDP;
(SEQ ID NO: 39)
ARRGIRGTVFDH;
(SEQ ID NO: 40)
ARGAVKYFRH;
(SEQ ID NO: 41)
ARRGGIVVAAPFDY;
(SEQ ID NO: 42)
ARRGHIATAAPFD;
(SEQ ID NO: 43)
ARRLAITSAAPFDY;
(SEQ ID NO: 44)
CARKGHYGSGTYYNPFGFD;
(SEQ ID NO: 45)
TRNWKIFLFDY;
(SEQ ID NO: 46)
CGKGGYCSSSGCRH;
(SEQ ID NO: 47)
AKGLYDSGNYYIDY;
(SEQ ID NO: 48)
ARKGHYGSGTYYNPFGFDF.
HC Amino Acid Sequence:
(SEQ ID NO: 97)
QVQLVESGGGVVQPGRSLRLSCTASGITFRNYGMHWVRQAPGKGLEWVAVMWYDGSNKYYADSVK
GRFTISGDNSKVYLQMNSLRAEDTAVYYCARRGTIRTAAPFDYWGQGTLVTVSSASTKGPSVFPLAPCSR
STSESTAALGCLVKDYFPEPVTVSWNSGALTSGVHTFPAVLQSSGLYSLSSVVTVPSSSLGTKTYTCNVDH
KPSNTKVDKRVESKYGPPCPPCPAPGGGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPVLDSDGSFFLYSRLTVDKSRW
QEGNVFSCSVMHEALHNHYTQKSLSLSLGK *underlined and bolded asparagine
(N) may be mutated to a glutamine (Q) for conjugation by transglutaminase;
(SEQ ID NO: 98)
QVQLQQWGAGLLKPSATLSRTCAVYGGSFSGYYWNWIRQSPGKGLEWIGEILHSGRTNYNPSLKSRVTI
SVDTSKNQFSLKLTSVTAADTAVYYCAGRIAARHGWFDPWGQGTLVTVSSASTKGPSVFPLAPCSRSTSE
STAALGCLVKDYFPEPVTVSWNSGALTSGVHTFPAVLQSSGLYSLSSVVTVPSSSLGTKTYTCNVDHKPSN
TKVDKRVESKYGPPCPPCPAPEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDSDGSFFLYSRLTVDKSRWQE
GNVFSCSVMHEALHNHYTQKSLSLSLGK underlined and bolded asparagine
(N) may be mutated to a glutamine (Q) for conjugation by transglutaminase;
(SEQ ID NO: 99)
QVQLVESGGGVVQPGRSLRLSCAASGFTFSTYGMHWVRQAPGKGLEWVAVIWHDGSDKYYVDSVKG
RFSIARDNSKNTLYLQMNSLRVEDTGIYYCARRGIRGTVFDHWGLGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKP
SNTKVDKRVESKYGPPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLDSDGSFFLYSRLTVDKSRWQ
EGNVFSCSVMHEALHNHYTQKSLSLSLGK *underlined and bolded asparagine
(N) may be mutated to a glutamine (Q) for conjugation by transglutaminase;
(SEQ ID NO: 100)
QVQLQESGPGLVKPSETLSLTCTVSGDSINNYYWTWLRQPPGKGLEWIGYIYYSGSANYNPSLKSRVTISV
DTSKNQFSLKLNSVTAADTAVYYCARGAVKYFRHWGQGTLVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSNTKVD
KRVESKYGPPCPPCPAPEFLGGPSVFLFPPKPKDTLMISRTPEVTCVVVDVSQEDPEVQFNWYVDGVEV
HNAKTKPREEQFNSTYRVVSVLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSFFLYSRLTVDKSRWQEGNVF
SCSVMHEALHNHYTQKSLSLSLGK *underlined and bolded asparagine (N)
may be mutated to a glutamine (Q) for conjugation by transglutaminase;
(SEQ ID NO: 101)
QVQLVESGGGVVQPGTSLRLSCAASGFTFSSYGMHWVRQAPGKGLEWVAVIWIDGSNKYYADSVKGR
FTISRDNSKNTLYLQMNSLRAEDTAVYYCARRGGIVVAAPFDYWGQGTLVTVSSASTKGPSVFPLAPCSR
STSESTAALGCLVKDYFPEPVTVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPVLDSDGSFFLYSRLTVDKSRW
QEGNVFSCSVMHEALHNHYTQKSLSLSLGK *underlined and bolded asparagine (N)
may be mutated to a glutamine (Q) for conjugation by transglutaminase;
(SEQ ID NO: 102)
QVQLVESGGGVVQPGRSLRLSCTASGFTFRSYGMHWVRQAPGKGLEWVSVIWIDGNNIYYADSVKGR
FTISRDNSKNTLYLQMDSLRAEDTAVYYCARRLAITSAAPFDYWGQGTLVTVSSASTKGPSVFPLAPCSRS
TSESTAALGCLVKDYFPEPVTVSWNSGALTSGVHTFPAVLQSSGLYSLSSVVTVPSSSLGTKTYTCNVDHK
PSNTKVDKRVESKYGPPCPPCPAPEFLGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPVLDSDGSFFLYSRLTVDKSRW
QEGNVFSCSVMHEALHNHYTQKSLSLSLGK *underlined and bolded asparagine (N)
may be mutated to a glutamine (Q) for conjugation by transglutaminase;
(SEQ ID NO: 103)
EVQLVESGGNLVQPGGSLRLSCAASGFTFTSHAMNWVRQAPGKGLEWVSVITGRGFDTHYADSVKGR
FTISRDISKNTLYLQMNSLRAEDTAVYYCAKGLYDSGNYYIDYWGQGTLVTVSSASTKGPSVFPLAPCSRS
TSESTAALGCLVKDYFPEPVTVSWNSGALTSGVHTFPAVLQSSGLYSLSSVVTVPSSSLGTKTYTCNVDHK
PSNTKVDKRVESKYGPPCPPCPAPEFLGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPVLDSDGSFFLYSRLTVDKSRW
QEGNVFSCSVMHEALHNHYTQKSLSLSLGK *underlined and bolded asparagine (N)
may be mutated to a glutamine (Q) for conjugation by transglutaminase;
(SEQ ID NO: 104)
QVQLVESGGGVVQPGRSLRLSCEASGITFRNYGMHWVRQAPGKGLEWVAVMWYDGSNKYYADSVK
GRFTISRDNSKNTVYLQMNSLRAEDTAVYYCARRGHIATAAPFDYWGQGTLVTVSSAKTTAPSVYPLAP
VCGDTTGSSVTLGCLVKGYFPEPVTLTWNSGSLSSGVHTFPAVLQSDLYTLSSSVTVTSSTWPSQSITCNV
AHPASSTKVDKKIEPRGPTIKPCPPCKCPAPNLLGGPSVFIFPPKIKDVLMISLSPIVTCVVVDVSEDDPDVQ
ISWFVNNVEVHTAQTQTHREDYNSTLRVVSALPIQHQDWMSGKEFKCKVNNKDLPAPIERTISKPKGSV
RAPQVYVLPPPEEEMTKKQVTLTCMVTDFMPEDIYVEWTNNGKTELNYKNTEPVLDSDGSYFMYSKLR
VEKKNWVERNSYSCSVVHEGLHNHHTTKSFSRTPGK;
(SEQ ID NO: 105)
QVQLVQSGAEVKKPGASVKVSCKASGYAFTTYGITWVRQAPGQGLEWMGWISAYNGNTNYAEKVQG
RFTMTTDTSTNTAYMELRSLRSDDTAVYFCARKGHYGSGTYYNPFGFDFWGQGTMVTVSSAKTTPPSV
YPLAPGSAAQTNSMVTLGCLVKGYFPEPVTVTWNSGSLSSGVHTFPAVLQSDLYTLSSSVTVPSSTWPSE
TVTCNVAHPASSTKVDKKIVPRDCGCKPCICTVPEVSSVFIFPPKPKDVLTITLTPKVTCVVVDISKDDPEVQ
FSWFVDDVEVHTAQTQPREEQFNSTFRSVSELPIMHQDWLNGKEFKCRVNSAAFPAPIEKTISKTKGRP
KAPQVYTIPPPKEQMAKDKVSLTCMITDFFPEDITVEWQWNGQPAENYKNTQPIMDTDGSYFVYSKLN
VQKSNWEAGNTFTCSVLHEGLHNHHTEKSLSHSPGK;
(SEQ ID NO: 106)
EVQLVESGGGLVQPGGSLKLSCTASGLTLSDSAMHWVRQASGKGLEWVGRIRNKANRYATEYAASVKG
RFTISRDDSKNTAYLQMNSLKTEDTAVYYCTRNWKIFLFDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKP
SNTKVDKRVESKYGPPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLDSDGSFFLYSRLTVDKSRWQ
EGNVFSCSVMHEALHNHYTQKSLSLSLGK;
(SEQ ID NO: 107)
EVQLLESGGGLVQPGGSLRLSCAASGFTFNNYGMSWVRQGPGKGLEWVSSISGSGGTTFYADSVKGRF
TISRDNSKNTLYLQMNSLRAEDTAVYYCGKGGYCSSSGCRHYGMDVWGQGTTVTVSSAKTTAPSVYPL
APVCGDTTGSSVTLGCLVKGYFPEPVTLTWNSGSLSSGVHTFPAVLQSDLYTLSSSVTVTSSTWPSQSITC
NVAHPASSTKVDKKIEPRGPTIKPCPPCKCPAPNLLGGPSVFIFPPKIKDVLMISLSPIVTCVVVDVSEDDPD
VQISWFVNNVEVHTAQTQTHREDYNSTLRVVSALPIQHQDWMSGKEFKCKVNNKDLPAPIERTISKPK
GSVRAPQVYVLPPPEEEMTKKQVTLTCMVTDFMPEDIYVEWTNNGKTELNYKNTEPVLDSDGSYFMYS
KLRVEKKNWVERNSYSCSVVHEGLHNHHTTKSFSRTPGK;
(SEQ ID NO: 108)
QVQLVQSGAEVKKPGASVKVSCKASGYAFTTYGITWVRQAPGQGLEWMGWISAYNGNTNYAEKVQG
RFTMTTDTSTNTAYMELRSLRSDDTAVYFCARKGHYGSGTYYNPFGFDFWGQGTMVTVSSASTKGPSV
FPLAPCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPENNYKTTPPVLDSDGSFFLYS
RLTVDKSRWQEGNVFSCSVMHEALHNHYTQKSLSLSLGK.
LCVR Amino Acid Sequences:
(SEQ ID NO: 49)
DIQMTQSPSSLSASVGDRVTITCRASQSISSYLNWYQQKPGKAPKLLIYAASSLQSGVPSRFSGSGSGTDF
TLTISSLQPEDFATYYCQQSYSTPPITFGQGTRLEIK;
(SEQ ID NO: 50)
DIQMTQSPSSVSTSVGDRVTISCRASQDIRKWLAWYQQKPGKAPKLLIYATSSLQSGVPSRFSGSGSGTD
FTLTISSLQPEDFATYFCQQANSFPFTFGQGTKLEIK;
(SEQ ID NO: 51)
DIQMTQSPSTLSASVGDRVTLTCRASQSISNKLAWYQQKPGKAPNLLIYKASNLESGVPSRFSGSGSGTEF
TLTISSLQPDDFATYYCQQYNSYSWTFGQGTKVEIK;
(SEQ ID NO: 52)
EIVLTQSPGTLSLSPGERATLSCRASQTINHNNLAWYQQRPGQAPRLLIYGASNRATAIPDRFSGSGSGTD
FTLTISRLEPEDFEVYSCQQYGSLPLTFGGGTKVEIK;
(SEQ ID NO: 53)
DIQMTQSPSSLSASVGDRVTITCRASQSISSYLNWYQQKPGKAPKLLIYAASSLQSGVPSRFSGSGSGTDF
TLTISSLQPEDFATYYCQQSYSTPPITFGQGTRLEIK;
(SEQ ID NO: 54)
DIQMTQSPSSLSASVGDRVTISCRASQSISSYLNWYQQKPGKAPKVLMYAASSLQSGVPSRFSGSGSGTD
FTLTISSLQPEDFATYYCQQSYSTPPITFGQGTRLEIK;
(SEQ ID NO: 55)
DIQMTQSPSSLSASVGDRVTITCRASQSISSYLNWYQQKPGKAPKLLIYAASSLQSGVPSRFSGSGSGTDF
TLTISSLQPEDFATYYCQQSYSTPPITFGQGTRLEIK;
(SEQ ID NO: 56)
EIMLMQSPGTLSLSPGERATLSCRASQSVSSSYLAWYQQKPGQAPRLLIYGASSRATDIPDRFSGSGSGT
DFTLTISRLEPEDFAVYFCQQYYGSPWTFGQGTKVEIK;
(SEQ ID NO: 57)
EIVLTQSPGTLTLSPGERATLSCRASQSVGSKYLAWFQQKRGQAPRLLIYGASSRTSGIPDRISGSGSGTDF
TLTISRLEPEDFAVYYCQQYGSSPWTFGQGTKVEIK;
(SEQ ID NO: 58)
QSVLTQPPSASGTPGQRVTISCSGSSSNIGNNYIYWYQRLPGTTPKLLIYRNNQRPSGVPDRFSGSKSGTS
ASLAISGLRSEDEADYYCAAWDDTLSGYVFGTGTKVTVL;
(SEQ ID NO: 59)
QSVLTQPPSVSAAPGQKVTISCSGSSSNIGNNYVSWYQQLPGTAPKLLIYDNNKRPSGIPDRFSGSKSGTS
ATLGITGLQTGDEADYYCGTWDLSLSFNWVFGGGTKLTVL;
(SEQ ID NO: 60)
EIVLTQSPGTLSLSPGERATLSCRASQSVSSSYLAWYQQKPGQAPRLLIYGASSRATGIPDRFSGSGSGTDF
TLTISRLEPEDFALYFCQQYYGSPWTFGQGTKVEIK.
LCDR1 Amino Acid Sequences:
(SEQ ID NO: 61)
QSISSY;
(SEQ ID NO: 62)
QDIRKW;
(SEQ ID NO: 63)
QSISNK;
(SEQ ID NO: 64)
QTINHNN;
(SEQ ID NO: 65)
QSISSY;
(SEQ ID NO: 66)
QSISSY;
(SEQ ID NO: 67)
QSISSY;
(SEQ ID NO: 68)
QSVSSSY;
(SEQ ID NO: 69)
QSVGSKY;
(SEQ ID NO: 70)
SSNIGNNY;
(SEQ ID NO: 71)
SSNIGNNY;
(SEQ ID NO: 72)
QSVSSSY.
LCDR2 Amino Acid Sequence:
(SEQ ID NO: 73)
AAS;
(SEQ ID NO: 74)
ATS;
(SEQ ID NO: 75)
KAS;
(SEQ ID NO: 76)
GAS;
(SEQ ID NO: 77)
AAS;
(SEQ ID NO: 78)
AAS;
(SEQ ID NO: 79)
AAS;
(SEQ ID NO: 80)
GA;
(SEQ ID NO: 81)
GAS;
(SEQ ID NO: 82)
RN;
(SEQ ID NO: 83)
DNN;
(SEQ ID NO: 84)
GAS.
LCDR3 Amino Acid Sequence:
(SEQ ID NO: 85)
QQSYSTPPIT;
(SEQ ID NO: 86)
QQANSFPFT;
(SEQ ID NO: 87)
QQYNSYSWT;
(SEQ ID NO: 88)
QQYGSLPLT;
(SEQ ID NO: 89)
QQSYSTPPIT;
(SEQ ID NO: 90)
QSYSTPPIT;
(SEQ ID NO: 91)
QSYSTPPIT;
(SEQ ID NO: 92)
CQQYYGSPW;
(SEQ ID NO: 93)
QQYGSSPWT;
(SEQ ID NO: 94)
CAAWDDTLSGY;
(SEQ ID NO: 95)
GTWDLSLSFNWV;
(SEQ ID NO: 96)
QQYYGSPWT.
LC Amino Acid Sequence:
(SEQ ID NO: 109)
DIQMTQSPSSLSASVGDRVTITCRASQSISSYLNWYQQKPGKAPKLLIYAASSLQSGVPSRFSGSGSGTDF
TLTISSLQPEDFATYYCQQSYSTPPITFGQGTRLEIKRTVAAPSVFIFPPSDEQLKSGTASVVCLLNNFYPRE
AKVQWKVDNALQSGNSQESVTEQDSKDSTYSLSSTLTLSKADYEKHKVYACEVTHQGLSSPVTKSFNRG
EC;
(SEQ ID NO: 110)
DIQMTQSPSSVSTSVGDRVTISCRASQDIRKWLAWYQQKPGKAPKLLIYATSSLQSGVPSRFSGSGSGTD
FTLTISSLQPEDFATYFCQQANSFPFTFGQGTKLEIKRTVAAPSVFIFPPSDEQLKSGTASVVCLLNNFYPRE
AKVQWKVDNALQSGNSQESVTEQDSKDSTYSLSSTLTLSKADYEKHKVYACEVTHQGLSSPVTKSFNRG
EC;
(SEQ ID NO: 111)
DIQMTQSPSTLSASVGDRVTLTCRASQSISNKLAWYQQKPGKAPNLLIYKASNLESGVPSRFSGSGSGTEF
TLTISSLQPDDFATYYCQQYNSYSWTFGQGTKVEIKRTVAAPSVFIFPPSDEQLKSGTASVVCLLNNFYPRE
AKVQWKVDNALQSGNSQESVTEQDSKDSTYSLSSTLTLSKADYEKHKVYACEVTHQGLSSPVTKSFNRG
EC;
(SEQ ID NO: 112)
EIVLTQSPGTLSLSPGERATLSCRASQTINHNNLAWYQQRPGQAPRLLIYGASNRATAIPDRFSGSGSGTD
FTLTISRLEPEDFEVYSCQQYGSLPLTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVYACEVTHQGLSSPVTKSFNRG
EC;
(SEQ ID NO: 113)
DIQMTQSPSSLSASVGDRVTITCRASQSISSYLNWYQQKPGKAPKLLIYAASSLQSGVPSRFSGSGSGTDF
TLTISSLQPEDFATYYCQQSYSTPPITFGQGTRLEIKRTVAAPSVFIFPPSDEQLKSGTASVVCLLNNFYPRE
AKVQWKVDNALQSGNSQESVTEQDSKDSTYSLSSTLTLSKADYEKHKVYACEVTHQGLSSPVTKSFNRG
EC;
(SEQ ID NO: 114)
DIQMTQSPSSLSASVGDRVTISCRASQSISSYLNWYQQKPGKAPKVLMYAASSLQSGVPSRFSGSGSGTD
FTLTISSLQPEDFATYYCQQSYSTPPITFGQGTRLEIKRADAAPTVSIFPPSSEQLTSGGASVVCFLNNFYPK
DINVKWKIDGSERQNGVLNSWTDQDSKDSTYSMSSTLTLTKDEYERHNSYTCEATHKTSTSPIVKSFNRG
EC;
(SEQ ID NO: 115)
DIQMTQSPSSLSASVGDRVTITCRASQSISSYLNWYQQKPGKAPKLLIYAASSLQSGVPSRFSGSGSGTDF
TLTISSLQPEDFATYYCQQSYSTPPITFGQGTRLEIKRTVAAPSVFIFPPSDEQLKSGTASVVCLLNNFYPRE
AKVQWKVDNALQSGNSQESVTEQDSKDSTYSLSSTLTLSKADYEKHKVYACEVTHQGLSSPVTKSFNRG
EC;
(SEQ ID NO: 116)
EIMLMQSPGTLSLSPGERATLSCRASQSVSSSYLAWYQQKPGQAPRLLIYGASSRATDIPDRFSGSGSGT
DFTLTISRLEPEDFAVYFCQQYYGSPWTFGQGTKVEIKRADAAPTVSIFPPSSEQLTSGGASVVCFLNNFY
PKDINVKWKIDGSERQNGVLNSWTDQDSKDSTYSMSSTLTLTKDEYERHNSYTCEATHKTSTSPIVKSFN
RGEC;
(SEQ ID NO: 117)
EIVLTQSPGTLTLSPGERATLSCRASQSVGSKYLAWFQQKRGQAPRLLIYGASSRTSGIPDRISGSGSGTDF
TLTISRLEPEDFAVYYCQQYGSSPWTFGQGTKVEIKRTVAAPSVFIFPPSDEQLKSGTASVVCLLNNFYPRE
AKVQWKVDNALQSGNSQESVTEQDSKDSTYSLSSTLTLSKADYEKHKVYACEVTHQGLSSPVTKSFNRG
EC;
(SEQ ID NO: 118)
QSVLTQPPSASGTPGQRVTISCSGSSSNIGNNYIYWYQRLPGTTPKLLIYRNNQRPSGVPDRFSGSKSGTS
ASLAISGLRSEDEADYYCAAWDDTLSGYVFGTGTKVTVLRADAAPTVSIFPPSSEQLTSGGASVVCFLNNF
YPKDINVKWKIDGSERQNGVLNSWTDQDSKDSTYSMSSTLTLTKDEYERHNSYTCEATHKTSTSPIVKSF
NRGEC;
(SEQ ID NO: 119)
QSVLTQPPSVSAAPGQKVTISCSGSSSNIGNNYVSWYQQLPGTAPKLLIYDNNKRPSGIPDRFSGSKSGTS
ATLGITGLQTGDEADYYCGTWDLSLSFNWVFGGGTKLTVLGQPKAAPSVTLFPPSSEELQANKATLVCLI
SDFYPGAVTVAWKADSSPVKAGVETTTPSKQSNNKYAASSYLSLTPEQWKSHRSYSCQVTHEGSTVEKT
VAPTECS;
(SEQ ID NO: 120)
EIVLTQSPGTLSLSPGERATLSCRASQSVSSSYLAWYQQKPGQAPRLLIYGASSRATGIPDRFSGSGSGTDF
TLTISRLEPEDFALYFCQQYYGSPWTFGQGTKVEIKRTVAAPSVFIFPPSDEQLKSGTASVVCLLNNFYPRE
AKVQWKVDNALQSGNSQESVTEQDSKDSTYSLSSTLTLSKADYEKHKVYACEVTHQGLSSPVTKSFNRG
EC.

In some embodiments described herein, administration of the FNIP1 inhibitor and/or FLCN inhibitor can be targeted to adipose tissue or adipose cells. In some embodiments, an adipose-specific FNIP1 siRNA can be used to promote lipid oxidation in white adipose tissue (WAT) for treatment. In some embodiments, the FNIP1 inhibitor and/or FLCN inhibitor can be injected into adipose tissue. In some embodiments, the adipose-specific FNIP1 siRNA and/or FLCN siRNA can be delivered to adipocytes by using lipid-based transfection reagents for siRNA transfection of mature white adipocyte, according to the reverse transfection protocol reported by Isidor et al., Adipocyte, 2016, 5, 175-185. Essentially, lipofection-based reverse siRNA transfection reported therein may effectively silence FNIP1 and/or FLCN in mature adipocyte of various origins. In some embodiments, the adipose specific FNIP1 siRNA and/or FLCN siRNA can be delivered to adipose tissue by adhering to the proposed “gold standard” illustrated in the published article by Romanelli et al., Diabetes, 2020, 69, 2581-2588, with proper adaption. The siRNA against FNIP1 or FLCN can be transferred via viral or non-viral vectors. Adipocyte-specific antibodies against adipocyte-selective targets such as the amino acid transporter ASC-1 or the brown cell surface markers to improve tropism are contemplated.

Antigen-binding fragment include, but are not limited to, a monovalent Fab′, a divalent Fab2, a F(ab)′3 fragment, a single-chain fragment variable (scFv), a bis-scFv, a (scFv)2, a diabody, a minibody, a nanobody, a triabody, a tetrabody, a disulfide stabilized Fv protein (dsFv), a single-domain antibody (sdAb), an Ig NAR, a bispecific antibody or binding fragment thereof, a bi-specific T-cell engager (BiTE), a trispecific antibody, and chemically modified derivatives thereof.

A bifunctional linker such as, for example, M3463 (purchased from Broadpharm, BP-22617) can be used as a linker to join the siRNA or antisense molecule to the antibody. The modified single strands can be synthesized by standard automated oligonucleotide synthesis. During the on-bead synthesis, a 5′ sense strand modification with a six carbon chain terminating with a primary amino group can be attached to the abasic group to provide a handle for the linker. Standard deprotection and cleavage of the strands followed by reverse phase high-performance liquid chromatography (HPLC) purification furnished the strands that can be annealed to form the duplex siRNA. The bifunctional linker (such as, for example, M3463) can be coupled to the terminal amino group of the 5′ sense strand modification using an excess of the linker. The mixture can then be purified by reverse phase HPLC to furnish the functionalized duplex siRNA ready for antibody conjugation. To conjugate the siRNA to the antibody, the antibody can be treated with 1 mM dithiothreitol or TCEP (tris(2-carboxyethyl)phosphine) at 37° C. for 30 minutes. After gel filtration (G-25, pH 4.5 sodium acetate), the bis-maleimido linker siRNA can be added to the reduced antibody and the mixture can be adjusted to pH 7.0 with 1 M HEPES (pH 7.4). After 1 hour, the conjugates can be purified by size exclusion chromatography and sterile filtered. Protein and linker payload concentrations can be determined by UV spectral analysis. Size-exclusion HPLC can be used to establish that the conjugates used are >95% monomeric, and reversed-phase high-performance liquid chromatography (RP-HPLC) can be used to establish that there is <0.5% unconjugated linker payload. UV (Hamblett et al., Cancer Res., 2004, 10, 7063) and hydrophobic interaction chromatography (HIC) can e used to determine the loadings to be 1-1.6 siRNAs/antibody.

The present disclosure also provides methods of identifying a subject having an increased risk of developing muscle disorder. In some embodiments, the method comprises determining or having determined in a biological sample obtained from the subject the presence or absence of an FNIP1 variant nucleic acid molecule and/or an FLCN variant nucleic acid molecule (such as a genomic nucleic acid molecule, mRNA molecule, and/or cDNA molecule). When the subject lacks an FNIP1 variant nucleic acid molecule and/or an FLCN variant nucleic acid molecule (i.e., the subject is genotypically categorized as FNIP1 reference and/or FLCN reference), then the subject has an increased risk of developing muscle disorder. When the subject has an FNIP1 variant nucleic acid molecule and/or an FLCN variant nucleic acid molecule (i.e., the subject is heterozygous or homozygous for an FNIP1 variant nucleic acid molecule and/or an FLCN variant nucleic acid molecule), then the subject has a decreased risk of developing muscle disorder. In some embodiments, the FNIP1 variant nucleic acid molecule and/or an FLCN variant nucleic acid molecule is an FICN1 variant genomic nucleic acid molecule or an FLCN variant genomic nucleic acid molecule that comprises any one or more of the genetic variations described herein, or is an mRNA molecule produced therefrom, or is a cDNA molecule produced from the mRNA molecule.

Having a single copy of an FNIP1 variant nucleic acid molecule and/or an FLCN variant nucleic acid molecule is more protective of a subject from developing muscle disorder than having no copies of an FNIP1 variant nucleic acid molecule and/or an FLCN variant nucleic acid molecule. Without intending to be limited to any particular theory or mechanism of action, it is believed that a single copy of an FNIP1 variant nucleic acid molecule and/or an FLCN variant nucleic acid molecule (i.e., heterozygous for an FNIP1 variant nucleic acid molecule and/or an FLCN variant nucleic acid molecule) is protective of a subject from developing muscle disorder and it is also believed that having two copies of an FNIP1 variant nucleic acid molecule and/or an FLCN variant nucleic acid molecule (i.e., homozygous for an FNIP1 variant nucleic acid molecule and/or an FLCN variant nucleic acid molecule) may be more protective of a subject from developing muscle disorder, relative to a subject with a single copy. Thus, in some embodiments, a single copy of an FNIP1 variant nucleic acid molecule and/or an FLCN variant nucleic acid molecule may not be completely protective, but instead, may be partially or incompletely protective of a subject from developing muscle disorder. While not desiring to be bound by any particular theory, there may be additional factors or molecules involved in the development of muscle disorder that are still present in a subject having a single copy of an FNIP1 variant nucleic acid molecule and/or an FLCN variant nucleic acid molecule, thus resulting in less than complete protection from the development of muscle disorder.

Determining whether a subject has an FNIP1 variant nucleic acid molecule and/or an FLCN variant nucleic acid molecule in a biological sample from a subject and/or determining whether a subject has an FNIP1 variant nucleic acid molecule and/or an FLCN variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing muscle disorder, the subject is administered a muscle disorder therapeutic agent and/or an FNIP1 inhibitor and/or an FLCN inhibitor, as described herein. For example, when the subject is FNIP1 reference and/or FLCN reference, and therefore has an increased risk of developing muscle disorder, the subject is administered a muscle disorder therapeutic agent in an amount that is the same as or less than a standard dosage amount, and/or is administered an FNIP1 inhibitor and/or an FLCN inhibitor. In some embodiments, when the subject is heterozygous for an FNIP1 variant nucleic acid molecule and/or an FLCN variant nucleic acid molecule, the subject is administered the muscle disorder therapeutic agent in an amount that is the same as or less than a standard dosage amount, and/or is administered an FNIP1 inhibitor and/or an FLCN inhibitor. In some embodiments, when the subject is homozygous for an FNIP1 variant nucleic acid molecule and/or an FLCN variant nucleic acid molecule, the subject is administered a muscle disorder therapeutic agent in an amount that is the same as or less than a standard dosage amount. In some embodiments, the subject is FNIP1 reference and/or FLCN reference. In some embodiments, the subject is heterozygous for an FNIP1 variant nucleic acid molecule and/or an FLCN variant nucleic acid molecule. In some embodiments, the subject is homozygous for an FNIP1 variant nucleic acid molecule and/or an FLCN variant nucleic acid molecule.

The present disclosure also provides methods of determining a subject's aggregate burden, or risk score, of having two or more FNIP1 variant nucleic acid molecule and/or an FLCN variant nucleic acid molecules, and/or two or more FNIP1 variant polypeptides or two or more FLCN variant polypeptides associated with a decreased risk of developing muscle disorder. The aggregate burden is the sum of two or more genetic variants that can be carried out in an association analysis with muscle disorder. In some embodiments, the subject is homozygous for one or more FNIP1 variant nucleic acid molecule and/or an FLCN variant nucleic acid molecules associated with a decreased risk of developing muscle disorder. In some embodiments, the subject is heterozygous for one or more FNIP1 variant nucleic acid molecule and/or an FLCN variant nucleic acid molecules associated with a decreased risk of developing muscle disorder. When the subject has a lower aggregate burden, the subject has an increased risk of developing muscle disorder, and the subject is administered or continued to be administered the muscle disorder therapeutic agent in an amount that is the same as or less than the standard dosage amount, and/or an FNIP1 inhibitor and/or an FLCN inhibitor. When the subject has a higher aggregate burden, the subject has a decreased risk of developing muscle disorder and the subject is administered or continued to be administered the muscle disorder therapeutic agent in an amount that is the same as or less than a standard dosage amount. The higher the aggregate burden, the lower the risk of developing muscle disorder.

In some embodiments, a subject's aggregate burden of having any two or more FNIP1 variant nucleic acid molecules and/or any two or more FLCN variant nucleic acid molecules represents a weighted sum of a plurality of any of the FNIP1 variant nucleic acid molecules and/or any of the FLCN variant nucleic acid molecules. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the FNIP1 gene or FLCN gene, where the genetic burden is the number of alleles multiplied by the association estimate with muscle disorder or related outcome for each allele (e.g., a weighted polygenic burden score). In some embodiments, when the subject has an aggregate burden higher than a desired threshold score, the subject has a decreased risk of developing muscle disorder. In some embodiments, when the subject has an aggregate burden lower than a desired threshold score, the subject has an increased risk of developing muscle disorder.

In some embodiments, the aggregate burden may be divided into quintiles, e.g., top quintile, second quintile, intermediate quintile, fourth quintile, and bottom quintile, wherein the top quintile of aggregate burden corresponds to the lowest risk group and the bottom quintile of aggregate burden corresponds to the highest risk group. In some embodiments, a subject having a higher aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with muscle disorder in the top 10%, top 20%, top 30%, top 40%, or top 50% of p-value range for the association. In some embodiments, each of the identified genetic variants comprise the genetic variants having association with muscle disorder with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with muscle disorder with p-value of less than 5×10⁻⁸. In some embodiments, the identified genetic variants comprise genetic variants having association with muscle disorder in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects have aggregate burdens in the bottom decile, quintile, or tertile in a reference population. The threshold of the aggregate burden can be determined on the basis of the nature of the intended practical application and the risk difference that would be considered meaningful for that practical application.

In embodiments where the aggregate burden is determined for FNIP1 genetic variants and/or FLCN genetic variants associated with muscle disorder, then the aggregate burden represents a subject's risk score for developing muscle disorder. In some embodiments, the aggregate burden or risk score includes the FNIP1 variant genomic nucleic acid molecule and/or FLCN variant genomic nucleic acid molecule that comprises any one or more of the genetic variations described herein, or is an mRNA molecule produced therefrom, or is a cDNA molecule produced from the mRNA molecule. In some embodiments, a subject's aggregate burden can be determined for FNIP1 genetic variants and/or FLCN genetic variants associated with muscle disorder in combination with additional genetic variants for other genes also associated with muscle disorder to produce a polygenic risk score (PRS) for developing muscle disorder. In some embodiments, the PRS includes the FNIP1 variant genomic nucleic acid molecule and/or FLCN variant genomic nucleic acid molecule that comprises any one or more of the genetic variations described herein, or is an mRNA molecule produced therefrom, or is a cDNA molecule produced from the mRNA molecule.

The present disclosure also provides methods of detecting the presence or absence of an FNIP1 variant nucleic acid molecule and/or an FLCN variant nucleic acid molecule (i.e., a genomic nucleic acid molecule, an mRNA molecule, or a cDNA molecule produced from an mRNA molecule) in a biological sample from a subject. It is understood that gene sequences within a population and mRNA molecules encoded by such genes can vary due to polymorphisms such as single-nucleotide polymorphisms.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any FNIP1 variant nucleic acid molecule and/or an FLCN variant nucleic acid molecule, preliminary processing designed to isolate or enrich the biological sample for the genomic DNA can be employed. A variety of techniques may be used for this purpose. When detecting the level of any FNIP1 variant nucleic acid molecule and/or an FLCN variant nucleic acid molecule, different techniques can be used enrich the biological sample with mRNA molecules. Various methods to detect the presence or level of an mRNA molecule or the presence of a particular variant genomic DNA locus can be used.

In some embodiments, detecting an FNIP1 variant nucleic acid molecule and/or an FLCN variant nucleic acid molecule in a subject comprises performing a sequence analysis on a biological sample obtained from the subject to determine whether an FNIP1 genomic nucleic acid molecule and/or FLCN genomic nucleic acid molecule in the biological sample, and/or an FNIP1 mRNA molecule and/or FLCN mRNA molecule in the biological sample, and/or an FNIP1 cDNA molecule and/or FLCN cDNA molecule produced from an mRNA molecule in the biological sample, is present in the sample. In some embodiments, the methods detect the FNIP1 variant genomic nucleic acid molecule and/or FLCN variant genomic nucleic acid molecule that comprises any one or more of the genetic variations described herein, or an mRNA molecule produced therefrom, or a cDNA molecule produced from the mRNA molecule.

In some embodiments, the methods of detecting the presence or absence of an FNIP1 variant nucleic acid molecule and/or an FLCN variant nucleic acid molecule (such as, for example, a genomic nucleic acid molecule, an mRNA molecule, and/or a cDNA molecule produced from an mRNA molecule) in a subject comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an FNIP1 genomic nucleic acid molecule or mRNA molecule and/or FLCN genomic nucleic acid molecule or mRNA molecule, and if mRNA, optionally reverse transcribing the mRNA into cDNA. Such assays can comprise, for example determining the identity of these positions of the particular FNIP1 nucleic acid molecule and/or FLCN nucleic acid molecule. In some embodiments, the method is an in vitro method.

In some embodiments, the determining step, detecting step, or sequence analysis comprises sequencing at least a portion of the nucleotide sequence of the FNIP1 genomic nucleic acid molecule and/or FLCN genomic nucleic acid molecule, the FNIP1 mRNA molecule and/or FLCN mRNA molecule, or the FNIP1 cDNA molecule and/or FLCN cDNA molecule in the biological sample that comprises a genetic variation compared to the corresponding FNIP1 reference molecule and/or FLCN reference molecule. In some embodiments, the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only an FNIP1 genomic nucleic acid molecule and/or FNIP1 genomic nucleic acid molecule is analyzed. In some embodiments, only an FNIP1 mRNA and/or FLCN mRNA is analyzed. In some embodiments, only an FNIP1 cDNA obtained from the FNIP1 mRNA and/or FLCN cDNA obtained from the FLCN mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to an FNIP1 variant genomic sequence, variant mRNA sequence, or variant cDNA sequence and not the corresponding FNIP1 reference sequence under stringent conditions and determining whether hybridization has occurred and/or contacting the biological sample with a primer or probe, such as an alteration-specific primer or alteration-specific probe, that specifically hybridizes to an FLCN variant genomic sequence, variant mRNA sequence, or variant cDNA sequence and not the corresponding FLCN reference sequence under stringent conditions and determining whether hybridization has occurred.

In some embodiments, the determining step, detecting step, or sequence analysis comprises: a) amplifying at least a portion of the FNIP1 nucleic acid molecule that encodes the FNIP1 polypeptide and/or amplifying at least a portion of the FLCN nucleic acid molecule that encodes the FLCN polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an FNIP1 variant genomic nucleic acid molecule, variant mRNA molecule, or variant cDNA molecule or FLCN variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to FNIP1 variant nucleic acid molecule and/or an FLCN variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to FNIP1 variant nucleic acid molecule or an FLCN variant nucleic acid molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the FNIP1 variant nucleic acid molecule and/or an FLCN variant nucleic acid molecules disclosed herein. The primers described herein can be used to amplify any FNIP1 variant nucleic acid molecule and/or an FLCN variant nucleic acid molecule, or a fragment thereof.

In the context of the disclosure “specifically hybridizes” means that the probe or primer (such as, for example, the alteration-specific probe or alteration-specific primer) does not hybridize to a nucleic acid sequence encoding an FNIP1 reference genomic nucleic acid molecule, an FNIP1 reference mRNA molecule, and/or an FNIP1 reference cDNA molecule or does not hybridize to a nucleic acid sequence encoding an FLCN reference genomic nucleic acid molecule, an FLCN reference mRNA molecule, and/or an FLCN reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×his or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides muscle disorder therapeutic agents that treat, prevent, or inhibit muscle disorder for use in the treatment or prevention of muscle disorder in a subject having an FNIP1 variant nucleic acid molecule and/or an FLCN variant nucleic acid molecule. Any of the muscle disorder therapeutic agents that treat, prevent, or inhibit muscle disorder described herein can be used herein. Any of the FNIP1 variant nucleic acid molecules and/or FLCN variant nucleic acid molecules disclosed herein can be used herein. In some embodiments, the FNIP1 variant nucleic acid molecule and/or the FLCN variant nucleic acid molecule is an FNIP1 variant genomic nucleic acid molecule or FLCN variant genomic nucleic acid molecule that comprises any one or more of the genetic variations described herein, or is an mRNA molecule produced therefrom, or is a cDNA molecule produced from the mRNA molecule.

The present disclosure also provides uses of muscle disorder therapeutic agents that treat, prevent, or inhibit muscle disorder for use in the preparation of a medicament for treating or preventing muscle disorder in a subject having an FNIP1 variant nucleic acid molecule and/or an FLCN variant nucleic acid molecule. Any of the muscle disorder therapeutic agents that treat, prevent, or inhibit muscle disorder described herein can be used herein. Any of the FNIP1 variant nucleic acid molecules and/or FLCN variant nucleic acid molecules disclosed herein can be used herein. In some embodiments, the FNIP1 variant nucleic acid molecule and/or the FLCN variant nucleic acid molecule is an FNIP1 variant genomic nucleic acid molecule or FLCN variant genomic nucleic acid molecule that comprises any one or more of the genetic variations described herein, or is an mRNA molecule produced therefrom, or is a cDNA molecule produced from the mRNA molecule.

The present disclosure also provides FNIP1 inhibitors for use in the treatment or prevention of muscle disorder in a subject that is FNIP1 reference or is heterozygous for an FNIP1 variant nucleic acid molecule. Any of the FNIP1 inhibitors described herein can be used herein. Any of the FNIP1 variant nucleic acid molecules disclosed herein can be used herein. In some embodiments, the FNIP1 variant nucleic acid molecule and/or the FLCN variant nucleic acid molecule is an FNIP1 variant genomic nucleic acid molecule or FLCN variant genomic nucleic acid molecule that comprises any one or more of the genetic variations described herein, or is an mRNA molecule produced therefrom, or is a cDNA molecule produced from the mRNA molecule.

The present disclosure also provides FNIP1 inhibitors in the preparation of a medicament for treating or preventing muscle disorder in a subject that is FNIP1 reference or is heterozygous for an FNIP1 variant nucleic acid molecule. Any of the FNIP1 inhibitors described herein can be used herein. Any of the FNIP1 variant nucleic acid molecules disclosed herein can be used herein. In some embodiments, the FNIP1 variant nucleic acid molecule and/or the FLCN variant nucleic acid molecule is an FNIP1 variant genomic nucleic acid molecule or FLCN variant genomic nucleic acid molecule that comprises any one or more of the genetic variations described herein, or is an mRNA molecule produced therefrom, or is a cDNA molecule produced from the mRNA molecule.

The present disclosure also provides FLCN inhibitors for use in the treatment or prevention of muscle disorder in a subject that is FLCN reference or is heterozygous for an FLCN variant nucleic acid molecule. Any of the FLCN inhibitors described herein can be used herein. Any of the FLCN variant nucleic acid molecules disclosed herein can be used herein. In some embodiments, the FLCN variant nucleic acid molecule is an FLCN variant genomic nucleic acid molecule that comprises any one or more of the genetic variations described herein, or is an mRNA molecule produced therefrom, or is a cDNA molecule produced from the mRNA molecule.

The present disclosure also provides FLCN inhibitors in the preparation of a medicament for treating or preventing muscle disorder in a subject that is FLCN reference or is heterozygous for an FLCN variant nucleic acid molecule. Any of the FLCN inhibitors described herein can be used herein. Any of the FLCN variant nucleic acid molecules disclosed herein can be used herein. In some embodiments, the FLCN variant nucleic acid molecule is a FLCN variant genomic nucleic acid molecule that comprises any one or more of the genetic variations described herein, or is an mRNA molecule produced therefrom, or is a cDNA molecule produced from the mRNA molecule.

In some embodiments, the FNIP1 inhibitor and/or the FLCN inhibitor, and the muscle disorder therapeutic agent are disposed within a pharmaceutical composition. In some embodiments, the FNIP1 inhibitor and/or the FLCN inhibitor are disposed within a first pharmaceutical composition and the muscle disorder therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: General Methods

### Participating Cohorts

Genetic association studies were performed in individuals of African or Admixed African, Admixed American, European, and East and South Asian ancestry from the United Kingdom Biobank (UKB) cohort (Bycroft et al., Nature, 2018, 562, 203-09; and Van Hout et al., Nature, 2020, 586, 749-56), the MyCode Community Health Initiative cohort from the Geisinger Health System (GHS) (Carey et al., Genet. Med., 2016, 18, 906-13), the University of Pennsylvania Medicine BioBank (UPENN-PMBB), the Malmo Diet and Cancer Study (MDCS) (Berglund et al., J. Int. Med., 1993, 233, 45-51), the Mount Sinai's BioMe Personalized Medicine Cohort (SINAI) (Gottesman et al., Genet. Med., 2013, 15, 761-71), the Indiana Biobank (INDIANA), the Colorado Biobanked and Prospective Collection Project (COLORADO), the UCLA Biobank (UCLA), the Mayo Clinic Biobank (MAYO-CLINIC), the University of Pennsylvania Highly Consanguineous Cohort of Pakistani individuals enrolled in the Center for Non-Communicable Diseases (CNCD), the National Institute of Diabetes and Digestive and Kidney Diseases Sequencing in Pima Indians (NIDDK) (Knowler et al., Am. J. Epidemiol., 1978, 108, 497-505), the Mexico City Prospective Study (MCPS) (Tapia-Conyer et al., Int. J. Epidemiol., 2006, 35, 243-9) and the University of Texas Southwestern Dallas Heart Expansion Study—Dallas Biobank (UTSW-COHEN).

The UKB is a population-based cohort study of people aged between 40 and 69 years recruited through 22 testing centers in the UK between 2006-2010. Exome sequencing and phenotype data were available up to 432,015 individuals. The GHS MyCode study is a health system-based cohort of patients from Central and Eastern Pennsylvania (USA) recruited in 2007-2019. A total of 134,731 participants from GHS with available whole-exome sequencing and phenotype data were included. SINAI is an electronic health record-linked clinical care cohort, of which 21,673 individuals had available phenotypes and exome sequencing data. The MDCS is a population-based prospective cohort that included 5,215 individuals with available phenotype and exome sequencing data. The UPENN-PMBB included a total of 26,022 individuals with available phenotype and exome sequencing data. The INDIANA biobank included 5,389 participants with exome sequencing and phenotypic data. The MCPS is a population-based prospective study that included 33,935 individuals with phenotype and exome sequencing data. The CNCD is a population-based study of Pakistani individuals and included 34,831 individuals with phenotype and exome sequencing data. The NIDDK included 5,175 with both phenotype and exome sequencing data. The COLORADO, UCLA and MAYO-CLINIC are hospital-based electronic health record data that provided genotypic and phenotypic data on 35,320, 27,932 and 81,132. The UTSW-COHEN is a population-based cohort of African- and Hispanic-Americans in the city of Dallas that included 13,888 individuals.

### Phenotype Definitions

Blood lipid levels were measured from samples drawn during the first visit to study center for population-based studies like UKB and MDCS, while they were obtained as the median measurement from cohorts using electronic healthcare record data. In general, LDL cholesterol levels were estimated using the Friedewald equation for the except for the UKB where direct LDL measurements were available. Individuals known to be on lipid lowering medication had their pre-treatment lipid levels estimated using a correction factor.

### Liver MRI Phenotypes

A subset of participants in UKB underwent magnetic resonance imaging (MRI) of the liver (Littlejohns et al., Nat. Commun., 2020, 11, 2624). For liver fat imaging acquisition, approximately 10,000 subjects were imaged under a Dixon gradient echo protocol, while the remaining set of individuals, who underwent imaging from 2016 onwards, were imaged using an IDEAL sequence (Iterative Decomposition of water and fat with Echo Asymmetry and Least-squares estimation) protocol. Data from this acquisition are provided as a series of complex-valued 2D images per subject. The in-plane pixel size is 2.5×2.5 mm; slice thickness is 6 mm. All images were acquired on Siemens MAGNETOM clinical MRI scanners.

Measurements of liver fat percentage (proton density liver fat fraction; PDFF), a measure of the proportion of fat content in the liver, were obtained by applying pre-defined mathematical models after segmenting the liver on liver MRI images (Hernando et al., Magn. Reason. Med., 2012, 67, 638-44). The implementation was validated using a publicly available phantom dataset containing vials of varying concentrations of fat (Hernando et al., Magn. Reason. Med., 2017, 77, 1516-24). PDFF was estimated as the fraction of fat signal relative to total fat plus water signal. Pixels belonging to the liver were segmented using a Li thresholding approach for PDFF maps to identify liver tissue. To obtain a summary measure of each trait per subject, all pixels within the liver were averaged for each parametric map. PDFF was then used as the outcome for the analysis. Further details on the derivation of these phenotypes have been published (O'Dushlaine et al., Genome-wide association study of liver fat, iron, and extracellular fluid fraction in the UK Biobank′, medRxiv: 2021.10.25.21265127).

### Phenotype Definitions

Clinical laboratory measurements for ALT, total cholesterol, LDL cholesterol, HDL cholesterol and triglycerides were extracted from electronic health records (EHRs) for participants from the GHS, UPENN-PMBB, SINAI, INDIANA, UCLA, COLORADO, and MAYO-CUNIC biobanks or measured at recruitment for UKB, MDCS, CNCD, NIDDK, UTSW-COHEN. GHS, UPENN-PMBB, SINAI, INDIANA, UCLA, COLORADO, and MAYO-CLINIC, median values were calculated for all participants with two or more measurements. In UKB, ALT, AST, total cholesterol, LDL cholesterol, HDL cholesterol and triglycerides were measured by IFCC (International Federation of Clinical Chemistry) analysis on a Beckman Coulter AU5800 at the baseline visit of the study and averaged in case of multiple measurements. Prior to genetic association analysis, continuous phenotype values were transformed by the inverse standard normal function, applied within each ancestry group and separately in men and women.

Disease outcomes were defined according to the International Classification of Diseases, Ninth and Tenth Revision (ICD-9 and ICD-10) and Read codes stored in EHRs, and self-reports were used when available; all of which and combined into single variables to classify individuals into cases or controls. Individuals with type 2 diabetes were identified using a previously described algorithm (Eastwood et al., PLoS One, 2016, 11, e0162388). Individuals with coronary artery disease or liver diseases were identified as described in Table 6, combining EHR records, self-reports and ALT measurements.

Individuals with or without disease were identified using EHR records and self-reports. OPCS4 codes (operation procedures), f.20002 (self-reported disease) and f.20004 (self-reported operation procedures) variables were specific to UKB. In each cohort, EHR records with ICD-9 or read codes were translated to ICD-10 codes.

TABLE 2

| Disease | Case definition | Control exclusion |
|---|---|---|
| Non−alcoholic liver | ICD10: | ICD10: |
| disease | K721, K740, K741, | K70, K71, K72, K73, |
| | K742, K746, | K74, K75, K76, K77, I81, |
| | K758, K760 | I85, I982, I983, I864, |
| | | T864, Z944, C220 |
| | | OPCS4: G10, G144, J01 |
| | | f.20002: 1604, 1158, 1141 |
| | | ALT: >33 IU/L for men |
| | | and >24 IU/L for |
| | | women |

Type 2 diabetes cases were adjudicated in each cohort on the basis of one or more of the following criteria: 1) an electronic health record of type 2 diabetes (using International Classification of Diseases, Tenth Revision (ICD-10) diagnosis codes E11 or O24.1 or corresponding Ninth Revision (ICD-9) codes), in at least one inpatient encounter or at least two outpatient encounters or if noted as a cause of death; 2) a glycemic biomarker value (HbA1c, random or fasting glucose) in the diabetic range (Diabetes Care, 2021, 44(Suppl 1), S15-S33); 3) a prescription record of anti-diabetic medication use; 4) a self-reported physician diagnosis of type 2 diabetes; 5) entry on a diabetes registry as a type 2 diabetes case. Where possible, individuals were excluded from the case pool if they had a potential diagnosis of type 1 diabetes mellitus (using ICD-10 codes E10 or 024.0, and a prescription record that included insulin only in the absence of other diabetic medication). Individuals not meeting any of the criteria for diabetes case status were used as controls. In addition, individuals were excluded from the control group if they met any of the following criteria: 1) an electronic health record diagnosis pertaining to any potential type of diabetes mellitus or a family history of diabetes; 2) a glycemic biomarker value in the prediabetic range; 3) any other cohort-specific phenotype that potentially indicated a diagnosis of diabetes mellitus (e.g., a disease registry entry or self-reported diagnosis of non-specific diabetes).

### Genotype Data

High coverage whole exome sequencing was performed as previously described (Science, 2016, 354:aaf6814; and Nature, 2020, 586, 749-756) and as summarized below. NimbleGen probes (VCRome; for part of the GHS cohort) or a modified version of the xGen design available from Integrated DNA Technologies (IDT; for the rest of GHS and other cohorts) were used for target sequence capture of the exome. A unique 6 base pair (bp) barcode (VCRome) or 10 bp barcode (IDT) was added to each DNA fragment during library preparation to facilitate multiplexed exome capture and sequencing. Equal amounts of sample were pooled prior to exome capture. Sequencing was performed using 75 bp paired-end reads on Illumina v4 HiSeq 2500 (for part of the GHS cohort) or NovaSeq (for the rest of GHS and other cohorts) instruments. Sequencing had a coverage depth (i.e., number of sequence-reads covering each nucleotide in the target areas of the genome) sufficient to provide greater than 20× coverage over 85% of targeted bases in 96% of VCRome samples and 20× coverage over 90% of targeted bases in 99% of IDT samples. Data processing steps included sample de-multiplexing using Illumina software, alignment to the GRCh38 Human Genome reference sequence including generation of binary alignment and mapping files (BAM), processing of BAM files (e.g., marking of duplicate reads and other read mapping evaluations). Variant calling was performed using the GLNexus system. Variant mapping and annotation were based on the GRCh38 Human Genome reference sequence and Ensembl v85 gene definitions using the snpEff software. The snpEff predictions that involve protein-coding transcripts with an annotated start and stop were then combined into a single functional impact prediction by selecting the most deleterious functional effect class for each gene. The hierarchy (from most to least deleterious) for these annotations was frameshift, stop-gain, stop-loss, splice acceptor, splice donor, stop-lost, in-frame indel, missense, other annotations. Predicted LOF genetic variants included: a) insertions or deletions resulting in a frameshift, b) insertions, deletions or single nucleotide variants resulting in the introduction of a premature stop codon or in the loss of the transcription start site or stop site, and c) variants in donor or acceptor splice sites. Missense variants were classified for likely functional impact according to the number of in silico prediction algorithms that predicted deleteriousness using SIFT (Adzhubei et al., Nat. Methods, 2010, 7, 248-9) and Polyphen2_HVAR (Adzhubei et al., Nat. Methods, 2010, 7, 248-9), LRT (Chun et al., Genome Res., 2009, 19, 1553-61) and MutationTaster (Schwarz et al., Nat. Methods, 2010, 7, 575-6). For each gene, the alternative allele frequency (AAF) and functional annotation of each variant determined inclusion into these 7 gene burden exposures: 1) pLOF variants with AAF<1%; 2) pLOF or missense variants predicted deleterious by 5/5 algorithms with AAF<1%; 3) pLOF or missense variants predicted deleterious by 5/5 algorithms with AAF<0.1%; 4) pLOF or missense variants predicted deleterious by at least 1/5 algorithms with AAF<1%; 5) pLOF or missense variants predicted deleterious by at least 1/5 algorithms with AAF<0.1%; 6) pLOF or any missense with AAF<1%; 7) pLOF or any missense variants with AAF<0.1%.

### Association Analysis of Gene Burden of Rare Loss of Function Variation

Association between the burden of rare predicted loss-of-function or missense variants in a given gene and phenotype was examined by fitting a linear (for quantitative traits) or firth bias-corrected logistic (for binary traits) regression model adjusted for a polygenic score that approximates a genomic kinship matrix using REGENIE v1.0. Analyses were stratified by ancestry and adjusted for age, age², sex, age-by-sex and age²-by-sex interaction terms, experimental batch-related covariates, 10 common variant-derived principal components, and 20 rare variant-derived principal components. Results across cohorts for each variant-phenotype association were combined using fixed effects inverse variance weighted meta-analysis. In gene burden tests, all individuals were labeled as heterozygotes if they carried one or more qualifying rare variant (as described above based on frequency and functional annotation) and as homozygotes if they carried any qualifying variant in the homozygous state. This “composite genotype” was then used to test for association.

### Example 2: Loss of Function of the Genes Encoding FNIP1 and FLCN is Associated with Lower Levels of Triglycerides, Alanine Aminotransferase, Liver Fat by MRI, HbA1c and Waist-to-Hip Ratio and Protection from Type 2 Diabetes

Exome sequencing data was analyzed for participants from the Geisinger Heath System MyCode Community Health Initiative study (GHS), Malmo Diet and Cancer Study (MDCS), Mount Sinai's BioMe personalized medicine cohort (SINAI), the UK Biobank (UKB), the University of Pennsylvania Penn Medicine Biobank (UPENN-PMBB), the Indiana University Biobank (INDIANA), the Colorado Biobanked and Prospective Collection Project (COLORADO), the UCLA Biobank (UCLA), the Mayo Clinic Biobank (MAYO-CLINIC), the University of Pennsylvania Highly Consanguineous Cohort (CNCD), the National Institute of Diabetes and Digestive and Kidney Diseases Sequencing in Pima Indians (NIDDK), the Mexico City Prospective Study (MCPS) and the University of Texas Southwestern Dallas Heart Expansion Study—Dallas Biobank (UTSW-COHEN). In a large exome-wide association analyses of rare coding variation with blood lipids, FNIP1 and FLCN loss-of-function variants were identified to be associated with a decrease in triglyceride, LDL cholesterol, and alanine aminotransferase levels (ALT).

Compared to non-carriers, carriers of rare predicted loss-of-function variants in FNIP1 and FLCN had lower levels triglycerides (−35 mg/dL and −21 mg/dL, respectively), LDL cholesterol (−7 mg/dL and −4 mg/dL, respectively) and apolipoprotein B (−7 mg/dL and −5 mg/dL, respectively (see, Table 3). Carriers of loss-of-function variants in both genes had lower levels of alanine aminotransferase, and those with FLCN loss-of-function variants had significantly lower liver fat and inflammation levels as measured by MRI (see, Table 4). FNIP1 rare predicted loss-of-function and deleterious missense were associated with lower odds of non-alcoholic liver disease (see, Table 5). Carriers of loss-of-function or deleterious missense variants in both genes had favorable fat distribution as indicated by lower wait-to-hip ratio, lower body fat percentage, lower HbA1c and lower odds of type 2 diabetes (see, Table 6 and Table 7).

TABLE 3
Rare predicted loss of function variants in FNIP1 and FLCN are associated with
lower levels of LDL cholesterol, triglycerides, and apolipoprotein levels

| | | Allele | | Effect size | |
|---|---|---|---|---|---|
| | Gene | frequency | Genotype count | (95% confidence interval) | |
| Trait | pLoF | of carriers | (RR\|RA\|AA) | (SD units/Clinical units) | P-value |
| Triglyceride/ | FNIP1 | 8 × 10⁻⁵ | 771,868/116/2 | −0.44 (−0.60, −0.27) SD/ | 2 × 10⁻⁷ |
| HDL Ratio | | | | −2.03 (−2.79, −1.27) ratio | |
| | FLCN | 2 × 10⁻⁴ | 795,771/286/0 | −0.21 (−0.32, −0.11) SD/ | 0.0001 |
| | | | | −0.99 (−1.49, −0.49) ratio | |
| Triglycerides | FNIP1 | 8 × 10⁻⁵ | 820,763/121/2 | −0.38 (−0.54, −0.22) SD/ | 3 × 10⁻⁶ |
| | | | | −34.78 (−49.47, −20.09) | |
| | | | | mg/dL | |
| | FLCN | 2 × 10⁻⁴ | 835,898/298/0 | −0.23 (−0.33, −0.12) SD/ | 2 × 10⁻⁵ |
| | | | | −20.93 (−30.61, −11.24) | |
| | | | | mg/dL | |
| HDL | FNIP1 | 8 × 10⁻⁵ | 778,162/119/2 | 0.41 (0.25, 0.57) SD/ | 4 × 10⁻⁷ |
| cholesterol | | | | 6.33 (3.88, 8.77) mg/dL | |
| | FLCN | 2 × 10⁻⁴ | 800,789/289/0 | 0.09 (−0.01, 0.20) SD/ | 0.09 |
| | | | | 1.42 (−0.20, 3.04) mg/dL | |
| LDL | FNIP1 | 8 × 10⁻⁵ | 814,013/120/2 | −0.19 (−0.35, −0.03) SD/ | 0.02 |
| cholesterol | | | | −7.03 (−12.84, −1.23) | |
| | | | | mg/dL | |
| | FLCN | 2 × 10⁻⁴ | 828,975/297/0 | −0.12 (−0.22, −0.01) SD/ | 0.03 |
| | | | | −4.33 (−8.15, −0.51) mg/dL | |
| Non-HDL | FNIP1 | 8 × 10⁻⁵ | 703,131/107/2 | −0.26 (−0.43, −0.10) SD/ | 0.002 |
| cholesterol | | | | −10.75 (−17.51, −4.00) | |
| | | | | mg/dL | |
| | FLCN | 2 × 10⁻⁴ | 726,992/252/0 | −0.20 (−0.31, −0.09) SD/ | 0.0006 |
| | | | | −8.05 (−12.63, −3.47) | |
| | | | | mg/dL | |
| Apolipoprote | FNIP1 | 7 × 10⁻⁵ | 489,524/72/0 | −0.30 (−0.50, −0.10) SD/ | 0.004 |
| in B | | | | −7.25 (−12.15, −2.34) | |
| | | | | mg/dL | |
| | FLCN | 2 × 10⁻⁴ | 490,948/156/0 | −0.19 (−0.33, −0.06) SD/ | 0.005 |
| | | | | −4.74 (−8.07, −1.42) mg/dL | |

Effect sizes are given in standard deviation (SD) and clinical units. RR: count of homozygous individuals for the reference allele, RA: count of heterozygous individuals carrying one pLOF variant, AA: count of homozygous individuals carrying pLOF variants in both alleles.

TABLE 4
Rare predicted loss of function variants in FNIP1 and FLCN are associated with lower levels of
liver enzymes and liver fat and inflammation as measured by MRI

| | | Allele | | Effect size | |
|---|---|---|---|---|---|
| | Gene | frequency | Genotype count | (95% confidence interval) | |
| Trait | pLoF | of carriers | (RR\|RA\|AA) | (SD units/Clinical units) | P-value |
| ALT | FNIP1 | 7 × 10⁻⁵ | 773,854/107/2 | −0.35 (−0.53, −0.18) SD/ | 7 × 10⁻⁵ |
| | | | | −7.07 (−10.56, −3.59) U/L | |
| | FLCN | 2 × 10⁻⁴ | 785,764/274/0 | −0.13 (−0.24, −0.02) SD/ | 0.02 |
| | | | | −2.63 (−4.89, −0.37) U/L | |
| AST | FNIP1 | 7 × 10⁻⁵ | 789,168/111/2 | −0.27 (−0.44, −0.10) SD/ | 0.002 |
| | | | | −4.22 (−6.87, −1.57) U/L | |
| | FLCN | 2 × 10⁻⁴ | 799,881/276/0 | −0.06 (−0.17, 0.06) SD/ | 0.33 |
| | | | | −0.87 (−2.62, 0.87) U/L | |
| Liver Fat | FNIP1 | 8 × 10⁻⁵ | 37,039/6/0 | −0.42 (−1.16, 0.33) SD/ | 0.27 |
| % by | | | | −2.05 (−5.71, 1.62) % | |
| MRI | FLCN | 2 × 10⁻⁴ | 36,749/11/0 | −0.72 (−1.30, −0.15) SD/ | 0.01 |
| | | | | −3.55 (−6.38, −0.72) % | |
| Liver | FNIP1 | 8 × 10⁻⁵ | 37,039/6/0 | −0.25 (−0.98, 0.48) SD/ | 0.5 |
| Inflammation | | | | −11.37 (−44.46, 21.72) | |
| by | FLCN | 4 × 10⁻⁴ | 36,734/26/0 | −0.43 (−0.80, −0.06) SD/ | 0.02 |
| MRI | | | | −19.64 (−36.57, −2.71) | |

Effect sizes are given in standard deviation (SD) and clinical units. RR: count of homozygous individuals for the reference allele, RA: count of heterozygous individuals carrying one pLOF variant, AA: count of homozygous individuals carrying pLOF variants in both alleles.

TABLE 5
Rare predicted loss of function and deleterious missense variants in
FNIP1 are associated with odds of non-alcoholic liver disease

| | Allele | | Genotype | Odds | |
|---|---|---|---|---|---|
| | frequency | Genotype | count | Ratio | |
| Gene | of | count cases | controls | (95% | |
| Variation | carriers | (RR\|RA\|AA) | (RR\|RA\|AA) | CI) | P value |
| FLCN pLOF | 2 × 10⁻⁴ | 33,886/13/0 | 636,050/ | 1.06 | 0.84 |
| | | | 236/0 | (0.61, | |
| | | | | 1.86) | |
| FLCN pLOF | 3 × 10⁻³ | 37,159/172/0 | 669,457/ | 0.97 | 0.67 |
| plus | | | 3,840/1 | (0.82, | |
| deleterious | | | | 1.13) | |
| missense | | | | | |
| FNIP1 pLOF | 9 × 10⁻⁵ | 27,265/2/1 | 564,649/96/1 | 1.17 | 0.7 |
| | | | | (0.52, | |
| | | | | 2.66) | |
| FNIP1 pLOF | 1 × 10⁻² | 37,286/906/3 | 660,137/ | 0.90 | 0.002 |
| plus | | | 17,473/39 | (0.83, | |
| deleterious | | | | 0.96) | |
| missense | | | | | |

RR: count of homozygous individuals for the reference allele, RA: count of heterozygous individuals carrying one pLOF or pLOF + deleterious missense variant, AA: count of homozygous individuals carrying pLOF + deleterious missense variants in both alleles.

TABLE 6
Rare predicted loss of function variants in FNIP1 and FLCN are associated
with lower waist to hip ratio, body fat percentage and HbA1c levels

| | | Allele | | Effect size | |
|---|---|---|---|---|---|
| | | fre- | | (95% confidence | |
| | | quency | Genotype | interval) | |
| | Gene | of | count | (SD units/Clinical | |
| Trait | pLoF | carriers | (RR\|RA\|AA) | units) | P-value |
| Waist Hip | FNIP1 | 8 × 10⁻⁵ | 670,295/105/0 | −0.24 (−0.42, | 0.008 |
| Ratio | | | | −0.06) SD/ | |
| adjusted | | | | −0.02 (−0.03, | |
| for | | | | −0.00) ratio | |
| BMI | FLCN | 2 × 10⁻⁴ | 671,779/217/0 | −0.11 (−0.23, | 0.09 |
| | | | | 0.02) SD/ | |
| | | | | −0.01 (−0.02, | |
| | | | | 0.00) ratio | |
| (BIA) | FNIP1 | 7 × 10⁻⁵ | 484,446/66/0 | −0.31 (−0.53, | 0.007 |
| Body | | | | −0.08) SD/ | |
| fat % | | | | −2.62 (−4.53, | |
| | | | | −0.71) % | |
| | FLCN | 2 × 10⁻⁴ | 485,932/157/0 | −0.18 (−0.32, | 0.02 |
| | | | | −0.03) SD/ | |
| | | | | −1.50 (−2.74, | |
| | | | | −0.27) % | |
| HbA1c | FNIP1 | 8 × 10⁻⁵ | 840,079/124/2 | −0.21 (−0.36, | 0.006 |
| | | | | −0.06) SD/ | |
| | | | | −0.22 (−0.37, | |
| | | | | −0.06) % | |
| | FLCN | 2 × 10⁻⁴ | 844,518/275/0 | −0.14 (−0.25, | 0.007 |
| | | | | −0.04) SD/ | |
| | | | | −0.15 (−0.26, | |
| | | | | −0.04) % | |

Effect sizes are given in standard deviation (SD) and clinical units. RR: count of homozygous individuals for the reference allele, RA: count of heterozygous individuals carrying one pLOF variant, AA: count of homozygous individuals carrying pLOF variants in both alleles.

TABLE 7
Rare predicted loss of function and deleterious missense variants in
FNIP1 and FLCN are associated with lower odds of type 2 diabetes

| | Allele | | Genotype | Odds | |
|---|---|---|---|---|---|
| | fre- | Genotype | count | Ratio | |
| Gene | quency of | count cases | controls | (95% | |
| Variation | carriers | (RR\|RA\|AA) | (RR \|RA\|AA) | CI) | P value |
| FLCN | 2 × 10⁻⁴ | 140,532/38/0 | 644,837/ | 0.68 | 0.03 |
| pLOF | | | 249/0 | (0.48, | |
| | | | | 0.97) | |
| FLCN | 3 × 10⁻³ | 153,579/729/2 | 689,124/ | 0.86 | 0.0006 |
| pLOF | | | 4,105/1 | (0.79, | |
| plus | | | | 0.94) | |
| deleterious | | | | | |
| missense | | | | | |
| FNIP1 | 8 × 10⁻⁵ | 115,620/17/0 | 563,973/88/0 | 0.78 | 0.36 |
| pLOF | | | | (0.47, | |
| | | | | 1.32) | |
| FNIP1 | 1 × 10⁻² | 153,603/ | 684,824/ | 0.95 | 0.02 |
| pLOF | | 4,025/15 | 16,901/36 | (0.92, | |
| plus | | | | 0.99) | |
| deleterious | | | | | |
| missense | | | | | |

RR: count of homozygous individuals for the reference allele, RA: count of heterozygous individuals carrying one pLOF or pLOF + deleterious missense variant, AA: count of homozygous individuals carrying pLOF + deleterious missense variants in both alleles.

TABLE 8
Predicted loss of function or deleterious missense variants in
FNIP1 identified by whole exome sequencing

| Variant | Effect | hgvsc | hgvsp |
|---|---|---|---|
| 5:131644687:A:G | stop_ | ENST00000510461.6: | ENSP00000421985.1: |
| | lost | c.3499T>C | p.Ter1167GlnextTer3 |
| 5:131644689:A:T | missense | ENST00000510461.6: | ENSP00000421985.1: |
| | | c.3497T>A | p.Leu1166His |
| 5:131644701:G:A | missense | ENST00000510461.6: | ENSP00000421985.1: |
| | | c.3485C>T | p.Ala1162Val |
| 5:131644702:C:T | missense | ENST00000510461.6: | ENSP00000421985.1: |
| | | c.3484G>A | p.Ala1162Thr |
| 5:131644707:T:C | missense | ENST00000510461.6: | ENSP00000421985.1: |
| | | c.3479A>G | p.Tyr1160Cys |
| 5:131644707:T:G | missense | ENST00000510461.6: | ENSP00000421985.1: |
| | | c.3479A>C | p.Tyr1160Ser |
| 5:131644708:A:C | missense | ENST00000510461.6: | ENSP00000421985.1: |
| | | c.3478T>G | p.Tyr1160Asp |
| 5:131644717:G:A | missense | ENST00000510461.6: | ENSP00000421985.1: |
| | | c.3469C>T | p.His1157Tyr |
| 5:131644717:G:T | missense | ENST00000510461.6: | ENSP00000421985.1: |
| | | c.3469C>A | p.His1157Asn |
| 5:131644720:T:C | missense | ENST00000510461.6: | ENSP00000421985.1: |
| | | c.3466A>G | p.Thr1156Ala |
| 5:131644721:G:C | missense | ENST00000510461.6: | ENSP00000421985.1: |
| | | c.3465C>G | p.Ser1155Arg |
| 5:131644725:G:A | missense | ENST00000510461.6: | ENSP00000421985.1: |
| | | c.3461C>T | p.Ala1154Val |
| 5:131644726:C:T | missense | ENST00000510461.6: | ENSP00000421985.1: |
| | | c.3460G>A | p.Ala1154Thr |
| 5:131644728:A:G | missense | ENST00000510461.6: | ENSP00000421985.1: |
| | | c.3458T>C | p.Val1153Ala |
| 5:131644734:G:A | missense | ENST00000510461.6: | ENSP00000421985.1: |
| | | c.3452C>T | p.Ala1151Val |
| 5:131644735:C:A | missense | ENST00000510461.6: | ENSP00000421985.1: |
| | | c.3451G>T | p.Ala1151Ser |
| 5:131644735:C:T | missense | ENST00000510461.6: | ENSP00000421985.1: |
| | | c.3451G>A | p.Ala1151Thr |
| 5:131644738:G:C | missense | ENST00000510461.6: | ENSP00000421985.1: |
| | | c.3448C>G | p.Leu1150Val |
| 5:131644744:G:A | missense | ENST00000510461.6: | ENSP00000421985.1: |
| | | c.3442C>T | p.Pro1148Ser |
| 5:131644759:C:T | missense | ENST00000510461.6: | ENSP00000421985.1: |
| | | c.3427G>A | p.Glu1143Lys |
| 5:131644761:A:G | missense | ENST00000510461.6: | ENSP00000421985.1: |
| | | c.3425T>C | p.Ile1142Thr |
| 5:131647089:C:T | splice_ | ENST00000510461.6: | NA |
| | donor | c.3422+1G>A | |
| 5:131647090:C:A | missense | ENST00000510461.6: | ENSP00000421985.1: |
| | | c.3422G>T | p.Gly1141Val |
| 5:131647090:C:T | missense | ENST00000510461.6: | ENSP00000421985.1: |
| | | c.3422G>A | p.Gly1141Glu |
| 5:131647091:C:A | missense | ENST00000510461.6: | ENSP00000421985.1: |
| | | c.3421G>T | p.Gly1141Trp |
| 5:131647093:A:G | missense | ENST00000510461.6: | ENSP00000421985.1: |
| | | c.3419T>C | p.Leu1140Pro |
| 5:131647093:A:T | missense | ENST00000510461.6: | ENSP00000421985.1: |
| | | c.3419T>A | p.Leu1140Gln |
| 5:131647100:C:A | missense | ENST00000510461.6: | ENSP00000421985.1: |
| | | c.3412G>T | p.Val1138Leu |
| 5:131647106:G:C | missense | ENST00000510461.6: | ENSP00000421985.1: |
| | | c.3406C>G | p.Leu1136Val |
| 5:131647107:C:A | missense | ENST00000510461.6: | ENSP00000421985.1: |
| | | c.3405G>T | p.Glu1135Asp |
| 5:131647111:T:C | missense | ENST00000510461.6: | ENSP00000421985.1: |
| | | c.3401A>G | p.Lys1134Arg |
| 5:131647112:T:C | missense | ENST00000510461.6: | ENSP00000421985.1: |
| | | c.3400A>G | p.Lys1134Glu |
| 5:131647117:T:G | missense | ENST00000510461.6: | ENSP00000421985.1: |
| | | c.3395A>C | p.His1132Pro |
| 5:131647118:G:A | missense | ENST00000510461.6: | ENSP00000421985.1: |
| | | c.3394C>T | p.His1132Tyr |
| 5:131647123:C:CG | frameshift | ENST00000510461.6: | ENSP00000421985.1: |
| | | c.3388dup | p.Arg1130ProfsTer14 |
| 5:131647123:C:G | missense | ENST00000510461.6: | ENSP00000421985.1: |
| | | c.3389G>C | p.Arg1130Pro |
| 5:131647123:C:T | missense | ENST00000510461.6: | ENSP00000421985.1: |
| | | c.3389G>A | p.Arg1130His |
| 5:131647124:G:A | missense | ENST00000510461.6: | ENSP00000421985.1: |
| | | c.3388C>T | p.Arg1130Cys |
| 5:131647124:G:C | missense | ENST00000510461.6: | ENSP00000421985.1: |
| | | c.3388C>G | p.Arg1130Gly |
| 5:131647125:C:T | missense | ENST00000510461.6: | ENSP00000421985.1: |
| | | c.3387G>A | p.Met1129Ile |
| 5:131647129:T:C | missense | ENST00000510461.6: | ENSP00000421985.1: |
| | | c.3383A>G | p.Gln1128Arg |
| 5:131647132:C:G | missense | ENST00000510461.6: | ENSP00000421985.1: |
| | | c.3380G>C | p.Gly1127Ala |
| 5:131647135:C:G | missense | ENST00000510461.6: | ENSP00000421985.1: |
| | | c.3377G>C | p.Arg1126Thr |
| 5:131647145:C:T | missense | ENST00000510461.6: | ENSP00000421985.1: |
| | | c.3367G>A | p.Glu1123Lys |
| 5:131647151:G:C | missense | ENST00000510461.6: | ENSP00000421985.1: |
| | | c.3361C>G | p.Leu1121Val |
| 5:131647154:T:G | missense | ENST00000510461.6: | ENSP00000421985.1: |
| | | c.3358A>C | p.Met1120Leu |
| 5:131647162:T:C | missense | ENST00000510461.6: | ENSP00000421985.1: |
| | | c.3350A>G | p.Lys1117Arg |
| 5:131647165:A:T | missense | ENST00000510461.6: | ENSP00000421985.1: |
| | | c.3347T>A | p.Phe1116Tyr |
| 5:131647166:A:G | missense | ENST00000510461.6: | ENSP00000421985.1: |
| | | c.3346T>C | p.Phe1116Leu |
| 5:131647174:T:C | missense | ENST00000510461.6: | ENSP00000421985.1: |
| | | c.3338A>G | p.Glu1113Gly |
| 5:131647175:C:T | missense | ENST00000510461.6: | ENSP00000421985.1: |
| | | c.3337G>A | p.Glu1113Lys |
| 5:131647183:C:A | missense | ENST00000510461.6: | ENSP00000421985.1: |
| | | c.3329G>T | p.Arg1110Leu |
| 5:131647183:C:T | missense | ENST00000510461.6: | ENSP00000421985.1: |
| | | c.3329G>A | p.Arg1110Gln |
| 5:131647184:G:A | missense | ENST00000510461.6: | ENSP00000421985.1: |
| | | c.3328C>T | p.Arg1110Trp |
| 5:131647187:C:G | missense | ENST00000510461.6: | ENSP00000421985.1: |
| | | c.3325G>C | p.Asp1109His |
| 5:131647188:T:G | missense | ENST00000510461.6: | ENSP00000421985.1: |
| | | c.3324A>C | p.Glu1108Asp |
| 5:131647190:C:T | missense | ENST00000510461.6: | ENSP00000421985.1: |
| | | c.3322G>A | p.Glu1108Lys |
| 5:131647195:T:C | missense | ENST00000510461.6: | ENSP00000421985.1: |
| | | c.3317A>G | p.His1106Arg |
| 5:131647195:T:G | missense | ENST00000510461.6: | ENSP00000421985.1: |
| | | c.3317A>C | p.His1106Pro |
| 5:131647196:G: | frameshift | ENST00000510461.6: | ENSP00000421985.1: |
| GCATT | | c.3312_ | p.His1106AsnfsTer4 |
| | | 3315dup | |
| 5:131647199:T:A | missense | ENST00000510461.6: | ENSP00000421985.1: |
| | | c.3313A>T | p.Met1105Leu |
| 5:131647199:T:C | missense | ENST00000510461.6: | ENSP00000421985.1: |
| | | c.3313A>G | p.Met1105Val |
| 5:131647202:C:T | missense | ENST00000510461.6: | ENSP00000421985.1: |
| | | c.3310G>A | p.Val1104Ile |
| 5:131647206:C:CTG | splice_ | ENST00000510461.6: | NA |
| | acceptor | c.3307-3_ | |
| | | 3307-2dup | |
| 5:131651807:T:C | missense | ENST00000510461.6: | ENSP00000421985.1: |
| | | c.3301A>G | p.Asn1101Asp |
| 5:131651810:G:A | missense | ENST00000510461.6: | ENSP00000421985.1: |
| | | c.3298C>T | p.Pro1100Ser |
| 5:131651810:G:T | missense | ENST00000510461.6: | ENSP00000421985.1: |
| | | c.3298C>A | p.Pro1100Thr |
| 5:131651814:C:G | missense | ENST00000510461.6: | ENSP00000421985.1: |
| | | c.3294G>C | p.Leu1098Phe |
| 5:131651814:CA:C | frameshift | ENST00000510461.6: | ENSP00000421985.1: |
| | | c.3293del | p.Leu1098CysfsTer7 |
| 5:131651817:GT:G | frameshift | ENST00000510461.6: | ENSP00000421985.1: |
| | | c.3290del | p.Asn1097ThrfsTer8 |
| 5:131651818:T:A | missense | ENST00000510461.6: | ENSP00000421985.1: |
| | | c.3290A>T | p.Asn1097Ile |
| 5:131651819:T:G | missense | ENST00000510461.6: | ENSP00000421985.1: |
| | | c.3289A>C | p.Asn1097His |
| 5:131651821:T:C | missense | ENST00000510461.6: | ENSP00000421985.1: |
| | | c.3287A>G | p.His1096Arg |
| 5:131651824:T:A | missense | ENST00000510461.6: | ENSP00000421985.1: |
| | | c.3284A>T | p.Lys1095Met |
| 5:131651824:T:C | missense | ENST00000510461.6: | ENSP00000421985.1: |
| | | c.3284A>G | p.Lys1095Arg |
| 5:131651825:T:C | missense | ENST00000510461.6: | ENSP00000421985.1: |
| | | c.3283A>G | p.Lys1095Glu |
| 5:131651828:A:G | missense | ENST00000510461.6: | ENSP00000421985.1: |
| | | c.3280T>C | p.Tyr1094His |
| 5:131651831:G:C | missense | ENST00000510461.6: | ENSP00000421985.1: |
| | | c.3277C>G | p.Leu1093Val |
| 5:131651834:G:T | missense | ENST00000510461.6: | ENSP00000421985.1: |
| | | c.3274C>A | p.Gln1092Lys |
| 5:131651834:GAA:G | frameshift | ENST00000510461.6: | ENSP00000421985.1: |
| | | c.3272_3273del | p.Leu1091ProfsTer4 |
| 5:131651837:G:C | missense | ENST00000510461.6: | ENSP00000421985.1: |
| | | c.3271C>G | p.Leu1091Val |
| 5:131651839:G:A | missense | ENST00000510461.6: | ENSP00000421985.1: |
| | | c.3269C>T | p.Thr1090Ile |
| 5:131651840:T:C | missense | ENST00000510461.6: | ENSP00000421985.1: |
| | | c.3268A>G | p.Thr1090Ala |
| 5:131651842:G:A | missense | ENST00000510461.6: | ENSP00000421985.1: |
| | | c.3266C>T | p.Ser1089Phe |
| 5:131651842:G:T | missense | ENST00000510461.6: | ENSP00000421985.1: |
| | | c.3266C>A | p.Ser1089Tyr |
| 5:131651844:A:T | missense | ENST00000510461.6: | ENSP00000421985.1: |
| | | c.3264T>A | p.His1088Gln |
| 5:131651849:G:A | missense | ENST00000510461.6: | ENSP00000421985.1: |
| | | c.3259C>T | p.Leu1087Phe |
| 5:131651854:T:C | missense | ENST00000510461.6: | ENSP00000421985.1: |
| | | c.3254A>G | p.Asn1085Ser |
| 5:131651863:A:G | missense | ENST00000510461.6: | ENSP00000421985.1: |
| | | c.3245T>C | p.Leu1082Pro |
| 5:131651866:C:A | missense | ENST00000510461.6: | ENSP00000421985.1: |
| | | c.3242G>T | p.Ser1081Ile |
| 5:131651875:A:G | missense | ENST00000510461.6: | ENSP00000421985.1: |
| | | c.3233T>C | p.Leu1078Ser |
| 5:131651878:A:G | missense | ENST00000510461.6: | ENSP00000421985.1: |
| | | c.3230T>C | p.Val1077Ala |
| 5:131651879:C:T | missense | ENST00000510461.6: | ENSP00000421985.1: |
| | | c.3229G>A | p.Val1077Ile |
| 5:131651880:T:A | missense | ENST00000510461.6: | ENSP00000421985.1: |
| | | c.3228A>T | p.Glu1076Asp |
| 5:131651885:T:C | missense | ENST00000510461.6: | ENSP00000421985.1: |
| | | c.3223A>G | p.Lys1075Glu |
| 5:131651887:C:G | missense | ENST00000510461.6: | ENSP00000421985.1: |
| | | c.3221G>C | p.Gly1074Ala |
| 5:131651896:T:C | missense | ENST00000510461.6: | ENSP00000421985.1: |
| | | c.3212A>G | p.Asn1071Ser |
| 5:131651908:C:T | missense | ENST00000510461.6: | ENSP00000421985.1: |
| | | c.3200G>A | p.Arg1067Gln |
| 5:131651909:G:A | stop_ | ENST00000510461.6: | ENSP00000421985.1: |
| | gained | c.3199C>T | p.Arg1067Ter |
| 5:131651909:G:C | missense | ENST00000510461.6: | ENSP00000421985.1: |
| | | c.3199C>G | p.Arg1067Gly |
| 5:131651911:C:G | missense | ENST00000510461.6: | ENSP00000421985.1: |
| | | c.3197G>C | p.Arg1066Thr |
| 5:131651914:T:C | missense | ENST00000510461.6: | ENSP00000421985.1: |
| | | c.3194A>G | p.Gln1065Arg |
| 5:131651921:T:C | missense | ENST00000510461.6: | ENSP00000421985.1: |
| | | c.3187A>G | p.Ser1063Gly |
| 5:131651927:C:T | missense | ENST00000510461.6: | ENSP00000421985.1: |
| | | c.3181G>A | p.Val1061Met |
| 5:131651930:G:A | stop_ | ENST00000510461.6: | ENSP00000421985.1: |
| | gained | c.3178C>T | p.Gln1060Ter |
| 5:131651930:G:C | missense | ENST00000510461.6: | ENSP00000421985.1: |
| | | c.3178C>G | p.Gln1060Glu |
| 5:131651930:GA:G | frameshift | ENST00000510461.6: | ENSP00000421985.1: |
| | | c.3177del | p.Gln1060LysfsTer9 |
| 5:131651933:C:T | missense | ENST00000510461.6: | ENSP00000421985.1: |
| | | c.3175G>A | p.Val1059Ile |
| 5:131651935:G:C | missense | ENST00000510461.6: | ENSP00000421985.1: |
| | | c.3173C>G | p.Thr1058Ser |
| 5:131651944:T:C | missense | ENST00000510461.6: | ENSP00000421985.1: |
| | | c.3164A>G | p.Asp1055Gly |
| 5:131651946:C:T | missense | ENST00000510461.6: | ENSP00000421985.1: |
| | | c.3162G>A | p.Met1054Ile |
| 5:131651947:A:C | missense | ENST00000510461.6: | ENSP00000421985.1: |
| | | c.3161T>G | p.Met1054Arg |
| 5:131651947:A:G | missense | ENST00000510461.6: | ENSP00000421985.1: |
| | | c.3161T>C | p.Met1054Thr |
| 5:131651948:T:C | missense | ENST00000510461.6: | ENSP00000421985.1: |
| | | c.3160A>G | p.Met1054Val |
| 5:131651956:A:G | missense | ENST00000510461.6: | ENSP00000421985.1: |
| | | c.3152T>C | p.Ile1051Thr |
| 5:131651963:A:G | missense | ENST00000510461.6: | ENSP00000421985.1: |
| | | c.3145T>C | p.Cys1049Arg |
| 5:131651963:A:T | missense | ENST00000510461.6: | ENSP00000421985.1: |
| | | c.3145T>A | p.Cys1049Ser |
| 5:131651966:C:G | missense | ENST00000510461.6: | ENSP00000421985.1: |
| | | c.3142G>C | p.Val1048Leu |
| 5:131651966:C:T | missense | ENST00000510461.6: | ENSP00000421985.1: |
| | | c.3142G>A | p.Val1048Ile |
| 5:131651968:G:C | missense | ENST00000510461.6: | ENSP00000421985.1: |
| | | c.3140C>G | p.Ala1047Gly |
| 5:131651969:C:T | missense | ENST00000510461.6: | ENSP00000421985.1: |
| | | c.3139G>A | p.Ala1047Thr |
| 5:131651972:C:T | missense | ENST00000510461.6: | ENSP00000421985.1: |
| | | c.3136G>A | p.Glu1046Lys |
| 5:131651978:T:C | missense | ENST00000510461.6: | ENSP00000421985.1: |
| | | c.3130A>G | p.Ile1044Val |
| 5:131651993:C:G | missense | ENST00000510461.6: | ENSP00000421985.1: |
| | | c.3115G>C | p.Val1039Leu |
| 5:131651995:G:A | missense | ENST00000510461.6: | ENSP00000421985.1: |
| | | c.3113C>T | p.Pro1038Leu |
| 5:131651999:G:T | missense | ENST00000510461.6: | ENSP00000421985.1: |
| | | c.3109C>A | p.His1037Asn |
| 5:131670467:A:G | missense | ENST00000510461.6: | ENSP00000421985.1: |
| | | c.3104T>C | p.Val1035Ala |
| 5:131670468:C:T | missense | ENST00000510461.6: | ENSP00000421985.1: |
| | | c.3103G>A | p.Val1035Met |
| 5:131670471:C:T | missense | ENST00000510461.6: | ENSP00000421985.1: |
| | | c.3100G>A | p.Ala1034Thr |
| 5:131670472:A:T | missense | ENST00000510461.6: | ENSP00000421985.1: |
| | | c.3099T>A | p.His1033Gln |
| 5:131670473:T:C | missense | ENST00000510461.6: | ENSP00000421985.1: |
| | | c.3098A>G | p.His1033Arg |
| 5:131670473:T:G | missense | ENST00000510461.6: | ENSP00000421985.1: |
| | | c.3098A>C | p.His1033Pro |
| 5:131670474:G:T | missense | ENST00000510461.6: | ENSP00000421985.1: |
| | | c.3097C>A | p.His1033Asn |
| 5:131670476:G:A | missense | ENST00000510461.6: | ENSP00000421985.1: |
| | | c.3095C>T | p.Ser1032Phe |
| 5:131670476:G:C | missense | ENST00000510461.6: | ENSP00000421985.1: |
| | | c.3095C>G | p.Ser1032Cys |
| 5:131670477:A:C | missense | ENST00000510461.6: | ENSP00000421985.1: |
| | | c.3094T>G | p.Ser1032Ala |
| 5:131670482:T:A | missense | ENST00000510461.6: | ENSP00000421985.1: |
| | | c.3089A>T | p.Asp1030Val |
| 5:131670483:C:G | missense | ENST00000510461.6: | ENSP00000421985.1: |
| | | c.3088G>C | p.Asp1030His |
| 5:131670483:C:T | missense | ENST00000510461.6: | ENSP00000421985.1: |
| | | c.3088G>A | p.Asp1030Asn |
| 5:131670487:C:T | missense | ENST00000510461.6: | ENSP00000421985.1: |
| | | c.3084G>A | p.Met1028Ile |
| 5:131670493:A:T | stop_ | ENST00000510461.6: | ENSP00000421985.1: |
| | gained | c.3078T>A | p.Cys1026Ter |
| 5:131670500:C:A | missense | ENST00000510461.6: | ENSP00000421985.1: |
| | | c.3071G>T | p.Arg1024Leu |
| 5:131670500:C:T | missense | ENST00000510461.6: | ENSP00000421985.1: |
| | | c.3071G>A | p.Arg1024His |
| 5:131670501:G:A | missense | ENST00000510461.6: | ENSP00000421985.1: |
| | | c.3070C>T | p.Arg1024Cys |
| 5:131670502:G:C | missense | ENST00000510461.6: | ENSP00000421985.1: |
| | | c.3069C>G | p.Phe1023Leu |
| 5:131670503:A:T | missense | ENST00000510461.6: | ENSP00000421985.1: |
| | | c.3068T>A | p.Phe1023Tyr |
| 5:131670505:C:G | missense | ENST00000510461.6: | ENSP00000421985.1: |
| | | c.3066G>C | p.Arg1022Ser |
| 5:131670506:C:T | missense | ENST00000510461.6: | ENSP00000421985.1: |
| | | c.3065G>A | p.Arg1022Lys |
| 5:131670510:C:T | missense | ENST00000510461.6: | ENSP00000421985.1: |
| | | c.3061G>A | p.Glu1021Lys |
| 5:131670511:A:C | missense | ENST00000510461.6: | ENSP00000421985.1: |
| | | c.3060T>G | p.Asp1020Glu |
| 5:131670514:A:C | missense | ENST00000510461.6: | ENSP00000421985.1: |
| | | c.3057T>G | p.Ser1019Arg |
| 5:131670519:C:A | missense | ENST00000510461.6: | ENSP00000421985.1: |
| | | c.3052G>T | p.Gly1018Trp |
| 5:131670521:A:G | missense | ENST00000510461.6: | ENSP00000421985.1: |
| | | c.3050T>C | p.Ile1017Thr |
| 5:131670540:C:G | missense | ENST00000510461.6: | ENSP00000421985.1: |
| | | c.3031G>C | p.Asp1011His |
| 5:131670543:G:T | missense | ENST00000510461.6: | ENSP00000421985.1: |
| | | c.3028C>A | p.Pro1010Thr |
| 5:131670546:C:T | missense | ENST00000510461.6: | ENSP00000421985.1: |
| | | c.3025G>A | p.Val1009Met |
| 5:131670550:A:AT | frameshift | ENST00000510461.6: | ENSP00000421985.1: |
| | | c.3020_ | p.Tyr1008LeufsTer4 |
| | | 3021insA | |
| 5:131670551:G:A | missense | ENST00000510461.6: | ENSP00000421985.1: |
| | | c.3020C>T | p.Ser1007Phe |
| 5:131670552:A:G | missense | ENST00000510461.6: | ENSP00000421985.1: |
| | | c.3019T>C | p.Ser1007Pro |
| 5:131670554:G:A | missense | ENST00000510461.6: | ENSP00000421985.1: |
| | | c.3017C>T | p.Ser1006Leu |
| 5:131670554:G:T | stop_ | ENST00000510461.6: | ENSP00000421985.1: |
| | gained | c.3017C>A | p.Ser1006Ter |
| 5:131670560:T:C | missense | ENST00000510461.6: | ENSP00000421985.1: |
| | | c.3011A>G | p.Tyr1004Cys |
| 5:131670561:A:G | missense | ENST00000510461.6: | ENSP00000421985.1: |
| | | c.3010T>C | p.Tyr1004His |
| 5:131670579:T:A | missense | ENST00000510461.6: | ENSP00000421985.1: |
| | | c.2992A>T | p.Arg998Trp |
| 5:131670582:C:T | missense | ENST00000510461.6: | ENSP00000421985.1: |
| | | c.2989G>A | p.Gly997Arg |
| 5:131670585:A:G | missense | ENST00000510461.6: | ENSP00000421985.1: |
| | | c.2986T>C | p.Phe996Leu |
| 5:131670593:A:G | missense | ENST00000510461.6: | ENSP00000421985.1: |
| | | c.2978T>C | p.Ile993Thr |
| 5:131670594:T:C | missense | ENST00000510461.6: | ENSP00000421985.1: |
| | | c.2977A>G | p.Ile993Val |
| 5:131670596:T:A | missense | ENST00000510461.6: | ENSP00000421985.1: |
| | | c.2975A>T | p.Asn992Ile |
| 5:131670605:A:T | missense | ENST00000510461.6: | ENSP00000421985.1: |
| | | c.2966T>A | p.Val989Asp |
| 5:131670606:C:G | missense | ENST00000510461.6: | ENSP00000421985.1: |
| | | c.2965G>C | p.Val989Leu |
| 5:131670606:C:T | missense | ENST00000510461.6: | ENSP00000421985.1: |
| | | c.2965G>A | p.Val989Ile |
| 5:131670610:A:C | missense | ENST00000510461.6: | ENSP00000421985.1: |
| | | c.2961T>G | p.Ser987Arg |
| 5:131670611:C:A | missense | ENST00000510461.6: | ENSP00000421985.1: |
| | | c.2960G>T | p.Ser987Ile |
| 5:131670611:C:T | missense | ENST00000510461.6: | ENSP00000421985.1: |
| | | c.2960G>A | p.Ser987Asn |
| 5:131670612:T:C | missense | ENST00000510461.6: | ENSP00000421985.1: |
| | | c.2959A>G | p.Ser987Gly |
| 5:131670614:A:G | missense | ENST00000510461.6: | ENSP00000421985.1: |
| | | c.2957T>C | p.Val986Ala |
| 5:131670618:C:T | missense | ENST00000510461.6: | ENSP00000421985.1: |
| | | c.2953G>A | p.Glu985Lys |
| 5:131670621:T:C | missense | ENST00000510461.6: | ENSP00000421985.1: |
| | | c.2950A>G | p.Ile984Val |
| 5:131670626:T:C | missense | ENST00000510461.6: | ENSP00000421985.1: |
| | | c.2945A>G | p.Lys982Arg |
| 5:131670632:C:T | splice_ | ENST00000510461.6: | NA |
| | acceptor | c.2940-1G>A | |
| 5:131671506:C:A | missense | ENST00000510461.6: | ENSP00000421985.1: |
| | | c.2938G>T | p.Gly980Trp |
| 5:131671509:G:A | missense | ENST00000510461.6: | ENSP00000421985.1: |
| | | c.2935C>T | p.Pro979Ser |
| 5:131671509:G:C | missense | ENST00000510461.6: | ENSP00000421985.1: |
| | | c.2935C>G | p.Pro979Ala |
| 5:131671514:G:C | missense | ENST00000510461.6: | ENSP00000421985.1: |
| | | c.2930C>G | p.Pro977Arg |
| 5:131671516:T:C | missense | ENST00000510461.6: | ENSP00000421985.1: |
| | | c.2928A>G | p.Ile976Met |
| 5:131671522:A:C | missense | ENST00000510461.6: | ENSP00000421985.1: |
| | | c.2922T>G | p.Asp974Glu |
| 5:131671522:A:T | missense | ENST00000510461.6: | ENSP00000421985.1: |
| | | c.2922T>A | p.Asp974Glu |
| 5:131671524:C:T | missense | ENST00000510461.6: | ENSP00000421985.1: |
| | | c.2920G>A | p.Asp974Asn |
| 5:131671527:C:T | missense | ENST00000510461.6: | ENSP00000421985.1: |
| | | c.2917G>A | p.Glu973Lys |
| 5:131671533:C:A | missense | ENST00000510461.6: | ENSP00000421985.1: |
| | | c.2911G>T | p.Ala971Ser |
| 5:131671533:C:G | missense | ENST00000510461.6: | ENSP00000421985.1: |
| | | c.2911G>C | p.Ala971Pro |
| 5:131671533:C:T | missense | ENST00000510461.6: | ENSP00000421985.1: |
| | | c.2911G>A | p.Ala971Thr |
| 5:131671534:C:A | missense | ENST00000510461.6: | ENSP00000421985.1: |
| | | c.2910G>T | p.Trp970Cys |
| 5:131671538:T:A | missense | ENST00000510461.6: | ENSP00000421985.1: |
| | | c.2906A>T | p.Asp969Val |
| 5:131671539:C:G | missense | ENST00000510461.6: | ENSP00000421985.1: |
| | | c.2905G>C | p.Asp969His |
| 5:131671543:T:G | missense | ENST00000510461.6: | ENSP00000421985.1: |
| | | c.2901A>C | p.Gln967His |
| 5:131671545:G:C | missense | ENST00000510461.6: | ENSP00000421985.1: |
| | | c.2899C>G | p.Gln967Glu |
| 5:131671548:C:G | missense | ENST00000510461.6: | ENSP00000421985.1: |
| | | c.2896G>C | p.Glu966Gln |
| 5:131671551:C:T | missense | ENST00000510461.6: | ENSP00000421985.1: |
| | | c.2893G>A | p.Gly965Arg |
| 5:131671554:C:T | missense | ENST00000510461.6: | ENSP00000421985.1: |
| | | c.2890G>A | p.Gly964Arg |
| 5:131671557:A:C | missense | ENST00000510461.6: | ENSP00000421985.1: |
| | | c.2887T>G | p.Tyr963Asp |
| 5:131671559:T:C | missense | ENST00000510461.6: | ENSP00000421985.1: |
| | | c.2885A>G | p.Tyr962Cys |
| 5:131671562:C:T | missense | ENST00000510461.6: | ENSP00000421985.1: |
| | | c.2882G>A | p.Ser961Asn |
| 5:131671569:C:T | missense | ENST00000510461.6: | ENSP00000421985.1: |
| | | c.2875G>A | p.Val959Ile |
| 5:131671572:G:C | missense | ENST00000510461.6: | ENSP00000421985.1: |
| | | c.2872C>G | p.Gln958Glu |
| 5:131671572:G:T | missense | ENST00000510461.6: | ENSP00000421985.1: |
| | | c.2872C>A | p.Gln958Lys |
| 5:131671574:C:T | missense | ENST00000510461.6: | ENSP00000421985.1: |
| | | c.2870G>A | p.Arg957Lys |
| 5:131671575:T:C | missense | ENST00000510461.6: | ENSP00000421985.1: |
| | | c.2869A>G | p.Arg957Gly |
| 5:131671584:C:T | missense | ENST00000510461.6: | ENSP00000421985.1: |
| | | c.2860G>A | p.Asp954Asn |
| 5:131671586:T:C | missense | ENST00000510461.6: | ENSP00000421985.1: |
| | | c.2858A>G | p.His953Arg |
| 5:131671589:C:A | missense | ENST00000510461.6: | ENSP00000421985.1: |
| | | c.2855G>T | p.Gly952Val |
| 5:131671592:G:A | missense | ENST00000510461.6: | ENSP00000421985.1: |
| | | c.2852C>T | p.Thr951Ile |
| 5:131671595:T:C | missense | ENST00000510461.6: | ENSP00000421985.1: |
| | | c.2849A>G | p.Asp950Gly |
| 5:131671595:T:G | missense | ENST00000510461.6: | ENSP00000421985.1: |
| | | c.2849A>C | p.Asp950Ala |
| 5:131671601:C:T | missense | ENST00000510461.6: | ENSP00000421985.1: |
| | | c.2843G>A | p.Ser948Asn |
| 5:131671604:T:C | missense | ENST00000510461.6: | ENSP00000421985.1: |
| | | c.2840A>G | p.Glu947Gly |
| 5:131671604:T:G | missense | ENST00000510461.6: | ENSP00000421985.1: |
| | | c.2840A>C | p.Glu947Ala |
| 5:131671610:T:C | missense | ENST00000510461.6: | ENSP00000421985.1: |
| | | c.2834A>G | p.Asp945Gly |
| 5:131671610:T:G | missense | ENST00000510461.6: | ENSP00000421985.1: |
| | | c.2834A>C | p.Asp945Ala |
| 5:131671613:C:T | missense | ENST00000510461.6: | ENSP00000421985.1: |
| | | c.2831G>A | p.Gly944Glu |
| 5:131671614:C:T | missense | ENST00000510461.6: | ENSP00000421985.1: |
| | | c.2830G>A | p.Gly944Arg |
| 5:131671617:G:A | missense | ENST00000510461.6: | ENSP00000421985.1: |
| | | c.2827C>T | p.Leu943Phe |
| 5:131671628:G:A | missense | ENST00000510461.6: | ENSP00000421985.1: |
| | | c.2816C>T | p.Ser939Leu |
| 5:131671632:T:C | missense | ENST00000510461.6: | ENSP00000421985.1: |
| | | c.2812A>G | p.Ser938Gly |
| 5:131671634:T:C | missense | ENST00000510461.6: | ENSP00000421985.1: |
| | | c.2810A>G | p.Glu937Gly |
| 5:131671634:T:G | missense | ENST00000510461.6: | ENSP00000421985.1: |
| | | c.2810A>C | p.Glu937Ala |
| 5:131671639:T:G | missense | ENST00000510461.6: | ENSP00000421985.1: |
| | | c.2805A>C | p.Arg935Ser |
| 5:131671646:A:T | missense | ENST00000510461.6: | ENSP00000421985.1: |
| | | c.2798T>A | p.Ile933Asn |
| 5:131671647:T:A | missense | ENST00000510461.6: | ENSP00000421985.1: |
| | | c.2797A>T | p.Ile933Phe |
| 5:131671647:T:C | missense | ENST00000510461.6: | ENSP00000421985.1: |
| | | c.2797A>G | p.Ile933Val |
| 5:131671647:T:G | missense | ENST00000510461.6: | ENSP00000421985.1: |
| | | c.2797A>C | p.Ile933Leu |
| 5:131671658:G:A | missense | ENST00000510461.6: | ENSP00000421985.1: |
| | | c.2786C>T | p.Thr929Ile |
| 5:131671658:G:C | missense | ENST00000510461.6: | ENSP00000421985.1: |
| | | c.2786C>G | p.Thr929Ser |
| 5:131671659:T:C | missense | ENST00000510461.6: | ENSP00000421985.1: |
| | | c.2785A>G | p.Thr929Ala |
| 5:131671661:C:T | missense | ENST00000510461.6: | ENSP00000421985.1: |
| | | c.2783G>A | p.Gly928Glu |
| 5:131671664:A:C | missense | ENST00000510461.6: | ENSP00000421985.1: |
| | | c.2780T>G | p.Val927Gly |
| 5:131671665:C:T | missense | ENST00000510461.6: | ENSP00000421985.1: |
| | | c.2779G>A | p.Val927Ile |
| 5:131671668:C:A | missense | ENST00000510461.6: | ENSP00000421985.1: |
| | | c.2776G>T | p.Ala926Ser |
| 5:131671669:A:C | missense | ENST00000510461.6: | ENSP00000421985.1: |
| | | c.2775T>G | p.Ile925Met |
| 5:131671670:A:AT | frameshift | ENST00000510461.6: | ENSP00000421985.1: |
| | | c.2773dup | p.Ile925AsnfsTer6 |
| 5:131671670:A:T | missense | ENST00000510461.6: | ENSP00000421985.1: |
| | | c.2774T>A | p.Ile925Asn |
| 5:131671673:T:C | missense | ENST00000510461.6: | ENSP00000421985.1: |
| | | c.2771A>G | p.Lys924Arg |
| 5:131671677:T:A | stop_ | ENST00000510461.6: | ENSP00000421985.1: |
| | gained | c.2767A>T | p.Lys923Ter |
| 5:131671680:C:T | missense | ENST00000510461.6: | ENSP00000421985.1: |
| | | c.2764G>A | p.Asp922Asn |
| 5:131671683:A:C | missense | ENST00000510461.6: | ENSP00000421985.1: |
| | | c.2761T>G | p.Ser921Ala |
| 5:131671685:C:A | missense | ENST00000510461.6: | ENSP00000421985.1: |
| | | c.2759G>T | p.Ser920Ile |
| 5:131671686:T:C | missense | ENST00000510461.6: | ENSP00000421985.1: |
| | | c.2758A>G | p.Ser920Gly |
| 5:131671691:T:C | missense | ENST00000510461.6: | ENSP00000421985.1: |
| | | c.2753A>G | p.Lys918Arg |
| 5:131671692:T:C | missense | ENST00000510461.6: | ENSP00000421985.1: |
| | | c.2752A>G | p.Lys918Glu |
| 5:131671694:T:A | missense | ENST00000510461.6: | ENSP00000421985.1: |
| | | c.2750A>T | p.Asp917Val |
| 5:131671697:C:A | missense | ENST00000510461.6: | ENSP00000421985.1: |
| | | c.2747G>T | p.Gly916Val |
| 5:131671700:T:C | missense | ENST00000510461.6: | ENSP00000421985.1: |
| | | c.2744A>G | p.His915Arg |
| 5:131671700:TG:T | frameshift | ENST00000510461.6: | ENSP00000421985.1: |
| | | c.2743del | p.His915MetfsTer13 |
| 5:131671701:G:A | missense | ENST00000510461.6: | ENSP00000421985.1: |
| | | c.2743C>T | p.His915Tyr |
| 5:131671703:G:A | missense | ENST00000510461.6: | ENSP00000421985.1: |
| | | c.2741C>T | p.Pro914Leu |
| 5:131671703:G:C | missense | ENST00000510461.6: | ENSP00000421985.1: |
| | | c.2741C>G | p.Pro914Arg |
| 5:131671704:G:A | missense | ENST00000510461.6: | ENSP00000421985.1: |
| | | c.2740C>T | p.Pro914Ser |
| 5:131671705:GA:G | frameshift | ENST00000510461.6: | ENSP00000421985.1: |
| | | c.2738del | p.Val913AlafsTer15 |
| 5:131671706:A:T | missense | ENST00000510461.6: | ENSP00000421985.1: |
| | | c.2738T>A | p.Val913Asp |
| 5:131671710:G:T | missense | ENST00000510461.6: | ENSP00000421985.1: |
| | | c.2734C>A | p.Leu912Ile |
| 5:131671712:A:C | missense | ENST00000510461.6: | ENSP00000421985.1: |
| | | c.2732T>G | p.Ile911Ser |
| 5:131671713:T:C | missense | ENST00000510461.6: | ENSP00000421985.1: |
| | | c.2731A>G | p.Ile911Val |
| 5:131671713:TG:T | frameshift | ENST00000510461.6: | ENSP00000421985.1: |
| | | c.2730del | p.Ile911PhefsTer17 |
| 5:131671718:A:G | missense | ENST00000510461.6: | ENSP00000421985.1: |
| | | c.2726T>C | p.Leu909Pro |
| 5:131671718:A:T | missense | ENST00000510461.6: | ENSP00000421985.1: |
| | | c.2726T>A | p.Leu909His |
| 5:131671726:T:A | missense | ENST00000510461.6: | ENSP00000421985.1: |
| | | c.2718A>T | p.Arg906Ser |
| 5:131671732:G:C | missense | ENST00000510461.6: | ENSP00000421985.1: |
| | | c.2712C>G | p.Asp904Glu |
| 5:131671734:C:T | missense | ENST00000510461.6: | ENSP00000421985.1: |
| | | c.2710G>A | p.Asp904Asn |
| 5:131671740:G:C | missense | ENST00000510461.6: | ENSP00000421985.1: |
| | | c.2704C>G | p.Gln902Glu |
| 5:131671742:G:A | missense | ENST00000510461.6: | ENSP00000421985.1: |
| | | c.2702C>T | p.Pro901Leu |
| 5:131671742:G:T | missense | ENST00000510461.6: | ENSP00000421985.1: |
| | | c.2702C>A | p.Pro901His |
| 5:131671745:A:G | missense | ENST00000510461.6: | ENSP00000421985.1: |
| | | c.2699T>C | p.Phe900Ser |
| 5:131671751:G:A | missense | ENST00000510461.6: | ENSP00000421985.1: |
| | | c.2693C>T | p.Thr898Ile |
| 5:131671751:G:T | missense | ENST00000510461.6: | ENSP00000421985.1: |
| | | c.2693C>A | p.Thr898Asn |
| 5:131671754:T:C | missense | ENST00000510461.6: | ENSP00000421985.1: |
| | | c.2690A>G | p.Lys897Arg |
| 5:131671755:T:A | stop_ | ENST00000510461.6: | ENSP00000421985.1: |
| | gained | c.2689A>T | p.Lys897Ter |
| 5:131671758:A:G | missense | ENST00000510461.6: | ENSP00000421985.1: |
| | | c.2686T>C | p.Cys896Arg |
| 5:131671760:G:A | missense | ENST00000510461.6: | ENSP00000421985.1: |
| | | c.2684C>T | p.Ser895Leu |
| 5:131671761:A:T | missense | ENST00000510461.6: | ENSP00000421985.1: |
| | | c.2683T>A | p.Ser895Thr |
| 5:131671762:A:C | missense | ENST00000510461.6: | ENSP00000421985.1: |
| | | c.2682T>G | p.Asp894Glu |
| 5:131671763:T:C | missense | ENST00000510461.6: | ENSP00000421985.1: |
| | | c.2681A>G | p.Asp894Gly |
| 5:131671764:C:G | missense | ENST00000510461.6: | ENSP00000421985.1: |
| | | c.2680G>C | p.Asp894His |
| 5:131671764:C:T | missense | ENST00000510461.6: | ENSP00000421985.1: |
| | | c.2680G>A | p.Asp894Asn |
| 5:131671767:G:C | missense | ENST00000510461.6: | ENSP00000421985.1: |
| | | c.2677C>G | p.Gln893Glu |
| 5:131671769:G:A | missense | ENST00000510461.6: | ENSP00000421985.1: |
| | | c.2675C>T | p.Pro892Leu |
| 5:131671770:G:A | missense | ENST00000510461.6: | ENSP00000421985.1: |
| | | c.2674C>T | p.Pro892Ser |
| 5:131671770:G:T | missense | ENST00000510461.6: | ENSP00000421985.1: |
| | | c.2674C>A | p.Pro892Thr |
| 5:131671772:A:C | missense | ENST00000510461.6: | ENSP00000421985.1: |
| | | c.2672T>G | p.Val891Gly |
| 5:131671772:A:G | missense | ENST00000510461.6: | ENSP00000421985.1: |
| | | c.2672T>C | p.Val891Ala |
| 5:131671773:C:A | missense | ENST00000510461.6: | ENSP00000421985.1: |
| | | c.2671G>T | p.Val891Phe |
| 5:131671775:G:A | missense | ENST00000510461.6: | ENSP00000421985.1: |
| | | c.2669C>T | p.Thr890Ile |
| 5:131671776:T:C | missense | ENST00000510461.6: | ENSP00000421985.1: |
| | | c.2668A>G | p.Thr890Ala |
| 5:131671778:T:C | missense | ENST00000510461.6: | ENSP00000421985.1: |
| | | c.2666A>G | p.Glu889Gly |
| 5:131671779:C:G | missense | ENST00000510461.6: | ENSP00000421985.1: |
| | | c.2665G>C | p.Glu889Gln |
| 5:131671779:C:T | missense | ENST00000510461.6: | ENSP00000421985.1: |
| | | c.2665G>A | p.Glu889Lys |
| 5:131671780:T:C | missense | ENST00000510461.6: | ENSP00000421985.1: |
| | | c.2664A>G | p.Ile888Met |
| 5:131671782:T:C | missense | ENST00000510461.6: | ENSP00000421985.1: |
| | | c.2662A>G | p.Ile888Val |
| 5:131671784:C:T | missense | ENST00000510461.6: | ENSP00000421985.1: |
| | | c.2660G>A | p.Cys887Tyr |
| 5:131671788:T:G | missense | ENST00000510461.6: | ENSP00000421985.1: |
| | | c.2656A>C | p.Lys886Gln |
| 5:131671788:TA:T | frameshift | ENST00000510461.6: | ENSP00000421985.1: |
| | | c.2655del | p.Cys885Ter |
| 5:131671794:A:G | missense | ENST00000510461.6: | ENSP00000421985.1: |
| | | c.2650T>C | p.Phe884Leu |
| 5:131671795:T:A | missense | ENST00000510461.6: | ENSP00000421985.1: |
| | | c.2649A>T | p.Glu883Asp |
| 5:131671796:T:C | missense | ENST00000510461.6: | ENSP00000421985.1: |
| | | c.2648A>G | p.Glu883Gly |
| 5:131671797:C:G | missense | ENST00000510461.6: | ENSP00000421985.1: |
| | | c.2647G>C | p.Glu883Gln |
| 5:131671797:C:T | missense | ENST00000510461.6: | ENSP00000421985.1: |
| | | c.2647G>A | p.Glu883Lys |
| 5:131671798:A:T | missense | ENST00000510461.6: | ENSP00000421985.1: |
| | | c.2646T>A | p.Asn882Lys |
| 5:131671802:T:A | missense | ENST00000510461.6: | ENSP00000421985.1: |
| | | c.2642A>T | p.Asn881Ile |
| 5:131671802:T:C | missense | ENST00000510461.6: | ENSP00000421985.1: |
| | | c.2642A>G | p.Asn881Ser |
| 5:131671812:T:C | missense | ENST00000510461.6: | ENSP00000421985.1: |
| | | c.2632A>G | p.Asn878Asp |
| 5:131671814:T:A | missense | ENST00000510461.6: | ENSP00000421985.1: |
| | | c.2630A>T | p.Asn877Ile |
| 5:131671814:T:C | missense | ENST00000510461.6: | ENSP00000421985.1: |
| | | c.2630A>G | p.Asn877Ser |
| 5:131671816:T:G | missense | ENST00000510461.6: | ENSP00000421985.1: |
| | | c.2628A>C | p.Lys876Asn |
| 5:131671821:T:C | missense | ENST00000510461.6: | ENSP00000421985.1: |
| | | c.2623A>G | p.Thr875Ala |
| 5:131671823:C:T | missense | ENST00000510461.6: | ENSP00000421985.1: |
| | | c.2621G>A | p.Cys874Tyr |
| 5:131671825:C:A | missense | ENST00000510461.6: | ENSP00000421985.1: |
| | | c.2619G>T | p.Leu873Phe |
| 5:131671825:C:G | missense | ENST00000510461.6: | ENSP00000421985.1: |
| | | c.2619G>C | p.Leu873Phe |
| 5:131671832:T:G | missense | ENST00000510461.6: | ENSP00000421985.1: |
| | | c.2612A>C | p.Lys871Thr |
| 5:131671842:C:G | missense | ENST00000510461.6: | ENSP00000421985.1: |
| | | c.2602G>C | p.Glu868Gln |
| 5:131671844:A:G | missense | ENST00000510461.6: | ENSP00000421985.1: |
| | | c.2600T>C | p.Leu867Ser |
| 5:131671846:C:T | missense | ENST00000510461.6: | ENSP00000421985.1: |
| | | c.2598G>A | p.Met866Ile |
| 5:131671847:A:G | missense | ENST00000510461.6: | ENSP00000421985.1: |
| | | c.2597T>C | p.Met866Thr |
| 5:131671847:A:T | missense | ENST00000510461.6: | ENSP00000421985.1: |
| | | c.2597T>A | p.Met866Lys |
| 5:131671848:T:C | missense | ENST00000510461.6: | ENSP00000421985.1: |
| | | c.2596A>G | p.Met866Val |
| 5:131671850:C:T | missense | ENST00000510461.6: | ENSP00000421985.1: |
| | | c.2594G>A | p.Cys865Tyr |
| 5:131671851:A:T | missense | ENST00000510461.6: | ENSP00000421985.1: |
| | | c.2593T>A | p.Cys865Ser |
| 5:131671853:C:T | missense | ENST00000510461.6: | ENSP00000421985.1: |
| | | c.2591G>A | p.Cys864Tyr |
| 5:131671854:A:T | missense | ENST00000510461.6: | ENSP00000421985.1: |
| | | c.2590T>A | p.Cys864Ser |
| 5:131671860:C:G | missense | ENST00000510461.6: | ENSP00000421985.1: |
| | | c.2584G>C | p.Asp862His |
| 5:131671866:T:C | missense | ENST00000510461.6: | ENSP00000421985.1: |
| | | c.2578A>G | p.Ser860Gly |
| 5:131671867:A:T | missense | ENST00000510461.6: | ENSP00000421985.1: |
| | | c.2577T>A | p.Asp859Glu |
| 5:131671868:T:C | missense | ENST00000510461.6: | ENSP00000421985.1: |
| | | c.2576A>G | p.Asp859Gly |
| 5:131671869:C:T | missense | ENST00000510461.6: | ENSP00000421985.1: |
| | | c.2575G>A | p.Asp859Asn |
| 5:131671874:C:T | missense | ENST00000510461.6: | ENSP00000421985.1: |
| | | c.2570G>A | p.Ser857Asn |
| 5:131671878:T:C | missense | ENST00000510461.6: | ENSP00000421985.1: |
| | | c.2566A>G | p.Thr856Ala |
| 5:131671882:A:T | missense | ENST00000510461.6: | ENSP00000421985.1: |
| | | c.2562T>A | p.Phe854Leu |
| 5:131671887:G:C | missense | ENST00000510461.6: | ENSP00000421985.1: |
| | | c.2557C>G | p.Pro853Ala |
| 5:131671889:A:G | missense | ENST00000510461.6: | ENSP00000421985.1: |
| | | c.2555T>C | p.Val852Ala |
| 5:131671889:A:T | missense | ENST00000510461.6: | ENSP00000421985.1: |
| | | c.2555T>A | p.Val852Asp |
| 5:131671890:C:A | missense | ENST00000510461.6: | ENSP00000421985.1: |
| | | c.2554G>T | p.Val852Phe |
| 5:131671895:T:A | missense | ENST00000510461.6: | ENSP00000421985.1: |
| | | c.2549A>T | p.Asp850Val |
| 5:131671896:C:T | missense | ENST00000510461.6: | ENSP00000421985.1: |
| | | c.2548G>A | p.Asp850Asn |
| 5:131671899:T:C | missense | ENST00000510461.6: | ENSP00000421985.1: |
| | | c.2545A>G | p.Ile849Val |
| 5:131671899:T:TA | frameshift | ENST00000510461.6: | ENSP00000421985.1: |
| | | c.2544dup | p.Ile849TyrfsTer2 |
| 5:131671905:T:C | missense | ENST00000510461.6: | ENSP00000421985.1: |
| | | c.2539A>G | p.Arg847Gly |
| 5:131671907:G:A | missense | ENST00000510461.6: | ENSP00000421985.1: |
| | | c.2537C>T | p.Thr846Ile |
| 5:131671908:T:C | missense | ENST00000510461.6: | ENSP00000421985.1: |
| | | c.2536A>G | p.Thr846Ala |
| 5:131671908:T:G | missense | ENST00000510461.6: | ENSP00000421985.1: |
| | | c.2536A>C | p.Thr846Pro |
| 5:131671911:C:T | missense | ENST00000510461.6: | ENSP00000421985.1: |
| | | c.2533G>A | p.Glu845Lys |
| 5:131671912:G:C | missense | ENST00000510461.6: | ENSP00000421985.1: |
| | | c.2532C>G | p.Ile844Met |
| 5:131671914:T:C | missense | ENST00000510461.6: | ENSP00000421985.1: |
| | | c.2530A>G | p.Ile844Val |
| 5:131671918:A:C | missense | ENST00000510461.6: | ENSP00000421985.1: |
| | | c.2526T>G | p.Asp842Glu |
| 5:131671922:T:A | missense | ENST00000510461.6: | ENSP00000421985.1: |
| | | c.2522A>T | p.Asp841Val |
| 5:131671922:T:C | missense | ENST00000510461.6: | ENSP00000421985.1: |
| | | c.2522A>G | p.Asp841Gly |
| 5:131671924:A:T | missense | ENST00000510461.6: | ENSP00000421985.1: |
| | | c.2520T>A | p.Asn840Lys |
| 5:131671925:T:C | missense | ENST00000510461.6: | ENSP00000421985.1: |
| | | c.2519A>G | p.Asn840Ser |
| 5:131671926:T:G | missense | ENST00000510461.6: | ENSP00000421985.1: |
| | | c.2518A>C | p.Asn840His |
| 5:131671928:A:G | missense | ENST00000510461.6: | ENSP00000421985.1: |
| | | c.2516T>C | p.Phe839Ser |
| 5:131671933:T:A | missense | ENST00000510461.6: | ENSP00000421985.1: |
| | | c.2511A>T | p.Glu837Asp |
| 5:131671935:C:T | missense | ENST00000510461.6: | ENSP00000421985.1: |
| | | c.2509G>A | p.Glu837Lys |
| 5:131671938:C:T | missense | ENST00000510461.6: | ENSP00000421985.1: |
| | | c.2506G>A | p.Asp836Asn |
| 5:131671939:G:C | missense | ENST00000510461.6: | ENSP00000421985.1: |
| | | c.2505C>G | p.Phe835Leu |
| 5:131671941:A:G | missense | ENST00000510461.6: | ENSP00000421985.1: |
| | | c.2503T>C | p.Phe835Leu |
| 5:131671946:C:T | missense | ENST00000510461.6: | ENSP00000421985.1: |
| | | c.2498G>A | p.Ser833Asn |
| 5:131671950:T:C | missense | ENST00000510461.6: | ENSP00000421985.1: |
| | | c.2494A>G | p.Met832Val |
| 5:131671954:T:G | missense | ENST00000510461.6: | ENSP00000421985.1: |
| | | c.2490A>C | p.Glu830Asp |
| 5:131671956:C:T | missense | ENST00000510461.6: | ENSP00000421985.1: |
| | | c.2488G>A | p.Glu830Lys |
| 5:131671959:G:C | missense | ENST00000510461.6: | ENSP00000421985.1: |
| | | c.2485C>G | p.Pro829Ala |
| 5:131671963:TG:T | frameshift | ENST00000510461.6: | ENSP00000421985.1: |
| | | c.2480del | p.Ser827Ter |
| 5:131671964:G:A | missense | ENST00000510461.6: | ENSP00000421985.1: |
| | | c.2480C>T | p.Ser827Leu |
| 5:131671965:A:C | missense | ENST00000510461.6: | ENSP00000421985.1: |
| | | c.2479T>G | p.Ser827Ala |
| 5:131671967:T:C | missense | ENST00000510461.6: | ENSP00000421985.1: |
| | | c.2477A>G | p.His826Arg |
| 5:131671969: | frameshift | ENST00000510461.6: | ENSP00000421985.1: |
| ATTCC:A | | c.2471_ | p.Trp824PhefsTer8 |
| | | 2474del | |
| 5:131671970:T:C | missense | ENST00000510461.6: | ENSP00000421985.1: |
| | | c.2474A>G | p.Asn825Ser |
| 5:131671979:G:A | missense | ENST00000510461.6: | ENSP00000421985.1: |
| | | c.2465C>T | p.Pro822Leu |
| 5:131671980:G:C | missense | ENST00000510461.6: | ENSP00000421985.1: |
| | | c.2464C>G | p.Pro822Ala |
| 5:131671988:C:T | missense | ENST00000510461.6: | ENSP00000421985.1: |
| | | c.2456G>A | p.Cys819Tyr |
| 5:131671991:G:A | missense | ENST00000510461.6: | ENSP00000421985.1: |
| | | c.2453C>T | p.Pro818Leu |
| 5:131671992:G:T | missense | ENST00000510461.6: | ENSP00000421985.1: |
| | | c.2452C>A | p.Pro818Thr |
| 5:131671997:T:G | missense | ENST00000510461.6: | ENSP00000421985.1: |
| | | c.2447A>C | p.Glu816Ala |
| 5:131671998:C:T | missense | ENST00000510461.6: | ENSP00000421985.1: |
| | | c.2446G>A | p.Glu816Lys |
| 5:131672000:G:A | missense | ENST00000510461.6: | ENSP00000421985.1: |
| | | c.2444C>T | p.Ser815Phe |
| 5:131672003:A:G | missense | ENST00000510461.6: | ENSP00000421985.1: |
| | | c.2441T>C | p.Val814Ala |
| 5:131672004:C:T | missense | ENST00000510461.6: | ENSP00000421985.1: |
| | | c.2440G>A | p.Val814\|le |
| 5:131672024:T:C | missense | ENST00000510461.6: | ENSP00000421985.1: |
| | | c.2420A>G | p.Glu807Gly |
| 5:131672025:C: | frameshift | ENST00000510461.6: | ENSP00000421985.1: |
| CTCCAGATTGG | | c.2409_ | p.Glu807ProfsTer6 |
| | | 2418dup | |
| 5:131672027:C:T | missense | ENST00000510461.6: | ENSP00000421985.1: |
| | | c.2417G>A | p.Gly806Glu |
| 5:131672028:C:G | missense | ENST00000510461.6: | ENSP00000421985.1: |
| | | c.2416G>C | p.Gly806Arg |
| 5:131672030:G:C | missense | ENST00000510461.6: | ENSP00000421985.1: |
| | | c.2414C>G | p.Ser805Cys |
| 5:131672031:A:G | missense | ENST00000510461.6: | ENSP00000421985.1: |
| | | c.2413T>C | p.Ser805Pro |
| 5:131672032:T:G | missense | ENST00000510461.6: | ENSP00000421985.1: |
| | | c.2412A>C | p.Gln804His |
| 5:131672033:TGG | frameshift | ENST00000510461.6: | ENSP00000421985.1: |
| TCC:T | | c.2406_ | p.Asp803 |
| | | 2410del | IlefsTer5 |
| 5:131672036:T:C | missense | ENST00000510461.6: | ENSP00000421985.1: |
| | | c.2408A>G | p.Asp803Gly |
| 5:131672037:C:T | missense | ENST00000510461.6: | ENSP00000421985.1: |
| | | c.2407G>A | p.Asp803Asn |
| 5:131672052:T:C | missense | ENST00000510461.6: | ENSP00000421985.1: |
| | | c.2392A>G | p.Lys798Glu |
| 5:131672054:G:A | missense | ENST00000510461.6: | ENSP00000421985.1: |
| | | c.2390C>T | p.Thr797Ile |
| 5:131672055:T:C | missense | ENST00000510461.6: | ENSP00000421985.1: |
| | | c.2389A>G | p.Thr797Ala |
| 5:131672055:T:G | missense | ENST00000510461.6: | ENSP00000421985.1: |
| | | c.2389A>C | p.Thr797Pro |
| 5:131672060:T:G | missense | ENST00000510461.6: | ENSP00000421985.1: |
| | | c.2384A>C | p.Gln795Pro |
| 5:131672062:A:T | missense | ENST00000510461.6: | ENSP00000421985.1: |
| | | c.2382T>A | p.His794Gln |
| 5:131672063:T:G | missense | ENST00000510461.6: | ENSP00000421985.1: |
| | | c.2381A>C | p.His794Pro |
| 5:131672064:G:A | missense | ENST00000510461.6: | ENSP00000421985.1: |
| | | c.2380C>T | p.His794Tyr |
| 5:131672064:G:T | missense | ENST00000510461.6: | ENSP00000421985.1: |
| | | c.2380C>A | p.His794Asn |
| 5:131672066:T:C | missense | ENST00000510461.6: | ENSP00000421985.1: |
| | | c.2378A>G | p.Gln793Arg |
| 5:131672067:G:A | stop_ | ENST00000510461.6: | ENSP00000421985.1: |
| | gained | c.2377C>T | p.Gln793Ter |
| 5:131672067:G:T | missense | ENST00000510461.6: | ENSP00000421985.1: |
| | | c.2377C>A | p.Gln793Lys |
| 5:131672070:C:T | missense | ENST00000510461.6: | ENSP00000421985.1: |
| | | c.2374G>A | p.Asp792Asn |
| 5:131672078:CCT:C | frameshift | ENST00000510461.6: | ENSP00000421985.1: |
| | | c.2364_ | p.Ala790TyrfsTer2 |
| | | 2365del | |
| 5:131672086:T:G | missense | ENST00000510461.6: | ENSP00000421985.1: |
| | | c.2358A>C | p.Glu786Asp |
| 5:131672087:T:C | missense | ENST00000510461.6: | ENSP00000421985.1: |
| | | c.2357A>G | p.Glu786Gly |
| 5:131672088:C:T | missense | ENST00000510461.6: | ENSP00000421985.1: |
| | | c.2356G>A | p.Glu786Lys |
| 5:131672096:G:A | missense | ENST00000510461.6: | ENSP00000421985.1: |
| | | c.2348C>T | p.Pro783Leu |
| 5:131672097:G:A | missense | ENST00000510461.6: | ENSP00000421985.1: |
| | | c.2347C>T | p.Pro783Ser |
| 5:131672100:T:C | missense | ENST00000510461.6: | ENSP00000421985.1: |
| | | c.2344A>G | p.Lys782Glu |
| 5:131672103:T:A | missense | ENST00000510461.6: | ENSP00000421985.1: |
| | | c.2341A>T | p.Thr781Ser |
| 5:131672106:G:A | missense | ENST00000510461.6: | ENSP00000421985.1: |
| | | c.2338C>T | p.His780Tyr |
| 5:131672111:C:G | missense | ENST00000510461.6: | ENSP00000421985.1: |
| | | c.2333G>C | p.Arg778Thr |
| 5:131672111:C:T | missense | ENST00000510461.6: | ENSP00000421985.1: |
| | | c.2333G>A | p.Arg778Lys |
| 5:131672112:T:C | missense | ENST00000510461.6: | ENSP00000421985.1: |
| | | c.2332A>G | p.Arg778Gly |
| 5:131672116:A:C | missense | ENST00000510461.6: | ENSP00000421985.1: |
| | | c.2328T>G | p.IIle776Met |
| 5:131672118:T:A | missense | ENST00000510461.6: | ENSP00000421985.1: |
| | | c.2326A>T | p.Ile776Phe |
| 5:131672120:T:C | missense | ENST00000510461.6: | ENSP00000421985.1: |
| | | c.2324A>G | p.Gln775Arg |
| 5:131672121:G:C | missense | ENST00000510461.6: | ENSP00000421985.1: |
| | | c.2323C>G | p.Gln775Glu |
| 5:131672126:A:G | missense | ENST00000510461.6: | ENSP00000421985.1: |
| | | c.2318T>C | p.Val773Ala |
| 5:131672127:C:T | missense | ENST00000510461.6: | ENSP00000421985.1: |
| | | c.2317G>A | p.Val773Met |
| 5:131672130:C:T | missense | ENST00000510461.6: | ENSP00000421985.1: |
| | | c.2314G>A | p.Val772Met |
| 5:131672132:G:A | missense | ENST00000510461.6: | ENSP00000421985.1: |
| | | c.2312C>T | p.Ala771Val |
| 5:131672132:G:T | missense | ENST00000510461.6: | ENSP00000421985.1: |
| | | c.2312C>A | p.Ala771Glu |
| 5:131672133:C:T | missense | ENST00000510461.6: | ENSP00000421985.1: |
| | | c.2311G>A | p.Ala771Thr |
| 5:131672135:T:A | missense | ENST00000510461.6: | ENSP00000421985.1: |
| | | c.2309A>T | p.Gln770Leu |
| 5:131672136:G:C | missense | ENST00000510461.6: | ENSP00000421985.1: |
| | | c.2308C>G | p.Gln770Glu |
| 5:131672141:C:G | missense | ENST00000510461.6: | ENSP00000421985.1: |
| | | c.2303G>C | p.Arg768Pro |
| 5:131672141:C:T | missense | ENST00000510461.6: | ENSP00000421985.1: |
| | | c.2303G>A | p.Arg768Gln |
| 5:131672142:G:A | stop_ | ENST00000510461.6: | ENSP00000421985.1: |
| | gained | c.2302C>T | p.Arg768Ter |
| 5:131672145:G:C | missense | ENST00000510461.6: | ENSP00000421985.1: |
| | | c.2299C>G | p.Leu767Val |
| 5:131672146:C:G | missense | ENST00000510461.6: | ENSP00000421985.1: |
| | | c.2298G>C | p.Glu766Asp |
| 5:131672153:T:A | missense | ENST00000510461.6: | ENSP00000421985.1: |
| | | c.2291A>T | p.Asp764Val |
| 5:131672156:G:C | stop_ | ENST00000510461.6: | ENSP00000421985.1: |
| | gained | c.2288C>G | p.Ser763Ter |
| 5:131672157:A:C | missense | ENST00000510461.6: | ENSP00000421985.1: |
| | | c.2287T>G | p.Ser763Ala |
| 5:131672158:A:C | missense | ENST00000510461.6: | ENSP00000421985.1: |
| | | c.2286T>G | p.Asp762Glu |
| 5:131672160:C:T | missense | ENST00000510461.6: | ENSP00000421985.1: |
| | | c.2284G>A | p.Asp762Asn |
| 5:131672162:G:A | missense | ENST00000510461.6: | ENSP00000421985.1: |
| | | c.2282C>T | p.Pro761Leu |
| 5:131672163:G:A | missense | ENST00000510461.6: | ENSP00000421985.1: |
| | | c.2281C>T | p.Pro761Ser |
| 5:131672163:G:C | missense | ENST00000510461.6: | ENSP00000421985.1: |
| | | c.2281C>G | p.Pro761Ala |
| 5:131672169:T:C | missense | ENST00000510461.6: | ENSP00000421985.1: |
| | | c.2275A>G | p.Met759Val |
| 5:131672171:G:A | missense | ENST00000510461.6: | ENSP00000421985.1: |
| | | c.2273C>T | p.Ser758Phe |
| 5:131672171:G:C | missense | ENST00000510461.6: | ENSP00000421985.1: |
| | | c.2273C>G | p.Ser758Cys |
| 5:131672172:A:C | missense | ENST00000510461.6: | ENSP00000421985.1: |
| | | c.2272T>G | p.Ser758Ala |
| 5:131672172:A:G | missense | ENST00000510461.6: | ENSP00000421985.1: |
| | | c.2272T>C | p.Ser758Pro |
| 5:131672172:A:T | missense | ENST00000510461.6: | ENSP00000421985.1: |
| | | c.2272T>A | p.Ser758Thr |
| 5:131672174:T:A | missense | ENST00000510461.6: | ENSP00000421985.1: |
| | | c.2270A>T | p.Asp757Val |
| 5:131672179:A:C | missense | ENST00000510461.6: | ENSP00000421985.1: |
| | | c.2265T>G | p.Ile755Met |
| 5:131672180:A:G | missense | ENST00000510461.6: | ENSP00000421985.1: |
| | | c.2264T>C | p.Ile755Thr |
| 5:131672181:T:A | missense | ENST00000510461.6: | ENSP00000421985.1: |
| | | c.2263A>T | p.Ile755Phe |
| 5:131672189:G:C | missense | ENST00000510461.6: | ENSP00000421985.1: |
| | | c.2255C>G | p.Thr752Ser |
| 5:131672192:A:G | missense | ENST00000510461.6: | ENSP00000421985.1: |
| | | c.2252T>C | p.Val751Ala |
| 5:131672193:C:T | missense | ENST00000510461.6: | ENSP00000421985.1: |
| | | c.2251G>A | p.Val751Ile |
| 5:131672195:T:C | missense | ENST00000510461.6: | ENSP00000421985.1: |
| | | c.2249A>G | p.Lys750Arg |
| 5:131672201:T:C | missense | ENST00000510461.6: | ENSP00000421985.1: |
| | | c.2243A>G | p.Gln748Arg |
| 5:131672212:A:C | missense | ENST00000510461.6: | ENSP00000421985.1: |
| | | c.2232T>G | p.Cys744Trp |
| 5:131672213:C:T | missense | ENST00000510461.6: | ENSP00000421985.1: |
| | | c.2231G>A | p.Cys744Tyr |
| 5:131672233:A:C | missense | ENST00000510461.6: | ENSP00000421985.1: |
| | | c.2211T>G | p.Ile737Met |
| 5:131672234:A:G | missense | ENST00000510461.6: | ENSP00000421985.1: |
| | | c.2210T>C | p.Ile737Thr |
| 5:131672238:T:C | missense | ENST00000510461.6: | ENSP00000421985.1: |
| | | c.2206A>G | p.Lys736Glu |
| 5:131672240:T:C | missense | ENST00000510461.6: | ENSP00000421985.1: |
| | | c.2204A>G | p.Asp735Gly |
| 5:131672240:T:G | missense | ENST00000510461.6: | ENSP00000421985.1: |
| | | c.2204A>C | p.Asp735Ala |
| 5:131672244:G:A | missense | ENST00000510461.6: | ENSP00000421985.1: |
| | | c.2200C>T | p.Pro734Ser |
| 5:131672246:G:A | missense | ENST00000510461.6: | ENSP00000421985.1: |
| | | c.2198C>T | p.Pro733Leu |
| 5:131672247:G:GT | frameshift | ENST00000510461.6: | ENSP00000421985.1: |
| | | c.2196dup | p.Pro733ThrfsTer4 |
| 5:131672247:G:T | missense | ENST00000510461.6: | ENSP00000421985.1: |
| | | c.2197C>A | p.Pro733Thr |
| 5:131672248:T:G | missense | ENST00000510461.6: | ENSP00000421985.1: |
| | | c.2196A>C | p.Lys732Asn |
| 5:131672249:T:A | missense | ENST00000510461.6: | ENSP00000421985.1: |
| | | c.2195A>T | p.Lys732Ile |
| 5:131672252:T:C | missense | ENST00000510461.6: | ENSP00000421985.1: |
| | | c.2192A>G | p.Lys731Arg |
| 5:131672253:T:C | missense | ENST00000510461.6: | ENSP00000421985.1: |
| | | c.2191A>G | p.Lys731Glu |
| 5:131672254:T:G | missense | ENST00000510461.6: | ENSP00000421985.1: |
| | | c.2190A>C | p.Glu730Asp |
| 5:131672255:T:C | missense | ENST00000510461.6: | ENSP00000421985.1: |
| | | c.2189A>G | p.Glu730Gly |
| 5:131672256:C:G | missense | ENST00000510461.6: | ENSP00000421985.1: |
| | | c.2188G>C | p.Glu730Gln |
| 5:131672256:C:T | missense | ENST00000510461.6: | ENSP00000421985.1: |
| | | c.2188G>A | p.Glu730Lys |
| 5:131672259:C:G | missense | ENST00000510461.6: | ENSP00000421985.1: |
| | | c.2185G>C | p.Val729Leu |
| 5:131672261:A:G | missense | ENST00000510461.6: | ENSP00000421985.1: |
| | | c.2183T>C | p.Val728Ala |
| 5:131672262:C:T | missense | ENST00000510461.6: | ENSP00000421985.1: |
| | | c.2182G>A | p.Val728Ile |
| 5:131672264:A:G | missense | ENST00000510461.6: | ENSP00000421985.1: |
| | | c.2180T>C | p.Met727Thr |
| 5:131672265:T:C | missense | ENST00000510461.6: | ENSP00000421985.1: |
| | | c.2179A>G | p.Met727Val |
| 5:131672267:C:A | missense | ENST00000510461.6: | ENSP00000421985.1: |
| | | c.2177G>T | p.Gly726Val |
| 5:131672268:C:T | missense | ENST00000510461.6: | ENSP00000421985.1: |
| | | c.2176G>A | p.Gly726Arg |
| 5:131672270:G:A | missense | ENST00000510461.6: | ENSP00000421985.1: |
| | | c.2174C>T | p.Thr725Ile |
| 5:131672271:T:C | missense | ENST00000510461.6: | ENSP00000421985.1: |
| | | c.2173A>G | p.Thr725Ala |
| 5:131672271:T:G | missense | ENST00000510461.6: | ENSP00000421985.1: |
| | | c.2173A>C | p.Thr725Pro |
| 5:131672273:G:A | missense | ENST00000510461.6: | ENSP00000421985.1: |
| | | c.2171C>T | p.Ser724Phe |
| 5:131672274:A:C | missense | ENST00000510461.6: | ENSP00000421985.1: |
| | | c.2170T>G | p.Ser724Ala |
| 5:131672274:A:T | missense | ENST00000510461.6: | ENSP00000421985.1: |
| | | c.2170T>A | p.Ser724Thr |
| 5:131672278:C:T | missense | ENST00000510461.6: | ENSP00000421985.1: |
| | | c.2166G>A | p.Met722Ile |
| 5:131672279:A:C | missense | ENST00000510461.6: | ENSP00000421985.1: |
| | | c.2165T>G | p.Met722Arg |
| 5:131672280:T:C | missense | ENST00000510461.6: | ENSP00000421985.1: |
| | | c.2164A>G | p.Met722Val |
| 5:131672283:C:T | missense | ENST00000510461.6: | ENSP00000421985.1: |
| | | c.2161G>A | p.Ala721Thr |
| 5:131672285:T:C | missense | ENST00000510461.6: | ENSP00000421985.1: |
| | | c.2159A>G | p.Lys720Arg |
| 5:131672291:G:A | missense | ENST00000510461.6: | ENSP00000421985.1: |
| | | c.2153C>T | p.Thr718Ile |
| 5:131672292:T:G | missense | ENST00000510461.6: | ENSP00000421985.1: |
| | | c.2152A>C | p.Thr718Pro |
| 5:131672296:ACT | frameshift | ENST00000510461.6: | ENSP00000421985.1: |
| | | c.2144_ | p.Asp715ValfsTer7 |
| GT:A | | 2147del | |
| 5:131672303:G:A | missense | ENST00000510461.6: | ENSP00000421985.1: |
| | | c.2141C>T | p.Ser714Leu |
| 5:131672311:C:A | missense | ENST00000510461.6: | ENSP00000421985.1: |
| | | c.2133G>T | p.Leu711Phe |
| 5:131672312:A:G | missense | ENST00000510461.6: | ENSP00000421985.1: |
| | | c.2132T>C | p.Leu711Ser |
| 5:131672320:A:C | missense | ENST00000510461.6: | ENSP00000421985.1: |
| | | c.2124T>G | p.Ser708Arg |
| 5:131672321:C:A | missense | ENST00000510461.6: | ENSP00000421985.1: |
| | | c.2123G>T | p.Ser708Ile |
| 5:131672321:C:T | missense | ENST00000510461.6: | ENSP00000421985.1: |
| | | c.2123G>A | p.Ser708Asn |
| 5:131672324:T:C | missense | ENST00000510461.6: | ENSP00000421985.1: |
| | | c.2120A>G | p.Gln707Arg |
| 5:131672328:A:G | missense | ENST00000510461.6: | ENSP00000421985.1: |
| | | c.2116T>C | p.Trp706Arg |
| 5:131672328:A:T | missense | ENST00000510461.6: | ENSP00000421985.1: |
| | | c.2116T>A | p.Trp706Arg |
| 5:131672342:G:A | missense | ENST00000510461.6: | ENSP00000421985.1: |
| | | c.2102C>T | p.Ser701Leu |
| 5:131672343:A:C | missense | ENST00000510461.6: | ENSP00000421985.1: |
| | | c.2101T>G | p.Ser701Ala |
| 5:131672345:T:C | missense | ENST00000510461.6: | ENSP00000421985.1: |
| | | c.2099A>G | p.Glu700Gly |
| 5:131672346:C:G | missense | ENST00000510461.6: | ENSP00000421985.1: |
| | | c.2098G>C | p.Glu700Gln |
| 5:131672346:C:T | missense | ENST00000510461.6: | ENSP00000421985.1: |
| | | c.2098G>A | p.Glu700Lys |
| 5:131672351:C:T | missense | ENST00000510461.6: | ENSP00000421985.1: |
| | | c.2093G>A | p.Gly698Asp |
| 5:131672352:C:T | missense | ENST00000510461.6: | ENSP00000421985.1: |
| | | c.2092G>A | p.Gly698Ser |
| 5:131672357:T:G | missense | ENST00000510461.6: | ENSP00000421985.1: |
| | | c.2087A>C | p.Glu696Ala |
| 5:131672363:A:C | missense | ENST00000510461.6: | ENSP00000421985.1: |
| | | c.2081T>G | p.Leu694Trp |
| 5:131672363:A:G | missense | ENST00000510461.6: | ENSP00000421985.1: |
| | | c.2081T>C | p.Leu694Ser |
| 5:131672367:C:T | missense | ENST00000510461.6: | ENSP00000421985.1: |
| | | c.2077G>A | p.Ala693Thr |
| 5:131672369:C:T | missense | ENST00000510461.6: | ENSP00000421985.1: |
| | | c.2075G>A | p.Cys692Tyr |
| 5:131672370:A:G | missense | ENST00000510461.6: | ENSP00000421985.1: |
| | | c.2074T>C | p.Cys692Arg |
| 5:131672372:T:A | missense | ENST00000510461.6: | ENSP00000421985.1: |
| | | c.2072A>T | p.Lys691Ile |
| 5:131672375:T:C | missense | ENST00000510461.6: | ENSP00000421985.1: |
| | | c.2069A>G | p.Asp690Gly |
| 5:131672376:C:T | missense | ENST00000510461.6: | ENSP00000421985.1: |
| | | c.2068G>A | p.Asp690Asn |
| 5:131672381:G:C | missense | ENST00000510461.6: | ENSP00000421985.1: |
| | | c.2063C>G | p.Pro688Arg |
| 5:131672382:G:C | missense | ENST00000510461.6: | ENSP00000421985.1: |
| | | c.2062C>G | p.Pro688Ala |
| 5:131672382:G:T | missense | ENST00000510461.6: | ENSP00000421985.1: |
| | | c.2062C>A | p.Pro688Thr |
| 5:131672385:C:T | missense | ENST00000510461.6: | ENSP00000421985.1: |
| | | c.2059G>A | p.Val687Ile |
| 5:131672387:G:C | missense | ENST00000510461.6: | ENSP00000421985.1: |
| | | c.2057C>G | p.Ser686Cys |
| 5:131672389:TC:T | frameshift | ENST00000510461.6: | ENSP00000421985.1: |
| | | c.2054del | p.Gly685AspfsTer5 |
| 5:131672390:C:T | missense | ENST00000510461.6: | ENSP00000421985.1: |
| | | c.2054G>A | p.Gly685Glu |
| 5:131672391:C:T | missense | ENST00000510461.6: | ENSP00000421985.1: |
| | | c.2053G>A | p.Gly685Arg |
| 5:131672393:G:A | missense | ENST00000510461.6: | ENSP00000421985.1: |
| | | c.2051C>T | p.Thr684Ile |
| 5:131672394:T:G | missense | ENST00000510461.6: | ENSP00000421985.1: |
| | | c.2050A>C | p.Thr684Pro |
| 5:131672396:CA:C | frameshift | ENST00000510461.6: | ENSP00000421985.1: |
| | | c.2047del | p.Cys683AlafsTer7 |
| 5:131672400:C:A | missense | ENST00000510461.6: | ENSP00000421985.1: |
| | | c.2044G>T | p.Val682Phe |
| 5:131672405:G:A | missense | ENST00000510461.6: | ENSP00000421985.1: |
| | | c.2039C>T | p.Thr680Ile |
| 5:131672406:T:C | missense | ENST00000510461.6: | ENSP00000421985.1: |
| | | c.2038A>G | p.Thr680Ala |
| 5:131672408:T:C | missense | ENST00000510461.6: | ENSP00000421985.1: |
| | | c.2036A>G | p.Glu679Gly |
| 5:131672409:C:G | missense | ENST00000510461.6: | ENSP00000421985.1: |
| | | c.2035G>C | p.Glu679Gln |
| 5:131672417:G:T | missense | ENST00000510461.6: | ENSP00000421985.1: |
| | | c.2027C>A | p.Ala676Asp |
| 5:131672418:C:A | missense | ENST00000510461.6: | ENSP00000421985.1: |
| | | c.2026G>T | p.Ala676Ser |
| 5:131672418:C:T | missense | ENST00000510461.6: | ENSP00000421985.1: |
| | | c.2026G>A | p.Ala676Thr |
| 5:131672420:T:C | missense | ENST00000510461.6: | ENSP00000421985.1: |
| | | c.2024A>G | p.Asp675Gly |
| 5:131672423:A:G | missense | ENST00000510461.6: | ENSP00000421985.1: |
| | | c.2021T>C | p.Phe674Ser |
| 5:131672424:A:G | missense | ENST00000510461.6: | ENSP00000421985.1: |
| | | c.2020T>C | p.Phe674Leu |
| 5:131672424:AG:A | frameshift | ENST00000510461.6: | ENSP00000421985.1: |
| | | c.2019del | p.Phe674LeufsTer5 |
| 5:131672427:A:C | missense | ENST00000510461.6: | ENSP00000421985.1: |
| | | c.2017T>G | p.Cys673Gly |
| 5:131672430:T:A | missense | ENST00000510461.6: | ENSP00000421985.1: |
| | | c.2014A>T | p.Thr672Ser |
| 5:131672430:T:C | missense | ENST00000510461.6: | ENSP00000421985.1: |
| | | c.2014A>G | p.Thr672Ala |
| 5:131672436:A:T | missense | ENST00000510461.6: | ENSP00000421985.1: |
| | | c.2008T>A | p.Leu670Ile |
| 5:131672438:T:G | missense | ENST00000510461.6: | ENSP00000421985.1: |
| | | c.2006A>C | p.Lys669Thr |
| 5:131672439:T:C | missense | ENST00000510461.6: | ENSP00000421985.1: |
| | | c.2005A>G | p.Lys669Glu |
| 5:131672440:A:T | missense | ENST00000510461.6: | ENSP00000421985.1: |
| | | c.2004T>A | p.Asp668Glu |
| 5:131672441:T:C | missense | ENST00000510461.6: | ENSP00000421985.1: |
| | | c.2003A>G | p.Asp668Gly |
| 5:131672442:C:T | missense | ENST00000510461.6: | ENSP00000421985.1: |
| | | c.2002G>A | p.Asp668Asn |
| 5:131672444:C:G | missense | ENST00000510461.6: | ENSP00000421985.1: |
| | | c.2000G>C | p.Arg667Thr |
| 5:131672447:T:C | missense | ENST00000510461.6: | ENSP00000421985.1: |
| | | c.1997A>G | p.Tyr666Cys |
| 5:131672448:A:G | missense | ENST00000510461.6: | ENSP00000421985.1: |
| | | c.1996T>C | p.Tyr666His |
| 5:131672449:C:G | missense | ENST00000510461.6: | ENSP00000421985.1: |
| | | c.1995G>C | p.Gln665His |
| 5:131672450:T:C | missense | ENST00000510461.6: | ENSP00000421985.1: |
| | | c.1994A>G | p.Gln665Arg |
| 5:131672454:T:C | missense | ENST00000510461.6: | ENSP00000421985.1: |
| | | c.1990A>G | p.Lys664Glu |
| 5:131672457:C:T | missense | ENST00000510461.6: | ENSP00000421985.1: |
| | | c.1987G>A | p.Val663Ile |
| 5:131672457:CA:C | frameshift | ENST00000510461.6: | ENSP00000421985.1: |
| | | c.1986del | p.Asp662GlufsTer9 |
| 5:131672460:C:T | missense | ENST00000510461.6: | ENSP00000421985.1: |
| | | c.1984G>A | p.Asp662Asn |
| 5:131672463:C:T | missense | ENST00000510461.6: | ENSP00000421985.1: |
| | | c.1981G>A | p.Val661Ile |
| 5:131672465:G:T | missense | ENST00000510461.6: | ENSP00000421985.1: |
| | | c.1979C>A | p.Ala660Asp |
| 5:131672466:C:T | missense | ENST00000510461.6: | ENSP00000421985.1: |
| | | c.1978G>A | p.Ala660Thr |
| 5:131672468:T:C | missense | ENST00000510461.6: | ENSP00000421985.1: |
| | | c.1976A>G | p.Asn659Ser |
| 5:131672471:T:C | missense | ENST00000510461.6: | ENSP00000421985.1: |
| | | c.1973A>G | p.Glu658Gly |
| 5:131672474:T:C | missense | ENST00000510461.6: | ENSP00000421985.1: |
| | | c.1970A>G | p.Glu657Gly |
| 5:131672475:C:T | missense | ENST00000510461.6: | ENSP00000421985.1: |
| | | c.1969G>A | p.Glu657Lys |
| 5:131672477:T:A | missense | ENST00000510461.6: | ENSP00000421985.1: |
| | | c.1967A>T | p.Gln656Leu |
| 5:131672478:G:A | stop_ | ENST00000510461.6: | ENSP00000421985.1: |
| | gained | c.1966C>T | p.Gln656Ter |
| 5:131672482:G:C | missense | ENST00000510461.6: | ENSP00000421985.1: |
| | | c.1962C>G | p.Asp654Glu |
| 5:131672486:G:A | missense | ENST00000510461.6: | ENSP00000421985.1: |
| | | c.1958C>T | p.Ser653Phe |
| 5:131672490:G:A | missense | ENST00000510461.6: | ENSP00000421985.1: |
| | | c.1954C>T | p.Pro652Ser |
| 5:131672502:G:A | stop_ | ENST00000510461.6: | ENSP00000421985.1: |
| | gained | c.1942C>T | p.Gln648Ter |
| 5:131672504:C:T | missense | ENST00000510461.6: | ENSP00000421985.1: |
| | | c.1940G>A | p.Cys647Tyr |
| 5:131672505:A:G | missense | ENST00000510461.6: | ENSP00000421985.1: |
| | | c.1939T>C | p.Cys647Arg |
| 5:131672507:T:G | missense | ENST00000510461.6: | ENSP00000421985.1: |
| | | c.1937A>C | p.Glu646Ala |
| 5:131672508:C:G | missense | ENST00000510461.6: | ENSP00000421985.1: |
| | | c.1936G>C | p.Glu646Gln |
| 5:131672509:A:C | missense | ENST00000510461.6: | ENSP00000421985.1: |
| | | c.1935T>G | p.Asp645Glu |
| 5:131672511:C:G | missense | ENST00000510461.6: | ENSP00000421985.1: |
| | | c.1933G>C | p.Asp645His |
| 5:131672517:T:C | missense | ENST00000510461.6: | ENSP00000421985.1: |
| | | c.1927A>G | p.Ile643Val |
| 5:131672519:C:T | missense | ENST00000510461.6: | ENSP00000421985.1: |
| | | c.1925G>A | p.Gly642Glu |
| 5:131672522:A:G | missense | ENST00000510461.6: | ENSP00000421985.1: |
| | | c.1922T>C | p.Leu641Pro |
| 5:131672527:C:G | missense | ENST00000510461.6: | ENSP00000421985.1: |
| | | c.1917G>C | p.Glu639Asp |
| 5:131672529:C:T | missense | ENST00000510461.6: | ENSP00000421985.1: |
| | | c.1915G>A | p.Glu639Lys |
| 5:131672534:G:A | missense | ENST00000510461.6: | ENSP00000421985.1: |
| | | c.1910C>T | p.Ser637Phe |
| 5:131672543:T:G | missense | ENST00000510461.6: | ENSP00000421985.1: |
| | | c.1901A>C | p.Gln634Pro |
| 5:131672552:T:C | missense | ENST00000510461.6: | ENSP00000421985.1: |
| | | c.1892A>G | p.Glu631Gly |
| 5:131672553:C:A | stop_ | ENST00000510461.6: | ENSP00000421985.1: |
| | gained | c.1891G>T | p.Glu631Ter |
| 5:131672553:C:G | missense | ENST00000510461.6: | ENSP00000421985.1: |
| | | c.1891G>C | p.Glu631Gln |
| 5:131672557:C:G | missense | ENST00000510461.6: | ENSP00000421985.1: |
| | | c.1887G>C | p.Glu629Asp |
| 5:131672559:C:G | missense | ENST00000510461.6: | ENSP00000421985.1: |
| | | c.1885G>C | p.Glu629Gln |
| 5:131672559:CTT:C | frameshift | ENST00000510461.6: | ENSP00000421985.1: |
| | | c.1883_ | p.Gln628ArgfsTer10 |
| | | 1884del | |
| 5:131672562:G:C | missense | ENST00000510461.6: | ENSP00000421985.1: |
| | | c.1882C>G | p.Gln628Glu |
| 5:131672562:G:T | missense | ENST00000510461.6: | ENSP00000421985.1: |
| | | c.1882C>A | p.Gln628Lys |
| 5:131672567:G:A | missense | ENST00000510461.6: | ENSP00000421985.1: |
| | | c.1877C>T | p.Ser626Leu |
| 5:131672568:A:T | missense | ENST00000510461.6: | ENSP00000421985.1: |
| | | c.1876T>A | p.Ser626Thr |
| 5:131672575:C:A | missense | ENST00000510461.6: | ENSP00000421985.1: |
| | | c.1869G>T | p.Glu623Asp |
| 5:131672582:T:G | missense | ENST00000510461.6: | ENSP00000421985.1: |
| | | c.1862A>C | p.Asn621Thr |
| 5:131672583:T:G | missense | ENST00000510461.6: | ENSP00000421985.1: |
| | | c.1861A>C | p.Asn621His |
| 5:131672588:C:G | missense | ENST00000510461.6: | ENSP00000421985.1: |
| | | c.1856G>C | p.Gly619Ala |
| 5:131672594:A:G | missense | ENST00000510461.6: | ENSP00000421985.1: |
| | | c.1850T>C | p.Leu617Pro |
| 5:131672595:G:A | missense | ENST00000510461.6: | ENSP00000421985.1: |
| | | c.1849C>T | p.Leu617Phe |
| 5:131672595:G:C | missense | ENST00000510461.6: | ENSP00000421985.1: |
| | | c.1849C>G | p.Leu617Val |
| 5:131672597:G:A | missense | ENST00000510461.6: | ENSP00000421985.1: |
| | | c.1847C>T | p.Pro616Leu |
| 5:131672598:G:A | missense | ENST00000510461.6: | ENSP00000421985.1: |
| | | c.1846C>T | p.Pro616Ser |
| 5:131672602:A:T | missense | ENST00000510461.6: | ENSP00000421985.1: |
| | | c.1842T>A | p.Ser614Arg |
| 5:131672603:C:T | missense | ENST00000510461.6: | ENSP00000421985.1: |
| | | c.1841G>A | p.Ser614Asn |
| 5:131672606:C:T | missense | ENST00000510461.6: | ENSP00000421985.1: |
| | | c.1838G>A | p.Cys613Tyr |
| 5:131672610:A:G | missense | ENST00000510461.6: | ENSP00000421985.1: |
| | | c.1834T>C | p.Tyr612His |
| 5:131672613:TAC:T | frameshift | ENST00000510461.6: | ENSP00000421985.1: |
| | | c.1829_ | p.Cys610Ter |
| | | 1830del | |
| 5:131672621:C:A | missense | ENST00000510461.6: | ENSP00000421985.1: |
| | | c.1823G>T | p.Cys608Phe |
| 5:131672621:C:G | missense | ENST00000510461.6: | ENSP00000421985.1: |
| | | c.1823G>C | p.Cys608Ser |
| 5:131672621:C:T | missense | ENST00000510461.6: | ENSP00000421985.1: |
| | | c.1823G>A | p.Cys608Tyr |
| 5:131672622:A:T | missense | ENST00000510461.6: | ENSP00000421985.1: |
| | | c.1822T>A | p.Cys608Ser |
| 5:131672627:G:A | missense | ENST00000510461.6: | ENSP00000421985.1: |
| | | c.1817C>T | p.Pro606Leu |
| 5:131672632:TC:T | frameshift | ENST00000510461.6: | ENSP00000421985.1: |
| | | c.1811del | p.Arg604LysfsTer19 |
| 5:131672633:C:G | missense | ENST00000510461.6: | ENSP00000421985.1: |
| | | c.1811G>C | p.Arg604Thr |
| 5:131672648:T:C | missense | ENST00000510461.6: | ENSP00000421985.1: |
| | | c.1796A>G | p.Glu599Gly |
| 5:131672649:C:G | missense | ENST00000510461.6: | ENSP00000421985.1: |
| | | c.1795G>C | p.Glu599Gln |
| 5:131672651:T:C | missense | ENST00000510461.6: | ENSP00000421985.1: |
| | | c.1793A>G | p.Lys598Arg |
| 5:131672651:T:G | missense | ENST00000510461.6: | ENSP00000421985.1: |
| | | c.1793A>C | p.Lys598Thr |
| 5:131672652:T:C | missense | ENST00000510461.6: | ENSP00000421985.1: |
| | | c.1792A>G | p.Lys598Glu |
| 5:131672654:A:G | missense | ENST00000510461.6: | ENSP00000421985.1: |
| | | c.1790T>C | p.Phe597Ser |
| 5:131672655:A:G | missense | ENST00000510461.6: | ENSP00000421985.1: |
| | | c.1789T>C | p.Phe597Leu |
| 5:131672658:G:A | missense | ENST00000510461.6: | ENSP00000421985.1: |
| | | c.1786C>T | p.Leu596Phe |
| 5:131672662:A:C | missense | ENST00000510461.6: | ENSP00000421985.1: |
| | | c.1782T>G | p.Ser594Arg |
| 5:131672664:T:A | missense | ENST00000510461.6: | ENSP00000421985.1: |
| | | c.1780A>T | p.Ser594Cys |
| 5:131672664:T:C | missense | ENST00000510461.6: | ENSP00000421985.1: |
| | | c.1780A>G | p.Ser594Gly |
| 5:131672665:G:C | missense | ENST00000510461.6: | ENSP00000421985.1: |
| | | c.1779C>G | p.Ser593Arg |
| 5:131672669:T:C | missense | ENST00000510461.6: | ENSP00000421985.1: |
| | | c.1775A>G | p.Lys592Arg |
| 5:131672678:T:C | missense | ENST00000510461.6: | ENSP00000421985.1: |
| | | c.1766A>G | p.His589Arg |
| 5:131672682:T:C | missense | ENST00000510461.6: | ENSP00000421985.1: |
| | | c.1762A>G | p.Met588Val |
| 5:131672684:G:T | missense | ENST00000510461.6: | ENSP00000421985.1: |
| | | c.1760C>A | p.Thr587Lys |
| 5:131672688:C:G | missense | ENST00000510461.6: | ENSP00000421985.1: |
| | | c.1756G>C | p.Val586Leu |
| 5:131672688:C:T | missense | ENST00000510461.6: | ENSP00000421985.1: |
| | | c.1756G>A | p.Val586Ile |
| 5:131672695:A:T | stop_ | ENST00000510461.6: | ENSP00000421985.1: |
| | gained | c.1749T>A | p.Tyr583Ter |
| 5:131672696:TAC | frameshift | ENST00000510461.6: | ENSP00000421985.1: |
| TCTGATTC:T | | c.1738_ | p.Glu580 |
| | | 1747del | MetfsTer40 |
| 5:131672699:T:A | missense | ENST00000510461.6: | ENSP00000421985.1: |
| | | c.1745A>T | p.Glu582Val |
| 5:131672699:T:G | missense | ENST00000510461.6: | ENSP00000421985.1: |
| | | c.1745A>C | p.Glu582Ala |
| 5:131672700:C:G | missense | ENST00000510461.6: | ENSP00000421985.1: |
| | | c.1744G>C | p.Glu582Gln |
| 5:131672710:T:C | missense | ENST00000510461.6: | ENSP00000421985.1: |
| | | c.1734A>G | p.IIle578Met |
| 5:131672711:A:T | missense | ENST00000510461.6: | ENSP00000421985.1: |
| | | c.1733T>A | p.Ile578Lys |
| 5:131672712:T:C | missense | ENST00000510461.6: | ENSP00000421985.1: |
| | | c.1732A>G | p.Ile578Val |
| 5:131672714:T:C | missense | ENST00000510461.6: | ENSP00000421985.1: |
| | | c.1730A>G | p.Glu577Gly |
| 5:131672717:C:T | missense | ENST00000510461.6: | ENSP00000421985.1: |
| | | c.1727G>A | p.Gly576Asp |
| 5:131672720:TTCT | frameshift | ENST00000510461.6: | ENSP00000421985.1: |
| | | c.1722_ | p.Lys575ArgfsTer2 |
| | | 1723del | |
| 5:131672725:T:G | missense | ENST00000510461.6: | ENSP00000421985.1: |
| | | c.1719A>C | p.Leu573Phe |
| 5:131672729:G:A | missense | ENST00000510461.6: | ENSP00000421985.1: |
| | | c.1715C>T | p.Thr572Ile |
| 5:131672730:T:C | missense | ENST00000510461.6: | ENSP00000421985.1: |
| | | c.1714A>G | p.Thr572Ala |
| 5:131672730:T:G | missense | ENST00000510461.6: | ENSP00000421985.1: |
| | | c.1714A>C | p.Thr572Pro |
| 5:131672732:G:A | missense | ENST00000510461.6: | ENSP00000421985.1: |
| | | c.1712C>T | p.Thr571Ile |
| 5:131672732:G:T | missense | ENST00000510461.6: | ENSP00000421985.1: |
| | | c.1712C>A | p.Thr571Asn |
| 5:131672733:T:C | missense | ENST00000510461.6: | ENSP00000421985.1: |
| | | c.1711A>G | p.Thr571Ala |
| 5:131672738:A:G | missense | ENST00000510461.6: | ENSP00000421985.1: |
| | | c.1706T>C | p.Ile569Thr |
| 5:131672742:C:A | missense | ENST00000510461.6: | ENSP00000421985.1: |
| | | c.1702G>T | p.Val568Leu |
| 5:131672744:G:C | missense | ENST00000510461.6: | ENSP00000421985.1: |
| | | c.1700C>G | p.Thr567Arg |
| 5:131672747:C:T | missense | ENST00000510461.6: | ENSP00000421985.1: |
| | | c.1697G>A | p.Gly566Asp |
| 5:131672748:C:G | missense | ENST00000510461.6: | ENSP00000421985.1: |
| | | c.1696G>C | p.Gly566Arg |
| 5:131672751:G:A | missense | ENST00000510461.6: | ENSP00000421985.1: |
| | | c.1693C>T | p.Pro565Ser |
| 5:131672753:A:G | missense | ENST00000510461.6: | ENSP00000421985.1: |
| | | c.1691T>C | p.Met564Thr |
| 5:131672757:C:T | missense | ENST00000510461.6: | ENSP00000421985.1: |
| | | c.1687G>A | p.Val563Ile |
| 5:131672758:G:C | missense | ENST00000510461.6: | ENSP00000421985.1: |
| | | c.1686C>G | p.Ile562Met |
| 5:131672762:G:A | missense | ENST00000510461.6: | ENSP00000421985.1: |
| | | c.1682C>T | p.Ala561Val |
| 5:131672767:A:C | missense | ENST00000510461.6: | ENSP00000421985.1: |
| | | c.1677T>G | p.Asp559Glu |
| 5:131672767:A:T | missense | ENST00000510461.6: | ENSP00000421985.1: |
| | | c.1677T>A | p.Asp559Glu |
| 5:131672768:T:C | missense | ENST00000510461.6: | ENSP00000421985.1: |
| | | c.1676A>G | p.Asp559Gly |
| 5:131672769:C:T | missense | ENST00000510461.6: | ENSP00000421985.1: |
| | | c.1675G>A | p.Asp559Asn |
| 5:131672770:T:G | missense | ENST00000510461.6: | ENSP00000421985.1: |
| | | c.1674A>C | p.Glu558Asp |
| 5:131672771:T:A | missense | ENST00000510461.6: | ENSP00000421985.1: |
| | | c.1673A>T | p.Glu558Val |
| 5:131672783:A:G | missense | ENST00000510461.6: | ENSP00000421985.1: |
| | | c.1661T>C | p.Leu554Ser |
| 5:131672784:A:C | missense | ENST00000510461.6: | ENSP00000421985.1: |
| | | c.1660T>G | p.Leu554Val |
| 5:131672787:G:C | missense | ENST00000510461.6: | ENSP00000421985.1: |
| | | c.1657C>G | p.Leu553Val |
| 5:131672792:G:A | missense | ENST00000510461.6: | ENSP00000421985.1: |
| | | c.1652C>T | p.Thr551Met |
| 5:131672793:T:C | missense | ENST00000510461.6: | ENSP00000421985.1: |
| | | c.1651A>G | p.Thr551Ala |
| 5:131672798:T:G | missense | ENST00000510461.6: | ENSP00000421985.1: |
| | | c.1646A>C | p.Gln549Pro |
| 5:131672799:G:T | missense | ENST00000510461.6: | ENSP00000421985.1: |
| | | c.1645C>A | p.Gln549Lys |
| 5:131672801:A:G | missense | ENST00000510461.6: | ENSP00000421985.1: |
| | | c.1643T>C | p.Leu548Pro |
| 5:131672802:G:A | missense | ENST00000510461.6: | ENSP00000421985.1: |
| | | c.1642C>T | p.Leu548Phe |
| 5:131672816:A:G | missense | ENST00000510461.6: | ENSP00000421985.1: |
| | | c.1628T>C | p.Ile543Thr |
| 5:131672817:T:A | missense | ENST00000510461.6: | ENSP00000421985.1: |
| | | c.1627A>T | p.Ile543Leu |
| 5:131672825:G:C | missense | ENST00000510461.6: | ENSP00000421985.1: |
| | | c.1619C>G | p.Thr540Ser |
| 5:131672826:T:A | missense | ENST00000510461.6: | ENSP00000421985.1: |
| | | c.1618A>T | p.Thr540Ser |
| 5:131672833:ATAAAG: | frameshift | ENST00000510461.6: | ENSP00000421985.1: |
| A | | c.1606_ | p.Leu536PhefsTer10 |
| | | 1610del | |
| 5:131672834:T:C | missense | ENST00000510461.6: | ENSP00000421985.1: |
| | | c.1610A>G | p.Tyr537Cys |
| 5:131672837:A:T | missense | ENST00000510461.6: | ENSP00000421985.1: |
| | | c.1607T>A | p.Leu536His |
| 5:131672841:G:C | missense | ENST00000510461.6: | ENSP00000421985.1: |
| | | c.1603C>G | p.Leu535Val |
| 5:131672843:C:A | missense | ENST00000510461.6: | ENSP00000421985.1: |
| | | c.1601G>T | p.Arg534Met |
| 5:131672843:C:T | missense | ENST00000510461.6: | ENSP00000421985.1: |
| | | c.1601G>A | p.Arg534Lys |
| 5:131672847:G:C | missense | ENST00000510461.6: | ENSP00000421985.1: |
| | | c.1597C>G | p.Gln533Glu |
| 5:131672850:C:T | missense | ENST00000510461.6: | ENSP00000421985.1: |
| | | c.1594G>A | p.Val532Ile |
| 5:131672853:T:C | missense | ENST00000510461.6: | ENSP00000421985.1: |
| | | c.1591A>G | p.Met531Val |
| 5:131672856:C:A | missense | ENST00000510461.6: | ENSP00000421985.1: |
| | | c.1588G>T | p.Asp530Tyr |
| 5:131672856:C:T | missense | ENST00000510461.6: | ENSP00000421985.1: |
| | | c.1588G>A | p.Asp530Asn |
| 5:131672857:T:G | missense | ENST00000510461.6: | ENSP00000421985.1: |
| | | c.1587A>C | p.Gln529His |
| 5:131672859:G:C | missense | ENST00000510461.6: | ENSP00000421985.1: |
| | | c.1585C>G | p.Gln529Glu |
| 5:131672859:G:T | missense | ENST00000510461.6: | ENSP00000421985.1: |
| | | c.1585C>A | p.Gln529Lys |
| 5:131672861:C:A | missense | ENST00000510461.6: | ENSP00000421985.1: |
| | | c.1583G>T | p.Arg528Leu |
| 5:131672861:C:T | missense | ENST00000510461.6: | ENSP00000421985.1: |
| | | c.1583G>A | p.Arg528Gln |
| 5:131672862:G:A | stop_ | ENST00000510461.6: | ENSP00000421985.1: |
| | gained | c.1582C>T | p.Arg528Ter |
| 5:131672864:T:C | missense | ENST00000510461.6: | ENSP00000421985.1: |
| | | c.1580A>G | p.Lys527Arg |
| 5:131672865:T:C | missense | ENST00000510461.6: | ENSP00000421985.1: |
| | | c.1579A>G | p.Lys527Glu |
| 5:131672870:A:G | missense | ENST00000510461.6: | ENSP00000421985.1: |
| | | c.1574T>C | p.Val525Ala |
| 5:131672873:A:G | missense | ENST00000510461.6: | ENSP00000421985.1: |
| | | c.1571T>C | p.Val524Ala |
| 5:131672876:A:G | missense | ENST00000510461.6: | ENSP00000421985.1: |
| | | c.1568T>C | p.Val523Ala |
| 5:131672877:C:G | missense | ENST00000510461.6: | ENSP00000421985.1: |
| | | c.1567G>C | p.Val523Leu |
| 5:131672879:G:C | missense | ENST00000510461.6: | ENSP00000421985.1: |
| | | c.1565C>G | p.Thr522Ser |
| 5:131672880:T:C | missense | ENST00000510461.6: | ENSP00000421985.1: |
| | | c.1564A>G | p.Thr522Ala |
| 5:131672886:C:T | missense | ENST00000510461.6: | ENSP00000421985.1: |
| | | c.1558G>A | p.Ala520Thr |
| 5:131672889:A:T | missense | ENST00000510461.6: | ENSP00000421985.1: |
| | | c.1555T>A | p.Leu519Ile |
| 5:131672891:C:T | missense | ENST00000510461.6: | ENSP00000421985.1: |
| | | c.1553G>A | p.Arg518Gln |
| 5:131672892:G:A | missense | ENST00000510461.6: | ENSP00000421985.1: |
| | | c.1552C>T | p.Arg518Trp |
| 5:131672895:C:A | missense | ENST00000510461.6: | ENSP00000421985.1: |
| | | c.1549G>T | p.Val517Leu |
| 5:131672895:C:T | missense | ENST00000510461.6: | ENSP00000421985.1: |
| | | c.1549G>A | p.Val517Ile |
| 5:131672900:G:A | missense | ENST00000510461.6: | ENSP00000421985.1: |
| | | c.1544C>T | p.Ser515Phe |
| 5:131672901:A:G | missense | ENST00000510461.6: | ENSP00000421985.1: |
| | | c.1543T>C | p.Ser515Pro |
| 5:131672903:C:A | missense | ENST00000510461.6: | ENSP00000421985.1: |
| | | c.1541G>T | p.Gly514Val |
| 5:131672903:C:G | missense | ENST00000510461.6: | ENSP00000421985.1: |
| | | c.1541G>C | p.Gly514Ala |
| 5:131672903:C:T | missense | ENST00000510461.6: | ENSP00000421985.1: |
| | | c.1541G>A | p.Gly514Asp |
| 5:131672906:A:C | missense | ENST00000510461.6: | ENSP00000421985.1: |
| | | c.1538T>G | p.Ile513Ser |
| 5:131672906:A:G | missense | ENST00000510461.6: | ENSP00000421985.1: |
| | | c.1538T>C | p.Ile513Thr |
| 5:131672910:C:A | missense | ENST00000510461.6: | ENSP00000421985.1: |
| | | c.1534G>T | p.Ala512Ser |
| 5:131672910:C:T | missense | ENST00000510461.6: | ENSP00000421985.1: |
| | | c.1534G>A | p.Ala512Thr |
| 5:131672912:C:G | missense | ENST00000510461.6: | ENSP00000421985.1: |
| | | c.1532G>C | p.Gly511Ala |
| 5:131672912:C:T | missense | ENST00000510461.6: | ENSP00000421985.1: |
| | | c.1532G>A | p.Gly511Asp |
| 5:131672915:T:C | missense | ENST00000510461.6: | ENSP00000421985.1: |
| | | c.1529A>G | p.Tyr510Cys |
| 5:131672916:A:G | missense | ENST00000510461.6: | ENSP00000421985.1: |
| | | c.1528T>C | p.Tyr510His |
| 5:131672918:A:G | missense | ENST00000510461.6: | ENSP00000421985.1: |
| | | c.1526T>C | p.Leu509Ser |
| 5:131672920:G:C | missense | ENST00000510461.6: | ENSP00000421985.1: |
| | | c.1524C>G | p.Asp508Glu |
| 5:131677702:C:G | splice_ | ENST00000510461.6: | NA |
| | donor | c.1519+1G>C | |
| 5:131677703:C:T | missense | ENST00000510461.6: | ENSP00000421985.1: |
| | | c.1519G>A | p.Gly507Arg |
| 5:131677713:C:T | stop_ | ENST00000510461.6: | ENSP00000421985.1: |
| | gained | c.1509G>A | p.Trp503Ter |
| 5:131677721:G:A | missense | ENST00000510461.6: | ENSP00000421985.1: |
| | | c.1501C>T | p.Pro501Ser |
| 5:131677723:T:C | missense | ENST00000510461.6: | ENSP00000421985.1: |
| | | c.1499A>G | p.Asn500Ser |
| 5:131677736:T:C | missense | ENST00000510461.6: | ENSP00000421985.1: |
| | | c.1486A>G | p.Thr496Ala |
| 5:131677738:T:C | missense | ENST00000510461.6: | ENSP00000421985.1: |
| | | c.1484A>G | p.Lys495Arg |
| 5:131677738:T:G | missense | ENST00000510461.6: | ENSP00000421985.1: |
| | | c.1484A>C | p.Lys495Thr |
| 5:131677742:C:A | missense | ENST00000510461.6: | ENSP00000421985.1: |
| | | c.1480G>T | p.Ala494Ser |
| 5:131677748:T:C | missense | ENST00000510461.6: | ENSP00000421985.1: |
| | | c.1474A>G | p.Met492Val |
| 5:131677751:C:T | missense | ENST00000510461.6: | ENSP00000421985.1: |
| | | c.1471G>A | p.Asp491Asn |
| 5:131677755:A:C | missense | ENST00000510461.6: | ENSP00000421985.1: |
| | | c.1467T>G | p.Ser489Arg |
| 5:131677756:C:T | missense | ENST00000510461.6: | ENSP00000421985.1: |
| | | c.1466G>A | p.Ser489Asn |
| 5:131677759:T:C | missense | ENST00000510461.6: | ENSP00000421985.1: |
| | | c.1463A>G | p.Gln488Arg |
| 5:131677768:T:A | missense | ENST00000510461.6: | ENSP00000421985.1: |
| | | c.1454A>T | p.His485Leu |
| 5:131677780:A:C | missense | ENST00000510461.6: | ENSP00000421985.1: |
| | | c.1442T>G | p.Phe481Cys |
| 5:131677780:A:G | missense | ENST00000510461.6: | ENSP00000421985.1: |
| | | c.1442T>C | p.Phe481Ser |
| 5:131677781:A:C | missense | ENST00000510461.6: | ENSP00000421985.1: |
| | | c.1441T>G | p.Phe481Val |
| 5:131677782:T:C | missense | ENST00000510461.6: | ENSP00000421985.1: |
| | | c.1440A>G | p.Ile480Met |
| 5:131677783:A:G | missense | ENST00000510461.6: | ENSP00000421985.1: |
| | | c.1439T>C | p.Ile480Thr |
| 5:131677790:T:C | missense | ENST00000510461.6: | ENSP00000421985.1: |
| | | c.1432A>G | p.Ile478Val |
| 5:131677790:T:G | missense | ENST00000510461.6: | ENSP00000421985.1: |
| | | c.1432A>C | p.Ile478Leu |
| 5:131677792:G:C | missense | ENST00000510461.6: | ENSP00000421985.1: |
| | | c.1430C>G | p.Pro477Arg |
| 5:131677793:G:A | missense | ENST00000510461.6: | ENSP00000421985.1: |
| | | c.1429C>T | p.Pro477Ser |
| 5:131677796:G:C | missense | ENST00000510461.6: | ENSP00000421985.1: |
| | | c.1426C>G | p.Pro476Ala |
| 5:131677804:T:C | missense | ENST00000510461.6: | ENSP00000421985.1: |
| | | c.1418A>G | p.Asn473Ser |
| 5:131677807:G:A | missense | ENST00000510461.6: | ENSP00000421985.1: |
| | | c.1415C>T | p.Pro472Leu |
| 5:131677811:T:C | missense | ENST00000510461.6: | ENSP00000421985.1: |
| | | c.1411A>G | p.Met471Val |
| 5:131677811:T:G | missense | ENST00000510461.6: | ENSP00000421985.1: |
| | | c.1411A>C | p.Met471Leu |
| 5:131677819:G:A | missense | ENST00000510461.6: | ENSP00000421985.1: |
| | | c.1403C>T | p.Pro468Leu |
| 5:131677823:C:T | missense | ENST00000510461.6: | ENSP00000421985.1: |
| | | c.1399G>A | p.Val467Ile |
| 5:131677829:C:T | missense | ENST00000510461.6: | ENSP00000421985.1: |
| | | c.1393G>A | p.Ala465Thr |
| 5:131677835:G:A | missense | ENST00000510461.6: | ENSP00000421985.1: |
| | | c.1387C>T | p.His463Tyr |
| 5:131677837:T:C | missense | ENST00000510461.6: | ENSP00000421985.1: |
| | | c.1385A>G | p.Asn462Ser |
| 5:131677838:T:A | missense | ENST00000510461.6: | ENSP00000421985.1: |
| | | c.1384A>T | p.Asn462Tyr |
| 5:131677840:G:A | missense | ENST00000510461.6: | ENSP00000421985.1: |
| | | c.1382C>T | p.Thr461Ile |
| 5:131677840:G:C | missense | ENST00000510461.6: | ENSP00000421985.1: |
| | | c.1382C>G | p.Thr461Ser |
| 5:131677847:C:G | missense | ENST00000510461.6: | ENSP00000421985.1: |
| | | c.1375G>C | p.Val459Leu |
| 5:131677849:G:A | missense | ENST00000510461.6: | ENSP00000421985.1: |
| | | c.1373C>T | p.Ala458Val |
| 5:131677856:T:C | missense | ENST00000510461.6: | ENSP00000421985.1: |
| | | c.1366A>G | p.Ile456Val |
| 5:131677856:T:G | missense | ENST00000510461.6: | ENSP00000421985.1: |
| | | c.1366A>C | p.Ile456Leu |
| 5:131677859:G:A | missense | ENST00000510461.6: | ENSP00000421985.1: |
| | | c.1363C>T | p.Leu455Phe |
| 5:131677859:G:C | missense | ENST00000510461.6: | ENSP00000421985.1: |
| | | c.1363C>G | p.Leu455Val |
| 5:131677865:G:A | missense | ENST00000510461.6: | ENSP00000421985.1: |
| | | c.1357C>T | p.Pro453Ser |
| 5:131677869:G:C | missense | ENST00000510461.6: | ENSP00000421985.1: |
| | | c.1353C>G | p.Phe451Leu |
| 5:131679030:G:T | missense | ENST00000510461.6: | ENSP00000421985.1: |
| | | c.1348C>A | p.Gln450Lys |
| 5:131679032:T:C | missense | ENST00000510461.6: | ENSP00000421985.1: |
| | | c.1346A>G | p.Asn449Ser |
| 5:131679032:T:G | missense | ENST00000510461.6: | ENSP00000421985.1: |
| | | c.1346A>C | p.Asn449Thr |
| 5:131679033:T:A | missense | ENST00000510461.6: | ENSP00000421985.1: |
| | | c.1345A>T | p.Asn449Tyr |
| 5:131679041:G:A | missense | ENST00000510461.6: | ENSP00000421985.1: |
| | | c.1337C>T | p.Ala446Val |
| 5:131679044:T:C | missense | ENST00000510461.6: | ENSP00000421985.1: |
| | | c.1334A>G | p.Asn445Ser |
| 5:131679048:C:G | missense | ENST00000510461.6: | ENSP00000421985.1: |
| | | c.1330G>C | p.Glu444Gln |
| 5:131679050:A:T | missense | ENST00000510461.6: | ENSP00000421985.1: |
| | | c.1328T>A | p.Met443Lys |
| 5:131679060:T:C | missense | ENST00000510461.6: | ENSP00000421985.1: |
| | | c.1318A>G | p.Thr440Ala |
| 5:131679061:G:C | missense | ENST00000510461.6: | ENSP00000421985.1: |
| | | c.1317C>G | p.Phe439Leu |
| 5:131679062:A:T | missense | ENST00000510461.6: | ENSP00000421985.1: |
| | | c.1316T>A | p.Phe439Tyr |
| 5:131679064:C:A | missense | ENST00000510461.6: | ENSP00000421985.1: |
| | | c.1314G>T | p.Glu438Asp |
| 5:131679066:C:G | missense | ENST00000510461.6: | ENSP00000421985.1: |
| | | c.1312G>C | p.Glu438Gln |
| 5:131679066:C:T | missense | ENST00000510461.6: | ENSP00000421985.1: |
| | | c.1312G>A | p.Glu438Lys |
| 5:131679069:T:C | missense | ENST00000510461.6: | ENSP00000421985.1: |
| | | c.1309A>G | p.Lys437Glu |
| 5:131679070:C:A | missense | ENST00000510461.6: | ENSP00000421985.1: |
| | | c.1308G>T | p.Met436Ile |
| 5:131679072:T:A | missense | ENST00000510461.6: | ENSP00000421985.1: |
| | | c.1306A>T | p.Met436Leu |
| 5:131679072:T:C | missense | ENST00000510461.6: | ENSP00000421985.1: |
| | | c.1306A>G | p.Met436Val |
| 5:131679073:G:C | missense | ENST00000510461.6: | ENSP00000421985.1: |
| | | c.1305C>G | p.Phe435Leu |
| 5:131679074:A:C | missense | ENST00000510461.6: | ENSP00000421985.1: |
| | | c.1304T>G | p.Phe435Cys |
| 5:131679077:C:T | missense | ENST00000510461.6: | ENSP00000421985.1: |
| | | c.1301G>A | p.Arg434His |
| 5:131679078:G:A | missense | ENST00000510461.6: | ENSP00000421985.1: |
| | | c.1300C>T | p.Arg434Cys |
| 5:131679078:G:T | missense | ENST00000510461.6: | ENSP00000421985.1: |
| | | c.1300C>A | p.Arg434Ser |
| 5:131679079:A:T | stop_ | ENST00000510461.6: | ENSP00000421985.1: |
| | gained | c.1299T>A | p.Tyr433Ter |
| 5:131679080:T:C | missense | ENST00000510461.6: | ENSP00000421985.1: |
| | | c.1298A>G | p.Tyr433Cys |
| 5:131679083:C:A | missense | ENST00000510461.6: | ENSP00000421985.1: |
| | | c.1295G>T | p.Cys432Phe |
| 5:131679084:A:C | missense | ENST00000510461.6: | ENSP00000421985.1: |
| | | c.1294T>G | p.Cys432Gly |
| 5:131679087:G:A | missense | ENST00000510461.6: | ENSP00000421985.1: |
| | | c.1291C>T | p.Leu431Phe |
| 5:131679087:G:C | missense | ENST00000510461.6: | ENSP00000421985.1: |
| | | c.1291C>G | p.Leu431Val |
| 5:131679088:G:T | missense | ENST00000510461.6: | ENSP00000421985.1: |
| | | c.1290C>A | p.His430Gln |
| 5:131679089:T:C | missense | ENST00000510461.6: | ENSP00000421985.1: |
| | | c.1289A>G | p.His430Arg |
| 5:131679092:T:C | missense | ENST00000510461.6: | ENSP00000421985.1: |
| | | c.1286A>G | p.Asn429Ser |
| 5:131679094:C:A | missense | ENST00000510461.6: | ENSP00000421985.1: |
| | | c.1284G>T | p.Lys428Asn |
| 5:131679102:G:A | missense | ENST00000510461.6: | ENSP00000421985.1: |
| | | c.1276C>T | p.Pro426Ser |
| 5:131679102:G:T | missense | ENST00000510461.6: | ENSP00000421985.1: |
| | | c.1276C>A | p.Pro426Thr |
| 5:131679104:G:A | missense | ENST00000510461.6: | ENSP00000421985.1: |
| | | c.1274C>T | p.Thr425Ile |
| 5:131679107:C:G | missense | ENST00000510461.6: | ENSP00000421985.1: |
| | | c.1271G>C | p.Gly424Ala |
| 5:131679107:C:T | missense | ENST00000510461.6: | ENSP00000421985.1: |
| | | c.1271G>A | p.Gly424Glu |
| 5:131679110:G:A | missense | ENST00000510461.6: | ENSP00000421985.1: |
| | | c.1268C>T | p.Ser423Leu |
| 5:131679112:C:T | missense | ENST00000510461.6: | ENSP00000421985.1: |
| | | c.1266G>A | p.Met422Ile |
| 5:131679113:A:G | missense | ENST00000510461.6: | ENSP00000421985.1: |
| | | c.1265T>C | p.Met422Thr |
| 5:131679114:T:A | missense | ENST00000510461.6: | ENSP00000421985.1: |
| | | c.1264A>T | p.Met422Leu |
| 5:131679117:T:A | missense | ENST00000510461.6: | ENSP00000421985.1: |
| | | c.1261A>T | p.Met421Leu |
| 5:131679117:T:C | missense | ENST00000510461.6: | ENSP00000421985.1: |
| | | c.1261A>G | p.Met421Val |
| 5:131679119:G:T | missense | ENST00000510461.6: | ENSP00000421985.1: |
| | | c.1259C>A | p.Thr420Lys |
| 5:131679120:T:C | missense | ENST00000510461.6: | ENSP00000421985.1: |
| | | c.1258A>G | p.Thr420Ala |
| 5:131679124:C:T | stop_ | ENST00000510461.6: | ENSP00000421985.1: |
| | gained | c.1254G>A | p.Trp418Ter |
| 5:131679126:A:G | missense | ENST00000510461.6: | ENSP00000421985.1: |
| | | c.1252T>C | p.Trp418Arg |
| 5:131679129:C:G | missense | ENST00000510461.6: | ENSP00000421985.1: |
| | | c.1249G>C | p.Val417Leu |
| 5:131679129:C:T | missense | ENST00000510461.6: | ENSP00000421985.1: |
| | | c.1249G>A | p.Val417Ile |
| 5:131679131:G:T | missense | ENST00000510461.6: | ENSP00000421985.1: |
| | | c.1247C>A | p.Pro416His |
| 5:131679132:G:A | missense | ENST00000510461.6: | ENSP00000421985.1: |
| | | c.1246C>T | p.Pro416Ser |
| 5:131679132:G:T | missense | ENST00000510461.6: | ENSP00000421985.1: |
| | | c.1246C>A | p.Pro416Thr |
| 5:131679133:T:G | missense | ENST00000510461.6: | ENSP00000421985.1: |
| | | c.1245A>C | p.Glu415Asp |
| 5:131679135:C:T | missense | ENST00000510461.6: | ENSP00000421985.1: |
| | | c.1243G>A | p.Glu415Lys |
| 5:131679138:C:A | stop_ | ENST00000510461.6: | ENSP00000421985.1: |
| | gained | c.1240G>T | p.Gly414Ter |
| 5:131679138:C:T | missense | ENST00000510461.6: | ENSP00000421985.1: |
| | | c.1240G>A | p.Gly414Arg |
| 5:131679143:C:T | missense | ENST00000510461.6: | ENSP00000421985.1: |
| | | c.1235G>A | p.Arg412Gln |
| 5:131679144:G:A | stop_ | ENST00000510461.6: | ENSP00000421985.1: |
| | gained | c.1234C>T | p.Arg412Ter |
| 5:131679147:G:C | missense | ENST00000510461.6: | ENSP00000421985.1: |
| | | c.1231C>G | p.Pro411Ala |
| 5:131679147:G:T | missense | ENST00000510461.6: | ENSP00000421985.1: |
| | | c.1231C>A | p.Pro411Thr |
| 5:131679149:A:G | missense | ENST00000510461.6: | ENSP00000421985.1: |
| | | c.1229T>C | p.Met410Thr |
| 5:131679150:T:A | missense | ENST00000510461.6: | ENSP00000421985.1: |
| | | c.1228A>T | p.Met410Leu |
| 5:131679152:G:A | missense | ENST00000510461.6: | ENSP00000421985.1: |
| | | c.1226C>T | p.Thr409Met |
| 5:131679152:G:T | missense | ENST00000510461.6: | ENSP00000421985.1: |
| | | c.1226C>A | p.Thr409Lys |
| 5:131679153:T:C | missense | ENST00000510461.6: | ENSP00000421985.1: |
| | | c.1225A>G | p.Thr409Ala |
| 5:131679154:G:T | stop_ | ENST00000510461.6: | ENSP00000421985.1: |
| | gained | c.1224C>A | p.Tyr408Ter |
| 5:131679156:A:G | missense | ENST00000510461.6: | ENSP00000421985.1: |
| | | c.1222T>C | p.Tyr408His |
| 5:131679161:T:G | missense | ENST00000510461.6: | ENSP00000421985.1: |
| | | c.1217A>C | p.Asn406Thr |
| 5:131679164:C:A | missense | ENST00000510461.6: | ENSP00000421985.1: |
| | | c.1214G>T | p.Cys405Phe |
| 5:131679164:C:G | missense | ENST00000510461.6: | ENSP00000421985.1: |
| | | c.1214G>C | p.Cys405Ser |
| 5:131679164:C:T | missense | ENST00000510461.6: | ENSP00000421985.1: |
| | | c.1214G>A | p.Cys405Tyr |
| 5:131679165:A:G | missense | ENST00000510461.6: | ENSP00000421985.1: |
| | | c.1213T>C | p.Cys405Arg |
| 5:131679168:T:C | missense | ENST00000510461.6: | ENSP00000421985.1: |
| | | c.1210A>G | p.Ile404Val |
| 5:131679170:G:A | missense | ENST00000510461.6: | ENSP00000421985.1: |
| | | c.1208C>T | p.Thr403Ile |
| 5:131679170:G:T | missense | ENST00000510461.6: | ENSP00000421985.1: |
| | | c.1208C>A | p.Thr403Lys |
| 5:131679171:T:A | missense | ENST00000510461.6: | ENSP00000421985.1: |
| | | c.1207A>T | p.Thr403Ser |
| 5:131679171:T:C | missense | ENST00000510461.6: | ENSP00000421985.1: |
| | | c.1207A>G | p.Thr403Ala |
| 5:131679173:G:C | missense | ENST00000510461.6: | ENSP00000421985.1: |
| | | c.1205C>G | p.Thr402Arg |
| 5:131679173:G:T | missense | ENST00000510461.6: | ENSP00000421985.1: |
| | | c.1205C>A | p.Thr402Lys |
| 5:131679173:GTTC:G | splice_ | ENST00000510461.6: | NA |
| | acc | c.1203-1_ | |
| | eptor | 1204del | |
| 5:131679175:T:G | missense | ENST00000510461.6: | ENSP00000421985.1: |
| | | c.1203A>C | p.Arg401Ser |
| 5:131679176:C:T | splice_ | ENST00000510461.6: | NA |
| | acceptor | c.1203-1G>A | |
| 5:131679177:T:A | splice_ | ENST00000510461.6: | NA |
| | acceptor | c.1203-2A>T | |
| 5:131698916:C:T | splice_ | ENST00000510461.6: | NA |
| | donor | c.1202+1G>A | |
| 5:131698919:G:C | missense | ENST00000510461.6: | ENSP00000421985.1: |
| | | c.1200C>G | p.Phe400Leu |
| 5:131698921:A:G | missense | ENST00000510461.6: | ENSP00000421985.1: |
| | | c.1198T>C | p.Phe400Leu |
| 5:131698930:G:C | missense | ENST00000510461.6: | ENSP00000421985.1: |
| | | c.1189C>G | p.Leu397Val |
| 5:131698932:G:A | missense | ENST00000510461.6: | ENSP00000421985.1: |
| | | c.1187C>T | p.Ala396Val |
| 5:131698932:G:C | missense | ENST00000510461.6: | ENSP00000421985.1: |
| | | c.1187C>G | p.Ala396Gly |
| 5:131698932:G:T | missense | ENST00000510461.6: | ENSP00000421985.1: |
| | | c.1187C>A | p.Ala396Asp |
| 5:131698933:C:A | missense | ENST00000510461.6: | ENSP00000421985.1: |
| | | c.1186G>T | p.Ala396Ser |
| 5:131698936:C:A | missense | ENST00000510461.6: | ENSP00000421985.1: |
| | | c.1183G>T | p.Asp395Tyr |
| 5:131698936:C:T | missense | ENST00000510461.6: | ENSP00000421985.1: |
| | | c.1183G>A | p.Asp395Asn |
| 5:131698942:T:C | missense | ENST00000510461.6: | ENSP00000421985.1: |
| | | c.1177A>G | p.IIle393Val |
| 5:131698944:C:A | missense | ENST00000510461.6: | ENSP00000421985.1: |
| | | c.1175G>T | p.Arg392Leu |
| 5:131698944:C:G | missense | ENST00000510461.6: | ENSP00000421985.1: |
| | | c.1175G>C | p.Arg392Pro |
| 5:131698944:C:T | missense | ENST00000510461.6: | ENSP00000421985.1: |
| | | c.1175G>A | p.Arg392Gln |
| 5:131698945:G:A | stop_ | ENST00000510461.6: | ENSP00000421985.1: |
| | gained | c.1174C>T | p.Arg392Ter |
| 5:131698947:T:A | missense | ENST00000510461.6: | ENSP00000421985.1: |
| | | c.1172A>T | p.Asn391Ile |
| 5:131698950:T:A | missense | ENST00000510461.6: | ENSP00000421985.1: |
| | | c.1169A>T | p.Tyr390Phe |
| 5:131698950:T:C | missense | ENST00000510461.6: | ENSP00000421985.1: |
| | | c.1169A>G | p.Tyr390Cys |
| 5:131698951:A:G | missense | ENST00000510461.6: | ENSP00000421985.1: |
| | | c.1168T>C | p.Tyr390His |
| 5:131698953:G:A | missense | ENST00000510461.6: | ENSP00000421985.1: |
| | | c.1166C>T | p.Ala389Val |
| 5:131698959:C:G | missense | ENST00000510461.6: | ENSP00000421985.1: |
| | | c.1160G>C | p.Ser387Thr |
| 5:131698963:T:C | missense | ENST00000510461.6: | ENSP00000421985.1: |
| | | c.1156A>G | p.Arg386Gly |
| 5:131698967:A:T | missense | ENST00000510461.6: | ENSP00000421985.1: |
| | | c.1152T>A | p.Ser384Arg |
| 5:131698968:C:A | missense | ENST00000510461.6: | ENSP00000421985.1: |
| | | c.1151G>T | p.Ser384Ile |
| 5:131698968:C:G | missense | ENST00000510461.6: | ENSP00000421985.1: |
| | | c.1151G>C | p.Ser384Thr |
| 5:131698969:T:C | missense | ENST00000510461.6: | ENSP00000421985.1: |
| | | c.1150A>G | p.Ser384Gly |
| 5:131698972:C:T | missense | ENST00000510461.6: | ENSP00000421985.1: |
| | | c.1147G>A | p.Ala383Thr |
| 5:131698974:T:C | missense | ENST00000510461.6: | ENSP00000421985.1: |
| | | c.1145A>G | p.Asp382Gly |
| 5:131698978:C:G | missense | ENST00000510461.6: | ENSP00000421985.1: |
| | | c.1141G>C | p.Ala381Pro |
| 5:131698979:TG:T | frameshift | ENST00000510461.6: | ENSP00000421985.1: |
| | | c.1139del | p.Ser380Ter |
| 5:131698981:A:C | missense | ENST00000510461.6: | ENSP00000421985.1: |
| | | c.1138T>G | p.Ser380Ala |
| 5:131698981:A:G | missense | ENST00000510461.6: | ENSP00000421985.1: |
| | | c.1138T>C | p.Ser380Pro |
| 5:131698983:C:T | missense | ENST00000510461.6: | ENSP00000421985.1: |
| | | c.1136G>A | p.Arg379Lys |
| 5:131698984:T:C | missense | ENST00000510461.6: | ENSP00000421985.1: |
| | | c.1135A>G | p.Arg379Gly |
| 5:131698986:C:T | missense | ENST00000510461.6: | ENSP00000421985.1: |
| | | c.1133G>A | p.Arg378Gln |
| 5:131698987:G:A | missense | ENST00000510461.6: | ENSP00000421985.1: |
| | | c.1132C>T | p.Arg378Trp |
| 5:131698991:C:T | missense | ENST00000510461.6: | ENSP00000421985.1: |
| | | c.1128G>A | p.Met376Ile |
| 5:131698993:T:C | missense | ENST00000510461.6: | ENSP00000421985.1: |
| | | c.1126A>G | p.Met376Val |
| 5:131698997:C:G | missense | ENST00000510461.6: | ENSP00000421985.1: |
| | | c.1122G>C | p.Met374Ile |
| 5:131698998:A:G | missense | ENST00000510461.6: | ENSP00000421985.1: |
| | | c.1121T>C | p.Met374Thr |
| 5:131698999:T:C | missense | ENST00000510461.6: | ENSP00000421985.1: |
| | | c.1120A>G | p.Met374Val |
| 5:131699001:G:A | missense | ENST00000510461.6: | ENSP00000421985.1: |
| | | c.1118C>T | p.Ala373Val |
| 5:131699001:G:T | missense | ENST00000510461.6: | ENSP00000421985.1: |
| | | c.1118C>A | p.Ala373Asp |
| 5:131704062:TA:T | splice_ | ENST00000510461.6: | NA |
| | donor | c.1116+2del | |
| 5:131704072:A:G | missense | ENST00000510461.6: | ENSP00000421985.1: |
| | | c.1109T>C | p.Ile370Thr |
| 5:131704073:T:C | missense | ENST00000510461.6: | ENSP00000421985.1: |
| | | c.1108A>G | p.Ile370Val |
| 5:131704075:G:A | missense | ENST00000510461.6: | ENSP00000421985.1: |
| | | c.1106C>T | p.Ala369Val |
| 5:131704079:T:A | missense | ENST00000510461.6: | ENSP00000421985.1: |
| | | c.1102A>T | p.Ser368Cys |
| 5:131704081:T:C | missense | ENST00000510461.6: | ENSP00000421985.1: |
| | | c.1100A>G | p.Lys367Arg |
| 5:131704090:T:C | missense | ENST00000510461.6: | ENSP00000421985.1: |
| | | c.1091A>G | p.Asn364Ser |
| 5:131704094:T:C | missense | ENST00000510461.6: | ENSP00000421985.1: |
| | | c.1087A>G | p.Met363Val |
| 5:131704096:T:A | missense | ENST00000510461.6: | ENSP00000421985.1: |
| | | c.1085A>T | p.His362Leu |
| 5:131704099:C:T | missense | ENST00000510461.6: | ENSP00000421985.1: |
| | | c.1082G>A | p.Ser361Asn |
| 5:131704108:A:T | missense | ENST00000510461.6: | ENSP00000421985.1: |
| | | c.1073T>A | p.Leu358His |
| 5:131704109:G:C | missense | ENST00000510461.6: | ENSP00000421985.1: |
| | | c.1072C>G | p.Leu358Val |
| 5:131704111:G:A | missense | ENST00000510461.6: | ENSP00000421985.1: |
| | | c.1070C>T | p.Pro357Leu |
| 5:131704112:G:A | missense | ENST00000510461.6: | ENSP00000421985.1: |
| | | c.1069C>T | p.Pro357Ser |
| 5:131704114:A:C | missense | ENST00000510461.6: | ENSP00000421985.1: |
| | | c.1067T>G | p.Phe356Cys |
| 5:131704120:G:GA | frameshift | ENST00000510461.6: | ENSP00000421985.1: |
| | | c.1060dup | p.Ser354PhefsTer7 |
| 5:131704124:A:G | missense | ENST00000510461.6: | ENSP00000421985.1: |
| | | c.1057T>C | p.Phe353Leu |
| 5:131704127:A:G | missense | ENST00000510461.6: | ENSP00000421985.1: |
| | | c.1054T>C | p.Phe352Leu |
| 5:131704128:G:T | missense | ENST00000510461.6: | ENSP00000421985.1: |
| | | c.1053C>A | p.Phe351Leu |
| 5:131704130:A:C | missense | ENST00000510461.6: | ENSP00000421985.1: |
| | | c.1051T>G | p.Phe351Val |
| 5:131704131:T:A | missense | ENST00000510461.6: | ENSP00000421985.1: |
| | | c.1050A>T | p.Glu350Asp |
| 5:131704132:T:A | missense | ENST00000510461.6: | ENSP00000421985.1: |
| | | c.1049A>T | p.Glu350Val |
| 5:131704135:T:C | missense | ENST00000510461.6: | ENSP00000421985.1: |
| | | c.1046A>G | p.Asn349Ser |
| 5:131704137:A:C | missense | ENST00000510461.6: | ENSP00000421985.1: |
| | | c.1044T>G | p.Phe348Leu |
| 5:131704138:A:G | missense | ENST00000510461.6: | ENSP00000421985.1: |
| | | c.1043T>C | p.Phe348Ser |
| 5:131704138:A:T | missense | ENST00000510461.6: | ENSP00000421985.1: |
| | | c.1043T>A | p.Phe348Tyr |
| 5:131704140:T:G | missense | ENST00000510461.6: | ENSP00000421985.1: |
| | | c.1041A>C | p.Lys347Asn |
| 5:131704141:T:G | missense | ENST00000510461.6: | ENSP00000421985.1: |
| | | c.1040A>C | p.Lys347Thr |
| 5:131704142:T:C | missense | ENST00000510461.6: | ENSP00000421985.1: |
| | | c.1039A>G | p.Lys347Glu |
| 5:131704144:T:C | missense | ENST00000510461.6: | ENSP00000421985.1: |
| | | c.1037A>G | p.Asn346Ser |
| 5:131704147:T:C | missense | ENST00000510461.6: | ENSP00000421985.1: |
| | | c.1034A>G | p.Asn345Ser |
| 5:131704153:T:A | missense | ENST00000510461.6: | ENSP00000421985.1: |
| | | c.1028A>T | p.Asp343Val |
| 5:131704157:C:T | missense | ENST00000510461.6: | ENSP00000421985.1: |
| | | c.1024G>A | p.Glu342Lys |
| 5:131704159:T:A | missense | ENST00000510461.6: | ENSP00000421985.1: |
| | | c.1022A>T | p.Asp341Val |
| 5:131704160:CT:C | frameshift | ENST00000510461.6: | ENSP00000421985.1: |
| | | c.1020del | p.Asp341MetfsTer23 |
| 5:131704162:T:C | missense | ENST00000510461.6: | ENSP00000421985.1: |
| | | c.1019A>G | p.Lys340Arg |
| 5:131704165:G:C | missense | ENST00000510461.6: | ENSP00000421985.1: |
| | | c.1016C>G | p.Ser339Cys |
| 5:131704166:A:G | missense | ENST00000510461.6: | ENSP00000421985.1: |
| | | c.1015T>C | p.Ser339Pro |
| 5:131704167:C:G | missense | ENST00000510461.6: | ENSP00000421985.1: |
| | | c.1014G>C | p.Leu338Phe |
| 5:131704171:G:A | missense | ENST00000510461.6: | ENSP00000421985.1: |
| | | c.1010C>T | p.Ser337Leu |
| 5:131704178:T:A | missense | ENST00000510461.6: | ENSP00000421985.1: |
| | | c.1003A>T | p.Ile335Phe |
| 5:131704178:T:C | missense | ENST00000510461.6: | ENSP00000421985.1: |
| | | c.1003A>G | p.Ile335Val |
| 5:131704181:C:A | missense | ENST00000510461.6: | ENSP00000421985.1: |
| | | c.1000G>T | p.Val334Leu |
| 5:131704186:A:G | missense | ENST00000510461.6: | ENSP00000421985.1: |
| | | c.995T>C | p.Ile332Thr |
| 5:131704187:T:C | missense | ENST00000510461.6: | ENSP00000421985.1: |
| | | c.994A>G | p.Ile332Val |
| 5:131704193:T:C | missense | ENST00000510461.6: | ENSP00000421985.1: |
| | | c.988A>G | p.Ile330Val |
| 5:131704202:T:G | missense | ENST00000510461.6: | ENSP00000421985.1: |
| | | c.979A>C | p.Lys327Gln |
| 5:131704204:C:T | missense | ENST00000510461.6: | ENSP00000421985.1: |
| | | c.977G>A | p.Arg326Gln |
| 5:131704205:G:A | missense | ENST00000510461.6: | ENSP00000421985.1: |
| | | c.976C>T | p.Arg326Trp |
| 5:131704207:A:C | missense | ENST00000510461.6: | ENSP00000421985.1: |
| | | c.974T>G | p.Val325Gly |
| 5:131704207:A:G | missense | ENST00000510461.6: | ENSP00000421985.1: |
| | | c.974T>C | p.Val325Ala |
| 5:131704208:C:T | missense | ENST00000510461.6: | ENSP00000421985.1: |
| | | c.973G>A | p.Val325Met |
| 5:131704211:T:C | missense | ENST00000510461.6: | ENSP00000421985.1: |
| | | c.970A>G | p.Ile324Val |
| 5:131704214:C:T | missense | ENST00000510461.6: | ENSP00000421985.1: |
| | | c.967G>A | p.Gly323Arg |
| 5:131704216:G:A | missense | ENST00000510461.6: | ENSP00000421985.1: |
| | | c.965C>T | p.Pro322Leu |
| 5:131704220:T:G | missense | ENST00000510461.6: | ENSP00000421985.1: |
| | | c.961A>C | p.Asn321His |
| 5:131704222:G:A | missense | ENST00000510461.6: | ENSP00000421985.1: |
| | | c.959C>T | p.Pro320Leu |
| 5:131704222:G:T | missense | ENST00000510461.6: | ENSP00000421985.1: |
| | | c.959C>A | p.Pro320His |
| 5:131704223:G:A | missense | ENST00000510461.6: | ENSP00000421985.1: |
| | | c.958C>T | p.Pro320Ser |
| 5:131704228:C:G | missense | ENST00000510461.6: | ENSP00000421985.1: |
| | | c.953G>C | p.Cys318Ser |
| 5:131704228:C:T | missense | ENST00000510461.6: | ENSP00000421985.1: |
| | | c.953G>A | p.Cys318Tyr |
| 5:131704229:A:G | missense | ENST00000510461.6: | ENSP00000421985.1: |
| | | c.952T>C | p.Cys318Arg |
| 5:131704230:G:C | missense | ENST00000510461.6: | ENSP00000421985.1: |
| | | c.951C>G | p.Ser317Arg |
| 5:131704231:C:T | missense | ENST00000510461.6: | ENSP00000421985.1: |
| | | c.950G>A | p.Ser317Asn |
| 5:131704232:T:G | missense | ENST00000510461.6: | ENSP00000421985.1: |
| | | c.949A>C | p.Ser317Arg |
| 5:131704236:A:T | missense | ENST00000510461.6: | ENSP00000421985.1: |
| | | c.945T>A | p.Asp315Glu |
| 5:131704237:T:A | missense | ENST00000510461.6: | ENSP00000421985.1: |
| | | c.944A>T | p.Asp315Val |
| 5:131704237:T:C | missense | ENST00000510461.6: | ENSP00000421985.1: |
| | | c.944A>G | p.Asp315Gly |
| 5:131704240:G:A | missense | ENST00000510461.6: | ENSP00000421985.1: |
| | | c.941C>T | p.Ser314Leu |
| 5:131704250:A:G | missense | ENST00000510461.6: | ENSP00000421985.1: |
| | | c.931T>C | p.Phe311Leu |
| 5:131704251:G:C | missense | ENST00000510461.6: | ENSP00000421985.1: |
| | | c.930C>G | p.Ser310Arg |
| 5:131704252:C:T | missense | ENST00000510461.6: | ENSP00000421985.1: |
| | | c.929G>A | p.Ser310Asn |
| 5:131704259:C:T | missense | ENST00000510461.6: | ENSP00000421985.1: |
| | | c.922G>A | p.Glu308Lys |
| 5:131704261:A:G | missense | ENST00000510461.6: | ENSP00000421985.1: |
| | | c.920T>C | p.Ile307Thr |
| 5:131704262:T:C | missense | ENST00000510461.6: | ENSP00000421985.1: |
| | | c.919A>G | p.Ile307Val |
| 5:131706409:A:G | splice_ | ENST00000510461.6: | NA |
| | donor | c.914+2T>C | |
| 5:131706412:A:G | missense | ENST00000510461.6: | ENSP00000421985.1: |
| | | c.913T>C | p.Trp305Arg |
| 5:131706414:C:T | missense | ENST00000510461.6: | ENSP00000421985.1: |
| | | c.911G>A | p.Arg304Lys |
| 5:131706418:G:A | missense | ENST00000510461.6: | ENSP00000421985.1: |
| | | c.907C>T | p.Pro303Ser |
| 5:131706424:C:T | missense | ENST00000510461.6: | ENSP00000421985.1: |
| | | c.901G>A | p.Val301Ile |
| 5:131706426:C:G | missense | ENST00000510461.6: | ENSP00000421985.1: |
| | | c.899G>C | p.Gly300Ala |
| 5:131706426:C:T | missense | ENST00000510461.6: | ENSP00000421985.1: |
| | | c.899G>A | p.Gly300Glu |
| 5:131706427:C:T | missense | ENST00000510461.6: | ENSP00000421985.1: |
| | | c.898G>A | p.Gly300Arg |
| 5:131706432:T:C | missense | ENST00000510461.6: | ENSP00000421985.1: |
| | | c.893A>G | p.Glu298Gly |
| 5:131706438:C:G | missense | ENST00000510461.6: | ENSP00000421985.1: |
| | | c.887G>C | p.Ser296Thr |
| 5:131706441:G:C | missense | ENST00000510461.6: | ENSP00000421985.1: |
| | | c.884C>G | p.Thr295Arg |
| 5:131706442:T:C | missense | ENST00000510461.6: | ENSP00000421985.1: |
| | | c.883A>G | p.Thr295Ala |
| 5:131706445:T:C | missense | ENST00000510461.6: | ENSP00000421985.1: |
| | | c.880A>G | p.Thr294Ala |
| 5:131706448:G:C | missense | ENST00000510461.6: | ENSP00000421985.1: |
| | | c.877C>G | p.Gln293Glu |
| 5:131706453:C:T | missense | ENST00000510461.6: | ENSP00000421985.1: |
| | | c.872G>A | p.Arg291His |
| 5:131706454:G:A | missense | ENST00000510461.6: | ENSP00000421985.1: |
| | | c.871C>T | p.Arg291Cys |
| 5:131706456:C:T | missense | ENST00000510461.6: | ENSP00000421985.1: |
| | | c.869G>A | p.Arg290Gln |
| 5:131706457:G:A | stop_ | ENST00000510461.6: | ENSP00000421985.1: |
| | gained | c.868C>T | p.Arg290Ter |
| 5:131706462:C:T | missense | ENST00000510461.6: | ENSP00000421985.1: |
| | | c.863G>A | p.Arg288His |
| 5:131706463:G:A | missense | ENST00000510461.6: | ENSP00000421985.1: |
| | | c.862C>T | p.Arg288Cys |
| 5:131706463:G:T | missense | ENST00000510461.6: | ENSP00000421985.1: |
| | | c.862C>A | p.Arg288Ser |
| 5:131706465:C:T | missense | ENST00000510461.6: | ENSP00000421985.1: |
| | | c.860G>A | p.Arg287Gln |
| 5:131706466:G:A | stop_ | ENST00000510461.6: | ENSP00000421985.1: |
| | gained | c.859C>T | p.Arg287Ter |
| 5:131706471:T:C | missense | ENST00000510461.6: | ENSP00000421985.1: |
| | | c.854A>G | p.Tyr285Cys |
| 5:131706480:G:A | missense | ENST00000510461.6: | ENSP00000421985.1: |
| | | c.845C>T | p.Ala282Val |
| 5:131706486:C:T | missense | ENST00000510461.6: | ENSP00000421985.1: |
| | | c.839G>A | p.Ser280Asn |
| 5:131706487:T:A | missense | ENST00000510461.6: | ENSP00000421985.1: |
| | | c.838A>T | p.Ser280Cys |
| 5:131706489:C:T | missense | ENST00000510461.6: | ENSP00000421985.1: |
| | | c.836G>A | p.Arg279Gln |
| 5:131706490:G:A | stop_ | ENST00000510461.6: | ENSP00000421985.1: |
| | gained | c.835C>T | p.Arg279Ter |
| 5:131706490:G:C | missense | ENST00000510461.6: | ENSP00000421985.1: |
| | | c.835C>G | p.Arg279Gly |
| 5:131706491:GGTAA: | frameshift | ENST00000510461.6: | ENSP00000421985.1: |
| G | | c.830_ | p.Leu277ProfsTer30 |
| | | 833del | |
| 5:131706501:G:A | missense | ENST00000510461.6: | ENSP00000421985.1: |
| | | c.824C>T | p.Ser275Phe |
| 5:131706502:A:C | missense | ENST00000510461.6: | ENSP00000421985.1: |
| | | c.823T>G | p.Ser275Ala |
| 5:131706504:T:G | missense | ENST00000510461.6: | ENSP00000421985.1: |
| | | c.821A>C | p.Asn274Thr |
| 5:131706507:G:C | missense | ENST00000510461.6: | ENSP00000421985.1: |
| | | c.818C>G | p.Pro273Arg |
| 5:131706508:G:C | missense | ENST00000510461.6: | ENSP00000421985.1: |
| | | c.817C>G | p.Pro273Ala |
| 5:131706516:A:C | missense | ENST00000510461.6: | ENSP00000421985.1: |
| | | c.809T>G | p.Phe270Cys |
| 5:131706519:G:A | missense | ENST00000510461.6: | ENSP00000421985.1: |
| | | c.806C>T | p.Pro269Leu |
| 5:131706519:G:C | missense | ENST00000510461.6: | ENSP00000421985.1: |
| | | c.806C>G | p.Pro269Arg |
| 5:131706520:G:C | missense | ENST00000510461.6: | ENSP00000421985.1: |
| | | c.805C>G | p.Pro269Ala |
| 5:131706523:T:A | missense | ENST00000510461.6: | ENSP00000421985.1: |
| | | c.802A>T | p.Thr268Ser |
| 5:131706523:T:C | missense | ENST00000510461.6: | ENSP00000421985.1: |
| | | c.802A>G | p.Thr268Ala |
| 5:131706524:G:C | missense | ENST00000510461.6: | ENSP00000421985.1: |
| | | c.801C>G | p.Ile267Met |
| 5:131706525:A:G | missense | ENST00000510461.6: | ENSP00000421985.1: |
| | | c.800T>C | p.Ile267Thr |
| 5:131706526:T:C | missense | ENST00000510461.6: | ENSP00000421985.1: |
| | | c.799A>G | p.Ile267Val |
| 5:131706541:G:A | missense | ENST00000510461.6: | ENSP00000421985.1: |
| | | c.784C>T | p.Leu262Phe |
| 5:131706548:T:C | splice_ | ENST00000510461.6: | NA |
| | acceptor | c.779-2A>G | |
| 5:131709206:C:T | missense | ENST00000510461.6: | ENSP00000421985.1: |
| | | c.773G>A | p.Arg258Gln |
| 5:131709207:G:A | missense | ENST00000510461.6: | ENSP00000421985.1: |
| | | c.772C>T | p.Arg258Trp |
| 5:131709210:C:A | missense | ENST00000510461.6: | ENSP00000421985.1: |
| | | c.769G>T | p.Ala257Ser |
| 5:131709210:C:G | missense | ENST00000510461.6: | ENSP00000421985.1: |
| | | c.769G>C | p.Ala257Pro |
| 5:131709210:C:T | missense | ENST00000510461.6: | ENSP00000421985.1: |
| | | c.769G>A | p.Ala257Thr |
| 5:131709213:T:C | missense | ENST00000510461.6: | ENSP00000421985.1: |
| | | c.766A>G | p.Ile256Val |
| 5:131709215:C:T | missense | ENST00000510461.6: | ENSP00000421985.1: |
| | | c.764G>A | p.Gly255Asp |
| 5:131709216:C:T | missense | ENST00000510461.6: | ENSP00000421985.1: |
| | | c.763G>A | p.Gly255Ser |
| 5:131709218:C:G | missense | ENST00000510461.6: | ENSP00000421985.1: |
| | | c.761G>C | p.Ser254Thr |
| 5:131709219:T:A | missense | ENST00000510461.6: | ENSP00000421985.1: |
| | | c.760A>T | p.Ser254Cys |
| 5:131709219:T:C | missense | ENST00000510461.6: | ENSP00000421985.1: |
| | | c.760A>G | p.Ser254Gly |
| 5:131709226:G:C | missense | ENST00000510461.6: | ENSP00000421985.1: |
| | | c.753C>G | p.Asp251Glu |
| 5:131709227:T:C | missense | ENST00000510461.6: | ENSP00000421985.1: |
| | | c.752A>G | p.Asp251Gly |
| 5:131709228:C:A | missense | ENST00000510461.6: | ENSP00000421985.1: |
| | | c.751G>T | p.Asp251Tyr |
| 5:131709233:T:C | missense | ENST00000510461.6: | ENSP00000421985.1: |
| | | c.746A>G | p.Asn249Ser |
| 5:131709234:T:C | missense | ENST00000510461.6: | ENSP00000421985.1: |
| | | c.745A>G | p.Asn249Asp |
| 5:131709234:T:G | missense | ENST00000510461.6: | ENSP00000421985.1: |
| | | c.745A>C | p.Asn249His |
| 5:131709237:G:A | missense | ENST00000510461.6: | ENSP00000421985.1: |
| | | c.742C>T | p.Leu248Phe |
| 5:131709239:T:C | missense | ENST00000510461.6: | ENSP00000421985.1: |
| | | c.740A>G | p.Glu247Gly |
| 5:131709240:C:G | missense | ENST00000510461.6: | ENSP00000421985.1: |
| | | c.739G>C | p.Glu247Gln |
| 5:131709243:T:C | missense | ENST00000510461.6: | ENSP00000421985.1: |
| | | c.736A>G | p.Arg246Gly |
| 5:131709245:C:T | missense | ENST00000510461.6: | ENSP00000421985.1: |
| | | c.734G>A | p.Gly245Glu |
| 5:131709248:G:A | missense | ENST00000510461.6: | ENSP00000421985.1: |
| | | c.731C>T | p.Pro244Leu |
| 5:131709248:G:C | missense | ENST00000510461.6: | ENSP00000421985.1: |
| | | c.731C>G | p.Pro244Arg |
| 5:131709252:T:C | missense | ENST00000510461.6: | ENSP00000421985.1: |
| | | c.727A>G | p.Met243Val |
| 5:131709258:T:C | missense | ENST00000510461.6: | ENSP00000421985.1: |
| | | c.721A>G | p.Met241Val |
| 5:131709258:T:G | missense | ENST00000510461.6: | ENSP00000421985.1: |
| | | c.721A>C | p.Met241Leu |
| 5:131709260:G:C | missense | ENST00000510461.6: | ENSP00000421985.1: |
| | | c.719C>G | p.Pro240Arg |
| 5:131709269:T:C | missense | ENST00000510461.6: | ENSP00000421985.1: |
| | | c.710A>G | p.His237Arg |
| 5:131709270:G:A | missense | ENST00000510461.6: | ENSP00000421985.1: |
| | | c.709C>T | p.His237Tyr |
| 5:131710581:C:A | missense | ENST00000510461.6: | ENSP00000421985.1: |
| | | c.703G>T | p.Ala235Ser |
| 5:131710581:C:T | missense | ENST00000510461.6: | ENSP00000421985.1: |
| | | c.703G>A | p.Ala235Thr |
| 5:131710582:A:C | missense | ENST00000510461.6: | ENSP00000421985.1: |
| | | c.702T>G | p.Phe234Leu |
| 5:131710584:A:C | missense | ENST00000510461.6: | ENSP00000421985.1: |
| | | c.700T>G | p.Phe234Val |
| 5:131710593:C:T | missense | ENST00000510461.6: | ENSP00000421985.1: |
| | | c.691G>A | p.Ala231Thr |
| 5:131710594:G:T | missense | ENST00000510461.6: | ENSP00000421985.1: |
| | | c.690C>A | p.Ser230Arg |
| 5:131710595:C:T | missense | ENST00000510461.6: | ENSP00000421985.1: |
| | | c.689G>A | p.Ser230Asn |
| 5:131710600:G:C | missense | ENST00000510461.6: | ENSP00000421985.1: |
| | | c.684C>G | p.Ile228Met |
| 5:131710604:A:G | missense | ENST00000510461.6: | ENSP00000421985.1: |
| | | c.680T>C | p.Leu227Pro |
| 5:131710605:G:C | missense | ENST00000510461.6: | ENSP00000421985.1: |
| | | c.679C>G | p.Leu227Val |
| 5:131710607:C:T | missense | ENST00000510461.6: | ENSP00000421985.1: |
| | | c.677G>A | p.Arg226His |
| 5:131710608:G:A | missense | ENST00000510461.6: | ENSP00000421985.1: |
| | | c.676C>T | p.Arg226Cys |
| 5:131710610:A:C | missense | ENST00000510461.6: | ENSP00000421985.1: |
| | | c.674T>G | p.Leu225Arg |
| 5:131710613:G:A | missense | ENST00000510461.6: | ENSP00000421985.1: |
| | | c.671C>T | p.Pro224Leu |
| 5:131710613:G:T | missense | ENST00000510461.6: | ENSP00000421985.1: |
| | | c.671C>A | p.Pro224Gln |
| 5:131710614:G:C | missense | ENST00000510461.6: | ENSP00000421985.1: |
| | | c.670C>G | p.Pro224Ala |
| 5:131710617:C:T | missense | ENST00000510461.6: | ENSP00000421985.1: |
| | | c.667G>A | p.Gly223Ser |
| 5:131710618:C:G | missense | ENST00000510461.6: | ENSP00000421985.1: |
| | | c.666G>C | p.Gln222His |
| 5:131710628:A:G | missense | ENST00000510461.6: | ENSP00000421985.1: |
| | | c.656T>C | p.Phe219Ser |
| 5:131710632:C:G | missense | ENST00000510461.6: | ENSP00000421985.1: |
| | | c.652G>C | p.Ala218Pro |
| 5:131710632:C:T | missense | ENST00000510461.6: | ENSP00000421985.1: |
| | | c.652G>A | p.Ala218Th |
| 5:131710634:C:T | missense | ENST00000510461.6: | ENSP00000421985.1: |
| | | c.650G>A | p.Arg217Gln |
| 5:131710635:G:A | missense | ENST00000510461.6: | ENSP00000421985.1: |
| | | c.649C>T | p.Arg217Trp |
| 5:131710637:C:A | missense | ENST00000510461.6: | ENSP00000421985.1: |
| | | c.647G>T | p.Arg216Met |
| 5:131710637:C:G | missense | ENST00000510461.6: | ENSP00000421985.1: |
| | | c.647G>C | p.Arg216Thr |
| 5:131710638:T:C | missense | ENST00000510461.6: | ENSP00000421985.1: |
| | | c.646A>G | p.Arg216Gly |
| 5:131710640:G:A | missense | ENST00000510461.6: | ENSP00000421985.1: |
| | | c.644C>T | p.Pro215Leu |
| 5:131710640:G:C | missense | ENST00000510461.6: | ENSP00000421985.1: |
| | | c.644C>G | p.Pro215Arg |
| 5:131710643:C:T | missense | ENST00000510461.6: | ENSP00000421985.1: |
| | | c.641G>A | p.Ser214Asn |
| 5:131710644:T:C | missense | ENST00000510461.6: | ENSP00000421985.1: |
| | | c.640A>G | p.Ser214Gly |
| 5:131710647:A:C | missense | ENST00000510461.6: | ENSP00000421985.1: |
| | | c.637T>G | p.Cys213Gly |
| 5:131710649:A:T | missense | ENST00000510461.6: | ENSP00000421985.1: |
| | | c.635T>A | p.Phe212Tyr |
| 5:131710653:G:A | stop_ | ENST00000510461.6: | ENSP00000421985.1: |
| | gained | c.631C>T | p.Gln211Ter |
| 5:131710655:G:A | missense | ENST00000510461.6: | ENSP00000421985.1: |
| | | c.629C>T | p.Ser210Leu |
| 5:131710656:A:G | missense | ENST00000510461.6: | ENSP00000421985.1: |
| | | c.628T>C | p.Ser210Pro |
| 5:131716564:C:G | splice_ | ENST00000510461.6: | NA |
| | donor | c.622+1G>C | |
| 5:131716566:T:C | missense | ENST00000510461.6: | ENSP00000421985.1: |
| | | c.621A>G | p.Ile207Met |
| 5:131716567:A:G | missense | ENST00000510461.6: | ENSP00000421985.1: |
| | | c.620T>C | p.Ile207Thr |
| 5:131716570:T:C | missense | ENST00000510461.6: | ENSP00000421985.1: |
| | | c.617A>G | p.Asn206Ser |
| 5:131716573:C:A | missense | ENST00000510461.6: | ENSP00000421985.1: |
| | | c.614G>T | p.Gly205Val |
| 5:131716582:C:A | missense | ENST00000510461.6: | ENSP00000421985.1: |
| | | c.605G>T | p.Gly202Val |
| 5:131716582:C:T | missense | ENST00000510461.6: | ENSP00000421985.1: |
| | | c.605G>A | p.Gly202Glu |
| 5:131716589:T:C | missense | ENST00000510461.6: | ENSP00000421985.1: |
| | | c.598A>G | p.Ile200Val |
| 5:131716592:C:A | missense | ENST00000510461.6: | ENSP00000421985.1: |
| | | c.595G>T | p.Val199Phe |
| 5:131716592:C:T | missense | ENST00000510461.6: | ENSP00000421985.1: |
| | | c.595G>A | p.Val199Ile |
| 5:131716592:CTG:C | frameshift | ENST00000510461.6: | ENSP00000421985.1: |
| | | c.593_594del | p.Thr198SerfsTer3 |
| 5:131716600:T:A | missense | ENST00000510461.6: | ENSP00000421985.1: |
| | | c.587A>T | p.Asn196Ile |
| 5:131716600:T:C | missense | ENST00000510461.6: | ENSP00000421985.1: |
| | | c.587A>G | p.Asn196Ser |
| 5:131716601:T:C | missense | ENST00000510461.6: | ENSP00000421985.1: |
| | | c.586A>G | p.Asn196Asp |
| 5:131716606:G:C | missense | ENST00000510461.6: | ENSP00000421985.1: |
| | | c.581C>G | p.Ala194Gly |
| 5:131716607:C:A | missense | ENST00000510461.6: | ENSP00000421985.1: |
| | | c.580G>T | p.Ala194Ser |
| 5:131716607:C:T | missense | ENST00000510461.6: | ENSP00000421985.1: |
| | | c.580G>A | p.Ala194Thr |
| 5:131716613:A:C | missense | ENST00000510461.6: | ENSP00000421985.1: |
| | | c.574T>G | p.Leu192Val |
| 5:131716615:G:T | missense | ENST00000510461.6: | ENSP00000421985.1: |
| | | c.572C>A | p.Thr191Lys |
| 5:131716615:GT:G | frameshift | ENST00000510461.6: | ENSP00000421985.1: |
| | | c.571del | p.Thr191HisfsTer2 |
| 5:131716616:T:C | missense | ENST00000510461.6: | ENSP00000421985.1: |
| | | c.571A>G | p.Thr191Ala |
| 5:131716617:A:C | missense | ENST00000510461.6: | ENSP00000421985.1: |
| | | c.570T>G | p.Asn190Lys |
| 5:131716618:T:C | missense | ENST00000510461.6: | ENSP00000421985.1: |
| | | c.569A>G | p.Asn190Ser |
| 5:131716621:T:C | missense | ENST00000510461.6: | ENSP00000421985.1: |
| | | c.566A>G | p.Asn189Ser |
| 5:131716622:T:A | missense | ENST00000510461.6: | ENSP00000421985.1: |
| | | c.565A>T | p.Asn189Tyr |
| 5:131716627:T:C | missense | ENST00000510461.6: | ENSP00000421985.1: |
| | | c.560A>G | p.Gln187Arg |
| 5:131716630:T:A | missense | ENST00000510461.6: | ENSP00000421985.1: |
| | | c.557A>T | p.Asn186Ile |
| 5:131716630:T:C | missense | ENST00000510461.6: | ENSP00000421985.1: |
| | | c.557A>G | p.Asn186Ser |
| 5:131716634:T:C | missense | ENST00000510461.6: | ENSP00000421985.1: |
| | | c.553A>G | p.Ile185Val |
| 5:131716634:T:G | missense | ENST00000510461.6: | ENSP00000421985.1: |
| | | c.553A>C | p.Ile185Leu |
| 5:131716638:T:A | missense | ENST00000510461.6: | ENSP00000421985.1: |
| | | c.549A>T | p.Glu183Asp |
| 5:131716648:T:C | missense | ENST00000510461.6: | ENSP00000421985.1: |
| | | c.539A>G | p.Asp180Gly |
| 5:131716651:T:G | missense | ENST00000510461.6: | ENSP00000421985.1: |
| | | c.536A>C | p.Gln179Pro |
| 5:131716652:G:A | stop_ | ENST00000510461.6: | ENSP00000421985.1: |
| | gained | c.535C>T | p.Gln179Ter |
| 5:131716655:G:T | missense | ENST00000510461.6: | ENSP00000421985.1: |
| | | c.532C>A | p.Leu178Ile |
| 5:131718984:A:T | splice_ | ENST00000510461.6: | NA |
| | donor | c.530+2T>A | |
| 5:131718985:C:T | splice_ | ENST00000510461.6: | NA |
| | donor | c.530+1G>A | |
| 5:131718986:G:A | missense | ENST00000510461.6: | ENSP00000421985.1: |
| | | c.530C>T | p.Thr177Met |
| 5:131718986:G:C | missense | ENST00000510461.6: | ENSP00000421985.1: |
| | | c.530C>G | p.Thr177Arg |
| 5:131718986:G:T | missense | ENST00000510461.6: | ENSP00000421985.1: |
| | | c.530C>A | p.Thr177Lys |
| 5:131718989:T:A | missense | ENST00000510461.6: | ENSP00000421985.1: |
| | | c.527A>T | p.Asn176Ile |
| 5:131718989:T:C | missense | ENST00000510461.6: | ENSP00000421985.1: |
| | | c.527A>G | p.Asn176Ser |
| 5:131718989:T:G | missense | ENST00000510461.6: | ENSP00000421985.1: |
| | | c.527A>C | p.Asn176Thr |
| 5:131718993:G:A | missense | ENST00000510461.6: | ENSP00000421985.1: |
| | | c.523C>T | p.Leu175Phe |
| 5:131718995:C:T | missense | ENST00000510461.6: | ENSP00000421985.1: |
| | | c.521G>A | p.Ser174Asn |
| 5:131718998:C:T | missense | ENST00000510461.6: | ENSP00000421985.1: |
| | | c.518G>A | p.Gly173Glu |
| 5:131719001:C:A | missense | ENST00000510461.6: | ENSP00000421985.1: |
| | | c.515G>T | p.Cys172Phe |
| 5:131719013:C:T | missense | ENST00000510461.6: | ENSP00000421985.1: |
| | | c.503G>A | p.Gly168Asp |
| 5:131719014:C:T | missense | ENST00000510461.6: | ENSP00000421985.1: |
| | | c.502G>A | p.Gly168Ser |
| 5:131719016:G:A | missense | ENST00000510461.6: | ENSP00000421985.1: |
| | | c.500C>T | p.Thr167Ile |
| 5:131719016:G:C | missense | ENST00000510461.6: | ENSP00000421985.1: |
| | | c.500C>G | p.Thr167Ser |
| 5:131719019:C:T | missense | ENST00000510461.6: | ENSP00000421985.1: |
| | | c.497G>A | p.Arg166Gln |
| 5:131719020:G:A | missense | ENST00000510461.6: | ENSP00000421985.1: |
| | | c.496C>T | p.Arg166Trp |
| 5:131719020:G:C | missense | ENST00000510461.6: | ENSP00000421985.1: |
| | | c.496C>G | p.Arg166Gly |
| 5:131719025:G:T | missense | ENST00000510461.6: | ENSP00000421985.1: |
| | | c.491C>A | p.Thr164Asn |
| 5:131719026:T:C | missense | ENST00000510461.6: | ENSP00000421985.1: |
| | | c.490A>G | p.Thr164Ala |
| 5:131719027:A:A | frameshift | ENST00000510461.6: | ENSP00000421985.1: |
| | | c.479_ | p.Phe163LeufsTer22 |
| AACACTTTGC | | 488dup | |
| 5:131719027:A:C | missense | ENST00000510461.6: | ENSP00000421985.1: |
| | | c.489T>G | p.Phe163Leu |
| 5:131719031:A:G | missense | ENST00000510461.6: | ENSP00000421985.1: |
| | | c.485T>C | p.Val162Ala |
| 5:131719032:C:T | missense | ENST00000510461.6: | ENSP00000421985.1: |
| | | c.484G>A | p.Val162Met |
| 5:131719035:T:G | missense | ENST00000510461.6: | ENSP00000421985.1: |
| | | c.481A>C | p.Lys161Gln |
| 5:131719041:G:A | missense | ENST00000510461.6: | ENSP00000421985.1: |
| | | c.475C>T | p.Leu159Phe |
| 5:131719042:C:T | missense | ENST00000510461.6: | ENSP00000421985.1: |
| | | c.474G>A | p.Met158Ile |
| 5:131719043:A:C | missense | ENST00000510461.6: | ENSP00000421985.1: |
| | | c.473T>G | p.Met158Arg |
| 5:131719044:T:G | missense | ENST00000510461.6: | ENSP00000421985.1: |
| | | c.472A>C | p.Met158Leu |
| 5:131719046:A:G | missense | ENST00000510461.6: | ENSP00000421985.1: |
| | | c.470T>C | p.Leu157Pro |
| 5:131719047:G:C | missense | ENST00000510461.6: | ENSP00000421985.1: |
| | | c.469C>G | p.Leu157Val |
| 5:131719048:C:G | missense | ENST00000510461.6: | ENSP00000421985.1: |
| | | c.468G>C | p.Gln156His |
| 5:131719049:T:C | missense | ENST00000510461.6: | ENSP00000421985.1: |
| | | c.467A>G | p.Gln156Arg |
| 5:131719050:G:A | stop_ | ENST00000510461.6: | ENSP00000421985.1: |
| | gained | c.466C>T | p.Gln156Ter |
| 5:131719052:G:A | missense | ENST00000510461.6: | ENSP00000421985.1: |
| | | c.464C>T | p.Pro155Leu |
| 5:131719056:G:A | missense | ENST00000510461.6: | ENSP00000421985.1: |
| | | c.460C>T | p.Pro154Ser |
| 5:131719058:G:A | missense | ENST00000510461.6: | ENSP00000421985.1: |
| | | c.458C>T | p.Ser153Phe |
| 5:131719058:G:T | missense | ENST00000510461.6: | ENSP00000421985.1: |
| | | c.458C>A | p.Ser153Tyr |
| 5:131719317:C:T | missense | ENST00000510461.6: | ENSP00000421985.1: |
| | | c.455G>A | p.Arg152His |
| 5:131719318:G:A | missense | ENST00000510461.6: | ENSP00000421985.1: |
| | | c.454C>T | p.Arg152Cys |
| 5:131719318:G:C | missense | ENST00000510461.6: | ENSP00000421985.1: |
| | | c.454C>G | p.Arg152Gly |
| 5:131719320:A:C | missense | ENST00000510461.6: | ENSP00000421985.1: |
| | | c.452T>G | p.Ile151Ser |
| 5:131719332:T:C | missense | ENST00000510461.6: | ENSP00000421985.1: |
| | | c.440A>G | p.Lys147Arg |
| 5:131719339:T:A | missense | ENST00000510461.6: | ENSP00000421985.1: |
| | | c.433A>T | p.Thr145Ser |
| 5:131719339:T:C | missense | ENST00000510461.6: | ENSP00000421985.1: |
| | | c.433A>G | p.Thr145Ala |
| 5:131719342:A:G | missense | ENST00000510461.6: | ENSP00000421985.1: |
| | | c.430T>C | p.Ser144Pro |
| 5:131719348:T:C | missense | ENST00000510461.6: | ENSP00000421985.1: |
| | | c.424A>G | p.Lys142Glu |
| 5:131719350:T:G | missense | ENST00000510461.6: | ENSP00000421985.1: |
| | | c.422A>C | p.Tyr141Ser |
| 5:131719353:C:A | missense | ENST00000510461.6: | ENSP00000421985.1: |
| | | c.419G>T | p.Ser140Ile |
| 5:131719356:A:C | missense | ENST00000510461.6: | ENSP00000421985.1: |
| | | c.416T>G | p.Met139Arg |
| 5:131719357:T:C | missense | ENST00000510461.6: | ENSP00000421985.1: |
| | | c.415A>G | p.Met139Val |
| 5:131719359:G:A | missense | ENST00000510461.6: | ENSP00000421985.1: |
| | | c.413C>T | p.Ala138Val |
| 5:131719363:C:T | missense | ENST00000510461.6: | ENSP00000421985.1: |
| | | c.409G>A | p.Val137Ile |
| 5:131719366:A:G | missense | ENST00000510461.6: | ENSP00000421985.1: |
| | | c.406T>C | p.Ser136Pro |
| 5:131719368:C:A | missense | ENST00000510461.6: | ENSP00000421985.1: |
| | | c.404G>T | p.Gly135Val |
| 5:131719368:C:G | missense | ENST00000510461.6: | ENSP00000421985.1: |
| | | c.404G>C | p.Gly135Ala |
| 5:131719374:A:G | missense | ENST00000510461.6: | ENSP00000421985.1: |
| | | c.398T>C | p.Met133Thr |
| 5:131719375:T:C | missense | ENST00000510461.6: | ENSP00000421985.1: |
| | | c.397A>G | p.Met133Val |
| 5:131719388:C:T | missense | ENST00000510461.6: | ENSP00000421985.1: |
| | | c.384G>A | p.Met128Ile |
| 5:131719390:T:C | missense | ENST00000510461.6: | ENSP00000421985.1: |
| | | c.382A>G | p.Met128Val |
| 5:131719392:T:C | missense | ENST00000510461.6: | ENSP00000421985.1: |
| | | c.380A>G | p.Asn127Ser |
| 5:131719393:T:C | missense | ENST00000510461.6: | ENSP00000421985.1: |
| | | c.379A>G | p.Asn127Asp |
| 5:131719396:C:T | missense | ENST00000510461.6: | ENSP00000421985.1: |
| | | c.376G>A | p.Ala126Thr |
| 5:131719402:A:G | missense | ENST00000510461.6: | ENSP00000421985.1: |
| | | c.370T>C | p.Ser124Pro |
| 5:131719404:G:A | missense | ENST00000510461.6: | ENSP00000421985.1: |
| | | c.368C>T | p.Ser123Phe |
| 5:131719405:A:G | missense | ENST00000510461.6: | ENSP00000421985.1: |
| | | c.367T>C | p.Ser123Pro |
| 5:131719407:C:A | missense | ENST00000510461.6: | ENSP00000421985.1: |
| | | c.365G>T | p.Cys122Phe |
| 5:131719410:C:T | missense | ENST00000510461.6: | ENSP00000421985.1: |
| | | c.362G>A | p.Arg121Gln |
| 5:131719411:G:A | missense | ENST00000510461.6: | ENSP00000421985.1: |
| | | c.361C>T | p.Arg121Trp |
| 5:131719415:AC:A | frameshift | ENST00000510461.6: | ENSP00000421985.1: |
| | | c.356del | p.Gly119ValfsTer14 |
| 5:131719416:C:T | missense | ENST00000510461.6: | ENSP00000421985.1: |
| | | c.356G>A | p.Gly119Asp |
| 5:131719417:C:G | missense | ENST00000510461.6: | ENSP00000421985.1: |
| | | c.355G>C | p.Gly119Arg |
| 5:131730903:C:G | splice_ | ENST00000510461.6: | NA |
| | donor | c.354+1G>C | |
| 5:131730906:G:T | missense | ENST00000510461.6: | ENSP00000421985.1: |
| | | c.352C>A | p.Gln118Lys |
| 5:131730908:T:C | missense | ENST00000510461.6: | ENSP00000421985.1: |
| | | c.350A>G | p.Tyr117Cys |
| 5:131730909:A:G | missense | ENST00000510461.6: | ENSP00000421985.1: |
| | | c.349T>C | p.Tyr117His |
| 5:131730915:G:C | missense | ENST00000510461.6: | ENSP00000421985.1: |
| | | c.343C>G | p.Leu115Val |
| 5:131730917:C:T | missense | ENST00000510461.6: | ENSP00000421985.1: |
| | | c.341G>A | p.Cys114Tyr |
| 5:131730921:G:C | missense | ENST00000510461.6: | ENSP00000421985.1: |
| | | c.337C>G | p.Gln113Glu |
| 5:131730921:G:T | missense | ENST00000510461.6: | ENSP00000421985.1: |
| | | c.337C>A | p.Gln113Lys |
| 5:131730922:G:C | missense | ENST00000510461.6: | ENSP00000421985.1: |
| | | c.336C>G | p.Asp112Glu |
| 5:131730922:G:T | missense | ENST00000510461.6: | ENSP00000421985.1: |
| | | c.336C>A | p.Asp112Glu |
| 5:131730924:C:G | missense | ENST00000510461.6: | ENSP00000421985.1: |
| | | c.334G>C | p.Asp112His |
| 5:131730926:T:A | missense | ENST00000510461.6: | ENSP00000421985.1: |
| | | c.332A>T | p.Lys111\|le |
| 5:131730927:T:C | missense | ENST00000510461.6: | ENSP00000421985.1: |
| | | c.331A>G | p.Lys111Glu |
| 5:131730929:A:G | missense | ENST00000510461.6: | ENSP00000421985.1: |
| | | c.329T>C | p.Ile110Thr |
| 5:131730932:T:C | missense | ENST00000510461.6: | ENSP00000421985.1: |
| | | c.326A>G | p.Asp109Gly |
| 5:131730933:C:A | missense | ENST00000510461.6: | ENSP00000421985.1: |
| | | c.325G>T | p.Asp109Tyr |
| 5:131730933:C:G | missense | ENST00000510461.6: | ENSP00000421985.1: |
| | | c.325G>C | p.Asp109His |
| 5:131730935:G:C | missense | ENST00000510461.6: | ENSP00000421985.1: |
| | | c.323C>G | p.Ser108Cys |
| 5:131730936:A:C | missense | ENST00000510461.6: | ENSP00000421985.1: |
| | | c.322T>G | p.Ser108Ala |
| 5:131730938:G:T | missense | ENST00000510461.6: | ENSP00000421985.1: |
| | | c.320C>A | p.Ser107Tyr |
| 5:131730939:A:G | missense | ENST00000510461.6: | ENSP00000421985.1: |
| | | c.319T>C | p.Ser107Pro |
| 5:131730945:T:C | missense | ENST00000510461.6: | ENSP00000421985.1: |
| | | c.313A>G | p.Thr105Ala |
| 5:131730948:C:A | missense | ENST00000510461.6: | ENSP00000421985.1: |
| | | c.310G>T | p.Val104Leu |
| 5:131730948:C:G | missense | ENST00000510461.6: | ENSP00000421985.1: |
| | | c.310G>C | p.Val104Leu |
| 5:131730948:C:T | missense | ENST00000510461.6: | ENSP00000421985.1: |
| | | c.310G>A | p.Val104Met |
| 5:131730950:G:C | missense | ENST00000510461.6: | ENSP00000421985.1: |
| | | c.308C>G | p.Ser103Cys |
| 5:131730950:G:T | missense | ENST00000510461.6: | ENSP00000421985.1: |
| | | c.308C>A | p.Ser103Tyr |
| 5:131730950:GAACT: | frameshift | ENST00000510461.6: | ENSP00000421985.1: |
| G | | c.304_ | p.Ser102LeufsTer2 |
| | | 307del | |
| 5:131730952:A:C | missense | ENST00000510461.6: | ENSP00000421985.1: |
| | | c.306T>G | p.Ser102Arg |
| 5:131730952:A:T | missense | ENST00000510461.6: | ENSP00000421985.1: |
| | | c.306T>A | p.Ser102Arg |
| 5:131730953:C:A | missense | ENST00000510461.6: | ENSP00000421985.1: |
| | | c.305G>T | p.Ser102Ile |
| 5:131730956:T:C | missense | ENST00000510461.6: | ENSP00000421985.1: |
| | | c.302A>G | p.Asp101Gly |
| 5:131730957:C:A | missense | ENST00000510461.6: | ENSP00000421985.1: |
| | | c.301G>T | p.Asp101Tyr |
| 5:131730959:A:G | missense | ENST00000510461.6: | ENSP00000421985.1: |
| | | c.299T>C | p.Leu100Ser |
| 5:131730960:A:C | missense | ENST00000510461.6: | ENSP00000421985.1: |
| | | c.298T>G | p.Leu100Val |
| 5:131730965:G:C | missense | ENST00000510461.6: | ENSP00000421985.1: |
| | | c.293C>G | p.Ser98Cys |
| 5:131730968:G:T | missense | ENST00000510461.6: | ENSP00000421985.1: |
| | | c.290C>A | p.Ser97Tyr |
| 5:131730969:A:T | missense | ENST00000510461.6: | ENSP00000421985.1: |
| | | c.289T>A | p.Ser97Thr |
| 5:131730971:C:G | missense | ENST00000510461.6: | ENSP00000421985.1: |
| | | c.287G>C | p.Ser96Thr |
| 5:131730971:C:T | missense | ENST00000510461.6: | ENSP00000421985.1: |
| | | c.287G>A | p.Ser96Asn |
| 5:131730972:T:C | missense | ENST00000510461.6: | ENSP00000421985.1: |
| | | c.286A>G | p.Ser96Gly |
| 5:131730972:T:G | missense | ENST00000510461.6: | ENSP00000421985.1: |
| | | c.286A>C | p.Ser96Arg |
| 5:131730974:T:C | missense | ENST00000510461.6: | ENSP00000421985.1: |
| | | c.284A>G | p.Asp95Gly |
| 5:131730975:C:T | missense | ENST00000510461.6: | ENSP00000421985.1: |
| | | c.283G>A | p.Asp95Asn |
| 5:131730978:C:A | stop_ | ENST00000510461.6: | ENSP00000421985.1: |
| | gained | c.280G>T | p.Gly94Ter |
| 5:131730978:C:T | missense | ENST00000510461.6: | ENSP00000421985.1: |
| | | c.280G>A | p.Gly94Arg |
| 5:131730980:C:T | missense | ENST00000510461.6: | ENSP00000421985.1: |
| | | c.278G>A | p.Gly93Glu |
| 5:131730981:C:T | missense | ENST00000510461.6: | ENSP00000421985.1: |
| | | c.277G>A | p.Gly93Arg |
| 5:131730983:G:A | missense | ENST00000510461.6: | ENSP00000421985.1: |
| | | c.275C>T | p.Pro92Leu |
| 5:131730984:G:C | missense | ENST00000510461.6: | ENSP00000421985.1: |
| | | c.274C>G | p.Pro92Ala |
| 5:131730986:T:G | missense | ENST00000510461.6: | ENSP00000421985.1: |
| | | c.272A>C | p.Lys91Thr |
| 5:131730987:T:G | missense | ENST00000510461.6: | ENSP00000421985.1: |
| | | c.271A>C | p.Lys91Gln |
| 5:131730989:A:G | missense | ENST00000510461.6: | ENSP00000421985.1: |
| | | c.269T>C | p.Leu90Pro |
| 5:131730991:T:G | missense | ENST00000510461.6: | ENSP00000421985.1: |
| | | c.267A>C | p.Gln89His |
| 5:131730992:T:C | missense | ENST00000510461.6: | ENSP00000421985.1: |
| | | c.266A>G | p.Gln89Arg |
| 5:131730993:G:A | stop_ | ENST00000510461.6: | ENSP00000421985.1: |
| | gained | c.265C>T | p.Gln89Ter |
| 5:131730995:C:T | missense | ENST00000510461.6: | ENSP00000421985.1: |
| | | c.263G>A | p.Cys88Tyr |
| 5:131730999:A:G | missense | ENST00000510461.6: | ENSP00000421985.1: |
| | | c.259T>C | p.Cys87Arg |
| 5:131731001:T:C | missense | ENST00000510461.6: | ENSP00000421985.1: |
| | | c.257A>G | p.Lys86Arg |
| 5:131731004:C:A | missense | ENST00000510461.6: | ENSP00000421985.1: |
| | | c.254G>T | p.Gly85Val |
| 5:131731004:C:G | missense | ENST00000510461.6: | ENSP00000421985.1: |
| | | c.254G>C | p.Gly85Ala |
| 5:131731007:A:C | missense | ENST00000510461.6: | ENSP00000421985.1: |
| | | c.251T>G | p.Phe84Cys |
| 5:131731010:A:C | missense | ENST00000510461.6: | ENSP00000421985.1: |
| | | c.248T>G | p.Val83Gly |
| 5:131731011:C:A | missense | ENST00000510461.6: | ENSP00000421985.1: |
| | | c.247G>T | p.Val83Phe |
| 5:131731011:C:T | missense | ENST00000510461.6: | ENSP00000421985.1: |
| | | c.247G>A | p.Val83Ile |
| 5:131731014:T:C | missense | ENST00000510461.6: | ENSP00000421985.1: |
| | | c.244A>G | p.Lys82Glu |
| 5:131731016:A:T | missense | ENST00000510461.6: | ENSP00000421985.1: |
| | | c.242T>A | p.Val81Asp |
| 5:131731019:T:G | missense | ENST00000510461.6: | ENSP00000421985.1: |
| | | c.239A>C | p.Gln80Pro |
| 5:131731020:G:A | stop_ | ENST00000510461.6: | ENSP00000421985.1: |
| | gained | c.238C>T | p.Gln80Ter |
| 5:131731022:G:C | missense | ENST00000510461.6: | ENSP00000421985.1: |
| | | c.236C>G | p.Ala79Gly |
| 5:131731023:C:T | missense | ENST00000510461.6: | ENSP00000421985.1: |
| | | c.235G>A | p.Ala79Thr |
| 5:131731026:C:T | missense | ENST00000510461.6: | ENSP00000421985.1: |
| | | c.232G>A | p.Asp78Asn |
| 5:131731028:C:T | missense | ENST00000510461.6: | ENSP00000421985.1: |
| | | c.230G>A | p.Ser77Asn |
| 5:131731029:T:C | missense | ENST00000510461.6: | ENSP00000421985.1: |
| | | c.229A>G | p.Ser77Gly |
| 5:131731031:C:A | missense | ENST00000510461.6: | ENSP00000421985.1: |
| | | c.227G>T | p.Gly76Val |
| 5:131731035:G:C | missense | ENST00000510461.6: | ENSP00000421985.1: |
| | | c.223C>G | p.Leu75Val |
| 5:131731039:C:G | splice_ | ENST00000510461.6: | NA |
| | acceptor | c.220−1G>C | |
| 5:131731040:T:C | splice_ | ENST00000510461.6: | NA |
| | acceptor | c.220−2A>G | |
| 5:131744563:C:A | splice_ | ENST00000510461.6: | NA |
| | donor | c.219+1G>T | |
| 5:131744565:G:A | missense | ENST00000510461.6: | ENSP00000421985.1: |
| | | c.218C>T | p.Ser73Leu |
| 5:131744565:G:T | stop_ | ENST00000510461.6: | ENSP00000421985.1: |
| | gained | c.218C>A | p.Ser73Ter |
| 5:131744568:A:T | missense | ENST00000510461.6: | ENSP00000421985.1: |
| | | c.215T>A | p.Val72Glu |
| 5:131744569:C:T | missense | ENST00000510461.6: | ENSP00000421985.1: |
| | | c.214G>A | p.Val72Ile |
| 5:131744571:G:A | missense | ENST00000510461.6: | ENSP00000421985.1: |
| | | c.212C>T | p.Ser71Leu |
| 5:131744572:A:AT | frameshift | ENST00000510461.6: | ENSP00000421985.1: |
| | | c.210dup | p.Ser71IlefsTer8 |
| 5:131744572:A:G | missense | ENST00000510461.6: | ENSP00000421985.1: |
| | | c.211T>C | p.Ser71Pro |
| 5:131744577:T:A | missense | ENST00000510461.6: | ENSP00000421985.1: |
| | | c.206A>T | p.Asp69Val |
| 5:131744577:T:C | missense | ENST00000510461.6: | ENSP00000421985.1: |
| | | c.206A>G | p.Asp69Gly |
| 5:131744578:C:A | missense | ENST00000510461.6: | ENSP00000421985.1: |
| | | c.205G>T | p.Asp69Tyr |
| 5:131744578:C:T | missense | ENST00000510461.6: | ENSP00000421985.1: |
| | | c.205G>A | p.Asp69Asn |
| 5:131744590:T:C | missense | ENST00000510461.6: | ENSP00000421985.1: |
| | | c.193A>G | p.Arg65Gly |
| 5:131744596:C:T | missense | ENST00000510461.6: | ENSP00000421985.1: |
| | | c.187G>A | p.Val63Ile |
| 5:131744598:C:A | missense | ENST00000510461.6: | ENSP00000421985.1: |
| | | c.185G>T | p.Ser62Ile |
| 5:131744599:T:C | missense | ENST00000510461.6: | ENSP00000421985.1: |
| | | c.184A>G | p.Ser62Gly |
| 5:131744601:G:C | missense | ENST00000510461.6: | ENSP00000421985.1: |
| | | c.182C>G | p.Ser61Cys |
| 5:131744605:C:G | missense | ENST00000510461.6: | ENSP00000421985.1: |
| | | c.178G>C | p.Asp60His |
| 5:131744613:A:G | missense | ENST00000510461.6: | ENSP00000421985.1: |
| | | c.170T>C | p.Val57Ala |
| 5:131744616:T:C | missense | ENST00000510461.6: | ENSP00000421985.1: |
| | | c.167A>G | p.Asn56Ser |
| 5:131744617:T:G | missense | ENST00000510461.6: | ENSP00000421985.1: |
| | | c.166A>C | p.Asn56His |
| 5:131744623:C:A | missense | ENST00000510461.6: | ENSP00000421985.1: |
| | | c.160G>T | p.Gly54Trp |
| 5:131744623:C:G | missense | ENST00000510461.6: | ENSP00000421985.1: |
| | | c.160G>C | p.Gly54Arg |
| 5:131744623:C:T | missense | ENST00000510461.6: | ENSP00000421985.1: |
| | | c.160G>A | p.Gly54Arg |
| 5:131744623:CT:C | frameshift | ENST00000510461.6: | ENSP00000421985.1: |
| | | c.159del | p.Arg55GlufsTer36 |
| 5:131744625:C:T | missense | ENST00000510461.6: | ENSP00000421985.1: |
| | | c.158G>A | p.Arg53Gln |
| 5:131744626:G:A | stop_ | ENST00000510461.6: | ENSP00000421985.1: |
| | gained | c.157C>T | p.Arg53Ter |
| 5:131744626:G:C | missense | ENST00000510461.6: | ENSP00000421985.1: |
| | | c.157C>G | p.Arg53Gly |
| 5:131744629:T:C | missense | ENST00000510461.6: | ENSP00000421985.1: |
| | | c.154A>G | p.Arg52Gly |
| 5:131744632:C:T | missense | ENST00000510461.6: | ENSP00000421985.1: |
| | | c.151G>A | p.Glu51Lys |
| 5:131744634:C:G | missense | ENST00000510461.6: | ENSP00000421985.1: |
| | | c.149G>C | p.Cys50Ser |
| 5:131744634:C:T | missense | ENST00000510461.6: | ENSP00000421985.1: |
| | | c.149G>A | p.Cys50Tyr |
| 5:131744635:A:G | missense | ENST00000510461.6: | ENSP00000421985.1: |
| | | c.148T>C | p.Cys50Arg |
| 5:131744642:A:C | stop_ | ENST00000510461.6: | ENSP00000421985.1: |
| | gained | c.141T>G | p.Tyr47Ter |
| 5:131744644:A:AT | frameshift | ENST00000510461.6: | ENSP00000421985.1: |
| | | c.138dup | p.Tyr47IlefsTer5 |
| 5:131744646:A:G | missense | ENST00000510461.6: | ENSP00000421985.1: |
| | | c.137T>C | p.Val46Ala |
| 5:131744647:C:T | missense | ENST00000510461.6: | ENSP00000421985.1: |
| | | c.136G>A | p.Val46Ile |
| 5:131744648:A:C | missense | ENST00000510461.6: | ENSP00000421985.1: |
| | | c.135T>G | p.Ile45Met |
| 5:131744650:T:C | missense | ENST00000510461.6: | ENSP00000421985.1: |
| | | c.133A>G | p.Ile45Val |
| 5:131744655:C:T | missense | ENST00000510461.6: | ENSP00000421985.1: |
| | | c.128G>A | p.Arg43Gln |
| 5:131744656:G:A | stop_ | ENST00000510461.6: | ENSP00000421985.1: |
| | gained | c.127C>T | p.Arg43Ter |
| 5:131744658:A:G | missense | ENST00000510461.6: | ENSP00000421985.1: |
| | | c.125T>C | p.Ile42Thr |
| 5:131744659:T:C | missense | ENST00000510461.6: | ENSP00000421985.1: |
| | | c.124A>G | p.Ile42Val |
| 5:131744660:C:G | missense | ENST00000510461.6: | ENSP00000421985.1: |
| | | c.123G>C | p.Gln41His |
| 5:131744662:G:A | stop_ | ENST00000510461.6: | ENSP00000421985.1: |
| | gained | c.121C>T | p.Gln41Ter |
| 5:131744662:G:C | missense | ENST00000510461.6: | ENSP00000421985.1: |
| | | c.121C>G | p.Gln41Glu |
| 5:131744668:G:T | missense | ENST00000510461.6: | ENSP00000421985.1: |
| | | c.115C>A | p.Pro39Thr |
| 5:131744670:T:G | missense | ENST00000510461.6: | ENSP00000421985.1: |
| | | c.113A>C | p.Asp38Ala |
| 5:131744671:C:G | missense | ENST00000510461.6: | ENSP00000421985.1: |
| | | c.112G>C | p.Asp38His |
| 5:131744671:C:T | missense | ENST00000510461.6: | ENSP00000421985.1: |
| | | c.112G>A | p.Asp38Asn |
| 5:131744673:A:G | missense | ENST00000510461.6: | ENSP00000421985.1: |
| | | c.110T>C | p.Phe37Ser |
| 5:131744675:C:A | missense | ENST00000510461.6: | ENSP00000421985.1: |
| | | c.108G>T | p.Glu36Asp |
| 5:131744676:T:C | missense | ENST00000510461.6: | ENSP00000421985.1: |
| | | c.107A>G | p.Glu36Gly |
| 5:131744676:T:G | missense | ENST00000510461.6: | ENSP00000421985.1: |
| | | c.107A>C | p.Glu36Ala |
| 5:131744677:C:G | missense | ENST00000510461.6: | ENSP00000421985.1: |
| | | c.106G>C | p.Glu36Gln |
| 5:131744679:G:A | missense | ENST00000510461.6: | ENSP00000421985.1: |
| | | c.104C>T | p.Pro35Leu |
| 5:131744680:G:A | missense | ENST00000510461.6: | ENSP00000421985.1: |
| | | c.103C>T | p.Pro35Ser |
| 5:131796828:A:G | splice_ | ENST00000510461.6: | NA |
| | donor | c.92+2T>C | |
| 5:131796829:C:T | splice_ | ENST00000510461.6: | NA |
| | donor | c.92+1G>A | |
| 5:131796830:C:G | missense | ENST00000510461.6: | ENSP00000421985.1: |
| | | c.92G>C | p.Ser31Thr |
| 5:131796834:A:G | missense | ENST00000510461.6: | ENSP00000421985.1: |
| | | c.88T>C | p.Phe30Leu |
| 5:131796838:G:C | missense | ENST00000510461.6: | ENSP00000421985.1: |
| | | c.84C>G | p.Cys28Trp |
| 5:131796843:C:A | missense | ENST00000510461.6: | ENSP00000421985.1: |
| | | c.79G>T | p.Asp27Tyr |
| 5:131796843:C:G | missense | ENST00000510461.6: | ENSP00000421985.1: |
| | | c.79G>C | p.Asp27His |
| 5:131796845:G:A | missense | ENST00000510461.6: | ENSP00000421985.1: |
| | | c.77C>T | p.Pro26Leu |
| 5:131796849:C:A | missense | ENST00000510461.6: | ENSP00000421985.1: |
| | | c.73G>T | p.Asp25Tyr |
| 5:131796851:C:G | missense | ENST00000510461.6: | ENSP00000421985.1: |
| | | c.71G>C | p.Arg24Pro |
| 5:131796851:C:T | missense | ENST00000510461.6: | ENSP00000421985.1: |
| | | c.71G>A | p.Arg24Gln |
| 5:131796852:G:A | missense | ENST00000510461.6: | ENSP00000421985.1: |
| | | c.70C>T | p.Arg24Trp |
| 5:131796852:G:C | missense | ENST00000510461.6: | ENSP00000421985.1: |
| | | c.70C>G | p.Arg24Gly |
| 5:131796858:C:A | missense | ENST00000510461.6: | ENSP00000421985.1: |
| | | c.64G>T | p.Asp22Tyr |
| 5:131796860:C:T | missense | ENST00000510461.6: | ENSP00000421985.1: |
| | | c.62G>A | p.Arg21His |
| 5:131796863:C:A | missense | ENST00000510461.6: | ENSP00000421985.1: |
| | | c.59G>T | p.Gly20Val |
| 5:131796863:C:T | missense | ENST00000510461.6: | ENSP00000421985.1: |
| | | c.59G>A | p.Gly20Asp |
| 5:131796866:G:C | missense | ENST00000510461.6: | ENSP00000421985.1: |
| | | c.56C>G | p.Pro19Arg |
| 5:131796867:G:A | missense | ENST00000510461.6: | ENSP00000421985.1: |
| | | c.55C>T | p.Pro19Ser |
| 5:131796872:C:A | missense | ENST00000510461.6: | ENSP00000421985.1: |
| | | c.50G>T | p.Gly17Val |
| 5:131796872:C:G | missense | ENST00000510461.6: | ENSP00000421985.1: |
| | | c.50G>C | p.Gly17Ala |
| 5:131796875:A:G | missense | ENST00000510461.6: | ENSP00000421985.1: |
| | | c.47T>C | p.Leu16Pro |
| 5:131796878:C:T | missense | ENST00000510461.6: | ENSP00000421985.1: |
| | | c.44G>A | p.Gly15Glu |
| 5:131796879:C:T | missense | ENST00000510461.6: | ENSP00000421985.1: |
| | | c.43G>A | p.Gly15Arg |
| 5:131796883:C:G | missense | ENST00000510461.6: | ENSP00000421985.1: |
| | | c.39G>C | p.Arg13Ser |
| 5:131796884:C:G | missense | ENST00000510461.6: | ENSP00000421985.1: |
| | | c.38G>C | p.Arg13Thr |
| 5:131796887:T:C | missense | ENST00000510461.6: | ENSP00000421985.1: |
| | | c.35A>G | p.Lys12Arg |
| 5:131796889:G:C | missense | ENST00000510461.6: | ENSP00000421985.1: |
| | | c.33C>G | p.Ser11Arg |
| 5:131796891:T:A | missense | ENST00000510461.6: | ENSP00000421985.1: |
| | | c.31A>T | p.Ser11Cys |
| 5:131796897:G:C | missense | ENST00000510461.6: | ENSP00000421985.1: |
| | | c.25C>G | p.Leu9Val |
| 5:131796899:T:C | missense | ENST00000510461.6: | ENSP00000421985.1: |
| | | c.23A>G | p.Lys8Arg |
| 5:131796900:T:C | missense | ENST00000510461.6: | ENSP00000421985.1: |
| | | c.22A>G | p.Lys8Glu |
| 5:131796901:C:G | missense | ENST00000510461.6: | ENSP00000421985.1: |
| | | c.21G>C | p.Gln7His |
| 5:131796904:G:C | missense | ENST00000510461.6: | ENSP00000421985.1: |
| | | c.18C>G | p.Phe6Leu |
| 5:131796906:A:G | missense | ENST00000510461.6: | ENSP00000421985.1: |
| | | c.16T>C | p.Phe6Leu |
| 5:131796911:G:A | missense | ENST00000510461.6: | ENSP00000421985.1: |
| | | c.11C>T | p.Thr4Met |
| 5:131796911:G:T | missense | ENST00000510461.6: | ENSP00000421985.1: |
| | | c.11C>A | p.Thr4Lys |
| 5:131796912:T:A | missense | ENST00000510461.6: | ENSP00000421985.1: |
| | | c.10A>T | p.Thr4Ser |
| 5:131796914:G:C | missense | ENST00000510461.6: | ENSP00000421985.1: |
| | | c.8C>G | p.Pro3Arg |
| 5:131796915:G:A | missense | ENST00000510461.6: | ENSP00000421985.1: |
| | | c.7C>T | p.Pro3Ser |
| 5:131796917:G:A | missense | ENST00000510461.6: | ENSP00000421985.1: |
| | | c.5C>T | p.Ala2Val |

TABLE 9
Predicted loss of function or deleterious missense
variants in FLCN identified by whole exome sequencing

| Variant | Effect | hgvsc | hgvsp |
|---|---|---|---|
| 17:17213659: | frameshift | ENST00000285071.9: | ENSP00000285071.4: |
| TTCCGAGAC:T | | c.17281735del | p.Ser577LeufsTer22 |
| 17:17213680:G:T | missense | ENST00000285071.9: | ENSP00000285071.4: |
| | | c.1715C>A | p.Pro572His |
| 17:17213686:C:G | missense | ENST00000285071.9: | ENSP00000285071.4: |
| | | c.1709G>C | p.Arg570Pro |
| 17:17213686:C:T | missense | ENST00000285071.9: | ENSP00000285071.4: |
| | | c.1709G>A | p.Arg570His |
| 17:17213686:CG:C | frameshift | ENST00000285071.9: | ENSP00000285071.4: |
| | | c.1708del | p.Arg570AlafsTer18 |
| 17:17213687:G:A | missense | ENST00000285071.9: | ENSP00000285071.4: |
| | | c.1708C>T | p.Arg570Cys |
| 17:17213695:G:A | missense | ENST00000285071.9: | ENSP00000285071.4: |
| | | c.1700C>T | p.Ser567Phe |
| 17:17213703:G:C | missense | ENST00000285071.9: | ENSP00000285071.4: |
| | | c.1692C>G | p.His564Gln |
| 17:17213705:G:A | missense | ENST00000285071.9: | ENSP00000285071.4: |
| | | c.1690C>T | p.His564Tyr |
| 17:17213705:GT:G | frameshift | ENST00000285071.9: | ENSP00000285071.4: |
| | | c.1689del | p.His564ThrfsTer24 |
| 17:17213711:T:C | missense | ENST00000285071.9: | ENSP00000285071.4: |
| | | c.1684A>G | p.Lys562Glu |
| 17:17213712:G:C | stop_ | ENST00000285071.9: | ENSP00000285071.4: |
| | gained | c.1683C>G | p.Tyr561Ter |
| 17:17213712:G:T | stop_ | ENST00000285071.9: | ENSP00000285071.4: |
| | gained | c.1683C>A | p.Tyr561Ter |
| 17:17213737:C:T | stop_ | ENST00000285071.9: | ENSP00000285071.4: |
| | gained | c.1658G>A | p.Trp553Ter |
| 17:17213738:A:G | missense | ENST00000285071.9: | ENSP00000285071.4: |
| | | c.1657T>C | p.Trp553Arg |
| 17:17213747:G:C | missense | ENST00000285071.9: | ENSP00000285071.4: |
| | | c.1648C>G | p.Leu550Val |
| 17:17213749:A:G | missense | ENST00000285071.9: | ENSP00000285071.4: |
| | | c.1646T>C | p.Leu549Pro |
| 17:17213758:T:C | missense | ENST00000285071.9: | ENSP00000285071.4: |
| | | c.1637A>G | p.Asn546Ser |
| 17:17213789:G:T | missense | ENST00000285071.9: | ENSP00000285071.4: |
| | | c.1606C>A | p.Leu536Met |
| 17:17213796:C:G | missense | ENST00000285071.9: | ENSP00000285071.4: |
| | | c.1599G>C | p.Gln533His |
| 17:17213796:CTG:C | frameshift | ENST00000285071.9: | ENSP00000285071.4: |
| | | c.1597_1598del | p.Gln533 |
| | | | GlufsTer68 |
| 17:17213798:G:A | stop_ | ENST00000285071.9: | ENSP00000285071.4: |
| | gained | c.1597 | p.Gln533 |
| | | C>T | Ter |
| 17:17213805:C:G | missense | ENST00000285071.9: | ENSP00000285071.4: |
| | | c.1590G>C | p.Glu530Asp |
| 17:17213815:C:CT | frameshift | ENST00000285071.9: | ENSP00000285071.4: |
| | | c.15791580insA | p.Arg527GlnfsTer75 |
| 17:17213815:C:T | missense | ENST00000285071.9: | ENSP00000285071.4: |
| | | c.1580G>A | p.Arg527Gln |
| 17:17213816:G:A | stop_ | ENST00000285071.9: | ENSP00000285071.4: |
| | gained | c.1579C>T | p.Arg527Ter |
| 17:17213823:C:CA | frameshift | ENST00000285071.9: | ENSP00000285071.4: |
| | | c.1571dup | p.Asp525GlyfsTer77 |
| 17:17213834:A:C | missense | ENST00000285071.9: | ENSP00000285071.4: |
| | | c.1561T>G | p.Phe521Val |
| 17:17213836:T:C | missense | ENST00000285071.9: | ENSP00000285071.4: |
| | | c.1559A>G | p.Lys520Arg |
| 17:17213848:T:C | missense | ENST00000285071.9: | ENSP00000285071.4: |
| | | c.1547A>G | p.Lys516Arg |
| 17:17213849:T:C | missense | ENST00000285071.9: | ENSP00000285071.4: |
| | | c.1546A>G | p.Lys516Glu |
| 17:17213852:C:T | missense | ENST00000285071.9: | ENSP00000285071.4: |
| | | c.1543G>A | p.Val515Met |
| 17:17213855:T:C | missense | ENST00000285071.9: | ENSP00000285071.4: |
| | | c.1540A>G | p.Lys514Glu |
| 17:17214984:C:G | splice_ | ENST00000285071.9: | NA |
| | donor | c.1538+1G>C | |
| 17:17214984:C:T | splice_ | ENST00000285071.9: | NA |
| | donor | c.1538+1G>A | |
| 17:17214993:C:G | missense | ENST00000285071.9: | ENSP00000285071.4: |
| | | c.1530G>C | p.Glu510Asp |
| 17:17214995:C:T | missense | ENST00000285071.9: | ENSP00000285071.4: |
| | | c.1528G>A | p.Glu510Lys |
| 17:17214996:C:A | missense | ENST00000285071.9: | ENSP00000285071.4: |
| | | c.1527G>T | p.Glu509Asp |
| 17:17215001:T:A | stop_ | ENST00000285071.9: | ENSP00000285071.4: |
| | gained | c.1522A>T | p.Lys508Ter |
| 17:17215001:T:G | missense | ENST00000285071.9: | ENSP00000285071.4: |
| | | c.1522A>C | p.Lys508Gln |
| 17:17215004:G:A | missense | ENST00000285071.9: | ENSP00000285071.4: |
| | | c.1519C>T | p.Leu507Phe |
| 17:17215006:C:T | missense | ENST00000285071.9: | ENSP00000285071.4: |
| | | c.1517G>A | p.Cys506Tyr |
| 17:17215013:G:A | missense | ENST00000285071.9: | ENSP00000285071.4: |
| | | c.1510C>T | p.Leu504Phe |
| 17:17215015:C:T | missense | ENST00000285071.9: | ENSP00000285071.4: |
| | | c.1508G>A | p.Cys503Tyr |
| 17:17215016:A:G | missense | ENST00000285071.9: | ENSP00000285071.4: |
| | | c.1507T>C | p.Cys503Arg |
| 17:17215022:C:A | missense | ENST00000285071.9: | ENSP00000285071.4: |
| | | c.1501G>T | p.Asp501Tyr |
| 17:17215024:A:C | missense | ENST00000285071.9: | ENSP00000285071.4: |
| | | c.1499T>G | p.Val500Gly |
| 17:17215036:G:A | missense | ENST00000285071.9: | ENSP00000285071.4: |
| | | c.1487C>T | p.Ser496Phe |
| 17:17215036:G:C | missense | ENST00000285071.9: | ENSP00000285071.4: |
| | | c.1487C>G | p.Ser496Cys |
| 17:17215037:A:C | missense | ENST00000285071.9: | ENSP00000285071.4: |
| | | c.1486T>G | p.Ser496Ala |
| 17:17215048:T:C | missense | ENST00000285071.9: | ENSP00000285071.4: |
| | | c.1475A>G | p.Asn492Ser |
| 17:17215057:G:C | missense | ENST00000285071.9: | ENSP00000285071.4: |
| | | c.1466C>G | p.Ala489Gly |
| 17:17215070:T:C | missense | ENST00000285071.9: | ENSP00000285071.4: |
| | | c.1453A>G | p.Lys485Glu |
| 17:17215073:T:G | missense | ENST00000285071.9: | ENSP00000285071.4: |
| | | c.1450A>C | p.Asn484His |
| 17:17215082:TG:T | frameshift | ENST00000285071.9: | ENSP00000285071.4: |
| | | c.1440del | p.Thr481ProfsTer3 |
| 17:17215087:C:T | missense | ENST00000285071.9: | ENSP00000285071.4: |
| | | c.1436G>A | p.Gly479Asp |
| 17:17215184:C:G | splice_ | ENST00000285071.9: | NA |
| | donor | c.1432+1G>C | |
| 17:17215184:C:T | splice_ | ENST00000285071.9: | NA |
| | donor | c.1432+1G>A | |
| 17:17215188:G:A | stop_ | ENST00000285071.9: | ENSP00000285071.4: |
| | gained | c.1429C>T | p.Arg477Ter |
| 17:17215190:T:TC | frameshift | ENST00000285071.9: | ENSP00000285071.4: |
| | | c.1426dup | p.Asp476GlyfsTer10 |
| 17:17215224:A:AC | frameshift | ENST00000285071.9: | ENSP00000285071.4: |
| | | c.1392dup | p.Phe465ValfsTer21 |
| 17:17215228:G:C | stop_ | ENST00000285071.9: | ENSP00000285071.4: |
| | gained | c.1389C>G | p.Tyr463Ter |
| 17:17215236:TGA:T | frameshift | ENST00000285071.9: | ENSP00000285071.4: |
| | | c.13791380del | p.Leu460GlnfsTer25 |
| 17:17215256:CA:C | frameshift | ENST00000285071.9: | ENSP00000285071.4: |
| | | c.1360del | p.Cys454ValfsTer14 |
| 17:17215257:AC:A | frameshift | ENST00000285071.9: | ENSP00000285071.4: |
| | | c.1359del | p.Cys454ValfsTer14 |
| 17:17215263:C: | frameshift | ENST00000285071.9: | ENSP00000285071.4: |
| CAGGGTGG | | c.13471353dup | p.Val452ProfsTer6 |
| 17:17215264:AGGGT | frameshift | ENST00000285071.9: | ENSP00000285071.4: |
| GGAGGGTGGAACGTG | | c.13331352del | p.Ala445CysfsTer4 |
| C:A | | | |
| 17:17215282:T:TG | frameshift | ENST00000285071.9: | ENSP00000285071.4: |
| CGGCTGCGTGGACCTC | | c.1318_ | p.Leu449GlnfsTer25 |
| | | 1334dup | |
| 17:17215302:C:T | missense | ENST00000285071.9: | ENSP00000285071.4: |
| | | c.1315G>A | p.Val439Met |
| 17:17215311:CA:C | frameshift | ENST00000285071.9: | ENSP00000285071.4: |
| | | c.1305del | p.Phe435LeufsTer33 |
| 17:17215313:A:C | missense | ENST00000285071.9: | ENSP00000285071.4: |
| | | c.1304T>G | p.Phe435Cys |
| 17:17215317: | splice_ | ENST00000285071.9: | NA |
| CTG:C | acceptor | c.1301−3_ | |
| | | 1301−2del | |
| 17:17216382:G:A | missense | ENST00000285071.9: | ENSP00000285071.4: |
| | | c.1298C>T | p.Ser433Leu |
| 17:17216394:T:TG | frameshift | ENST00000285071.9: | ENSP00000285071.4: |
| | | c.1285dup | p.His429ProfsTer27 |
| 17:17216394:TG:T | frameshift | ENST00000285071.9: | ENSP00000285071.4: |
| | | c.1285del | p.His429ThrfsTer39 |
| 17:17216395:G:T | missense | ENST00000285071.9: | ENSP00000285071.4: |
| | | c.1285C>A | p.His429Asn |
| 17:17216400:G:A | missense | ENST00000285071.9: | ENSP00000285071.4: |
| | | c.1280C>T | p.Pro427Leu |
| 17:17216400:G:C | missense | ENST00000285071.9: | ENSP00000285071.4: |
| | | c.1280C>G | p.Pro427Arg |
| 17:17216400:G:T | missense | ENST00000285071.9: | ENSP00000285071.4: |
| | | c.1280C>A | p.Pro427His |
| 17:17216401:G:A | missense | ENST00000285071.9: | ENSP00000285071.4: |
| | | c.1279C>T | p.Pro427Ser |
| 17:17216401:G:T | missense | ENST00000285071.9: | ENSP00000285071.4: |
| | | c.1279C>A | p.Pro427Thr |
| 17:17216418:C:A | missense | ENST00000285071.9: | ENSP00000285071.4: |
| | | c.1262G>T | p.Ser421Ile |
| 17:17216418:C:CT | frameshift | ENST00000285071.9: | ENSP00000285071.4: |
| | | c.1261dup | p.Ser421LysfsTer35 |
| 17:17216424:C:A | missense | ENST00000285071.9: | ENSP00000285071.4: |
| | | c.1256G>T | p.Gly419Val |
| 17:17216424:C:T | missense | ENST00000285071.9: | ENSP00000285071.4: |
| | | c.1256G>A | p.Gly419Glu |
| 17:17216426:C:CA | frameshift | ENST00000285071.9: | ENSP00000285071.4: |
| | | c.1253dup | p.Leu420AlafsTer36 |
| 17:17216427:AG:A | frameshift | ENST00000285071.9: | ENSP00000285071.4: |
| | | c.1252del | p.Leu418TrpfsTer50 |
| 17:17216433:T:C | missense | ENST00000285071.9: | ENSP00000285071.4: |
| | | c.1247A>G | p.Asn416Ser |
| 17:17216435:G:T | stop_ | ENST00000285071.9: | ENSP00000285071.4: |
| | gained | c.1245C>A | p.Cys415Ter |
| 17:17216440:G:A | missense | ENST00000285071.9: | ENSP00000285071.4: |
| | | c.1240C>T | p.Arg414Trp |
| 17:17216443:A:T | missense | ENST00000285071.9: | ENSP00000285071.4: |
| | | c.1237T>A | p.Tyr413Asn |
| 17:17216453:G:C | stop_ | ENST00000285071.9: | ENSP00000285071.4: |
| | gained | c.1227C>G | p.Tyr409Ter |
| 17:17216454:T:C | missense | ENST00000285071.9: | ENSP00000285071.4: |
| | | c.1226A>G | p.Tyr409Cys |
| 17:17216458:G:A | stop_ | ENST00000285071.9: | ENSP00000285071.4: |
| | gained | c.1222C>T | p.Gln408Ter |
| 17:17216464:T:C | missense | ENST00000285071.9: | ENSP00000285071.4: |
| | | c.1216A>G | p.Ser406Gly |
| 17:17216466:T:C | missense | ENST00000285071.9: | ENSP00000285071.4: |
| | | c.1214A>G | p.Tyr405Cys |
| 17:17216466:T:TA | frameshift | ENST00000285071.9: | ENSP00000285071.4: |
| | | c.1213dup | p.Tyr405LeufsTer51 |
| 17:17216476:T:TG | frameshift | ENST00000285071.9: | ENSP00000285071.4: |
| | | c.1203dup | p.Ile402HisfsTer54 |
| 17:17216478:C:A | missense | ENST00000285071.9: | ENSP00000285071.4: |
| | | c.1202G>T | p.Arg401Leu |
| 17:17216479:G:C | missense | ENST00000285071.9: | ENSP00000285071.4: |
| | | c.1201C>G | p.Arg401Gly |
| 17:17216482:C:A | missense | ENST00000285071.9: | ENSP00000285071.4: |
| | | c.1198G>T | p.Val400Phe |
| 17:17216484:C:A | missense | ENST00000285071.9: | ENSP00000285071.4: |
| | | c.1196G>T | p.Cys399Phe |
| 17:17216484:C:T | missense | ENST00000285071.9: | ENSP00000285071.4: |
| | | c.1196G>A | p.Cys399Tyr |
| 17:17216485:A:G | missense | ENST00000285071.9: | ENSP00000285071.4: |
| | | c.1195T>C | p.Cys399Arg |
| 17:17216487:C:G | missense | ENST00000285071.9: | ENSP00000285071.4: |
| | | c.1193G>C | p.Gly398Ala |
| 17:17216491:C:CG | frameshift | ENST00000285071.9: | ENSP00000285071.4: |
| | | c.1188dup | p.Val397ArgfsTer59 |
| 17:17217068:C:G | splice_ | ENST00000285071.9: | NA |
| | donor | c.1176+1G>C | |
| 17:17217068:CCC | frameshift | ENST00000285071.9: | ENSP00000285071.4: |
| GAAGTACTTCAAAAG | | c.11541176del | p.Gln385HisfsTer63 |
| CTGACT:C | | | |
| 17:17217074:G:T | missense | ENST00000285071.9: | ENSP00000285071.4: |
| | | c.1171C>A | p.Leu391Ile |
| 17:17217086:C:G | missense | ENST00000285071.9: | ENSP00000285071.4: |
| | | c.1159G>C | p.Ala387Pro |
| 17:17217092:G:A | stop_ | ENST00000285071.9: | ENSP00000285071.4: |
| | gained | c.1153C>T | p.Gln385Ter |
| 17:17217112:C:CT | frameshift | ENST00000285071.9: | ENSP00000285071.4: |
| | | c.1132dup | p.Ser378LysfsTer12 |
| 17:17217116:TC:T | frameshift | ENST00000285071.9: | ENSP00000285071.4: |
| | | c.1128del | p.Trp376Ter |
| 17:17217124:A:T | missense | ENST00000285071.9: | ENSP00000285071.4: |
| | | c.1121T>A | p.Val374Glu |
| 17:17217127:TG:T | frameshift | ENST00000285071.9: | ENSP00000285071.4: |
| | | c.1117del | p.Gln373ArgfsTer2 |
| 17:17217128:G:A | stop_ | ENST00000285071.9: | ENSP00000285071.4: |
| | gained | c.1117C>T | p.Gln373Ter |
| 17:17217133:C:T | missense | ENST00000285071.9: | ENSP00000285071.4: |
| | | c.1112G>A | p.Gly371Glu |
| 17:17217147:C:T | stop_ | ENST00000285071.9: | ENSP00000285071.4: |
| | gained | c.1098G>A | p.Trp366Ter |
| 17:17217161:G:A | missense | ENST00000285071.9: | ENSP00000285071.4: |
| | | c.1084C>T | p.Arg362Cys |
| 17:17217163:A:G | missense | ENST00000285071.9: | ENSP00000285071.4: |
| | | c.1082T>C | p.Phe361Ser |
| 17:17217168:AG:A | frameshift | ENST00000285071.9: | ENSP00000285071.4: |
| | | c.1076del | p.Pro359LeufsTer16 |
| 17:17217174:AC:A | frameshift | ENST00000285071.9: | ENSP00000285071.4: |
| | | c.1070del | p.Gly357ValfsTer18 |
| 17:17217175:C:A | missense | ENST00000285071.9: | ENSP00000285071.4: |
| | | c.1070G>T | p.Gly357Val |
| 17:17217183:C:A | splice_ | ENST00000285071.9: | NA |
| | acceptor | c.1063−1G>T | |
| 17:17217184:T:A | splice_ | ENST00000285071.9: | NA |
| | acceptor | c.1063−2A>T | |
| 17:17217184:T:C | splice_ | ENST00000285071.9: | NA |
| | acceptor | c.1063−2A>G | |
| 17:17219017:A:C | splice_ | ENST00000285071.9: | NA |
| | donor | c.1062+2T>G | |
| 17:17219018:C:T | splice_ | ENST00000285071.9: | NA |
| | donor | c.1062+1G>A | |
| 17:17219030:G:A | missense | ENST00000285071.9: | ENSP00000285071.4: |
| | | c.1051C>T | p.His351Tyr |
| 17:17219032:C:T | missense | ENST00000285071.9: | ENSP00000285071.4: |
| | | c.1049G>A | p.Arg350Gln |
| 17:17219033:G:A | missense | ENST00000285071.9: | ENSP00000285071.4: |
| | | c.1048C>T | p.Arg350Trp |
| 17:17219038:G:A | missense | ENST00000285071.9: | ENSP00000285071.4: |
| | | c.1043C>T | p.Ser348Phe |
| 17:17219038:G:T | missense | ENST00000285071.9: | ENSP00000285071.4: |
| | | c.1043C>A | p.Ser348Tyr |
| 17:17219051:G: | frameshift | ENST00000285071.9: | ENSP00000285071.4: |
| GCAGATTCCGGGGC | | c.10291030ins | p.Pro344AlafsTer51 |
| TGC | | GCAGCCCCGGAATCTG | |
| 17:17219082: | frameshift | ENST00000285071.9: | ENSP00000285071.4: |
| TGAGA:T | | c.995_ | p.Leu332GlnfsTer20 |
| | | 998del | |
| 17:17219126:C:CT | frameshift | ENST00000285071.9: | ENSP00000285071.4: |
| TCTGTACTCTCTGGCA | | c.927954dup | p.Gly319SerfsTer80 |
| ACACAGGGGCT | | | |
| 17:17219133:ACT:A | frameshift | ENST00000285071.9: | ENSP00000285071.4: |
| | | c.946947del | p.Ser316TyrfsTer73 |
| 17:17219147:CAG:C | frameshift | ENST00000285071.9: | ENSP00000285071.4: |
| | | c.932933del | p.Pro311ArgfsTer78 |
| 17:17219148:A:AG | frameshift | ENST00000285071.9: | ENSP00000285071.4: |
| | | c.932dup | p.Val312CysfsTer78 |
| 17:17219174:C:CA | frameshift | ENST00000285071.9: | ENSP00000285071.4: |
| | | c.906dup | p.Glu303Ter |
| 17:17219176:G:C | missense | ENST00000285071.9: | ENSP00000285071.4: |
| | | c.905C>G | p.Ser302Cys |
| 17:17219177:A:G | missense | ENST00000285071.9: | ENSP00000285071.4: |
| | | c.904T>C | p.Ser302Pro |
| 17:17219183:C:G | missense | ENST00000285071.9: | ENSP00000285071.4: |
| | | c.898G>C | p.Asp300His |
| 17:17219187: | frameshift | ENST00000285071.9: | ENSP00000285071.4: |
| GCTTT:G | | c.890893del | p.Glu297AlafsTer25 |
| 17:17219190:TTC:T | frameshift | ENST00000285071.9: | ENSP00000285071.4: |
| | | c.889890del | p.Glu297LysfsTer6 |
| 17:17219194:G:C | stop_ | ENST00000285071.9: | ENSP00000285071.4: |
| | gained | c.887C>G | p.Ser296Ter |
| 17:17219194:G:T | stop_ | ENST00000285071.9: | ENSP00000285071.4: |
| | gained | c.887C>A | p.Ser296Ter |
| 17:17219201:C:G | missense | ENST00000285071.9: | ENSP00000285071.4: |
| | | c.880G>C | p.Glu294Gln |
| 17:17219206:A:C | stop_ | ENST00000285071.9: | ENSP00000285071.4: |
| | gained | c.875T>G | p.Leu292Ter |
| 17:17221535:A:G | splice_ | ENST00000285071.9: | NA |
| | donor | c.871+2T>C | |
| 17:17221549:C:T | missense | ENST00000285071.9: | ENSP00000285071.4: |
| | | c.859G>A | p.Glu287Lys |
| 17:17221555:G:A | stop_ | ENST00000285071.9: | ENSP00000285071.4: |
| | gained | c.853C>T | p.Gln285Ter |
| 17:17221567:C:A | missense | ENST00000285071.9: | ENSP00000285071.4: |
| | | c.841G>T | p.Asp281Tyr |
| 17:17221570:C:T | missense | ENST00000285071.9: | ENSP00000285071.4: |
| | | c.838G>A | p.Glu280Lys |
| 17:17221575:G:A | missense | ENST00000285071.9: | ENSP00000285071.4: |
| | | c.833C>T | p.Pro278Leu |
| 17:17221576:G:C | missense | ENST00000285071.9: | ENSP00000285071.4: |
| | | c.832C>G | p.Pro278Ala |
| 17:17221597:C:T | missense | ENST00000285071.9: | ENSP00000285071.4: |
| | | c.811G>A | p.Glu271Lys |
| 17:17221599:G:A | missense | ENST00000285071.9: | ENSP00000285071.4: |
| | | c.809C>T | p.Thr270Ile |
| 17:17221605:C:T | missense | ENST00000285071.9: | ENSP00000285071.4: |
| | | c.803G>A | p.Arg268Gln |
| 17:17221612:C:T | missense | ENST00000285071.9: | ENSP00000285071.4: |
| | | c.796G>A | p.Gly266Ser |
| 17:17221614:C:T | missense | ENST00000285071.9: | ENSP00000285071.4: |
| | | c.794G>A | p.Cys265Tyr |
| 17:17221617:G:A | missense | ENST00000285071.9: | ENSP00000285071.4: |
| | | c.791C>T | p.Ala264Val |
| 17:17221617:G:C | missense | ENST00000285071.9: | ENSP00000285071.4: |
| | | c.791C>G | p.Ala264Gly |
| 17:17221618:C:A | missense | ENST00000285071.9: | ENSP00000285071.4: |
| | | c.790G>T | p.Ala264Ser |
| 17:17221618:C:G | missense | ENST00000285071.9: | ENSP00000285071.4: |
| | | c.790G>C | p.Ala264Pro |
| 17:17221627:G:A | missense | ENST00000285071.9: | ENSP00000285071.4: |
| | | c.781C>T | p.Leu261Phe |
| 17:17221628:C:T | stop_ | ENST00000285071.9: | ENSP00000285071.4: |
| | gained | c.780G>A | p.Trp260Ter |
| 17:17221630:T:C | splice_ | ENST00000285071.9: | NA |
| | acceptor | c.780−2A>G | |
| 17:17222500:C:A | splice_ | ENST00000285071.9: | NA |
| | donor | c.779+1G>T | |
| 17:17222501:C:G | missense | ENST00000285071.9: | ENSP00000285071.4: |
| | | c.779G>C | p.Trp260Ser |
| 17:17222516:T:G | missense | ENST00000285071.9: | ENSP00000285071.4: |
| | | c.764A>C | p.His255Pro |
| 17:17222517:G:A | missense | ENST00000285071.9: | ENSP00000285071.4: |
| | | c.763C>T | p.His255Tyr |
| 17:17222519:A:G | missense | ENST00000285071.9: | ENSP00000285071.4: |
| | | c.761T>C | p.Leu254Pro |
| 17:17222525:G:GC | frameshift | ENST00000285071.9: | ENSP00000285071.4: |
| | | c.754dup | p.Ala252GlyfsTer40 |
| 17:17222532:G:T | missense | ENST00000285071.9: | ENSP00000285071.4: |
| | | c.748C>A | p.Leu250Met |
| 17:17222540:T:C | missense | ENST00000285071.9: | ENSP00000285071.4: |
| | | c.740A>G | p.Asp247Gly |
| 17:17222541: | frameshift | ENST00000285071.9: | ENSP00000285071.4: |
| CACTT:C | | c.735738del | p.Ser246MetfsTer16 |
| 17:17222546:G:T | missense | ENST00000285071.9: | ENSP00000285071.4: |
| | | c.734C>A | p.Thr245Lys |
| 17:17222558:A:C | missense | ENST00000285071.9: | ENSP00000285071.4: |
| | | c.722T>G | p.Leu241Arg |
| 17:17222558:A:G | missense | ENST00000285071.9: | ENSP00000285071.4: |
| | | c.722T>C | p.Leu241Pro |
| 17:17222559:G:C | missense | ENST00000285071.9: | ENSP00000285071.4: |
| | | c.721C>G | p.Leu241Val |
| 17:17222561:G:A | missense | ENST00000285071.9: | ENSP00000285071.4: |
| | | c.719C>T | p.Ser240Leu |
| 17:17222564:C:T | missense | ENST00000285071.9: | ENSP00000285071.4: |
| | | c.716G>A | p.Arg239His |
| 17:17222565:G:A | missense | ENST00000285071.9: | ENSP00000285071.4: |
| | | c.715C>T | p.Arg239Cys |
| 17:17222565:G:C | missense | ENST00000285071.9: | ENSP00000285071.4: |
| | | c.715C>G | p.Arg239Gly |
| 17:17222582:C:T | missense | ENST00000285071.9: | ENSP00000285071.4: |
| | | c.698G>A | p.Arg233Lys |
| 17:17222590:T:TA | frameshift | ENST00000285071.9: | ENSP00000285071.4: |
| | | c.689dup | p.His231ThrfsTer17 |
| 17:17222615:A:C | missense | ENST00000285071.9: | ENSP00000285071.4: |
| | | c.665T>G | p.Met222Arg |
| 17:17222618:C:A | missense | ENST00000285071.9: | ENSP00000285071.4: |
| | | c.662G>T | p.Arg221Met |
| 17:17222628:G:A | missense | ENST00000285071.9: | ENSP00000285071.4: |
| | | c.652C>T | p.Arg218Cys |
| 17:17222632:TG:T | frameshift | ENST00000285071.9: | ENSP00000285071.4: |
| | | c.647del | p.Pro216HisfsTer7 |
| 17:17222636:C:T | missense | ENST00000285071.9: | ENSP00000285071.4: |
| | | c.644G>A | p.Cys215Tyr |
| 17:17222649:C:T | missense | ENST00000285071.9: | ENSP00000285071.4: |
| | | c.631G>A | p.Glu211Lys |
| 17:17222657:A:C | missense | ENST00000285071.9: | ENSP00000285071.4: |
| | | c.623T>G | p.Phe208Cys |
| 17:17222662:C:T | splice_ | ENST00000285071.9: | NA |
| | acceptor | c.619−1G>A | |
| 17:17222663:T:G | splice_ | ENST00000285071.9: | NA |
| | acceptor | c.619−2A>C | |
| 17:17223929:G:A | missense | ENST00000285071.9: | ENSP00000285071.4: |
| | | c.611C>T | p.Ala204Val |
| 17:17223930:C:T | missense | ENST00000285071.9: | ENSP00000285071.4: |
| | | c.610G>A | p.Ala204Th |
| 17:17223937:C:A | missense | ENST00000285071.9: | ENSP00000285071.4: |
| | | c.603G>T | p.Gln201His |
| 17:17223937:C:G | missense | ENST00000285071.9: | ENSP00000285071.4: |
| | | c.603G>C | p.Gln201His |
| 17:17223942:G:A | missense | ENST00000285071.9: | ENSP00000285071.4: |
| | | c.598C>T | p.Leu200Phe |
| 17:17223951:T:A | missense | ENST00000285071.9: | ENSP00000285071.4: |
| | | c.589A>T | p.Ile197Phe |
| 17:17223955:TC:T | frameshift | ENST00000285071.9: | ENSP00000285071.4: |
| | | c.584del | p.Gly195GlufsTer28 |
| 17:17223959:C:A | missense | ENST00000285071.9: | ENSP00000285071.4: |
| | | c.581G>T | p.Arg194Leu |
| 17:17223960:G:A | missense | ENST00000285071.9: | ENSP00000285071.4: |
| | | c.580C>T | p.Arg194Trp |
| 17:17223966:T:A | stop_ | ENST00000285071.9: | ENSP00000285071.4: |
| | gained | c.574A>T | p.Lys192Ter |
| 17:17223975:G:C | missense | ENST00000285071.9: | ENSP00000285071.4: |
| | | c.565C>G | p.Leu189Val |
| 17:17223978:A:T | missense | ENST00000285071.9: | ENSP00000285071.4: |
| | | c.562T>A | p.Phe188Ile |
| 17:17223980:G:A | missense | ENST00000285071.9: | ENSP00000285071.4: |
| | | c.560C>T | p.Pro187Leu |
| 17:17223981:G:T | missense | ENST00000285071.9: | ENSP00000285071.4: |
| | | c.559C>A | p.Pro187Thr |
| 17:17223983:C:A | missense | ENST00000285071.9: | ENSP00000285071.4: |
| | | c.557G>T | p.Trp186Leu |
| 17:17223986:G:A | missense | ENST00000285071.9: | ENSP00000285071.4: |
| | | c.554C>T | p.Ser185Phe |
| 17:17223989:T:G | missense | ENST00000285071.9: | ENSP00000285071.4: |
| | | c.551A>C | p.Asn184Thr |
| 17:17223995:A:G | missense | ENST00000285071.9: | ENSP00000285071.4: |
| | | c.545T>C | p.Leu182Pro |
| 17:17223996:G:A | missense | ENST00000285071.9: | ENSP00000285071.4: |
| | | c.544C>T | p.Leu182Phe |
| 17:17223998:T:C | missense | ENST00000285071.9: | ENSP00000285071.4: |
| | | c.542A>G | p.Tyr181Cys |
| 17:17224001:A:G | missense | ENST00000285071.9: | ENSP00000285071.4: |
| | | c.539T>C | p.Ile180Thr |
| 17:17224001:A:T | missense | ENST00000285071.9: | ENSP00000285071.4: |
| | | c.539T>A | p.Ile180Asn |
| 17:17224002:T:A | missense | ENST00000285071.9: | ENSP00000285071.4: |
| | | c.538A>T | p.Ile180Phe |
| 17:17224004:C:T | missense | ENST00000285071.9: | ENSP00000285071.4: |
| | | c.536G>A | p.Arg179Gln |
| 17:17224005:G:A | missense | ENST00000285071.9: | ENSP00000285071.4: |
| | | c.535C>T | p.Arg179Trp |
| 17:17224008:C:A | missense | ENST00000285071.9: | ENSP00000285071.4: |
| | | c.532G>T | p.Asp178Tyr |
| 17:17224012: | frameshift | ENST00000285071.9: | ENSP00000285071.4: |
| CATGATGG:C | | c.521527del | p.Thr174ArgfsTer47 |
| 17:17224017:T:A | missense | ENST00000285071.9: | ENSP00000285071.4: |
| | | c.523A>T | p.Ile175Phe |
| 17:17224021:G:C | missense | ENST00000285071.9: | ENSP00000285071.4: |
| | | c.519C>G | p.Ile173Met |
| 17:17224030:G:T | stop_ | ENST00000285071.9: | ENSP00000285071.4: |
| | gained | c.510C>A | p.Tyr170Ter |
| 17:17224032:A:G | missense | ENST00000285071.9: | ENSP00000285071.4: |
| | | c.508T>C | p.Tyr170His |
| 17:17224032:A:T | missense | ENST00000285071.9: | ENSP00000285071.4: |
| | | c.508T>A | p.Tyr170Asn |
| 17:17224035:A:G | missense | ENST00000285071.9: | ENSP00000285071.4: |
| | | c.505T>C | p.Trp169Arg |
| 17:17224037:C:T | missense | ENST00000285071.9: | ENSP00000285071.4: |
| | | c.503G>A | p.Arg168His |
| 17:17224038:G:A | missense | ENST00000285071.9: | ENSP00000285071.4: |
| | | c.502C>T | p.Arg168Cys |
| 17:17224039:C:G | missense | ENST00000285071.9: | ENSP00000285071.4: |
| | | c.501G>C | p.Gln167His |
| 17:17224041:G:A | stop_ | ENST00000285071.9: | ENSP00000285071.4: |
| | gained | c.499C>T | p.Gln167Ter |
| 17:17224053:C:A | missense | ENST00000285071.9: | ENSP00000285071.4: |
| | | c.487G>T | p.Ala163Ser |
| 17:17224062:C:A | missense | ENST00000285071.9: | ENSP00000285071.4: |
| | | c.478G>T | p.Asp160Tyr |
| 17:17224073:A:G | missense | ENST00000285071.9: | ENSP00000285071.4: |
| | | c.467T>C | p.Phe156Ser |
| 17:17224076:G:T | missense | ENST00000285071.9: | ENSP00000285071.4: |
| | | c.464C>A | p.Thr155Asn |
| 17:17224077:T:A | missense | ENST00000285071.9: | ENSP00000285071.4: |
| | | c.463A>T | p.Thr155Ser |
| 17:17224083:T:C | missense | ENST00000285071.9: | ENSP00000285071.4: |
| | | c.457A>G | p.Ser153Gly |
| 17:17224088:A:G | missense | ENST00000285071.9: | ENSP00000285071.4: |
| | | c.452T>C | p.Val151Ala |
| 17:17224089:C:T | missense | ENST00000285071.9: | ENSP00000285071.4: |
| | | c.451G>A | p.Val151Met |
| 17:17224091:A:G | missense | ENST00000285071.9: | ENSP00000285071.4: |
| | | c.449T>C | p.Phe150Ser |
| 17:17224092:A:C | missense | ENST00000285071.9: | ENSP00000285071.4: |
| | | c.448T>G | p.Phe150Val |
| 17:17224095: | missense | ENST00000285071.9: | ENSP00000285071.4: |
| C:T | | c.445G>A | p.Gly149Ser |
| 17:17224099: | frameshift | ENST00000285071.9: | ENSP00000285071.4: |
| C:CTG | | c.439440dup | p.Gln147HisfsTer31 |
| 17:17224099: | frameshift | ENST00000285071.9: | ENSP00000285071.4: |
| CT:C | | c.440del | p.Gln147ArgfsTer30 |
| 17:17224104: | missense | ENST00000285071.9: | ENSP00000285071.4: |
| C:T | | c.436G>A | p.Glu146Lys |
| 17:17224107: | missense | ENST00000285071.9: | ENSP00000285071.4: |
| C:T | | c.433G>A | p.Asp145Asn |
| 17:17224110:C:T | missense | ENST00000285071.9: | ENSP00000285071.4: |
| | | c.430G>A | p.Gly144Arg |
| 17:17224111:G:C | missense | ENST00000285071.9: | ENSP00000285071.4: |
| | | c.429C>G | p.Phe143Leu |
| 17:17224111:G:T | missense | ENST00000285071.9: | ENSP00000285071.4: |
| | | c.429C>A | p.Phe143Leu |
| 17:17224115:A:C | missense | ENST00000285071.9: | ENSP00000285071.4: |
| | | c.425T>G | p.Phe142Cys |
| 17:17224121:G:A | missense | ENST00000285071.9: | ENSP00000285071.4: |
| | | c.419C>T | p.Pro140Leu |
| 17:17224121:G:C | missense | ENST00000285071.9: | ENSP00000285071.4: |
| | | c.419C>G | p.Pro140Arg |
| 17:17224124:C:A | missense | ENST00000285071.9: | ENSP00000285071.4: |
| | | c.416G>T | p.Gly139Val |
| 17:17224125:C:T | missense | ENST00000285071.9: | ENSP00000285071.4: |
| | | c.415G>A | p.Gly139Ser |
| 17:17224128:C:T | missense | ENST00000285071.9: | ENSP00000285071.4: |
| | | c.412G>A | p.Glu138Lys |
| 17:17224130:C:A | missense | ENST00000285071.9: | ENSP00000285071.4: |
| | | c.410G>T | p.Arg137Leu |
| 17:17224130:C:T | missense | ENST00000285071.9: | ENSP00000285071.4: |
| | | c.410G>A | p.Arg137His |
| 17:17224131:G:A | missense | ENST00000285071.9: | ENSP00000285071.4: |
| | | c.409C>T | p.Arg137Cys |
| 17:17224136:G:A | missense | ENST00000285071.9: | ENSP00000285071.4: |
| | | c.404C>T | p.Pro135Leu |
| 17:17224136:G:C | missense | ENST00000285071.9: | ENSP00000285071.4: |
| | | c.404C>G | p.Pro135Arg |
| 17:17224137:G:T | missense | ENST00000285071.9: | ENSP00000285071.4: |
| | | c.403C>A | p.Pro135Thr |
| 17:17224143:C:A | missense | ENST00000285071.9: | ENSP00000285071.4: |
| | | c.397G>T | p.Val133Phe |
| 17:17224143:C:T | missense | ENST00000285071.9: | ENSP00000285071.4: |
| | | c.397G>A | p.Val133Ile |
| 17:17226174:A:C | splice_ | ENST00000285071.9: | NA |
| | donor | c.396+2T>G | |
| 17:17226186:A:C | missense | ENST00000285071.9: | ENSP00000285071.4: |
| | | c.386T>G | p.Leu129Arg |
| 17:17226187:G:C | missense | ENST00000285071.9: | ENSP00000285071.4: |
| | | c.385C>G | p.Leu129Val |
| 17:17226192:C:T | missense | ENST00000285071.9: | ENSP00000285071.4: |
| | | c.380G>A | p.Arg127Gln |
| 17:17226193:G:A | missense | ENST00000285071.9: | ENSP00000285071.4: |
| | | c.379C>T | p.Arg127Trp |
| 17:17226193:G:C | missense | ENST00000285071.9: | ENSP00000285071.4: |
| | | c.379C>G | p.Arg127Gly |
| 17:17226202:C:T | missense | ENST00000285071.9: | ENSP00000285071.4: |
| | | c.370G>A | p.Ala124Thr |
| 17:17226207:C:G | missense | ENST00000285071.9: | ENSP00000285071.4: |
| | | c.365G>C | p.Arg122Pro |
| 17:17226208:G:A | missense | ENST00000285071.9: | ENSP00000285071.4: |
| | | c.364C>T | p.Arg122Cys |
| 17:17226210:A:G | missense | ENST00000285071.9: | ENSP00000285071.4: |
| | | c.362T>C | p.Val121Ala |
| 17:17226220:A:C | missense | ENST00000285071.9: | ENSP00000285071.4: |
| | | c.352T>G | p.Phe118Val |
| 17:17226224:C:CT | frameshift | ENST00000285071.9: | ENSP00000285071.4: |
| | | c.347dup | p.Leu117AlafsTer16 |
| 17:17226224:C:G | missense | ENST00000285071.9: | ENSP00000285071.4: |
| | | c.348G>C | p.Gln116His |
| 17:17226243:T:A | missense | ENST00000285071.9: | ENSP00000285071.4: |
| | | c.329A>T | p.Gln110Leu |
| 17:17226245:G:GT | frameshift | ENST00000285071.9: | ENSP00000285071.4: |
| | | c.326dup | p.His109GlnfsTer24 |
| 17:17226247:G:A | missense | ENST00000285071.9: | ENSP00000285071.4: |
| | | c.325C>T | p.His109Tyr |
| 17:17226250:T:TG | frameshift | ENST00000285071.9: | ENSP00000285071.4: |
| | | c.321dup | p.Ser108GlnfsTer25 |
| 17:17226252:A:T | missense | ENST00000285071.9: | ENSP00000285071.4: |
| | | c.320T>A | p.Val107Asp |
| 17:17226262:T:C | missense | ENST00000285071.9: | ENSP00000285071.4: |
| | | c.310A>G | p.Ile104Val |
| 17:17226270:T:C | missense | ENST00000285071.9: | ENSP00000285071.4: |
| | | c.302A>G | p.Glu101Gly |
| 17:17226274:T:A | stop_ | ENST00000285071.9: | ENSP00000285071.4: |
| | gained | c.298A>T | p.Lys100Ter |
| 17:17226276:T:C | missense | ENST00000285071.9: | ENSP00000285071.4: |
| | | c.296A>G | p.Asp99Gly |
| 17:17226277:C:G | missense | ENST00000285071.9: | ENSP00000285071.4: |
| | | c.295G>C | p.Asp99His |
| 17:17226282:C:T | missense | ENST00000285071.9: | ENSP00000285071.4: |
| | | c.290G>A | p.Ser97Asn |
| 17:17226291:C:G | missense | ENST00000285071.9: | ENSP00000285071.4: |
| | | c.281G>C | p.Gly94Ala |
| 17:17226294:G:A | missense | ENST00000285071.9: | ENSP00000285071.4: |
| | | c.278C>T | p.Pro93Leu |
| 17:17226294:G:C | missense | ENST00000285071.9: | ENSP00000285071.4: |
| | | c.278C>G | p.Pro93Arg |
| 17:17226295:G:C | missense | ENST00000285071.9: | ENSP00000285071.4: |
| | | c.277C>G | p.Pro93Ala |
| 17:17226297:T:G | missense | ENST00000285071.9: | ENSP00000285071.4: |
| | | c.275A>C | p.His92Pro |
| 17:17226298:G:A | missense | ENST00000285071.9: | ENSP00000285071.4: |
| | | c.274C>T | p.His92Tyr |
| 17:17226301:C:T | missense | ENST00000285071.9: | ENSP00000285071.4: |
| | | c.271G>A | p.Gly91Arg |
| 17:17226315:C:T | missense | ENST00000285071.9: | ENSP00000285071.4: |
| | | c.257G>A | p.Arg86Gln |
| 17:17226316:G:A | missense | ENST00000285071.9: | ENSP00000285071.4: |
| | | c.256C>T | p.Arg86Trp |
| 17:17226318:C:T | missense | ENST00000285071.9: | ENSP00000285071.4: |
| | | c.254G>A | p.Cys85Tyr |
| 17:17226321:C:A | missense | ENST00000285071.9: | ENSP00000285071.4: |
| | | c.251G>T | p.Gly84Val |
| 17:17226324:T:C | splice_ | ENST00000285071.9: | NA |
| | acceptor | c.250−2A>G | |
| 17:17227887:G:A | splice_ | ENST00000285071.9: | NA |
| | donor | c.249+2C>T | |
| 17:17227887:G:T | splice_ | ENST00000285071.9: | NA |
| | donor | c.249+2C>A | |
| 17:17227893:C:T | missense | ENST00000285071.9: | ENSP00000285071.4: |
| | | c.245G>A | p.Cys82Tyr |
| 17:17227899:TCCGA:T | frameshift | ENST00000285071.9: | ENSP00000285071.4: |
| | | c.235238del | p.Ser79ThrfsTer50 |
| 17:17227904:CT:C | frameshift | ENST00000285071.9: | ENSP00000285071.4: |
| | | c.233del | p.Lys78SerfsTer52 |
| 17:17227923:CT:C | frameshift | ENST00000285071.9: | ENSP00000285071.4: |
| | | c.214del | p.Ser72AlafsTer58 |
| 17:17227930:C:A | stop_ | ENST00000285071.9: | ENSP00000285071.4: |
| | gained | c.208G>T | p.Glu70Ter |
| 17:17227948:CG:C | frameshift | ENST00000285071.9: | ENSP00000285071.4: |
| | | c.189del | p.Ala64GlnfsTer66 |
| 17:17227950:G:T | missense | ENST00000285071.9: | ENSP00000285071.4: |
| | | c.188C>A | p.Pro63His |
| 17:17227979:CT:C | frameshift | ENST00000285071.9: | ENSP00000285071.4: |
| | | c.158del | p.Gln53ArgfsTer2 |
| 17:17228025:C:CT | frameshift | ENST00000285071.9: | ENSP00000285071.4: |
| | | c.112dup | p.Ser38LysfsTer6 |
| 17:17228040:TC:T | frameshift | ENST00000285071.9: | ENSP00000285071.4: |
| | | c.97del | p.Asp33MetfsTer22 |
| 17:17228073:G:A | missense | ENST00000285071.9: | ENSP00000285071.4: |
| | | c.65C>T | p.Thr22Met |
| 17:17228077:A:G | missense | ENST00000285071.9: | ENSP00000285071.4: |
| | | c.61T>C | p.Cys21Arg |
| 17:17228079:A:T | missense | ENST00000285071.9: | ENSP00000285071.4: |
| | | c.59T>A | p.Phe20Tyr |
| 17:17228082:A:G | missense | ENST00000285071.9: | ENSP00000285071.4: |
| | | c.56T>C | p.Leu19Pro |
| 17:17228085:G:T | missense | ENST00000285071.9: | ENSP00000285071.4: |
| | | c.53C>A | p.Thr18Asn |
| 17:17228088:C:A | missense | ENST00000285071.9: | ENSP00000285071.4: |
| | | c.50G>T | p.Arg17Leu |
| 17:17228088:C:T | missense | ENST00000285071.9: | ENSP00000285071.4: |
| | | c.50G>A | p.Arg17His |
| 17:17228089:G:A | missense | ENST00000285071.9: | ENSP00000285071.4: |
| | | c.49C>T | p.Arg17Cys |
| 17:17228091:G:A | missense | ENST00000285071.9: | ENSP00000285071.4: |
| | | c.47C>T | p.Pro16Leu |
| 17:17228095:C:T | missense | ENST00000285071.9: | ENSP00000285071.4: |
| | | c.43G>A | p.Gly15Ser |
| 17:17228104:C:G | missense | ENST00000285071.9: | ENSP00000285071.4: |
| | | c.34G>C | p.Glu12Gln |
| 17:17228104:C:T | missense | ENST00000285071.9: | ENSP00000285071.4: |
| | | c.34G>A | p.Glu12Lys |
| 17:17228105:G:C | missense | ENST00000285071.9: | ENSP00000285071.4: |
| | | c.33C>G | p.Cys11Trp |
| 17:17228105:G:T | stop_ | ENST00000285071.9: | ENSP00000285071.4: |
| | gained | c.33C>A | p.Cys11Ter |
| 17:17228109:A:T | missense | ENST00000285071.9: | ENSP00000285071.4: |
| | | c.29T>A | p.Phe10Tyr |
| 17:17228112:T:C | missense | ENST00000285071.9: | ENSP00000285071.4: |
| | | c.26A>G | p.His9Arg |
| 17:17228114:G:T | stop_ | ENST00000285071.9: | ENSP00000285071.4: |
| | gained | c.24C>A | p.Cys8Ter |
| 17:17228115:C:T | missense | ENST00000285071.9: | ENSP00000285071.4: |
| | | c.23G>A | p.Cys8Tyr |
| 17:17228119:G:C | missense | ENST00000285071.9: | ENSP00000285071.4: |
| | | c.19C>G | p.Leu7Val |
| 17:17228124:A:C | missense | ENST00000285071.9: | ENSP00000285071.4: |
| | | c.14T>G | p.Val5Gly |
| 17:17228133:T:C | missense | ENST00000285071.9: | ENSP00000285071.4: |
| | | c.5A>G | p.Asn2Ser |
| 17:17228136:A:G | start_ | ENST00000285071.9: | ENSP00000285071.4: |
| | lost | c.2T>C | p.Met1? |
| 17:17228137:T:C | start_ | ENST00000285071.9: | ENSP00000285071.4: |
| | lost | c.1A>G | p.Met1? |

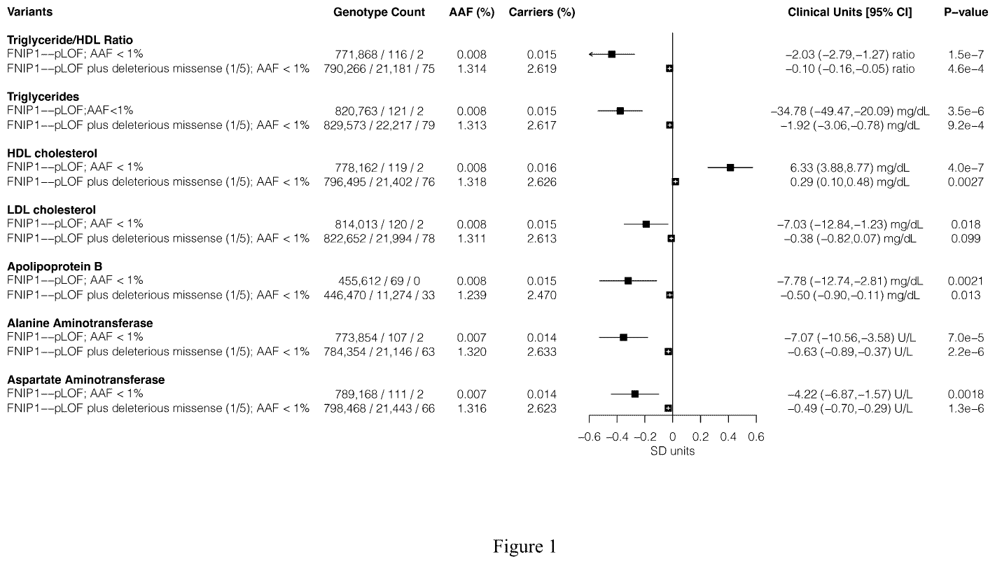

Additional data is shown in FIGS. 1-18.

FIG. 1 shows identification of FNIP1 rare coding variation associated with blood lipids and liver enzymes in ExWAS; the largest effect was observed with lower triglycerides and ALT.

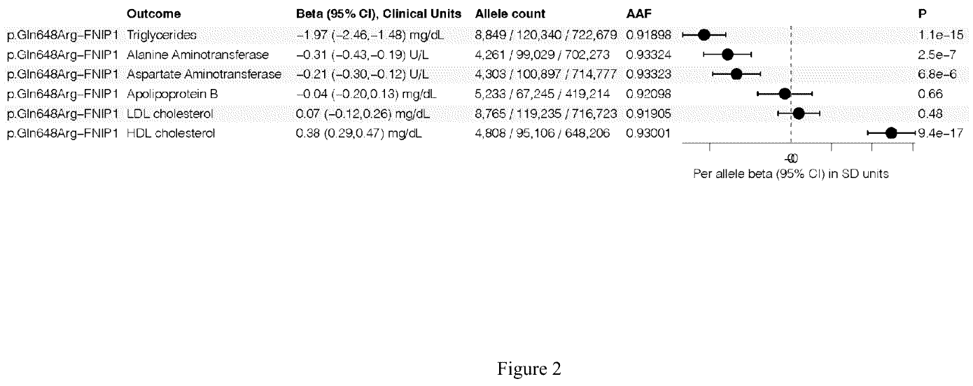

FIG. 2 shows identification of genome-wide association analysis with a common missense (5:131672501:T:C—Gln648Arg) variant in FNIP1 to be associated with TGL:HDL ratio.

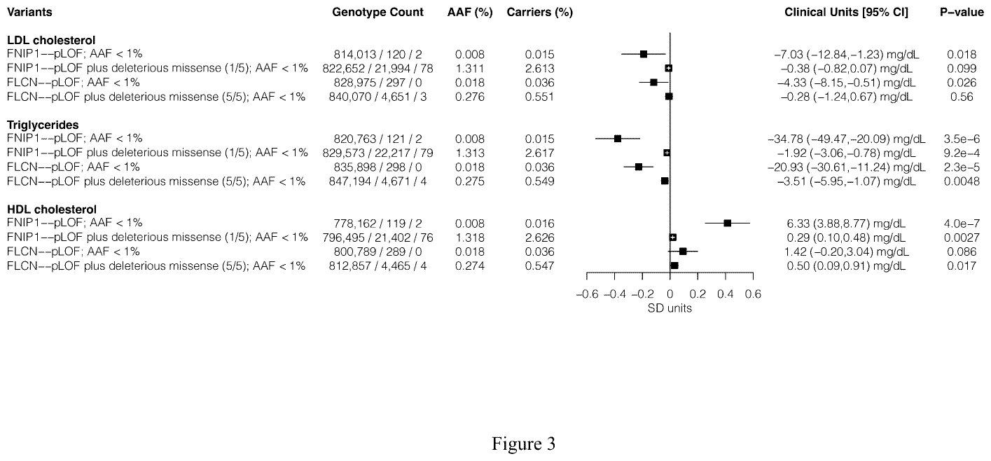

FIG. 3 shows association of FNIP1 rare coding variants with higher HDLC and lower triglycerides and LDLC levels; FLCN shows similar pattern of associations.

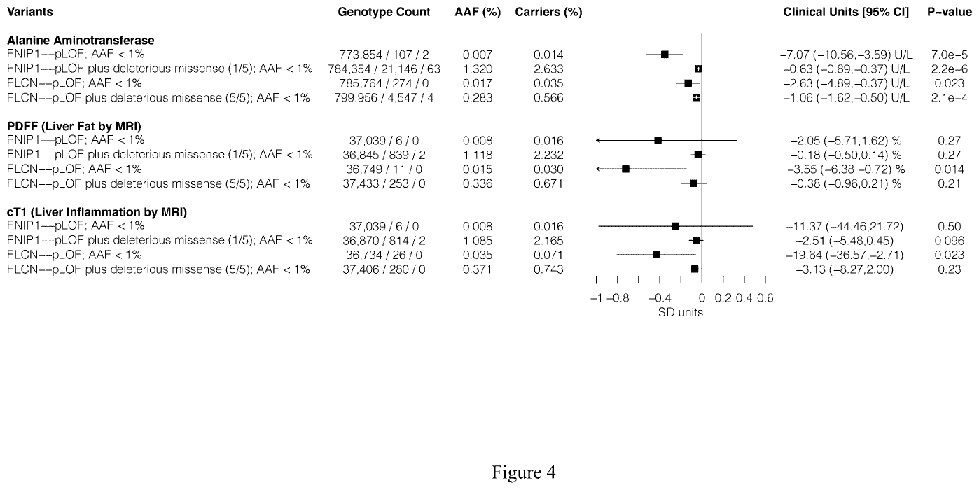

FIG. 4 shows association of FNIP1 and FLCN rare coding variants with lower ALT levels; FLCN pLoFs are associated with lower MRI derived liver fat and inflammation.

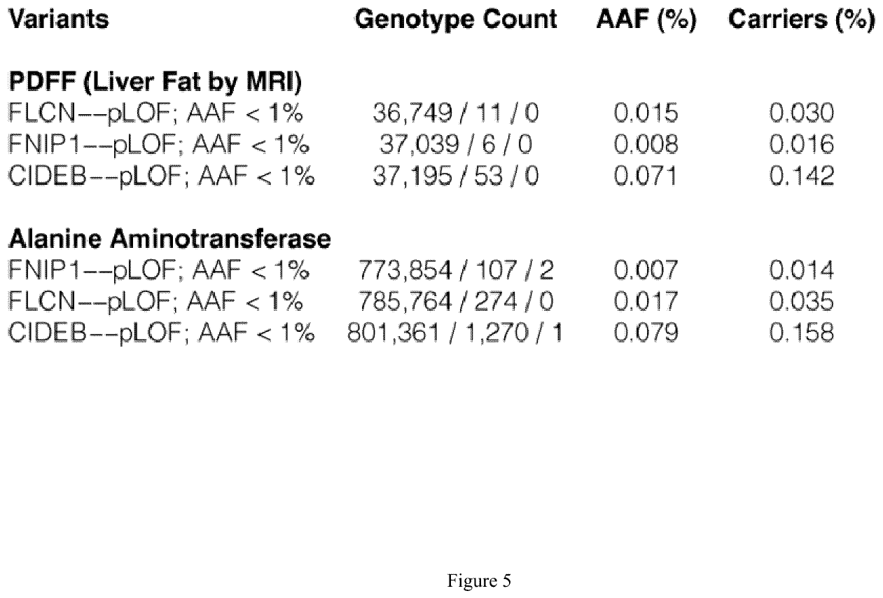

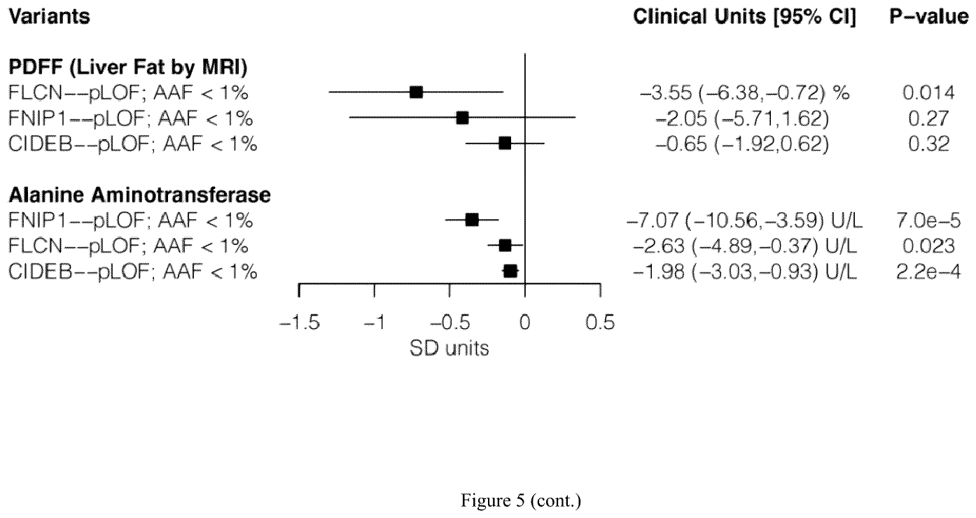

FIG. 5 shows the impact of FNIP1 and FLCN rare coding variants on ALT and PDFF.

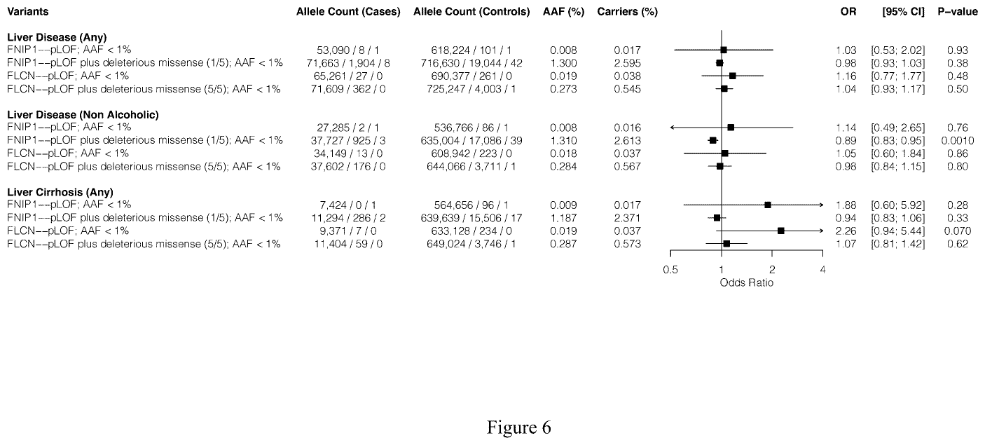

FIG. 6 shows association of FNIP1 rare coding variants with lower odds for NALD.

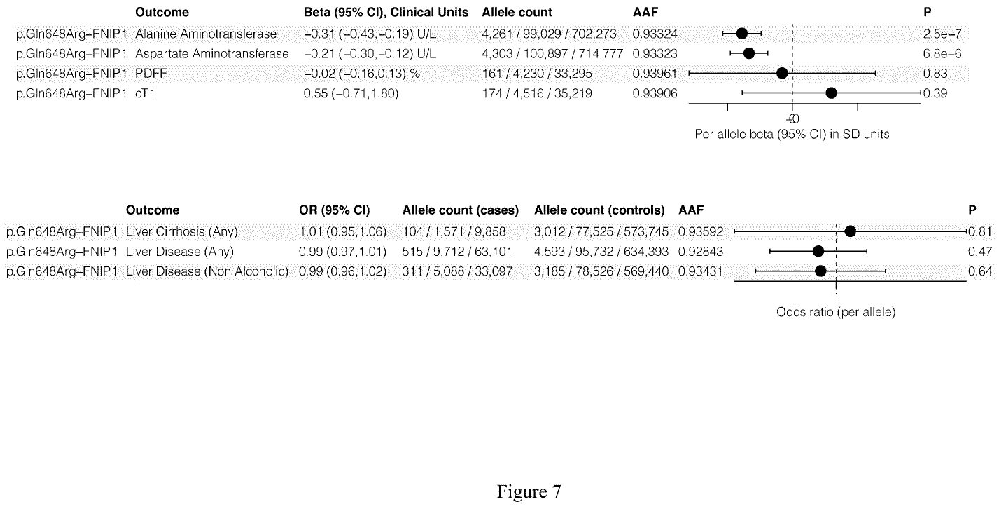

FIG. 7 shows association of common FNIP1 missense variant with liver enzymes.

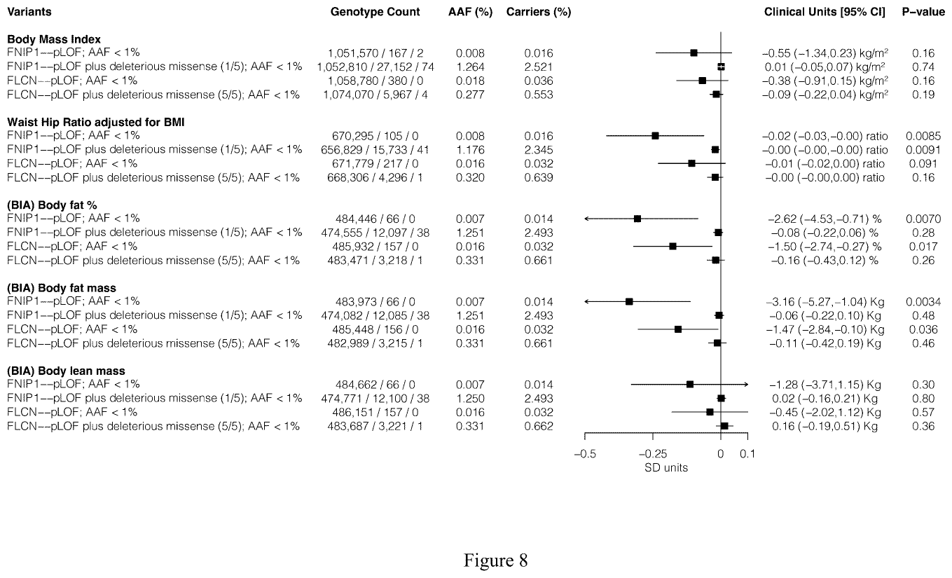

FIG. 8 shows association of FNIP1 and FLCN rare coding variants with lower waist to hip ratio and body fat percentage.

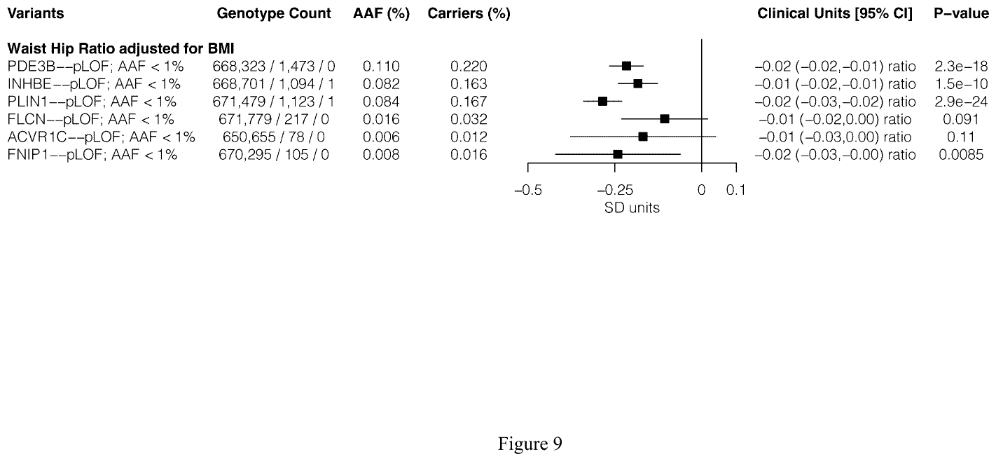

FIG. 9 shows the impact of rare variation in FNIP1 on body fat distribution.

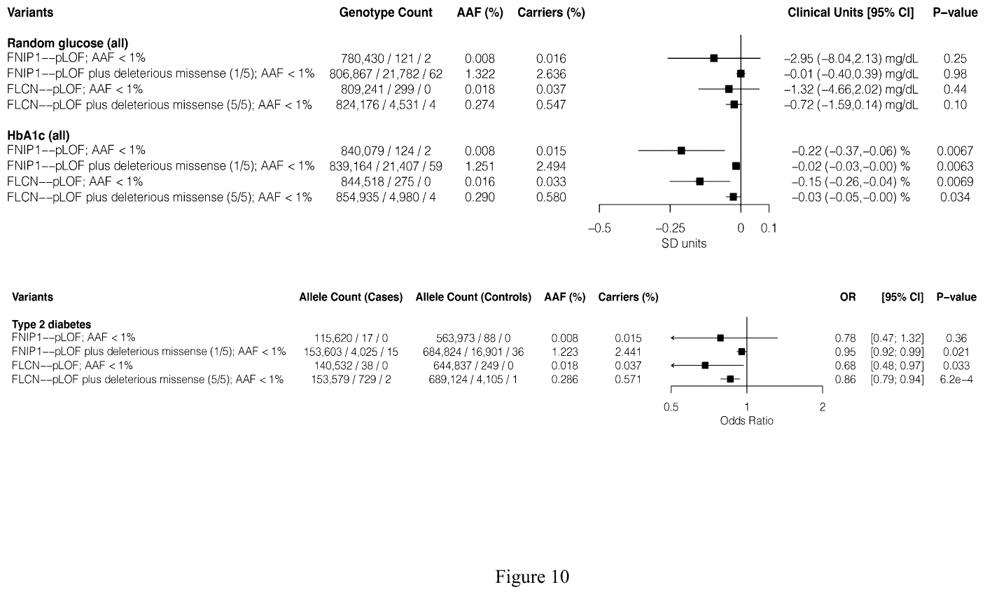

FIG. 10 shows association of FNIP1 and FLCN rare coding variants with lower HbA1c levels and lower odds of type 2 diabetes.

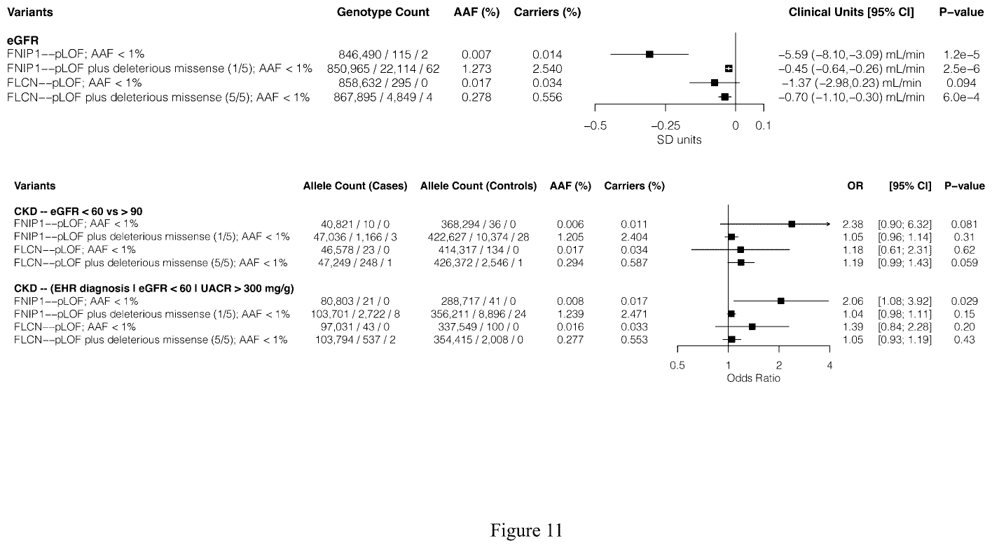

FIG. 11 shows association of FNIP1 and FLCN rare coding variants with lower eGFR.

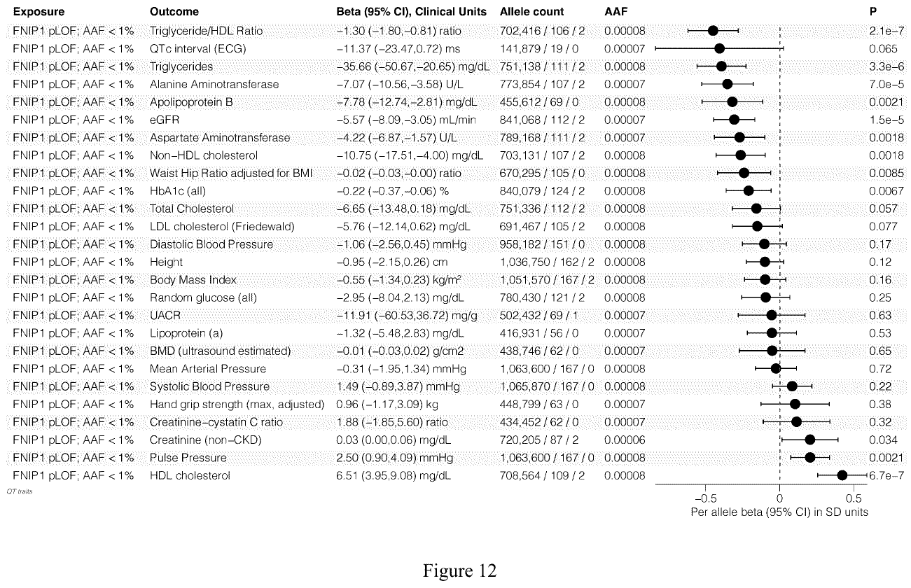

FIG. 12 shows FNIP1 pLoF had general protective cardiometabolic associations.

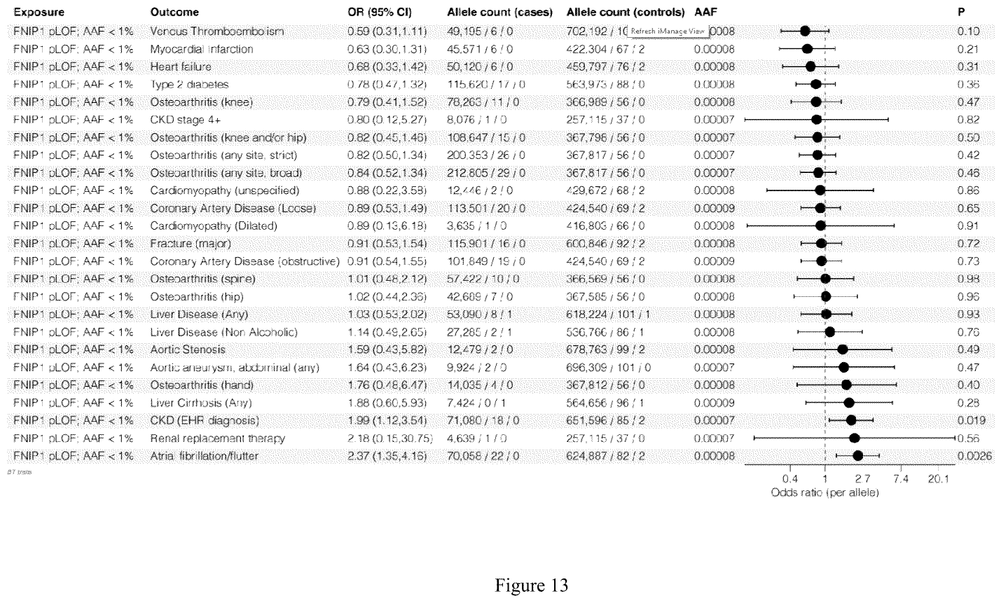

FIG. 13 shows FNIP1 pLoF had general protective cardiometabolic associations.

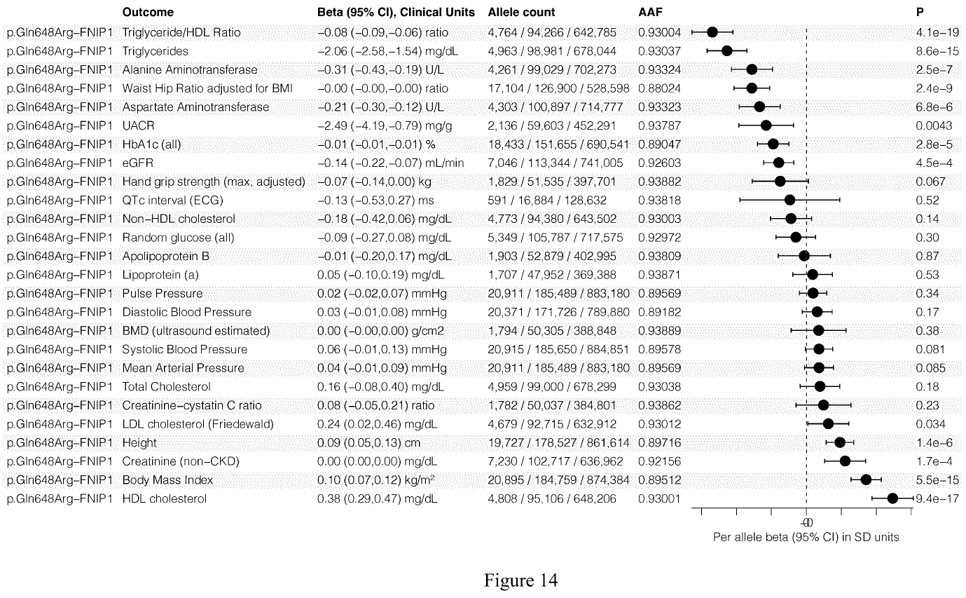

FIG. 14 shows FNIP1 common missense variant phenome-wide associations are similar to FNIP1 rare coding associations.

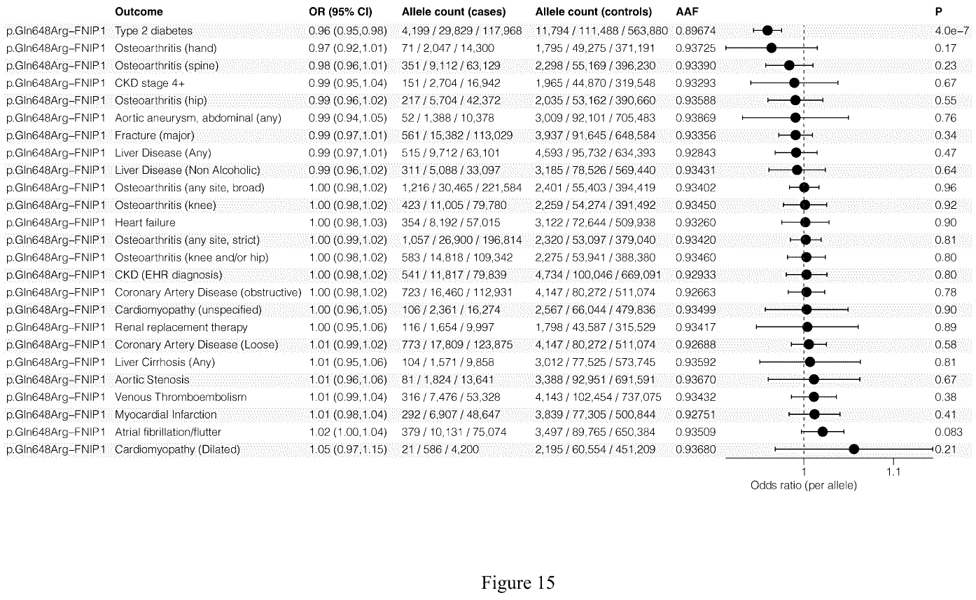

FIG. 15 shows FNIP1 common missense variant shows strongest association with lower odds for T2D.

### Example 3: FNIP1 or FLCN Knockdown Effects in Skeletal Muscles

In this example, intramuscular delivery of FNIP1 or FLCN gRNAs was produced through AAV-myo2A vector by pooling multiple gRNAs at the same time to achieve efficient gene perturbation, followed by observation of their knockdown effect on muscle fiber switches, and various mitochondrial gene expression patterns.

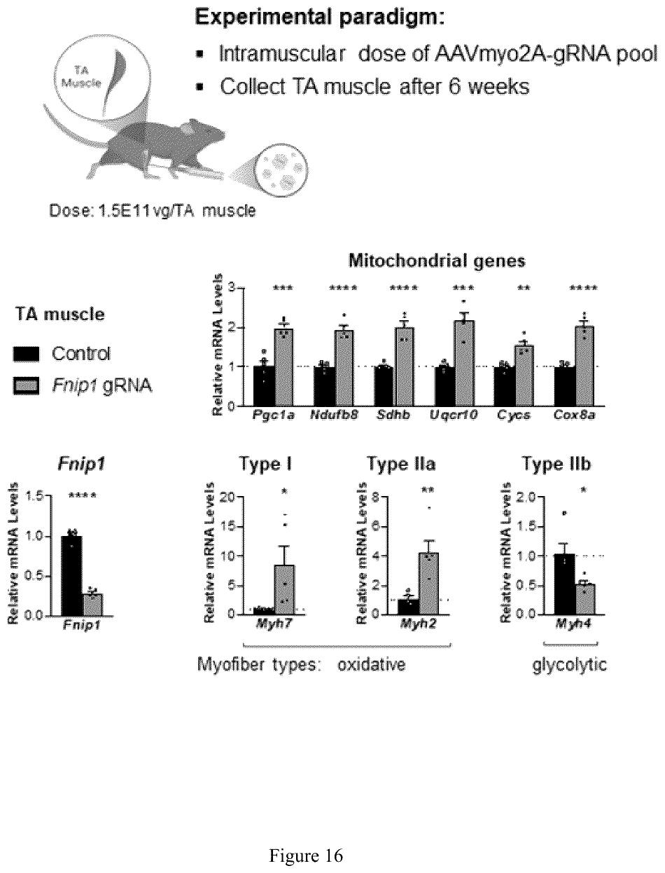

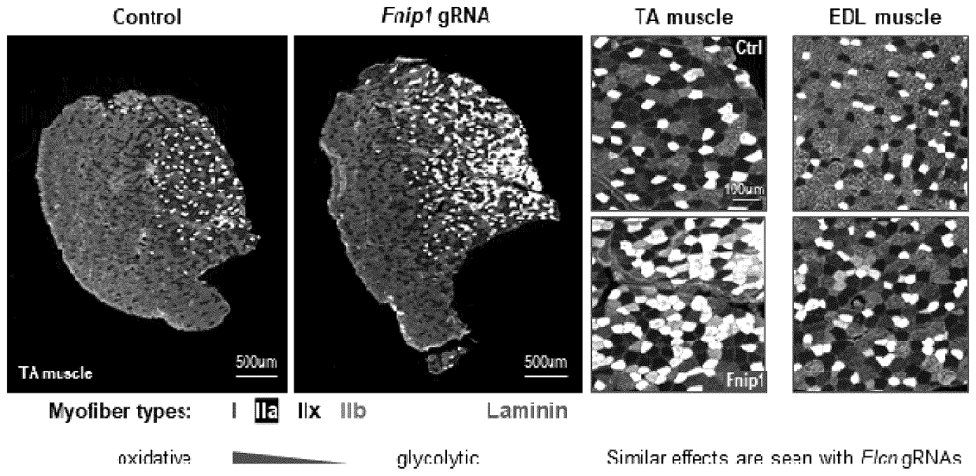

FIG. 16 shows FNIP1 inhibition in skeletal muscle induced an increase in oxidative muscle fibers and mitochondrial gene expression. Pools of FNIP1gRNAs were co-packaged into AAVmyo2A, which was obtained from SignaGen Laboratories (Catalog #SL100862). The AAV-gRNA pools were then delivered to Cas9 male mice via intramuscular injection at a dose of 1.5E11 vg per tibialis anterior (TA) muscle, which leads to efficient gene perturbations in TA and extensor digitorum longus (EDL) muscles. At 6 weeks after AAV administration, the following phenotype was observed: a conversion of glycolytic muscle fibers to oxidative muscle fibers with increased expression of mitochondrial genes in TA and EDL muscle. The data are consistent with literature on muscle-specific FNIP1 cKO mice.

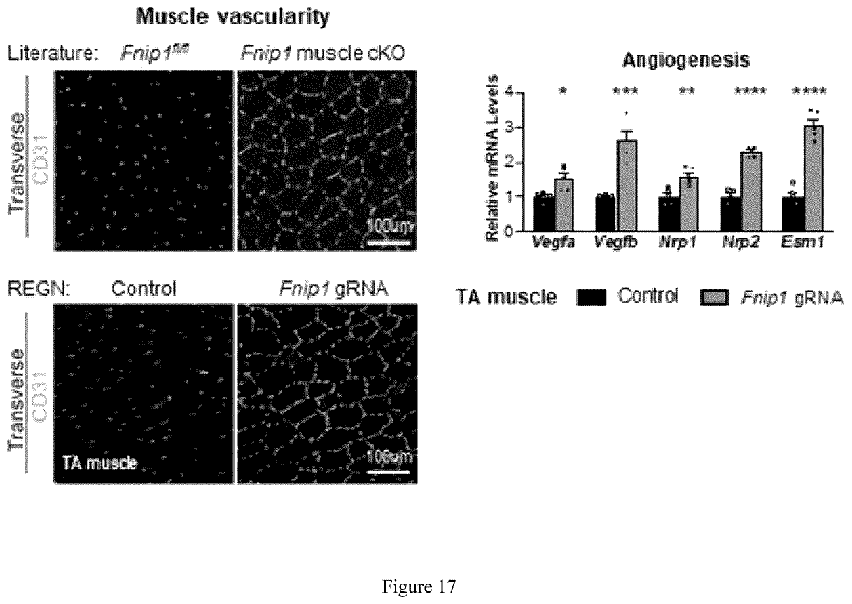

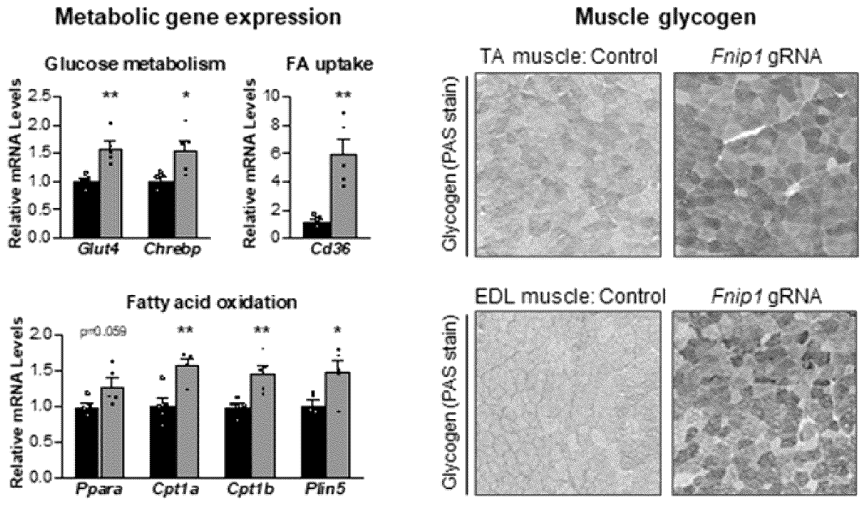

FIG. 17 shows FNIP1 inhibition in skeletal muscle enhanced vascularization and muscle glycogen content, and increased the expression of genes related to glucose and fatty acid uptake and metabolism. These results is suggest: a) FNIP1 gRNAs increase muscle vascularity and enhance nutrient uptake in muscle, consistent with the oxidative myofiber phenotype; and b) skeletal muscle-specific targeting of FNIP1 in adult individuals induces exercise-mimetic effects, which may in part explain the beneficial human genetics associations for FNIP1 pLOF variants: lower HbA1c and T2D risk, lower plasma lipids, and reduced adiposity.

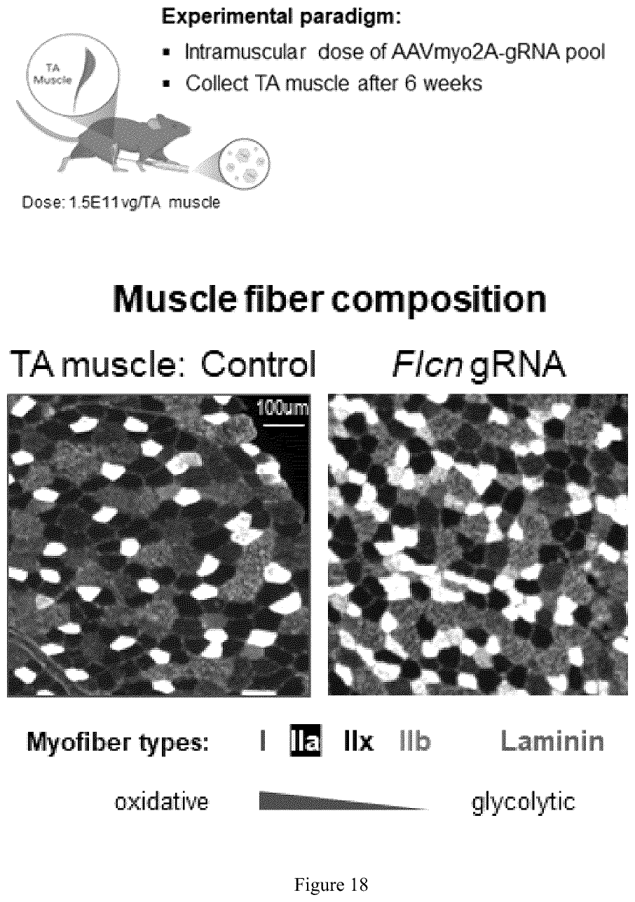

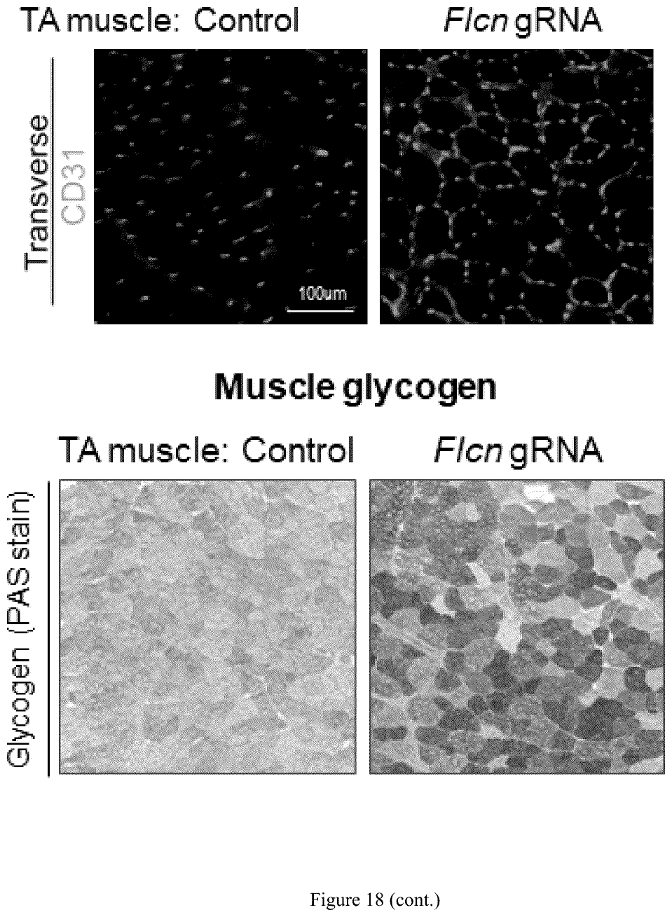

FIG. 18 shows FLCN inhibition in skeletal muscle induced an increase in oxidative muscle fibers, enhanced vascularity, and increased glycogen content. Pools of FLCN gRNAs were co-packaged into AAVmyo2A, which was obtained from SignaGen Laboratories (Catalog #SL100862). The AAV-gRNA pools were then delivered to Cas9 male mice via intramuscular injection at a dose of 1.5E11 vg per tibialis anterior (TA) muscle. At 6 weeks after AAV administration, the following phenotype was observed: a conversion of glycolytic muscle fibers to oxidative muscle fibers with increased muscle capillary density and glycogen storage in TA muscle. The data are consistent with literature on muscle-specific FLCN cKO mice, and may in part explain the beneficial human genetics associations for FLCN pLOF variants: lower HbA1c and T2D risk, lower plasma lipids, and reduced adiposity.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having muscle disorder or at risk of developing muscle disorder, the method comprising administering a Folliculin Interacting Protein 1 (FNIP1) inhibitor and/or a Folliculin (FLCN) inhibitor to the subject.

2. The method of claim 1, wherein the muscle disorder comprises sarcopenia, Duchenne Muscular Dystrophy, or Pompe disease.

3. The method of claim 1, wherein the FNIP1 inhibitor comprises an inhibitory nucleic acid molecule that hybridizes to an FNIP1 nucleic acid molecule.

4. The method of claim 3, wherein the inhibitory nucleic acid molecule comprises an antisense nucleic acid molecule, a small interfering RNA (siRNA), and/or a short hairpin RNA (shRNA).

5-6. (canceled)

7. The method of any one of claim 1, wherein the FLCN inhibitor comprises an inhibitory nucleic acid molecule that hybridizes to an FLCN nucleic acid molecule.

8. The method of claim 7, wherein the inhibitory nucleic acid molecule comprises an antisense nucleic acid molecule, a small interfering RNA (siRNA), and/or a short hairpin RNA (shRNA).

9-10. (canceled)

11. The method of claim 1, the method further comprising administering a muscle disorder therapeutic agent.

12. The method of claim 1, further comprising detecting the presence or absence of an FNIP1 variant nucleic acid molecule and/or an FLCN variant nucleic acid molecule in a biological sample from the subject.

13-14. (canceled)

15. The method of claim 12, wherein the FNIP1 variant nucleic acid molecule and/or an FLCN variant nucleic acid molecule comprises a splice-site variant, a stop-gain variant, a start-loss variant, a stop-loss variant, a frameshift variant, a missense variant, an in-frame indel variant, and/or a variant that encodes a truncated FNIP1 variant polypeptide or a truncated FLCN variant polypeptide.

16-17. (canceled)

18. A method of treating a subject having muscle disorder or at risk of developing muscle disorder by administering a muscle disorder therapeutic agent, the method comprising:
determining or having determined whether the subject has a Folliculin Interacting Protein 1 (FNIP1) variant nucleic acid molecule and/or a Folliculin (FLCN) variant nucleic acid molecule, by:
obtaining or having obtained a biological sample from the subject; and
performing or having performed a sequence analysis on the biological sample to determine if the subject has a genotype comprising an FNIP1 variant nucleic acid molecule and/or an FLCN variant nucleic acid molecule; and
administering or continuing to administer the muscle disorder therapeutic agent in an amount that is the same as or less than a standard dosage amount, and/or an FNIP1 inhibitor and/or an FLCN inhibitor to a subject that is FNIP1 reference and/or FLCN reference;
administering or continuing to administer the muscle disorder therapeutic agent in an amount that is the same as or less than a standard dosage amount, and/or an FNIP1 inhibitor and/or an FLCN inhibitor to a subject that is heterozygous for the FNIP1 variant nucleic acid molecule and/or an FLCN variant nucleic acid molecule; or
administering or continuing to administer the muscle disorder therapeutic agent in a standard dosage amount to a subject that is homozygous for the FNIP1 variant nucleic acid molecule and/or an FLCN variant nucleic acid molecule;
wherein the presence of the FNIP1 variant nucleic acid molecule and/or an FLCN variant nucleic acid molecule indicates the subject has a decreased risk of developing muscle disorder.

19. The method of claim 18, wherein the muscle disorder comprises sarcopenia, Duchenne Muscular Dystrophy, or Pompe disease.

20. The method of claim 18, wherein the FNIP1 inhibitor comprises an inhibitory nucleic acid molecule that hybridizes to an FNIP1 nucleic acid molecule.

21. The method of claim 20, wherein the inhibitory nucleic acid molecule comprises an antisense nucleic acid molecule, a small interfering RNA (siRNA), and/or a short hairpin RNA (shRNA).

22-23. (canceled)

24. The method of claim 18, wherein the FLCN inhibitor comprises an inhibitory nucleic acid molecule that hybridizes to an FLCN nucleic acid molecule.

25. The method of claim 24, wherein the inhibitory nucleic acid molecule comprises an antisense nucleic acid molecule, a small interfering RNA (siRNA), and/or a short hairpin RNA (shRNA).

26-27. (canceled)

28. The method of claim 18, wherein the subject is heterozygous for the FNIP1 variant nucleic acid molecule and/or an FLCN variant nucleic acid molecule, and the subject is administered or continued to be administered the muscle disorder therapeutic agent in an amount that is the same as or less than a standard dosage amount and the FNIP1 inhibitor and/or FLCN inhibitor.

29. The method of claim 18, wherein the subject is FNIP1 reference and/or FLCN reference, and the subject is administered or continued to be administered the muscle disorder therapeutic agent in an amount that is the same as or less than a standard dosage amount and the FNIP1 inhibitor and/or FLCN inhibitor.

30. The method of claim 18, wherein the FNIP1 variant nucleic acid molecule and/or an FLCN variant nucleic acid molecule comprises a splice-site variant, a stop-gain variant, a start-loss variant, a stop-loss variant, a frameshift variant, a missense variant, an in-frame indel variant, and/or a variant that encodes a truncated FNIP1 variant polypeptide or a truncated FLCN variant polypeptide.

31-32. (canceled)

33. A method of identifying a subject having an increased risk of developing muscle disorder, the method comprising:
determining or having determined the presence or absence of a Folliculin Interacting Protein 1 (FNIP1) variant nucleic acid molecule and/or a Folliculin (FLCN) variant nucleic acid molecule in a biological sample obtained from the subject;
wherein:
when the subject is FNIP1 reference and/or FLCN reference, then the subject has an increased risk of developing the muscle disorder; and
when the subject is heterozygous or homozygous for the FNIP1 variant nucleic acid molecule and/or an FLCN variant nucleic acid molecule, then the subject has a decreased risk of developing the muscle disorder.

34-67. (canceled)


SEQUENCE LISTING (120 sequences)

SEQ ID NO: 1 - protein, 119 aa, Homo sapiens
QVQLVESGGGVVQPGRSLRLSCTASGITFRNYGMHWVRQAPGKGLEWVAVMWYDGSNKYYADSVKGRFTISGDNSKVYLQMNSLRAEDTAVYYCARRGTIRTAAPFDYWGQGTLVTVSS

SEQ ID NO: 2 - protein, 119 aa, Homo sapiens
QVQLQQWGAGLLKPSATLSRTCAVYGGSFSGYYWNWIRQSPGKGLEWIGEILHSGRTNYNPSLKSRVTISVDTSKNQFSLKLTSVTAADTAVYYCAGRIAARHGWFDPWGQGTLVTVSS

SEQ ID NO: 3 - protein, 119 aa, Homo sapiens
QVQLVESGGGVVQPGRSLRLSCAASGFTFSTYGMHWVRQAPGKGLEWVAVIWHDGSDKYYVDSVKGRFSIARDNSKNTLYLQMNSLRVEDTGIYYCARRGIRGTVFDHWGLGTLVTVSS

SEQ ID NO: 4 - protein, 116 aa, Homo sapiens
QVQLQESGPGLVKPSETLSLTCTVSGDSINNYYWTWLRQPPGKGLEWIGYIYYSGSANYNPSLKSRVTISVDTSKNQFSLKLNSVTAADTAVYYCARGAVKYFRHWGQGTLVTVSS

SEQ ID NO: 5 - protein, 121 aa, Homo sapiens
QVQLVESGGGVVQPGTSLRLSCAASGFTFSSYGMHWVRQAPGKGLEWVAVIWIDGSNKYYADSVKGRFTISRDNSKNTLYLQMNSLRAEDTAVYYCARRGGIVVAAPFDYWGQGTLVTVSS

SEQ ID NO: 6 - protein, 121 aa, Homo sapiens
QVQLVESGGGVVQPGRSLRLSCEASGITFRNYGMHWVRQAPGKGLEWVAVMWYDGSNKYYADSVKGRFTISRDNSKNTVYLQMNSLRAEDTAVYYCARRGHIATAAPFDYWGQGTLVTVSS

SEQ ID NO: 7 - protein, 121 aa, Homo sapiens
QVQLVESGGGVVQPGRSLRLSCTASGFTFRSYGMHWVRQAPGKGLEWVSVIWIDGNNIYYADSVKGRFTISRDNSKNTLYLQMDSLRAEDTAVYYCARRLAITSAAPFDYWGQGTLVTVSS

SEQ ID NO: 8 - protein, 126 aa, Homo sapiens
QVQLVQSGAEVKKPGASVKVSCKASGYAFTTYGITWVRQAPGQGLEWMGWISAYNGNTNYAEKVQGRFTMTTDTSTNTAYMELRSLRSDDTAVYFCARKGHYGSGTYYNPFGFDFWGQGTMVTVSS

SEQ ID NO: 9 - protein, 120 aa, Homo sapiens
EVQLVESGGGLVQPGGSLKLSCTASGLTLSDSAMHWVRQASGKGLEWVGRIRNKANRYATEYAASVKGRFTISRDDSKNTAYLQMNSLKTEDTAVYYCTRNWKIFLFDYWGQGTLVTVSS

SEQ ID NO: 10 - protein, 125 aa, Homo sapiens
EVQLLESGGGLVQPGGSLRLSCAASGFTFNNYGMSWVRQGPGKGLEWVSSISGSGGTTFYADSVKGRFTISRDNSKNTLYLQMNSLRAEDTAVYYCGKGGYCSSSGCRHYGMDVWGQGTTVTVSS

SEQ ID NO: 11 - protein, 121 aa, Homo sapiens
EVQLVESGGNLVQPGGSLRLSCAASGFTFTSHAMNWVRQAPGKGLEWVSVITGRGFDTHYADSVKGRFTISRDISKNTLYLQMNSLRAEDTAVYYCAKGLYDSGNYYIDYWGQGTLVTVSS

SEQ ID NO: 12 - protein, 126 aa, Homo sapiens; identical to SEQ ID NO: 8

SEQ ID NO: 13 - protein, 8 aa, Homo sapiens
GITFRNYG

SEQ ID NO: 14 - protein, 8 aa, Homo sapiens
GGSFSGYY

SEQ ID NO: 15 - protein, 8 aa, Homo sapiens
GFTFSTYG

SEQ ID NO: 16 - protein, 8 aa, Homo sapiens
GDSINNYY

SEQ ID NO: 17 - protein, 8 aa, Homo sapiens
GFTFSSYG

SEQ ID NO: 18 - protein, 8 aa, Homo sapiens
GITFRNYG

SEQ ID NO: 19 - protein, 8 aa, Homo sapiens
GFTFRSYG

SEQ ID NO: 20 - protein, 8 aa, Homo sapiens
GYAFTTYG

SEQ ID NO: 21 - protein, 8 aa, Homo sapiens
GLTLSDSA

SEQ ID NO: 22 - protein, 8 aa, Homo sapiens
GFTFNNYG

SEQ ID NO: 23 - protein, 8 aa, Homo sapiens
GFTFTSHA

SEQ ID NO: 24 - protein, 8 aa, Homo sapiens
GYAFTTYG

SEQ ID NO: 25 - protein, 8 aa, Homo sapiens
MWYDGSNK

SEQ ID NO: 26 - protein, 7 aa, Homo sapiens
ILHSGRT

SEQ ID NO: 27 - protein, 8 aa, Homo sapiens
IWHDGSDK

SEQ ID NO: 28 - protein, 7 aa, Homo sapiens
IYYSGSA

SEQ ID NO: 29 - protein, 8 aa, Homo sapiens
IWIDGSNK

SEQ ID NO: 30 - protein, 7 aa, Homo sapiens
MWYDGSN

SEQ ID NO: 31 - protein, 8 aa, Homo sapiens
IWIDGNNI

SEQ ID NO: 32 - protein, 7 aa, Homo sapiens
ISAYNGN

SEQ ID NO: 33 - protein, 10 aa, Homo sapiens
IRNKANRYAT

SEQ ID NO: 34 - protein, 6 aa, Homo sapiens
SGSGGT

SEQ ID NO: 35 - protein, 8 aa, Homo sapiens
ITGRGFDT

SEQ ID NO: 36 - protein, 8 aa, Homo sapiens
ISAYNGNT

SEQ ID NO: 37 - protein, 14 aa, Homo sapiens
ARRGTIRTAAPFDY

SEQ ID NO: 38 - protein, 13 aa, Homo sapiens
AGRIAARHGWFDP

SEQ ID NO: 39 - protein, 12 aa, Homo sapiens
ARRGIRGTVFDH

SEQ ID NO: 40 - protein, 10 aa, Homo sapiens
ARGAVKYFRH

SEQ ID NO: 41 - protein, 14 aa, Homo sapiens
ARRGGIVVAAPFDY

SEQ ID NO: 42 - protein, 13 aa, Homo sapiens
ARRGHIATAAPFD

SEQ ID NO: 43 - protein, 14 aa, Homo sapiens
ARRLAITSAAPFDY

SEQ ID NO: 44 - protein, 19 aa, Homo sapiens
CARKGHYGSGTYYNPFGFD

SEQ ID NO: 45 - protein, 11 aa, Homo sapiens
TRNWKIFLFDY

SEQ ID NO: 46 - protein, 14 aa, Homo sapiens
CGKGGYCSSSGCRH

SEQ ID NO: 47 - protein, 14 aa, Homo sapiens
AKGLYDSGNYYIDY

SEQ ID NO: 48 - protein, 19 aa, Homo sapiens
ARKGHYGSGTYYNPFGFDF

SEQ ID NO: 49 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 50 - protein, 107 aa, Homo sapiens
DIQMTQSPSSVSTSVGDRVTISCRASQDIRKWLAWYQQKPGKAPKLLIYATSSLQSGVPSRFSGSGSGTDFTLTISSLQPEDFATYFCQQANSFPFTFGQGTKLEIK

SEQ ID NO: 51 - protein, 107 aa, Homo sapiens
DIQMTQSPSTLSASVGDRVTLTCRASQSISNKLAWYQQKPGKAPNLLIYKASNLESGVPSRFSGSGSGTEFTLTISSLQPDDFATYYCQQYNSYSWTFGQGTKVEIK

SEQ ID NO: 52 - protein, 108 aa, Homo sapiens
EIVLTQSPGTLSLSPGERATLSCRASQTINHNNLAWYQQRPGQAPRLLIYGASNRATAIPDRFSGSGSGTDFTLTISRLEPEDFEVYSCQQYGSLPLTFGGGTKVEIK

SEQ ID NO: 53 - protein, 108 aa, Homo sapiens; identical to SEQ ID NO: 49

SEQ ID NO: 54 - protein, 108 aa, Homo sapiens
DIQMTQSPSSLSASVGDRVTISCRASQSISSYLNWYQQKPGKAPKVLMYAASSLQSGVPSRFSGSGSGTDFTLTISSLQPEDFATYYCQQSYSTPPITFGQGTRLEIK

SEQ ID NO: 55 - protein, 108 aa, Homo sapiens; identical to SEQ ID NO: 49

SEQ ID NO: 56 - protein, 108 aa, Homo sapiens
EIMLMQSPGTLSLSPGERATLSCRASQSVSSSYLAWYQQKPGQAPRLLIYGASSRATDIPDRFSGSGSGTDFTLTISRLEPEDFAVYFCQQYYGSPWTFGQGTKVEIK

SEQ ID NO: 57 - protein, 108 aa, Homo sapiens
EIVLTQSPGTLTLSPGERATLSCRASQSVGSKYLAWFQQKRGQAPRLLIYGASSRTSGIPDRISGSGSGTDFTLTISRLEPEDFAVYYCQQYGSSPWTFGQGTKVEIK

SEQ ID NO: 58 - protein, 110 aa, Homo sapiens
QSVLTQPPSASGTPGQRVTISCSGSSSNIGNNYIYWYQRLPGTTPKLLIYRNNQRPSGVPDRFSGSKSGTSASLAISGLRSEDEADYYCAAWDDTLSGYVFGTGTKVTVL

SEQ ID NO: 59 - protein, 111 aa, Homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDLSLSFNWVFGGGTKLTVL

SEQ ID NO: 60 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFALYFCQQYYGSPWTFGQGTKVEIK

SEQ ID NO: 61 - protein, 6 aa, Homo sapiens
QSISSY

SEQ ID NO: 62 - protein, 6 aa, Homo sapiens
QDIRKW

SEQ ID NO: 63 - protein, 6 aa, Homo sapiens
QSISNK

SEQ ID NO: 64 - protein, 7 aa, Homo sapiens
QTINHNN

SEQ ID NO: 65 - protein, 6 aa, Homo sapiens
QSISSY

SEQ ID NO: 66 - protein, 6 aa, Homo sapiens
QSISSY

SEQ ID NO: 67 - protein, 6 aa, Homo sapiens
QSISSY

SEQ ID NO: 68 - protein, 7 aa, Homo sapiens
QSVSSSY

SEQ ID NO: 69 - protein, 7 aa, Homo sapiens
QSVGSKY

SEQ ID NO: 70 - protein, 8 aa, Homo sapiens
SSNIGNNY

SEQ ID NO: 71 - protein, 8 aa, Homo sapiens
SSNIGNNY

SEQ ID NO: 72 - protein, 7 aa, Homo sapiens
QSVSSSY

SEQ ID NO: 73-84 - skipped (12 entries)

SEQ ID NO: 85 - protein, 10 aa, Homo sapiens
QQSYSTPPIT

SEQ ID NO: 86 - protein, 9 aa, Homo sapiens
QQANSFPFT

SEQ ID NO: 87 - protein, 9 aa, Homo sapiens
QQYNSYSWT

SEQ ID NO: 88 - protein, 9 aa, Homo sapiens
QQYGSLPLT

SEQ ID NO: 89 - protein, 10 aa, Homo sapiens
QQSYSTPPIT

SEQ ID NO: 90 - protein, 9 aa, Homo sapiens
QSYSTPPIT

SEQ ID NO: 91 - protein, 9 aa, Homo sapiens
QSYSTPPIT

SEQ ID NO: 92 - protein, 9 aa, Homo sapiens
CQQYYGSPW

SEQ ID NO: 93 - protein, 9 aa, Homo sapiens
QQYGSSPWT

SEQ ID NO: 94 - protein, 11 aa, Homo sapiens
CAAWDDTLSGY

SEQ ID NO: 95 - protein, 12 aa, Homo sapiens
GTWDLSLSFNWV

SEQ ID NO: 96 - protein, 9 aa, Homo sapiens
QQYYGSPWT

SEQ ID NO: 97 - protein, 445 aa, Homo sapiens
QVQLVESGGGVVQPGRSLRLSCTASGITFRNYGMHWVRQAPGKGLEWVAVMWYDGSNKYYADSVKGRFTISGDNSKVYLQMNSLRAEDTAVYYCARRGTIRTAAPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 98 - protein, 446 aa, Homo sapiens
QVQLQQWGAGLLKPSATLSRTCAVYGGSFSGYYWNWIRQSPGKGLEWIGEILHSGRTNYNPSLKSRVTISVDTSKNQFSLKLTSVTAADTAVYYCAGRIAARHGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 446 aa, Homo sapiens
QVQLVESGGGVVQPGRSLRLSCAASGFTFSTYGMHWVRQAPGKGLEWVAVIWHDGSDKYYVDSVKGRFSIARDNSKNTLYLQMNSLRVEDTGIYYCARRGIRGTVFDHWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - protein, 443 aa, Homo sapiens
QVQLQESGPGLVKPSETLSLTCTVSGDSINNYYWTWLRQPPGKGLEWIGYIYYSGSANYNPSLKSRVTISVDTSKNQFSLKLNSVTAADTAVYYCARGAVKYFRHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 101 - protein, 448 aa, Homo sapiens
QVQLVESGGGVVQPGTSLRLSCAASGFTFSSYGMHWVRQAPGKGLEWVAVIWIDGSNKYYADSVKGRFTISRDNSKNTLYLQMNSLRAEDTAVYYCARRGGIVVAAPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - protein, 448 aa, Homo sapiens
QVQLVESGGGVVQPGRSLRLSCTASGFTFRSYGMHWVRQAPGKGLEWVSVIWIDGNNIYYADSVKGRFTISRDNSKNTLYLQMDSLRAEDTAVYYCARRLAITSAAPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 103 - protein, 448 aa, Homo sapiens
EVQLVESGGNLVQPGGSLRLSCAASGFTFTSHAMNWVRQAPGKGLEWVSVITGRGFDTHYADSVKGRFTISRDISKNTLYLQMNSLRAEDTAVYYCAKGLYDSGNYYIDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 104 - protein, 451 aa, Homo sapiens
QVQLVESGGGVVQPGRSLRLSCEASGITFRNYGMHWVRQAPGKGLEWVAVMWYDGSNKYYADSVKGRFTISRDNSKNTVYLQMNSLRAEDTAVYYCARRGHIATAAPFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 105 - protein, 450 aa, Homo sapiens
QVQLVQSGAEVKKPGASVKVSCKASGYAFTTYGITWVRQAPGQGLEWMGWISAYNGNTNYAEKVQGRFTMTTDTSTNTAYMELRSLRSDDTAVYFCARKGHYGSGTYYNPFGFDFWGQGTMVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 106 - protein, 447 aa, Homo sapiens
EVQLVESGGGLVQPGGSLKLSCTASGLTLSDSAMHWVRQASGKGLEWVGRIRNKANRYATEYAASVKGRFTISRDDSKNTAYLQMNSLKTEDTAVYYCTRNWKIFLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 107 - protein, 455 aa, Homo sapiens
EVQLLESGGGLVQPGGSLRLSCAASGFTFNNYGMSWVRQGPGKGLEWVSSISGSGGTTFYADSVKGRFTISRDNSKNTLYLQMNSLRAEDTAVYYCGKGGYCSSSGCRHYGMDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 108 - protein, 453 aa, Homo sapiens
QVQLVQSGAEVKKPGASVKVSCKASGYAFTTYGITWVRQAPGQGLEWMGWISAYNGNTNYAEKVQGRFTMTTDTSTNTAYMELRSLRSDDTAVYFCARKGHYGSGTYYNPFGFDFWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 109 - protein, 215 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 214 aa, Homo sapiens
DIQMTQSPSSVSTSVGDRVTISCRASQDIRKWLAWYQQKPGKAPKLLIYATSSLQSGVPSRFSGSGSGTDFTLTISSLQPEDFATYFCQQANSFPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 214 aa, Homo sapiens
DIQMTQSPSTLSASVGDRVTLTCRASQSISNKLAWYQQKPGKAPNLLIYKASNLESGVPSRFSGSGSGTEFTLTISSLQPDDFATYYCQQYNSYSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 215 aa, Homo sapiens
EIVLTQSPGTLSLSPGERATLSCRASQTINHNNLAWYQQRPGQAPRLLIYGASNRATAIPDRFSGSGSGTDFTLTISRLEPEDFEVYSCQQYGSLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 215 aa, Homo sapiens; identical to SEQ ID NO: 109

SEQ ID NO: 114 - protein, 215 aa, Homo sapiens
DIQMTQSPSSLSASVGDRVTISCRASQSISSYLNWYQQKPGKAPKVLMYAASSLQSGVPSRFSGSGSGTDFTLTISSLQPEDFATYYCQQSYSTPPITFGQGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 115 - protein, 215 aa, Homo sapiens; identical to SEQ ID NO: 109

SEQ ID NO: 116 - protein, 215 aa, Homo sapiens
EIMLMQSPGTLSLSPGERATLSCRASQSVSSSYLAWYQQKPGQAPRLLIYGASSRATDIPDRFSGSGSGTDFTLTISRLEPEDFAVYFCQQYYGSPWTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 117 - protein, 215 aa, Homo sapiens
EIVLTQSPGTLTLSPGERATLSCRASQSVGSKYLAWFQQKRGQAPRLLIYGASSRTSGIPDRISGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 217 aa, Homo sapiens
QSVLTQPPSASGTPGQRVTISCSGSSSNIGNNYIYWYQRLPGTTPKLLIYRNNQRPSGVPDRFSGSKSGTSASLAISGLRSEDEADYYCAAWDDTLSGYVFGTGTKVTVLRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 119 - protein, 217 aa, Homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDLSLSFNWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 120 - protein, 215 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFALYFCQQYYGSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC